(19) United States
(12) Patent Application Publication
(10) Pub. No.: US 2009/0246774 A1
(43) Pub. Date: Oct. 1, 2009

(54) Chromosomal Blocks as Markers for Traits

(76) Inventors: Mehar Singh Khatkar, New South Wales (AU); Hermanus Willem Raadsma, New South Wales (AU)

Correspondence Address: FISH & RICHARDSON P.C., PO BOX 1022, MINNEAPOLIS, MN, 55440-1022, US

(21) Appl. No.: 12/295,164

(22) PCT Filed: Mar. 30, 2007
(86) PCT No.: PCT/AU07/00416

§ 371 Date: Dec. 16, 2008

(30) Foreign Application Priority Data
Mar. 30, 2006 (AU) 2006901648

(51) Int. Cl. C12Q 1/68; C07H 21/04

(52) U.S. Cl. 435/6; 536/23.1

(57) ABSTRACT

The present invention provided a method for predicting a phenotype in a bovine animal, the method comprising analysing a nucleic acid sample from said animal for the presence of at least one genetic marker known to reside in an Linkage Disequilibrium (LD) block in any one of bovine chromosomes BTA-I to BTA-29, wherein said LD block is associated with said phenotype. The phenotype can be Australia profit ranking (APR), Australian selection index (ASR), protein yield (PROT), protein percent (PROT %), milk volume (MILK), fat yield (FAT), fat percent (FAT %), breeding value overall type (Overall Type), somatic cell count (SCC), and/or breeding value cow fertility (Cow Fertility). Also provided is a linkage disequilibrium unit (LDU) map of any one or more of bovine chromosomes BTA-I to BTA-29, the map comprising a plurality of chromosomal regions, and the regions defined by their co-inheritance across generations substantially as entire linkage disequilibrium (LD) blocks.

### TECHNICAL FIELD

The present invention relates to linkage disequilibrium unit maps and methods for predicting phenotypes as traits in domestic animals. In particular, the present invention relates to predicting phenotypes based upon the association of chromosomal linkage disequilibrium blocks with traits.

### BACKGROUND

Population-wide association studies using a high density of genetic markers provide a powerful means for identifying common genetic variants that underlie complex traits. To be useful, markers tested for associations must be either the causal allele, the so-called quantitative trait nucleotide (QTN) or quantitative trait locus (QTL), or highly correlated (in linkage disequilibrium) with the QTN/QTL.

Linkage disequilibrium (LD) describes a situation in which some combinations of alleles of two or more different loci (haplotypes) occur more or less frequently within a population than would be expected by random chance alone. Information on the structure of LD and marker-marker association at the population level is, therefore, crucial to understand the circumstances under which a genome-wide association approach might be interpreted and applicable. Furthermore, such information is critical for the design of panels of optimally-spaced markers for high-powered, comprehensive genome-wide association studies which are capable of simultaneously minimizing cost and genotyping effort.

The extent and pattern of LD has been vigorously studied in humans. The LD structure in humans has been found to be quite complex, with significant variation between populations and genomes. This has led to many problems including spurious results when using LD methods in genome mapping studies. A major problem is the large variance in LD between markers across the genome irrespective of physical distance. This situation has the effect of producing false or misleading haplotype boundaries because LD varies amongst markers as a result of marker age (mutation) and population history. When using these haplotypes to map and track QTNs/QTLs, spurious results may emerge due to the lack of information of true haplotype boundaries and LD structure.

Traditional haplotype block methodologies utilizing literally millions of single nucleotide polymorphism (SNP) markers have been successfully applied in relation to the human genome, resulting in extensive haplotype block coverage of over 80% of the genome. However, in other species where less SNP marker information is available, traditional haplotype block methodologies are correspondingly limited, resulting in poor coverage of the genome by haplotype blocks.

A solution to these problems is to define regions of the genome which correspond to both LD structure and a physical location (that is, “blocks” of LD in the genome). To help understand this concept, LD unit (LDU) maps have been developed. Such maps facilitate association mapping, extend the resolution of the linkage map, allow comparison across populations, and are useful to detect selective sweeps and other events of evolutionary interest. One LD unit corresponds to one ‘swept radius’. This average distance in kilobases (Kb) over LD is useful for gene mapping in a particular genomic region. The distance varies substantially across the chromosome with some regions having very extensive LD (where one LD unit spans a large physical distance) and other regions where LD breaks down quickly (where there is a high LDU/Kb ratio). LDU map distances are therefore analogous to the centimorgan scale of linkage maps. At marker densities which are high enough to fully delimit the LD structure, LDU map distances are additive, being another property shared with the linkage map.

When cumulative LD unit distances are plotted against the physical map, a pattern of plateaus (reflecting regions of high LD or “LD blocks”) and steps (which represent regions of low LD) emerge. The intensity of recombination is related to the height (increase in LDU) of the step. However, the close correspondence between LD structure and recombination can be distorted to some extent by other factors such as mutation, drift, and selection, which operate over the many generations during which the pattern of LD is determined. To the extent that these phenomena are important, both the physical and linkage maps are unreliable guides to LD structure. LDU maps also identify ‘holes’ or gaps within which greater marker density is required to fully determine the LD structure and therefore define the optimal spacing of markers for association mapping and positional cloning.

The present invention is based on the development of LDU maps across the entire bovine genome with the aid of a panel of high density SNP markers. These maps partition the genome to account for both physical location of markers and varying LD levels between these markers. Furthermore, the LDU maps provide an understanding of LD structure and recombination patterns, or “chromosomal LD block structures”, which can be used to predict phenotypes based upon the association of such co-inherited chromosomal LD block structures with traits.

LDU mapping methodology can be applied to, but is not limited to, (a) association analysis and MAS practices (ie., using true allelic variants in LD blocks to track traits) and (b) obtaining regions for targeted fine mapping with known chromosome LD block tracking QTL (ie., discrete physical boundaries).

### SUMMARY OF THE INVENTION

According to one aspect, there is provided a method for predicting a phenotype in a bovine animal, the method comprising analysing a nucleic acid sample from said animal for the presence of at least one genetic marker known to reside in an LD block in any one of bovine chromosomes BTA-1 to BTA-29, wherein said LD block is associated with said phenotype, and wherein the phenotype is selected from the group consisting of Australian profit ranking (APR), Australian selection index (ASR), protein yield (PROT), protein percent (PROT %), milk volume (MILK), fat yield (FAT), fat percent (FAT %), breeding value overall type (Overall Type), somatic cell count (SCC), and breeding value cow fertility (Cow Fertility).

According to another aspect, there is provided a method of selecting a bovine animal for a phenotype comprising analysing a nucleic acid sample from said animal for the presence of at least one genetic marker known to reside in an LD block in any one of bovine chromosomes BTA-1 to BTA-29, wherein said LD block is associated with said phenotype, and wherein the phenotype is selected from the group consisting of Australian profit ranking (APR), Australian selection index (ASR), protein yield (PROT), protein percent (PROT %), milk volume (MILK), fat yield (FAT), fat percent (FAT %), breeding value overall type (Overall Type), somatic cell count (SCC), and breeding value cow fertility (Cow Fertility), and selecting the animal based on the presence or absence of the at least one genetic marker.

In one embodiment of the above aspects the phenotype is Australian profit ranking (APR) and the LD block is selected from the group consisting of C1L1.0B_{—}59.93-83.90, C2L1.0B_{—}113.49-125.27, C3L1.0B_{—}86.79-102.66, C4L1.0B_{—}38.59-65.77, C5L1.0B_{—}6.53-12.83, C6L1.0B_{—}59.88-79.54, C7L1.0B_{—}42.20-64.05, C8L1.0B_{—}33.47-59.35, C9L1.0B_{—}52.17-73.73, C10L1.0B_{—}20.04-41.39, C11L1.0B_{—}83.65-93.53, C12L1.0B_{—}11.23-20.94, C13L1.0B_{—}38.61-56.34, C14L1.0B_{—}18.56-37.78, C15L1.0B_{—}34.73-54.95, C16L1.0B_{—}28.33-44.56, C17L1.0B_{—}32.00-45.41, C18L1.0B_{—}13.92-25.33, C19L1.0B_{—}18.67-30.73, C20L1.0B_{—}28.06-43.47, C21L1.0B_{—}11.93-24.10, C22L1.0B_{—}34.48-46.43, C23L1.0B_{—}14.14-27.73, C24L1.0B_{—}35.09-47.57, C25L1.0B_{—}27.70-36.65, C26L1.0B_{—}10.99-30.90, C27L1.0B_{—}24.14-35.66, C28L1.0B_{—}30.30-37.62 and C29L1.0B_{—}23.81-31.74.

In another embodiment of the above aspects the phenotype is Australian Selection Index (ASI) and the LD block is selected from the group consisting of C1L1.0B_{—}59.93-83.90, C2L1.0B_{—}101.03-113.49, C3L1.0B_{—}86.79-102.66, C4L1.0B_{—}38.59-65.77, C5L1.0B_{—}22.90-42.32, C6L1.0B_{—}59.88-79.54, C7L1.0B_{—}42.20-64.05, C8L1.0B_{—}33.47-59.35, C9L1.0B_{—}52.17-73.73, C10L1.0B_{—}20.04-41.39, C11L1.0B_{—}9.99-28.73, C12L1.0B_{—}34.61-53.14, C13L1.0B_{—}38.61-56.34, C14L1.0B_{—}18.56-37.78, C15L1.0B_{—}34.73-54.95, C16L1.0B_{—}28.33-44.56, C17L1.0B_{—}32.00-45.41, C18L1.0B_{—}38.17-52.45, C19L1.0B_{—}18.67-30.73, C20L1.0B_{—}14.58-28.06, C21L1.0B_{—}11.93-24.10, C22L1.0B_{—}34.48-46.43, C23L1.0B_{—}14.14-27.73, C24L1.0B_{—}35.09-47.57, C25L1.0B_{—}27.70-36.65, C26L1.0B_{—}10.99-30.90, C27L1.0B_{—}24.14-35.66, C28L1.0B_{—}30.30-37.62 and C29L1.0B_{—}23.81-31.74.

In another embodiment of the above aspects the phenotype is protein yield (PROT) and the LD block is selected from the group consisting of C1L1.0B_{—}59.93-83.90, C2L1.0B_{—}101.03-113.49, C3L1.0B_{—}86.79-102.66, C4L1.0B_{—}38.59-65.77, C5L1.0B_{—}22.90-42.32, C6L1.0B_{—}59.88-79.54, C7L1.0B_{—}42.20-64.05, C8L1.0B_{—}33.47-59.35, C9L1.0B_{—}52.17-73.73, C10L1.0B_{—}20.04-41.39, C11L1.0B_{—}9.99-28.73, C12L1.0B_{—}34.61-53.14, C13L1.0B_{—}14.82-27.97, C14L1.0B_{—}18.56-37.78, C15L1.0B_{—}34.73-54.95, C16L1.0B_{—}28.33-44.56, C17L1.0B_{—}32.00-45.41, C18L1.0B_{—}38.17-52.45, C19L1.0B_{—}18.67-30.73, C20L1.0B_{—}14.58-28.06, C21L1.0B_{—}11.93-24.10, C22L1.0B_{—}20.60-34.48, C23L1.0B_{—}14.14-27.73, C24L1.0B_{—}35.09-47.57, C25L1.0B_{—}27.70-36.65, C26L1.0B_{—}10.99-30.90, C27L1.0B_{—}24.14-35.66, C28L1.0B_{—}30.30-37.62 and C29L1.0B_{—}23.81-31.74.

In another embodiment of the above aspects the phenotype is protein percent (PROT %) and the LD block is selected from the group consisting of C1L1.0B_{—}59.93-83.90, C2L1.0B_{—}101.03-113.49, C3L1.0B_{—}10.54-22.10, C4L1.0B_{—}65.77-81.49, C5L1.0B_{—}69.19-87.51, C6L1.0B_{—}79.54-93.65, C7L1.0B_{—}42.20-64.05, C8L1.0B_{—}33.47-59.35, C9L1.0B_{—}52.17-73.73, C10L1.0B_{—}20.04-41.39, C11L1.0B_{—}64.26-83.65, C12L1.0B_{—}11.23-20.94, C13L1.0B_{—}64.29-72.65, C14L1.0B_{—}54.46-69.52, C15L1.0B_{—}17.86-34.73, C16L1.0B_{—}14.51-28.33, C17L1.0B_{—}55.81-62.12, C18L1.0B_{—}0.65-13.92, C19L1.0B_{—}18.67-30.73, C20L1.0B_{—}28.06-43.47, C21L1.0B_{—}11.93-24.10, C22L1.0B_{—}34.48-46.43, C23L1.0B_{—}37.77-48.55, C24L1.0B_{—}47.57-55.04, C25L1.0B_{—}27.70-36.65, C26L1.0B_{—}30.90-43.28, C27L1.0B_{—}13.12-24.14, C28L1.0B_{—}30.30-37.62 and C29L1.0B_{—}23.81-31.74.

In another embodiment the phenotype is milk volume (MILK) and the LD block is selected from the group consisting of C1L1.0B_{—}59.93-83.90, C2L1.0B_{—}16.97-37.73, C3L1.0B_{—}36.27-52.80, C4L1.0B_{—}38.59-65.77, C5L1.0B_{—}22.90-42.32, C6L1.0B_{—}59.88-79.54, C7L1.0B_{—}64.05-77.97, C8L1.0B_{—}33.47-59.35, C9L1.0B_{—}29.65-52.17, C10L1.0B_{—}20.04-41.39, C11L1.0B_{—}83.65-93.53, C12L1.0B_{—}61.59-74.02, C13L1.0B_{—}14.82-27.97, C14L1.0B_{—}18.56-37.78, C15L1.0B_{—}34.73-54.95, C16L1.0B_{—}28.33-44.56, C17L1.0B_{—}32.00-45.41, C18L1.0B_{—}0.65-13.92, C19L1.0B_{—}18.67-30.73, C20L1.0B_{—}28.06-43.47, C21L1.0B_{—}11.93-24.10, C22L1.0B_{—}20.60-34.48, C23L1.0B_{—}14.14-27.73, C24L1.0B_{—}47.57-55.04, C25L1.0B_{—}27.70-36.65, C26L1.0B_{—}10.99-30.90, C27L1.0B_{—}24.14-35.66, C28L1.0B_{—}30.30-37.62 and C29L1.0B_{—}23.81-31.74.

In another embodiment of the above aspects the phenotype is fat yield (FAT) and the LD block is selected from the group consisting of C1L1.0B_{—}37.64-59.93, C2L1.0B_{—}101.03-113.49, C3L1.0B_{—}86.79-102.66, C4L1.0B_{—}38.59-65.77, C5L1.0B_{—}87.51-102.44, C6L1.0B_{—}12.78-27.80, C7L1.0B_{—}42.20-64.05, C8L1.0B_{—}33.47-59.35, C9L1.0B_{—}52.17-73.73, C10L1.0B_{—}20.04-41.39, C11L1.0B_{—}9.99-28.73, C12L1.0B_{—}34.61-53.14, C13L1.0B_{—}38.61-56.34, C14L1.0B_{—}0.03-7.93, C15L1.0B_{—}34.73-54.95, C16L1.0B_{—}28.33-44.56, C17L1.0B_{—}55.81-62.12, C18L1.0B_{—}13.92-25.33, C19L1.0B_{—}18.67-30.73, C20L1.0B_{—}14.58-28.06, C21L1.0B_{—}11.93-24.10, C22L1.0B_{—}1.32-10.14, C23L1.0B_{—}14.14-27.73, C24L1.0B_{—}47.57-55.04, C25L1.0B_{—}18.85-27.70, C26L1.0B_{—}10.99-30.90, C27L1.0B_{—}24.14-35.66, C28L1.0B_{—}30.30-37.62 and C29L1.0B_{—}23.81-31.74.

In another embodiment of the above aspects the phenotype is fat percent (FAT %) and the LD block is selected from the group consisting of C1L1.0B_{—}37.64-59.93, C2L1.0B_{—}16.93-30.73, C3L1.0B_{—}10.54-23.10, C4L1.0B_{—}38.59-65.77, C5L1.0B_{—}87.51-102.44, C6L1.0B_{—}79.54-93.65, C7L1.0B_{—}64.05-77.97, C8L1.0B_{—}78.41-95.65, C9L1.0B_{—}52.17-73.73, C10L1.0B_{—}41.39-63.85, C11L1.0B_{—}0.03-9.99, C12L1.0B_{—}61.59-74.02, C13L1.0B_{—}10.82-27.97, C14L1.0B_{—}0.03-7.93, C15L1.0B_{—}34.73-54.95, C16L1.0B_{—}14.51-28.33, C17L1.0B_{—}55.81-62.12, C18L1.0B_{—}0.65-13.92, C19L1.0B_{—}30.73-47.39, C20L1.0B_{—}28.06-43.47, C21L1.0B_{—}24.10-40.24, C22L1.0B_{—}1.32-10.14, C23L1.0B_{—}14.14-27.73, C24L1.0B_{—}47.57-55.04, C25L1.0B_{—}0.12-11.08, C26L1.0B_{—}10.99-30.90, C27L1.0B_{—}13.12-24.14, C28L1.0B_{—}11.38-21.51 and C29L1.0B_{—}23.81-31.74.

In another embodiment of the above aspects the phenotype is breeding value overall type (Overall Type) and the LD block is selected from the group consisting of C1L1.0B_{—}59.93-83.90, C2L1.0B_{—}113.49-125.27, C3L1.0B_{—}86.79-102.66, C4L1.0B_{—}38.59-65.77, C5L1.0B_{—}22.90-42.32, C6L1.0B_{—}59.88-79.54, C7L1.0B_{—}42.20-64.05, C8L1.0B_{—}33.47-59.35, C9L1.0B_{—}29.65-52.17, C10L1.0B_{—}20.04-41.39, C11L1.0B_{—}64.26-83.65, C12L1.0B_{—}11.23-20.94, C13L1.0B_{—}64.29-72.65, C14L1.0B_{—}37.78-54.46, C15L1.0B_{—}34.73-54.95, C16L1.0B_{—}28.33-44.56, C17L1.0B_{—}55.81-62.12, C18L1.0B_{—}38.17-52.45, C19L1.0B_{—}30.73-47.39, C20L1.0B_{—}28.06-43.47, C21L1.0B_{—}24.10-40.24, C22L1.0B_{—}20.60-34.48, C23L1.0B_{—}14.14-27.73, C24L1.0B_{—}35.09-47.57, C25L1.0B_{—}0.12-11.08, C26L1.0B_{—}10.99-30.90, C27L1.0B_{—}24.14-35.66, C28L1.0B_{—}0.03-11.38 and C29L1.0B_{—}13.31-23.81.

In another embodiment of the above aspects the phenotype is somatic cell count (SCC) and the LD block is selected from the group consisting of C1L1.0B_{—}37.64-59.93, C2L1.0B_{—}42.97-70.21, C3L1.0B_{—}86.79-102.66, C4L1.0B_{—}38.59-65.77, C5L1.0B_{—}22.90-42.32, C6L1.0B_{—}42.84-59.88, C7L1.0B_{—}25.69-42.20, C8L1.0B_{—}78.41-95.65, C9L1.0B_{—}52.17-73.73, C10L1.0B_{—}41.39-63.85, C11L1.0B_{—}83.65-93.53, C12L1.0B_{—}20.94-34.61, C13L1.0B_{—}56.34-64.29, C14L1.0B_{—}7.93-18.56, C15L1.0B_{—}34.73-54.95, C16L1.0B_{—}44.56-58.07, C17L1.0B_{—}0.05-8.52, C18L1.0B_{—}0.65-13.92, C19L1.0B_{—}30.73-47.39, C20L1.0B_{—}14.58-28.06, C21L1.0B_{—}11.93-24.10, C22L1.0B_{—}20.60-34.48, C23L1.0B_{—}14.14-27.73, C24L1.0B_{—}35.09-47.57, C25L1.0B_{—}0.12-11.08, C26L1.0B_{—}10.99-30.90, C27L1.0B_{—}24.14-35.66, C28L1.0B_{—}30.30-37.62 and C29L1.0B_{—}31.74-40.84.

In another embodiment of the above aspects the phenotype is breeding value cow fertility (Cow Fertility) and the LD block is selected from the group consisting of C1L1.0B_{—}59.93-83.90, C2L1.0B_{—}16.97-30.73, C3L1.0B_{—}52.80-72.96, C4L1.0B_{—}16.30-38.59, C5L1.0B_{—}22.90-42.32, C6L1.0B_{—}59.88-79.54, C7L1.0B_{—}42.20-64.05, C8L1.0B_{—}33.47-59.35, C9L1.0B_{—}29.65-52.17, C10L1.0B_{—}20.04-41.39, C11L1.0B_{—}64.26-83.65, C12L1.0B_{—}61.59-74.02, C13L1.0B_{—}64.29-72.65, C14L1.0B_{—}18.56-37.78, C15L1.0B_{—}34.73-54.95, C16L1.0B_{—}28.33-44.56, C17L1.0B_{—}55.81-62.12, C18L1.0B_{—}0.65-13.92, C19L1.0B_{—}18.67-30.73, C20L1.0B_{—}28.06-43.47, C21L1.0B_{—}11.93-24.10, C22L1.0B_{—}1.32-10.14, C23L1.0B_{—}14.14-27.73, C24L1.0B_{—}47.57-55.04, C25L1.0B_{—}11.08-18.85, C26L1.0B_{—}10.99-30.90, C27L1.0B_{—}24.14-35.66, C28L1.0B_{—}0.03-11.38 and C29L1.0B_{—}7.33-13.31.

In particular embodiments of the above aspects, the at least one genetic marker known to reside in an LD block is selected from the group consisting of a single nucleotide polymorphism (SNP), a haplotype, a microsatellite (simple tandem repeat STR, simple sequence repeat SSR), a restriction fragment length polymorphism (RFLP), an amplified fragment length polymorphism (AFLP), and an insertion-deletion polymorphism (INDEL).

In certain embodiments of the above aspects, the step of analysing the nucleic acid sample for the presence of at least one genetic marker known to reside in an LD block comprises random amplified polymorphic DNA (RAPD), ligase chain reaction, insertion/deletion analysis or direct sequencing of the gene.

The bovine may be selected from the group comprising Angus, Shorthorn, Limosin, Friesian, Wagyu, Jersey and Holstein or a cross of any two or more thereof. In particular embodiments, the bovine may be a Holstein or a Holstein/Friesian.

According to another aspect, there is provided a linkage disequilibrium unit (LDU) map of any one or more of bovine chromosomes BTA-1 to BTA-29, wherein said map comprises a plurality of chromosomal regions, and wherein said regions are defined by their co-inheritance across generations substantially as entire linkage disequilibrium (LD) blocks.

The chromosomal regions may comprise a plurality of genetic markers. The plurality of genetic markers may be of high density across the chromosomal regions.

The relative order and orientation of the genetic markers within each LD block may be substantially conserved across generations.

The map may have an LDU stringency of 1.0.

The maps may comprise a plurality of chromosomal regions as set out in Table 1.

In particular embodiments, the breeding worth of an animal reflected by genetic merit, phenotype, and performance of future progeny for a defined purpose may be predicted and selected using the methods described herein.

### BRIEF DESCRIPTION OF THE FIGURES

The present invention will now be described, by way of example only, with reference to the following figures.

FIG. 1: Distribution of the coefficient of co-ancestry (kinship) between bulls used in this study.

FIG. 2: Distribution of the inbreeding coefficient of the bulls used in this study.

FIG. 3: Distribution of SNP spacing: the distance in base pairs (kb) from one SNP marker to the next SNP marker on the chromosome.

FIG. 4: Frequency distribution of MAF of the SNP used for construction of LDU maps of all bovine chromosomes BTA-1 to BTA-29.

FIG. 5: Frequency distribution of the observed heterozygosity of the SNPs used for construction of LDU maps of bovine chromosomes BTA-1 to BTA-29.

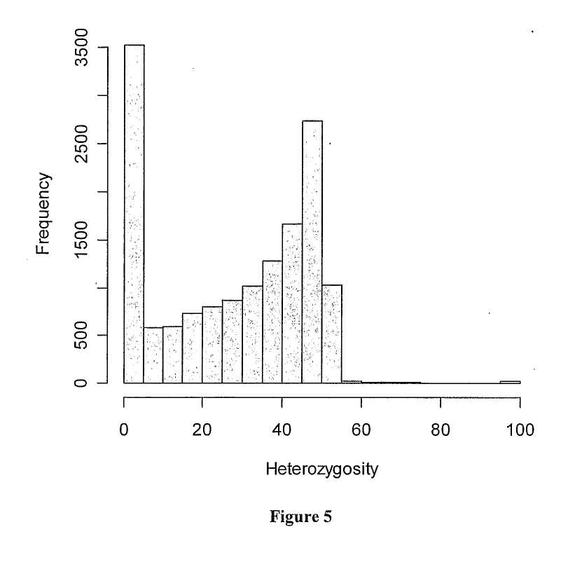

FIG. 6: Distribution of D′ values observed between SNP pairs in relation to the physical distance between them (Mb), pooled over all autosomes. The thin upper line shows the average D′ in each 500 kb sliding window. The thicker lower line shows the theoretical distribution from the fitted Malecot model.

FIG. 7: Frequency distribution of the LD block size (with LDU=1) for bovine chromosomes BTA-1 to BTA-29.

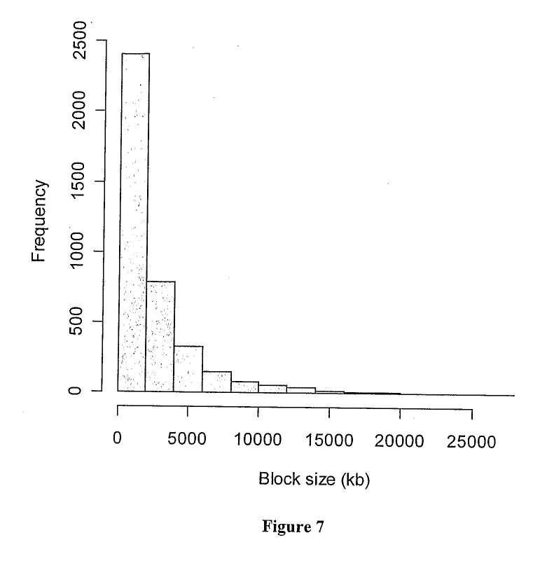

### DEFINITIONS

In the context of this specification, the term “comprising” means “including, but not necessarily solely including”. Furthermore, variations of the word “comprising”, such as “comprise” and “comprises”, have correspondingly varied meanings.

The term “primer” as used herein means a single-stranded oligonucleotide capable of acting as a point of initiation of template-directed DNA synthesis. An “oligonucleotide” is a single-stranded nucleic acid typically ranging in length from 2 to about 500 bases. The precise length of a primer will vary according to the particular application, but typically ranges from 15 to 30 nucleotides. A primer need not reflect the exact sequence of the template but must be sufficiently complementary to hybridize to the template.

The term “genotype” as used herein means the genetic constitution of an organism. This may be considered in total, or as in the present application, with respect to the alleles of a single gene (that is, at a given genetic locus).

The term “homozygote” refers to an organism that has identical alleles at a given locus on homologous chromosomes.

The term “heterozygote” refers to an organism in which different alleles are found on homologous alleles for a given locus.

The term “genetic marker” refers to a variant at DNA sequence level linked to a specific chromosomal location unique to an individual's genotype, inherited in a predictable manner, and measured as a direct DNA sequence variant or polymorphism, such as at least one Single Nucleotide Polymorphism (SNP), Restriction Fragment Length Polymorphism (RFLP), or Short Tandem Repeat (STR), or as measured indirectly as a DNA sequence variant (eg. Single-strand conformation polymorphism (SSCP), Denaturing Detergent Gradient Gel Electrophoresis (DDGE). A marker can also be a variant at the level of a DNA derived product such as RNA polymorphism/abundance, protein polymorphism or cell metabolite polymorphism, or any other biological characteristics which have a direct relationship with the underlying DNA variants or gene product. Where a genetic marker is known to reside in an LD block, the DNA sequence variation associated with the genetic marker is known to reside in a particular LD block. The ability to determine whether the DNA sequence variation associated with the genetic marker resides within a particular block requires knowledge of the location of the borders of the particular LD block on the chromosome in which the LD block resides, and knowledge of the location of the DNA variation within that chromosome.

The term “base pair” as used herein means a pair of nitrogenous bases, each in a separate nucleotide, in which each base is present on a separate strand of DNA and the bonding of these bases joins the component DNA strands. Typically a DNA molecule contains four bases; A (adenine), G (guanine), C (cytosine), and T (thymidine). A and G are purine bases, typically designated by the letter “R”, whereas C and T are pyrimidine bases, typically designated by the letter “Y”. The term “base pair” is abbreviated to “bp”, and the term “kilobase pair” is abbreviated to Kb.

The term “single nucleotide polymorphism” (SNP) refers to nucleic acid sequence variations that occur when a single nucleotide in the genome sequence is altered. For example, a SNP may alter the sequence AAGGCTAA to ATGGCTAA. For a variation to be considered a SNP, it must occur in at least 1% of the population. The nucleotides involved in SNPs are called alleles (see FIG. 8). It has been observed that for almost all SNPs, only two different alleles are present, wherein the SNP is refereed to as “biallelic”. In other cases, where more than two different alleles are involved in a SNP, the SNP is referred to as “multiallelic”.

The term “minor allelic frequency” (MAF) when used in relation to a particular biallelic locus represents the proportion of alleles with the lower frequency in the population. SNP inclusion criteria in the studies disclosed herein were SNPs with a frequency of greater than 0.05 in the population.

“LD blocks” are discussed below, and refer to discrete regions of a chromosome. The term “linkage disequilibrium unit” (LDU) refers to one unit on a Linkage Disequilibrium Map.

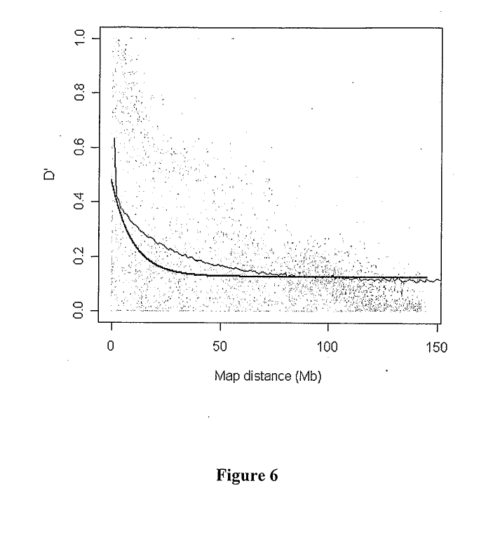

Briefly, an LDU map scale can be used to identify LD blocks as suggested by Tapper et al. 2003. LDU blocks can be formed by combining intervals between adjacent markers with LDU widths equal to one. An LDU of 1 corresponds to the number of kilobases in which substantial LD is conserved. At an LDU=1 markers within an LD block have greater linkage disequilibrium than markers outside the block. One LDU corresponds to one “swept radius”, which is the average distance in kilobases which is useful for gene mapping in a particular chromosomal region (Morton et al. 2001; Zhang et al. 2002a; Morton 2003). With reference to FIG. 6, an LDU=1 represents the point where linkage disequilibrium declines, so that markers with a higher D′ value are within 1 LDU, while markers with lower D′ outside the block further than 1 LDU distant, are considered to be outside the block. For an LDU=1 D′ values are in the range of from 0.37 to 1.0.

The term “LDU stringency” refers to the value of LDU used to define the LD blocks on the chromosome. A stringency of LDU=1 means that there is one increase in LDU on the LDU map within an LD block. In the present context, an LDU=1 is used as a threshold to discriminate genetic markers within a genomic region (an LD block with an LDU of 1) which are more closely associated with each other by linkage disequilibrium than markers outside the block.

The term “high density” when used in reference to genetic markers refers to closely spaced markers on a map.

### BEST MODE OF PERFORMING THE INVENTION

The present invention discloses construction of LD maps in LDU units for the whole of the bovine chromosome based on phase-unknown genotypes of dense SNP markers. These maps describe the LD structure over the whole of each chromosome and identify regions of high and low LD. The maps provide an unparalleled tool for optimal marker placement for association mapping.

LD across the bovine genome can be investigated using high density SNP markers. Characterizing the empirical patterns of LD across the genome is important to enhance our understanding of the biological processes of recombination and selection in the bovine genome. Furthermore, an understanding of the genomic landscape of bovine LD and variation in recombination rate facilitates the efficient design and analysis of association studies and greatly improve inferences from DNA marker polymorphism data based on population studies. Marker polymorphisms within a block are more closely associated with variation of specific traits than markers outside the block. There will be redundancy of markers within a block because of strong LD, and therefore new markers are not required to explain variation of traits within a block.

Maniatis et al. 2002 proposed that one LDU on an LD block map is a good measure of useful LD for association mapping, as it represents the “swept radius”. Equal spacing of SNPs on the LDU scale is required for coverage of a region with a minimum of one SNP per one LDU but with the expectation that more markers spanning a range of frequencies will be required to detect variants of unknown frequency.

Accordingly, the inventors have constructed metric linkage disequilibrium unit (LDU) maps of bovine chromosomes 1 to 29 based on data of 15,380 SNPs genotyped on 1,546 Australian dairy bulls using the LDMAP software (Maniatis et al. 2002) and SNP positions based on the bovine genome assembly 3.1 (National Centre for Biotechnology Information build 3.1, based on Btau_{—}3.1, National Library of Medicine, Building 38A, Bethesda, Md., 20894). The sequence of the bovine genome assembly was available from GenBank (NCBI), EMBL (http://www.embl.org/, EMBL Heidelberg, Meyerhofstraβe 1, 69117 Heidelberg, Germany), and DDBJ (DNA Data Bank of Japan, http://www.ddbj.nig.ac.jp/, 1111 Yata, Mishima, Shizuoka 411-8540, JAPAN) databases. Of these 15,380 SNPs, 344 were found to be redundant or duplicates. The sequences of each of the 15,380 SNPs are provided as SEQ ID NOS: 1 to 15,380. Each sequence presented was designed to contain sufficient flanking sequence information such that the sequence would be unique such that the position of the SNP within the bovine genome could be unequivocally identified without undue experimentation, for example by BLAST searching.

The SNP markers represented a mean spacing of 251.8±4.0 kb and mean minor allelic frequency (MAF) of 0.286±0.001. These metric LDU maps have map distances in LD units (LDUs) which are analogous to the centimorgan scale of linkage maps. The constructed maps have an average length of 6.2 LDUs. Within any given LDU map, regions of high LD (represented as blocks) and regions of low LD (represented as steps) could be observed, when plotted against the integrated map in kb. The block and step structure of the metric LD maps of BTA-1 to BTA-29 corresponds to regions of low and high recombination on the LDU maps, respectively.

It will be understood that the term “animal” as used herein refers to an individual at any stage of life, or after death, including an entity prior to birth such as a fertilised ovum, either before fusion of the male and female pro-nucleus or after the pro-nuclei have fused to form a zygote, an embryo (created by any means including somatic cell nuclear transfer) or an individual cell (N, 2N or greater); for the avoidance of doubt, this also includes a cell or a cluster of cells including stem cells and stem cell-like cells, cell line, haploid gametes and their progenitor cell lines, as well as products resulting from the gametes, including embryos. DNA from the animal to be assessed may be extracted by a number of suitable methods known to those skilled in the art. Most typically, DNA is extracted from a blood or semen sample, and in particular from peripheral blood leucocytes.

### EXAMPLES

### Example 1

### Methods and Materials

### 1.1 DNA Samples and Selection of Bulls

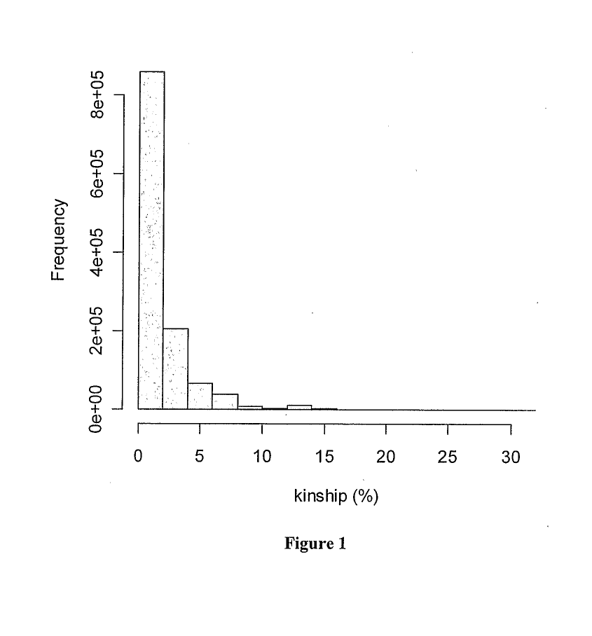

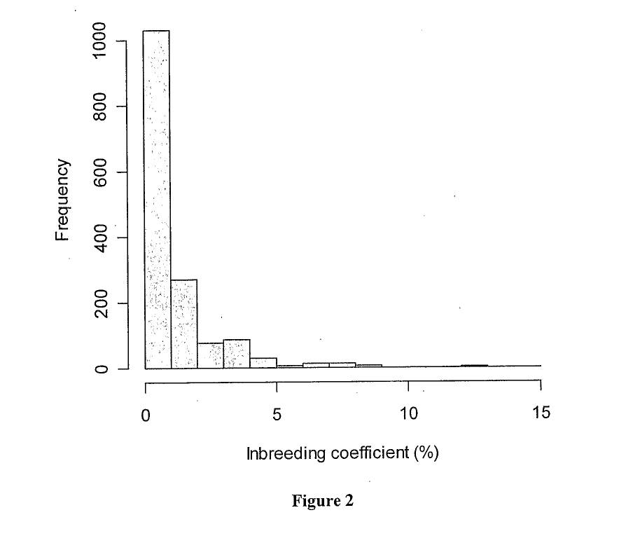

A panel of 1,546 Holstein Friesian bulls born between 1955 and 2001 was selected for genotyping. Most of these bulls were born in Australia (1,435) with smaller numbers being born in USA (53), Canada (35), New Zealand (8), Netherlands (8), Great Britain (3), France (3) and Germany (1). There were more bulls from the recent cohorts than from older cohorts. This panel of bulls represents near-to-normal distributions for Australian Breeding Values (ABVs) for the most common production traits recorded through the Australian Dairy Herd Improvement Scheme (ADHIS; http://www.adhis.com.au/). From ADHIS pedigree information (http://www.adhis.com.au/, ADHIS Pty. Ltd, Level 6 84 William Street, Melbourne 3000 Victoria Australia) and using FORTRAN programs in the PEDIG package of D. Boichard (http://dga.jouy.inra.fr/sgqa/diffusions/pedig/pedigE.htm), kinship (coefficient of coancestry) was calculated for each pairwise combinations of bulls. On this basis, the least-related 1,000 bulls were chosen for this analysis, from the original 1,546 bulls. The mean kinship (coefficient of coancestry) among these 1,000 bulls is 0.012, with 0 and 0.017 for the first and third quartiles, respectively. FIG. 1 illustrates the distribution of the coefficient of co-ancestry (kinship) between the bulls used in this study. FIG. 2 illustrates the distribution of the inbreeding coefficient of the bulls used in this study. These bulls were assumed unrelated for the purpose of the present analysis.

1.2 Extraction and Amplification of DNA from Semen Samples

Semen samples for most of these bulls, obtained from Genetics Australia (Bacchus Marsh, Vic, Australia), were the source of genomic DNA. DNA was extracted from straws of frozen semen by a salting-out method adapted from Heyen et al. (1997). As the yields of some genomic DNA per straw were limited, all DNA samples were amplified using a Whole Genome Amplification (WGA) kit (Repli-G, Molecular Staging Inc. USA). A comparison of the genotypes of genomic DNA and the WGA DNA, for 9,710 of the SNP markers genotyped in the present study, showed an average inconsistency of less than 1%. All genotyping on which the present analysis is based was carried out using WGA DNA.

### 1.3 Identification and Source of SNPs

A genome-wide high density panel of 15,036 SNPs was assembled for genotyping across the panel of bulls. Of these SNPs, 10,000 (MegAllele Genotyping Bovine 10,000 SNP Panel, ParAllele) were generated as part of the community project of the International Bovine Genome Sequencing Consortium (IBGSC) (http://www.hgsc.bcm.tmc.edu/projects/bovine/, Human Genome Sequencing Centre, Baylor College of Medicine, One Baylor Plaza, MSC-226 Houston, Tx 77030 USA). The remaining 5,036 custom SNPs were selected from the Interactive Bovine In Silico SNP (IBISS) database (Hawken et al. 2004) (http://www.livestockgenomics.csiro.au/ibiss/, CSIRO Livestock Industries, Level 3, Gehrmann Laboratories, University of Queensland), from in-house sequencing, and from publications (Heaton et al. 1999; Prinzenberg et al. 1999; Grosse et al. 1999; Olsen et al. 2000; Cohen et al. 2004; Olsen et al. 2005). IBISS is a database application constructed by clustering all publicly available bovine ESTs. From each cluster, a consensus sequence was obtained. When a base in an EST differed from the corresponding base in the consensus sequence, the position was recorded as a SNP candidate. SNP candidates were organized according to their proximity to other SNP candidates and the number of ESTs exhibiting the alternate base at that same location. The custom SNPs described above were taken from a pool of what were considered to be the “best” SNP candidates in IBISS. The “best” SNP candidates are those where the alternate base occurs in at least 30% of the ESTs in that alignment and where no more than two SNP candidates occur in a sliding window of 10 bases. Bovine QTL (quantitative trait loci) regions of interest (Khatkar et al. 2004) were translated to the human genome. The 5,036 custom SNPs were those with predicted human locations most closely corresponding to the QTL regions of interest and/or from key candidate genes.

### 1.4 SNP Genotyping

A high-throughput SNP assay service provided by Affimetrix, Inc. was used for genotyping. A highly multiplexed Molecular Inversion Probe technology (MIP) developed by ParAllele Bioscience Inc. (Hardenbol et al. 2005) was applied. MIPs are unimolecular oligonucleotide SNP-specific probes that are insensitive to cross-reactivity among multiple probe molecules. MIPs hybridize to genomic DNA, and an enzymatic “gap fill” process produces an allele-specific signature. The resulting circularized probe can be separated from cross-reacted or unreacted probes by a simple exonuclease reaction, and then amplified with a universal set of primers for all probes. Each specific SNP assay is detected via hybridization to an Affymetrix gene chip which has a unique physical position (Hardenbol et al. 2003; Hardenbol et al. 2005). To ensure strict data integrity, concealed duplicated SNP assays and duplicated DNA samples were included throughout the entire genotyping process.

### 1.5 Estimation of SNP Locations on BTA-1 to BTA-29

The locations of the SNPs were determined on the bovine sequence assembly Btau 3.1 (ftp://ftp.hgsc.bcm.tmc.edu/pub/data/Btaurus/fasta/Btau20060815-freeze/). The SNPs were placed on chromosomal linearized scaffolds using sequence similarity. The FASTA sequence data for each candidate SNP were generated by taking 100 bases of flanking consensus (EST) sequence from either side of the SNP. These FASTA sequences were compared with sequences in the 3.1 assembly using BLAT (Kent 2002) similarity searching specifying a minimum of 95% identity. SNP positions within the flanking sequence were converted to “exact” positions within the assembly using the BLAT output. The positions for all the 15,036 genotyping assays on this sequence map could be estimated. However, only 13,705 SNPs were placed on sequence scaffolds which have been assigned to a real chromosome; the rest (1,331 SNPs) were on chromosomally unanchored scaffolds. After screening out SNPs with low MAF (MAF<0.05), deviations from Hardy-Weinberg Equilibrium (as detected by Fisher's exact test, P<0.0001) and other quality measures, 9,195 SNPs mapped on autosomes were used in this analysis.

### 1.6 Testing for Hardy Weinberg Equilibrium

The number of alleles observed and expected heterozygosity under Hardy-Weinberg Equilibrium (HWE) were computed for each SNP using genetics (Warnes and Leisch, 2005) package in R statistical software (R Development Core Team, 2005). The P-values of Fisher's exact test for derivations from HWE were computed, and derivations (P<0.0001) were excluded from the analysis.

### 1.7 Construction of Metric LD Maps

The LDMAP program (http://cedar.genetics.soton.ac.uk/pub/PROGRAMS/LDMAP; described and developed by Maniatis et al. 2002) was used to construct LD maps from phase-unknown diplotypes. Variation in the extent of LD between adjacent SNPs was calculated and expressed in LDUs. In this regard, the LDMAP software is designed to fit the Malécot model (Malecot 1948; Maniatis et al. 2002) on multiple pair-wise association measures p which, in unrelated individuals, equates to the absolute value of D′. The Malécot model predicts the decline of association with distance as follows:

ρ=(1−L)Me^{-\acute{ε}d}+L

where L is the residual association at large distances, M is the proportion of the youngest haplotype that is monophyletic, and \acute{ε} is the exponential decline of ρ with distance d.

LDMAP estimates the Malécot \acute{ε} parameter in each map interval using data from pairs that include the interval in sliding windows. The length of the i^{th} interval is \acute{ε}ᵢdᵢ LDUs, where \acute{ε}ᵢ is the Malécot parameter, and dᵢ is the length of the interval on the physical map in kb. Thus, a chromosome has a total Σ\acute{ε}ᵢdᵢ LDUs.

Maniatis et al. 2002 proposed that 1/\acute{ε} is a good measure of useful LD for mapping, as it represents the “swept radius”. Equal spacing of SNPs on the LDU scale is required for coverage of a region with a minimum of one SNP per LDU but with the expectation that more markers spanning a range of frequencies will be required to detect variants of unknown frequency.

### 1.8 Identification of LD Blocks

An LDU map scale was used to identify LD blocks as proposed by Tapper et al. 2003. LD blocks were formed by combining intervals between adjacent markers with LDU widths equal to one. The criterion for LD block definition was determined with reference to the LDU bandwidth for the LD block. LD blocks for each chromosome were constructed using a stringency of LDU=1. The location of the blocks which were identified within each chromosome is set out in Table 1. In Tables 1 to 5, “Block Label” is defined as follows: C=chromosome number; L=LDU stringency; B=physical location of that LD block in the chromosome within that LDU stringency. For example, C1L1.0B_{—}59.93-83.90 denotes an LD block located between 59.93-83.90 at LDU stringency (L) of 1.0 within bovine chromosome (C) 1. The physical location represented by (B) is further explained in Table 1. Thus for C1L1.0B_{—}59.93-83.90, the block boundary begins at position 59934.667 Mb and concludes at 83899.039 Mb as defined in the Btau_{—}3.1 bovine genome release (supra).

Thus “Block Start” and “Block Stop” positions are defined in the Btau_{—}3.1 bovine genome scaffold release.

“Block Length (kb)” is the distance in kb between the “Block Start” and “Block Stop” positions

“Block Length (LDU)” is the distance in LDU between the “Block Start” and “Block Stop” positions

“nSNPs” is the number of SNPs found between the “Block Start” and “Block Stop” positions.

### 1.9 Stepwise Regression Analysis

Step-wise regression of the SNPs within a block for each of 10 traits was performed and a model with the minimum number of SNPs which contributed for the variation within each trait (R²) was identified. For each trait the block on each chromosome which contributed the greatest variation was identified (highlighted blocks in Tables 2, 3 and 4).

Stepwise regression is a standard and an automatic regression procedure for statistical model selection in cases where there are a large number of potential explanatory variables. The procedure is used primarily in regression analysis. At each stage in the process, after a new variable is added, a test is made to check if some variables can be deleted without appreciably increasing the Residual Sum of Squares (RSS). The procedure terminates when the measure is (locally) maximized, or when the available improvement falls below some critical value. The preferred model was the one with the lowest Akaike information criterion (AIC) value. The AIC methodology attempts to find the model that best explains the data with a minimum of free parameters. This penalty discourages overfitting.

The R² value explaining the percentage variation in each trait was compiled for each block. Table 2 provides a summary of the total number of SNPs which were present in each of the blocks, and the R² value of the block associated with each of the traits Australian profit ranking (APR), Australian selection index (ASR), protein yield (PROT), protein percent (PROT %), milk volume (MILK), fat yield (FAT), fat percent (FAT %), breeding value overall type (Overall Type), somatic cell count (SCC), and breeding value cow fertility (Cow Fertility).

This process was carried independently for all the blocks within each chromosome for each of the 10 traits. The block in each chromosome with highest R² for trait in question was considered as block of interest (highlighted in Tables 3 and 4). The model from this block was taken as base model, and all SNPs required to obtain a significant R² value from this model were retained for the next model. The identity of each SNP which was selected in the model was also compiled and is presented in Table 5. For each block with the highest R-squared value for each trait as identified in Table 2, SNPs are listed. These SNPs represent the number of SNPs used in the base model (see Table 4) to generate R² value for a particular trait.

For each Block in Table 5 the SNPs order listed corresponds to their relative physical map positions. The SNPs listed in Table 5 are the minimal number of SNPs which explain the R² value for a particular trait.

Table 5 shows that SNPs identified have redundancy in their utility as markers for different traits. It should be noted that the SNPs identified in Table 5 which are related to a trait are provided for illustrative purposes only, and that the predictive value of the blocks for a particular trait is not dependent on individual SNPs. As discussed, individual SNPs within a block may be substituted without significantly affecting the predictive value provided by the block. The block is defined by the chromosomal positions set out in Table 1, and not by the SNPs which are present within the block boarders.

Additional significant SNPs ie a number of SNPs which were required to make a contribution to the R² value as measured by stepwise regression analysis were then added from the nearest adjoining block by step-wise regression. The first block added was always the adjacent block closer to the chromosome origin (the block above the selected block in Tables 2 to 4). Then this new model became the base model for adding SNPs from the next adjoining block (the adjacent block closer to the chromosome termination (the block below the selected block in Tables 2 to 4)). The remaining blocks were added in a similar stepwise fashion. The resultant R² and Delta R² (the increment in the R² over previous base model) were compiled. This data is presented in Table 3, with the base model block R² value highlighted and the Delta R² values presented for the remaining blocks.

Table 4 presents the total number of SNPs in each block (nSNP), and the minimum number of SNPs for each block which were required to calculate base R² values for each trait. It can be seen from this table that there was a considerable redundancy of SNPs for each block, in that only a proportion of SNPs from each block were required in order to arrive at an R² value for each block for each trait. The addition of further SNPs in each block or additional blocks within a chromosome did not significantly contribute to the R² value for each trait. It should also be noted that the identity of the minimum number of significant SNPs within each block for each trait was not fixed, and that particular SNPs which were selected for this model could be substituted for others SNPs within the block without significantly altering the outcome of this analysis. Furthermore additional blocks from multiple chromosomes could be used to maximise the variation accounted for in a particular trait Thus it is LD block structure, rather than the presence of specific SNPs within the block, which provides the predictive power of this technique.

Traits may be predicted on the basis of the main block with highest R², and so it is this block which may be considered of principal importance. The addition of further SNPs from adjoining blocks does not contribute significantly, as indicated by the Delta R² values in the Table 3. Hence the main block provides sufficient predictive power for the trait of interest and any addition of SNPs within this block and from the adjoining blocks will not provide additional predictive advantage.

It will be understood that multiple blocks across the genome may be used to predict performance.

### Example 2

### Development of LD Maps for BTA-1 to BTA-29

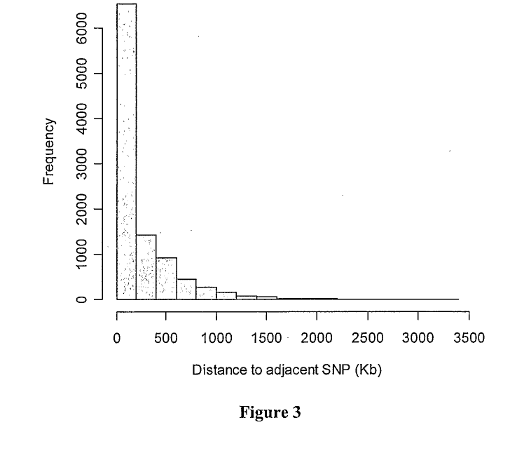

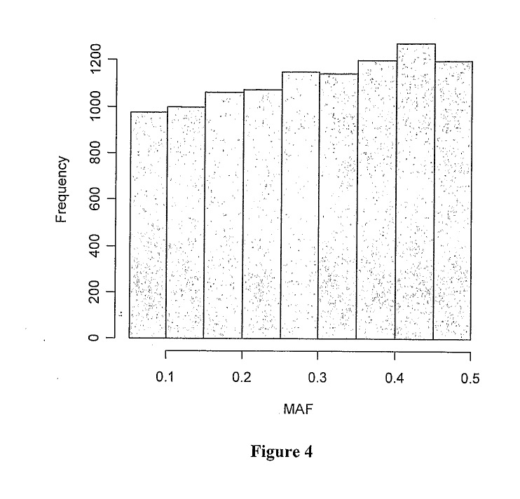

Of the 15,036 SNPs which were genotyped, 13,049 (87%) were polymorphic (minor allele frequency (MAF)>0) in the bulls included in this study. A further 1,776 (14% of the biallelic) SNPs had less than 0.05 MAF. Of the polymorphic SNPs on the autosomes, 824 (7.0%) showed deviation from Hardy-Weinberg Equilibrium (P<0.0001), and were excluded from this analysis. The SNPs (232) typed in less than 50% of animals were also removed from the analysis. Of the remaining SNPs, 9,195 were able to be located on autosomes in the bovine sequence assembly Btau 3.1 and were included in the present analysis. Of these, 7,057 (77%) of SNPs are from the MegAllele 10 k SNP panel and 2,138 (23%) from the custom SNP panel. The number of SNPs on chromosomes varied from 158 on BTA-27 to 528 on BTA-1. The average inter-marker spacing for the entire genome was 251.8±4.0 kb with a median spacing of 93.9 kb. The distribution of SNP spacing over the genome is shown in FIG. 3. The overall MAF of the SNPs used in the present analyses was 0.286±0.001 (FIG. 4).

TABLE 1

| | | | Block | Block | |
|---|---|---|---|---|---|
| | Block | Block | Length | Length | n |
| Block Label | Start | Stop | (kb) | (LDU) | SNPs |
| C1L1.0B_0.44-8.17 | 442.449 | 8165.028 | 7722.579 | 0.917 | 56 |
| C1L1.0B_8.17-17.03 | 8165.028 | 17034.364 | 8869.336 | 0.907 | 43 |
| C1L1.0B_17.03-37.64 | 17034.364 | 37638.059 | 20603.695 | 0.998 | 44 |
| C1L1.0B_37.64-59.93 | 37638.059 | 59934.667 | 22296.608 | 0.993 | 84 |
| C1L1.0B_59.93-83.90 | 59934.667 | 83899.039 | 23964.372 | 0.997 | 101 |
| C1L1.0B_83.90-98.99 | 83899.039 | 98989.577 | 15090.538 | 0.948 | 61 |
| C1L1.0B_98.99-114.96 | 98989.577 | 114964.408 | 15974.831 | 0.990 | 72 |
| C1L1.0B_114.96-133.55 | 114964.408 | 133545.71 | 18581.302 | 0.931 | 50 |
| C1L1.0B_133.55-144.87 | 133545.71 | 144874.559 | 11328.849 | 0.993 | 54 |
| C1L1.0B_144.87-145.26 | 144874.559 | 145261.378 | 386.819 | 0.508 | 5 |
| C2L1.0B_0.39-8.83 | 392.427 | 8829.878 | 8437.451 | 0.872 | 47 |
| C2L1.0B_8.83-16.97 | 8829.878 | 16967.499 | 8137.621 | 0.931 | 32 |
| C2L1.0B_16.97-30.73 | 16967.499 | 30730.971 | 13763.472 | 0.829 | 64 |
| C2L1.0B_30.73-42.97 | 30730.971 | 42969.564 | 12238.593 | 0.941 | 45 |
| C2L1.0B_42.97-70.21 | 42969.564 | 70207.961 | 27238.397 | 0.957 | 71 |
| C2L1.0B_70.21-85.72 | 70207.961 | 85716.683 | 15508.722 | 0.910 | 50 |
| C2L1.0B_85.72-101.03 | 85716.683 | 101025.529 | 15308.846 | 0.957 | 53 |
| C2L1.0B_101.03-113.49 | 101025.529 | 113494.27 | 12468.741 | 0.983 | 89 |
| C2L1.0B_113.49-125.27 | 113494.27 | 125272.472 | 11778.202 | 0.871 | 65 |
| C3L1.0B_0.46-10.54 | 460.844 | 10541.213 | 10080.369 | 0.833 | 45 |
| C3L1.0B_10.54-23.10 | 10541.213 | 23099.226 | 12558.013 | 0.933 | 76 |
| C3L1.0B_23.10-36.27 | 23099.226 | 36271.936 | 13172.71 | 0.998 | 65 |
| C3L1.0B_36.27-52.80 | 36271.936 | 52804.935 | 16532.999 | 0.981 | 78 |
| C3L1.0B_52.80-72.96 | 52804.935 | 72956.163 | 20151.228 | 0.938 | 81 |
| C3L1.0B_72.96-86.79 | 72956.163 | 86787.839 | 13831.676 | 0.994 | 34 |
| C3L1.0B_86.79-102.66 | 86787.839 | 102659.933 | 15872.094 | 0.934 | 78 |
| C3L1.0B_102.66-110.09 | 102659.933 | 110085.851 | 7425.918 | 0.976 | 33 |
| C3L1.0B_110.09-116.30 | 110085.851 | 116302.351 | 6216.5 | 0.733 | 25 |
| C4L1.0B_1.88-16.30 | 1883.029 | 16296.678 | 14413.649 | 0.910 | 59 |
| C4L1.0B_16.30-38.59 | 16296.678 | 38586.137 | 22289.459 | 0.996 | 68 |
| C4L1.0B_38.59-65.77 | 38586.137 | 65772.569 | 27186.432 | 0.881 | 108 |
| C4L1.0B_65.77-81.49 | 65772.569 | 81486.012 | 15713.443 | 0.909 | 47 |
| C4L1.0B_81.49-98.47 | 81486.012 | 98469.561 | 16983.549 | 0.950 | 70 |
| C4L1.0B_98.47-106.84 | 98469.561 | 106836.502 | 8366.941 | 0.862 | 45 |
| C4L1.0B_106.84-110.70 | 106836.502 | 110702.4 | 3865.898 | 0.423 | 28 |
| C5L1.0B_2.48-6.53 | 2480.292 | 6525.925 | 4045.633 | 0.993 | 16 |
| C5L1.0B_6.53-12.83 | 6525.925 | 12826.701 | 6300.776 | 0.754 | 35 |
| C5L1.0B_12.83-22.90 | 12826.701 | 22898.117 | 10071.416 | 0.983 | 27 |
| C5L1.0B_22.90-42.32 | 22898.117 | 42324.752 | 19426.635 | 0.940 | 78 |
| C5L1.0B_42.32-55.41 | 42324.752 | 55411.112 | 13086.36 | 0.884 | 36 |
| C5L1.0B_55.41-69.19 | 55411.112 | 69192.069 | 13780.957 | 0.966 | 65 |
| C5L1.0B_69.19-87.51 | 69192.069 | 87508.71 | 18316.641 | 0.919 | 65 |
| C5L1.0B_87.51-102.44 | 87508.71 | 102439.626 | 14930.916 | 0.987 | 55 |
| C5L1.0B_102.44-111.66 | 102439.626 | 111658.288 | 9218.662 | 0.946 | 50 |
| C5L1.0B_111.66-118.51 | 111658.288 | 118509.072 | 6850.784 | 0.384 | 25 |
| C6L1.0B_0.27-12.78 | 273.919 | 12776.218 | 12502.299 | 0.912 | 66 |
| C6L1.0B_12.78-27.80 | 12776.218 | 27804.746 | 15028.528 | 0.962 | 68 |
| C6L1.0B_27.80-42.84 | 27804.746 | 42844.908 | 15040.162 | 0.993 | 65 |
| C6L1.0B_42.84-59.88 | 42844.908 | 59877.799 | 17032.891 | 0.960 | 61 |
| C6L1.0B_59.88-79.54 | 59877.799 | 79541.054 | 19663.255 | 0.897 | 81 |
| C6L1.0B_79.54-93.65 | 79541.054 | 93653.247 | 14112.193 | 0.947 | 72 |
| C6L1.0B_93.65-111.65 | 93653.247 | 111654.84 | 18001.593 | 0.893 | 69 |
| C7L1.0B_0.33-6.82 | 326.752 | 6819.333 | 6492.581 | 0.990 | 27 |
| C7L1.0B_6.82-14.57 | 6819.333 | 14573.565 | 7754.232 | 0.985 | 53 |
| C7L1.0B_14.57-25.69 | 14573.565 | 25690.098 | 11116.533 | 0.947 | 45 |
| C7L1.0B_25.69-42.20 | 25690.098 | 42201.45 | 16511.352 | 0.919 | 52 |
| C7L1.0B_42.20-64.05 | 42201.45 | 64047.834 | 21846.384 | 0.978 | 93 |
| C7L1.0B_64.05-77.97 | 64047.834 | 77969.1 | 13921.266 | 0.962 | 42 |
| C7L1.0B_77.97-88.71 | 77969.1 | 88713.047 | 10743.947 | 0.956 | 40 |
| C7L1.0B_88.71-95.83 | 88713.047 | 95834.822 | 7121.775 | 0.977 | 20 |
| C7L1.0B_95.83-100.74 | 95834.822 | 100738.774 | 4903.952 | 0.458 | 21 |
| C8L1.0B_0.39-6.05 | 387.116 | 6050.924 | 5663.808 | 0.674 | 17 |
| C8L1.0B_6.05-17.58 | 6050.924 | 17578.728 | 11527.804 | 0.986 | 50 |
| C8L1.0B_17.58-33.47 | 17578.728 | 33466.595 | 15887.867 | 0.799 | 49 |
| C8L1.0B_33.47-59.35 | 33466.595 | 59354.085 | 25887.49 | 0.910 | 106 |
| C8L1.0B_59.35-78.41 | 59354.085 | 78406.007 | 19051.922 | 0.972 | 74 |
| C8L1.0B_78.41-95.65 | 78406.007 | 95645.195 | 17239.188 | 0.988 | 78 |
| C8L1.0B_95.65-103.23 | 95645.195 | 103230.951 | 7585.756 | 0.956 | 53 |
| C9L1.0B_0.25-6.86 | 247.511 | 6860.174 | 6612.663 | 0.956 | 9 |
| C9L1.0B_6.86-16.54 | 6860.174 | 16537.052 | 9676.878 | 0.863 | 28 |
| C9L1.0B_16.54-29.65 | 16537.052 | 29646.426 | 13109.374 | 0.964 | 39 |
| C9L1.0B_29.65-52.17 | 29646.426 | 52170.367 | 22523.941 | 0.966 | 65 |
| C9L1.0B_52.17-73.73 | 52170.367 | 73726.551 | 21556.184 | 0.921 | 74 |
| C9L1.0B_73.73-84.14 | 73726.551 | 84137.08 | 10410.529 | 0.997 | 43 |
| C9L1.0B_84.14-94.57 | 84137.08 | 94566.701 | 10429.621 | 0.583 | 37 |
| C10L1.0B_0.03-5.39 | 31.47 | 5393.172 | 5361.702 | 0.583 | 25 |
| C10L1.0B_5.39-13.84 | 5393.172 | 13835.122 | 8441.95 | 0.918 | 55 |
| C10L1.0B_13.84-20.04 | 13835.122 | 20043.671 | 6208.549 | 0.796 | 29 |
| C10L1.0B_20.04-41.39 | 20043.671 | 41394.454 | 21350.783 | 0.998 | 97 |
| C10L1.0B_41.39-63.85 | 41394.454 | 63847.277 | 22452.823 | 0.941 | 91 |
| C10L1.0B_63.85-77.05 | 63847.277 | 77053.796 | 13206.519 | 0.934 | 37 |
| C10L1.0B_77.05-87.99 | 77053.796 | 87992.77 | 10938.974 | 0.756 | 42 |
| C10L1.0B_87.99-94.67 | 87992.77 | 94674.987 | 6682.217 | 0.751 | 45 |
| C10L1.0B_94.67-95.75 | 94674.987 | 95751.991 | 1077.004 | 0.648 | 5 |
| C11L1.0B_0.03-9.99 | 29.554 | 9992.443 | 9962.889 | 0.979 | 72 |
| C11L1.0B_9.99-28.73 | 9992.443 | 28733.183 | 18740.74 | 0.908 | 62 |
| C11L1.0B_28.73-45.68 | 28733.183 | 45677.09 | 16943.907 | 0.926 | 82 |
| C11L1.0B_45.68-64.26 | 45677.09 | 64262.22 | 18585.13 | 0.991 | 105 |
| C11L1.0B_64.26-83.65 | 64262.22 | 83652.339 | 19390.119 | 0.984 | 73 |
| C11L1.0B_83.65-93.53 | 83652.339 | 93526.695 | 9874.356 | 0.892 | 48 |
| C11L1.0B_93.53-101.13 | 93526.695 | 101125.856 | 7599.161 | 0.681 | 47 |
| C12L1.0B_1.00-7.24 | 1003.413 | 7244.452 | 6241.039 | 0.971 | 16 |
| C12L1.0B_7.24-11.23 | 7244.452 | 11225.344 | 3980.892 | 0.829 | 29 |
| C12L1.0B_11.23-20.94 | 11225.344 | 20939.265 | 9713.921 | 0.985 | 54 |
| C12L1.0B_20.94-34.61 | 20939.265 | 34606.973 | 13667.708 | 0.942 | 48 |
| C12L1.0B_34.61-53.14 | 34606.973 | 53142.923 | 18535.95 | 0.949 | 48 |
| C12L1.0B_53.14-61.59 | 53142.923 | 61590.188 | 8447.265 | 0.994 | 36 |
| C12L1.0B_61.59-74.02 | 61590.188 | 74018.336 | 12428.148 | 0.997 | 53 |
| C12L1.0B_74.02-77.41 | 74018.336 | 77412.38 | 3394.044 | 0.588 | 17 |
| C13L1.0B_0.49-5.70 | 489.37 | 5698.826 | 5209.456 | 0.912 | 39 |
| C13L1.0B_5.70-14.82 | 5698.826 | 14824.35 | 9125.524 | 0.903 | 38 |
| C13L1.0B_14.82-27.97 | 14824.35 | 27972.401 | 13148.051 | 0.829 | 79 |
| C13L1.0B_27.97-38.61 | 27972.401 | 38612.256 | 10639.855 | 0.987 | 48 |
| C13L1.0B_38.61-56.34 | 38612.256 | 56342.234 | 17729.978 | 0.710 | 101 |
| C13L1.0B_56.34-64.29 | 56342.234 | 64293.399 | 7951.165 | 0.835 | 40 |
| C13L1.0B_64.29-72.65 | 64293.399 | 72647.412 | 8354.013 | 0.919 | 65 |
| C13L1.0B_72.65-81.39 | 72647.412 | 81390.4 | 8742.988 | 0.987 | 39 |
| C13L1.0B_81.39-82.75 | 81390.4 | 82747.057 | 1356.657 | 0.137 | 7 |
| C14L1.0B_0.03-7.93 | 32.23 | 7926.227 | 7893.997 | 0.932 | 35 |
| C14L1.0B_7.93-18.56 | 7926.227 | 18556.005 | 10629.778 | 0.916 | 68 |
| C14L1.0B_18.56-37.78 | 18556.005 | 37781.238 | 19225.233 | 0.883 | 64 |
| C14L1.0B_37.78-54.46 | 37781.238 | 54461.775 | 16680.537 | 0.890 | 59 |
| C14L1.0B_54.46-69.52 | 54461.775 | 69523.893 | 15062.118 | 0.979 | 59 |
| C14L1.0B_69.52-77.33 | 69523.893 | 77328.439 | 7804.546 | 0.892 | 37 |
| C14L1.0B_77.33-81.90 | 77328.439 | 81900.996 | 4572.557 | 0.727 | 16 |
| C15L1.0B_0.47-9.13 | 465.307 | 9131.039 | 8665.732 | 0.883 | 27 |
| C15L1.0B_9.13-17.86 | 9131.039 | 17860.321 | 8729.282 | 0.901 | 50 |
| C15L1.0B_17.86-34.73 | 17860.321 | 34732.176 | 16871.855 | 0.976 | 74 |
| C15L1.0B_34.73-54.95 | 34732.176 | 54948.447 | 20216.271 | 0.934 | 111 |
| C15L1.0B_54.95-64.43 | 54948.447 | 64434.113 | 9485.666 | 0.734 | 51 |
| C15L1.0B_64.43-71.14 | 64434.113 | 71137.355 | 6703.242 | 0.833 | 19 |
| C15L1.0B_71.14-75.12 | 71137.355 | 75119.895 | 3982.54 | 0.438 | 13 |
| C16L1.0B_0.01-8.72 | 6.913 | 8718.309 | 8711.396 | 0.538 | 29 |
| C16L1.0B_8.72-14.51 | 8718.309 | 14507.021 | 5788.712 | 0.780 | 34 |
| C16L1.0B_14.51-28.33 | 14507.021 | 28326.188 | 13819.167 | 0.931 | 80 |
| C16L1.0B_28.33-44.56 | 28326.188 | 44560.437 | 16234.249 | 0.929 | 68 |
| C16L1.0B_44.56-58.07 | 44560.437 | 58068.291 | 13507.854 | 0.871 | 63 |
| C16L1.0B_58.07-64.59 | 58068.291 | 64589.097 | 6520.806 | 0.866 | 35 |
| C16L1.0B_64.59-72.02 | 64589.097 | 72023.86 | 7434.763 | 0.765 | 32 |
| C17L1.0B_0.05-8.52 | 54.279 | 8521.805 | 8467.526 | 0.973 | 34 |
| C17L1.0B_8.52-18.79 | 8521.805 | 18794.445 | 10272.64 | 0.941 | 28 |
| C17L1.0B_18.79-32.00 | 18794.445 | 32002.926 | 13208.481 | 0.961 | 53 |
| C17L1.0B_32.00-45.41 | 32002.926 | 45414.194 | 13411.268 | 0.879 | 50 |
| C17L1.0B_45.41-55.81 | 45414.194 | 55806.548 | 10392.354 | 0.868 | 82 |
| C17L1.0B_55.81-62.12 | 55806.548 | 62124.084 | 6317.536 | 0.868 | 42 |
| C17L1.0B_62.12-69.54 | 62124.084 | 69541.496 | 7417.412 | 0.947 | 37 |
| C18L1.0B_0.65-13.92 | 647.403 | 13915.099 | 13267.696 | 0.974 | 88 |
| C18L1.0B_13.92-25.33 | 13915.099 | 25333.733 | 11418.634 | 0.994 | 59 |
| C18L1.0B_25.33-38.17 | 25333.733 | 38172.549 | 12838.816 | 0.994 | 64 |
| C18L1.0B_38.17-52.45 | 38172.549 | 52452.884 | 14280.335 | 0.955 | 78 |
| C18L1.0B_52.45-62.46 | 52452.884 | 62464.616 | 10011.732 | 0.975 | 31 |
| C19L1.0B_0.16-3.42 | 160.198 | 3417.327 | 3257.129 | 0.809 | 8 |
| C19L1.0B_3.42-11.54 | 3417.327 | 11538.169 | 8120.842 | 0.955 | 39 |
| C19L1.0B_11.54-18.67 | 11538.169 | 18673.703 | 7135.534 | 0.989 | 58 |
| C19L1.0B_18.67-30.73 | 18673.703 | 30727.393 | 12053.69 | 0.910 | 91 |
| C19L1.0B_30.73-47.39 | 30727.393 | 47392.25 | 16664.857 | 0.989 | 88 |
| C19L1.0B_47.39-57.41 | 47392.25 | 57405.298 | 10013.048 | 0.977 | 53 |
| C19L1.0B_57.41-63.02 | 57405.298 | 63020.385 | 5615.087 | 0.966 | 31 |
| C20L1.0B_0.19-4.70 | 189.698 | 4703.728 | 4514.03 | 0.864 | 20 |
| C20L1.0B_4.70-14.58 | 4703.728 | 14578.313 | 9874.585 | 0.919 | 46 |
| C20L1.0B_14.58-28.06 | 14578.313 | 28058.233 | 13479.92 | 0.779 | 44 |
| C20L1.0B_28.06-43.47 | 28058.233 | 43470.894 | 15412.661 | 0.915 | 63 |
| C20L1.0B_43.47-57.23 | 43470.894 | 57229.095 | 13758.201 | 0.902 | 56 |
| C20L1.0B_57.23-65.67 | 57229.095 | 65668.369 | 8439.274 | 1.000 | 30 |
| C20L1.0B_65.67-67.29 | 65668.369 | 67291.271 | 1622.902 | 0.311 | 13 |
| C21L1.0B_0.73-11.93 | 727.627 | 11933.742 | 11206.115 | 0.869 | 24 |
| C21L1.0B_11.93-24.10 | 11933.742 | 24098.048 | 12164.306 | 0.877 | 52 |
| C21L1.0B_24.10-40.24 | 24098.048 | 40242.822 | 16144.774 | 0.977 | 53 |
| C21L1.0B_40.24-53.06 | 40242.822 | 53064.532 | 12821.71 | 0.970 | 43 |
| C21L1.0B_53.06-59.93 | 53064.532 | 59934.113 | 6869.581 | 0.950 | 19 |
| C21L1.0B_59.93-62.91 | 59934.113 | 62907.972 | 2973.859 | 0.138 | 7 |
| C22L1.0B_1.32-10.14 | 1317.404 | 10140.583 | 8823.179 | 0.983 | 51 |
| C22L1.0B_10.14-20.60 | 10140.583 | 20601.876 | 10461.293 | 0.994 | 32 |
| C22L1.0B_20.60-34.48 | 20601.876 | 34480.55 | 13878.674 | 0.981 | 80 |
| C22L1.0B_34.48-46.43 | 34480.55 | 46425.66 | 11945.11 | 0.919 | 52 |
| C22L1.0B_46.43-52.20 | 46425.66 | 52196.929 | 5771.269 | 0.888 | 30 |
| C22L1.0B_52.20-58.18 | 52196.929 | 58184.214 | 5987.285 | 0.851 | 19 |
| C22L1.0B_58.18-59.02 | 58184.214 | 59018.855 | 834.641 | 0.338 | 2 |
| C23L1.0B_1.16-5.31 | 1159.395 | 5306.477 | 4147.082 | 0.999 | 18 |
| C23L1.0B_5.31-14.14 | 5306.477 | 14143.899 | 8837.422 | 0.945 | 44 |
| C23L1.0B_14.14-27.73 | 14143.899 | 27727.459 | 13583.56 | 0.980 | 126 |
| C23L1.0B_27.73-37.77 | 27727.459 | 37771.904 | 10044.445 | 0.850 | 49 |
| C23L1.0B_37.77-48.55 | 37771.904 | 48553.02 | 10781.116 | 0.852 | 58 |
| C24L1.0B_0.14-10.98 | 136.357 | 10979.924 | 10843.567 | 0.949 | 37 |
| C24L1.0B_10.98-23.20 | 10979.924 | 23197.952 | 12218.028 | 0.780 | 43 |
| C24L1.0B_23.20-35.09 | 23197.952 | 35088.438 | 11890.486 | 0.938 | 69 |
| C24L1.0B_35.09-47.57 | 35088.438 | 47572.921 | 12484.483 | 0.825 | 59 |
| C24L1.0B_47.57-55.04 | 47572.921 | 55036.657 | 7463.736 | 0.961 | 23 |
| C24L1.0B_55.04-60.03 | 55036.657 | 60030.383 | 4993.726 | 0.293 | 18 |
| C25L1.0B_0.12-11.08 | 118.717 | 11075.395 | 10956.678 | 0.931 | 48 |
| C25L1.0B_11.08-18.85 | 11075.395 | 18848.452 | 7773.057 | 0.970 | 48 |
| C25L1.0B_18.85-27.70 | 18848.452 | 27698.782 | 8850.33 | 0.999 | 58 |
| C25L1.0B_27.70-36.65 | 27698.782 | 36649.208 | 8950.426 | 0.902 | 50 |
| C25L1.0B_36.65-41.76 | 36649.208 | 41757.451 | 5108.243 | 0.791 | 35 |
| C26L1.0B_0.76-10.99 | 762.656 | 10994.985 | 10232.329 | 0.856 | 55 |
| C26L1.0B_10.99-30.90 | 10994.985 | 30897.399 | 19902.414 | 0.976 | 72 |
| C26L1.0B_30.90-43.28 | 30897.399 | 43280.337 | 12382.938 | 0.969 | 61 |
| C26L1.0B_43.28-47.50 | 43280.337 | 47496.426 | 4216.089 | 0.710 | 11 |
| C27L1.0B_0.23-8.39 | 233.474 | 8393.259 | 8159.785 | 0.464071 | 18 |
| C27L1.0B_8.39-13.12 | 8393.259 | 13117.911 | 4724.652 | 0.872 | 29 |
| C27L1.0B_13.12-24.14 | 13117.911 | 24142.059 | 11024.148 | 0.997 | 42 |
| C27L1.0B_24.14-35.66 | 24142.059 | 35661.957 | 11519.898 | 0.946 | 52 |
| C27L1.0B_35.66-43.22 | 35661.957 | 43224.783 | 7562.826 | 0.632 | 33 |
| C28L1.0B_0.03-11.38 | 30.17 | 11375.159 | 11344.989 | 0.955 | 60 |
| C28L1.0B_11.38-21.51 | 11375.159 | 21514.967 | 10139.808 | 0.938 | 35 |
| C28L1.0B_21.51-30.30 | 21514.967 | 30301.818 | 8786.851 | 0.989 | 42 |
| C28L1.0B_30.30-37.62 | 30301.818 | 37620.053 | 7318.235 | 0.930 | 46 |
| C28L1.0B_37.62-39.37 | 37620.053 | 39371.001 | 1750.948 | 0.169 | 8 |
| C29L1.0B_0.19-7.33 | 194.808 | 7332.452 | 7137.644 | 0.954 | 30 |
| C29L1.0B_7.33-13.31 | 7332.452 | 13305.056 | 5972.604 | 0.985 | 47 |
| C29L1.0B_13.31-23.81 | 13305.056 | 23812.365 | 10507.309 | 0.986 | 26 |
| C29L1.0B_23.81-31.74 | 23812.365 | 31741.879 | 7929.514 | 0.980 | 33 |
| C29L1.0B_31.74-40.84 | 31741.879 | 40838.639 | 9096.76 | 0.981 | 44 |
| C29L1.0B_40.84-44.88 | 40838.639 | 44882.572 | 4043.933 | 0.817 | 16 |

TABLE 2

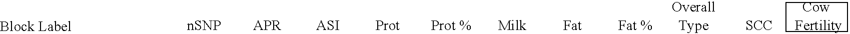

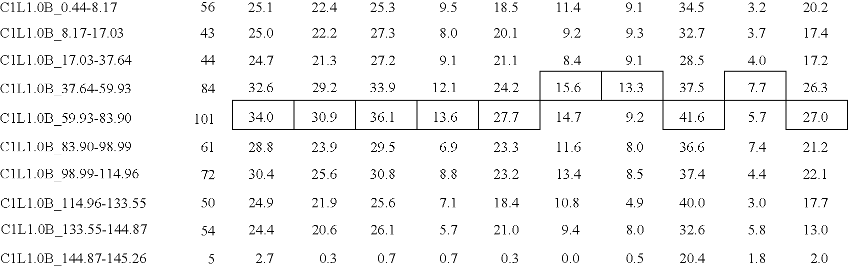

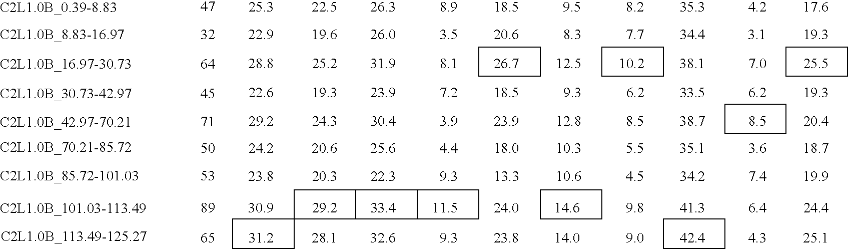

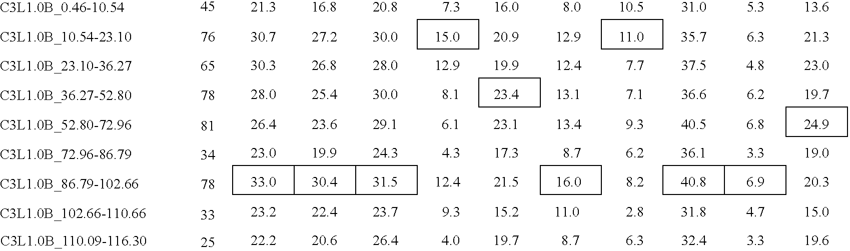

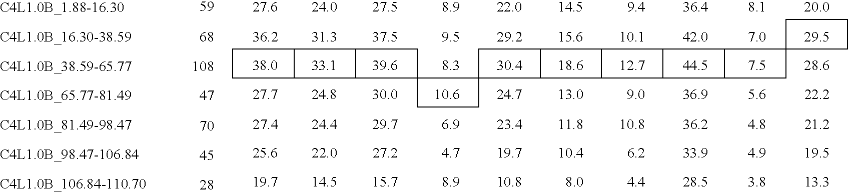

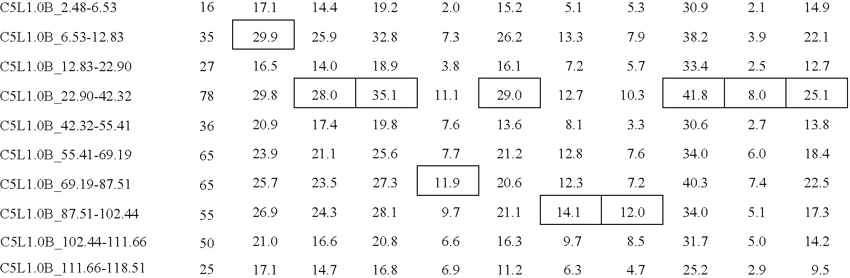

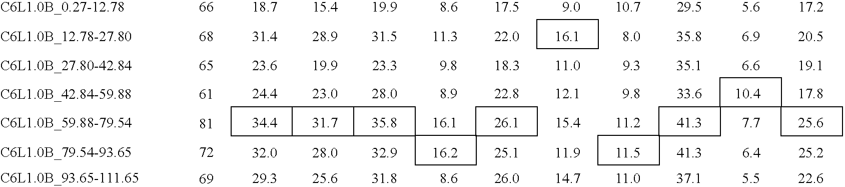

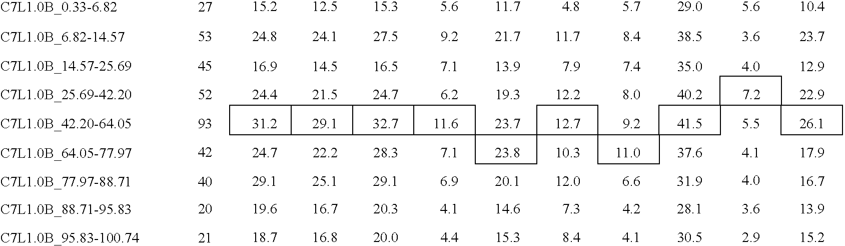

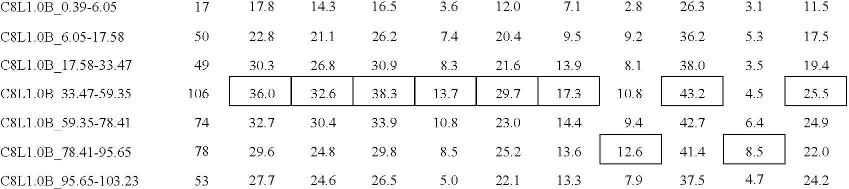

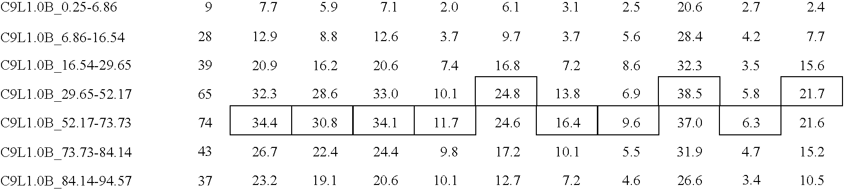

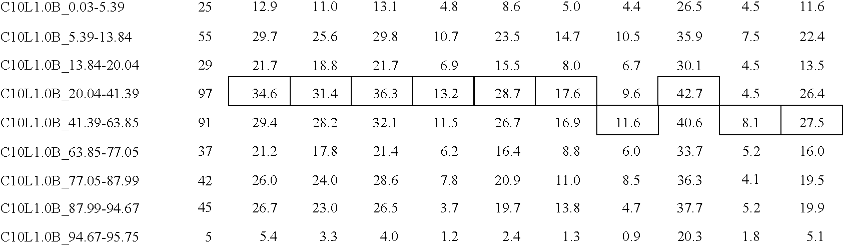

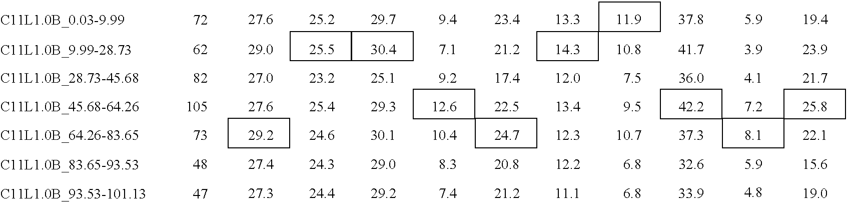

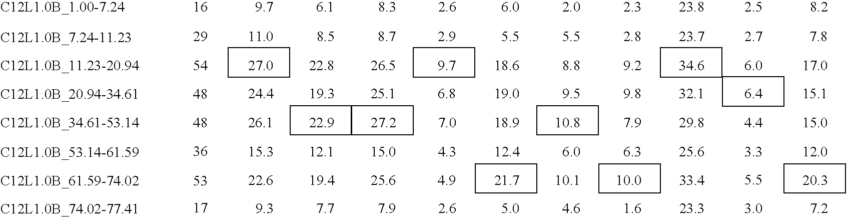

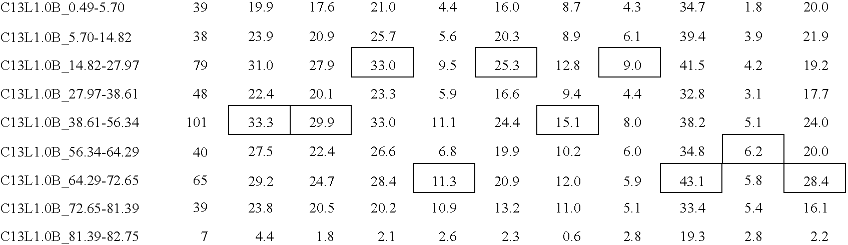

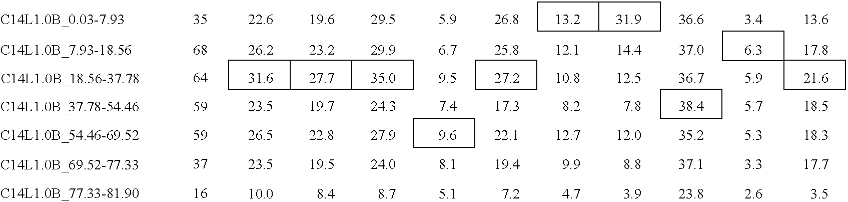

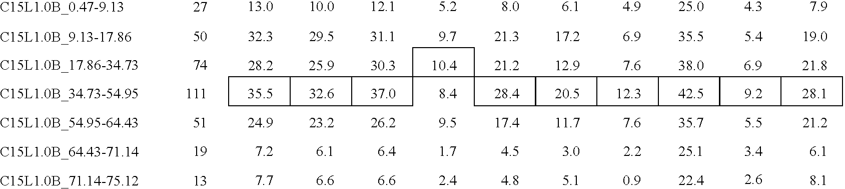

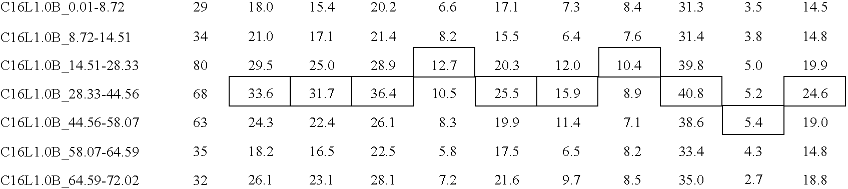

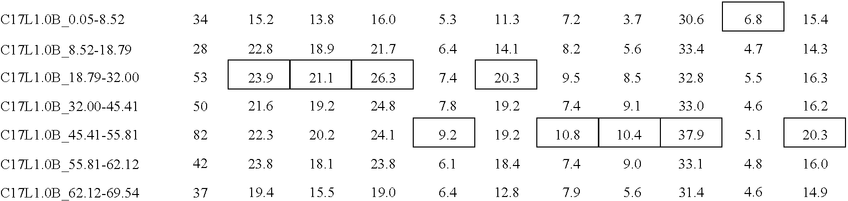

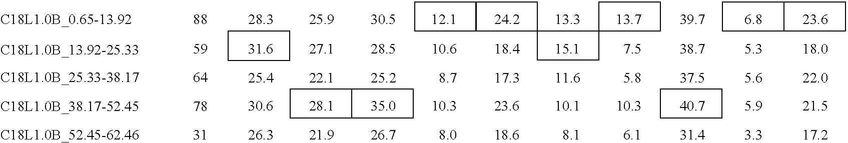

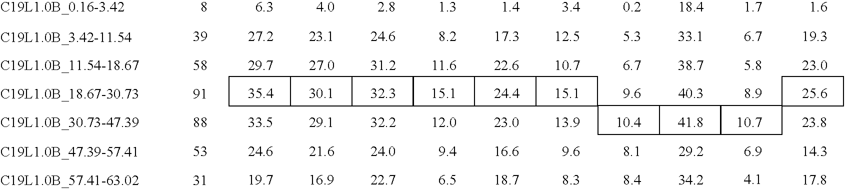

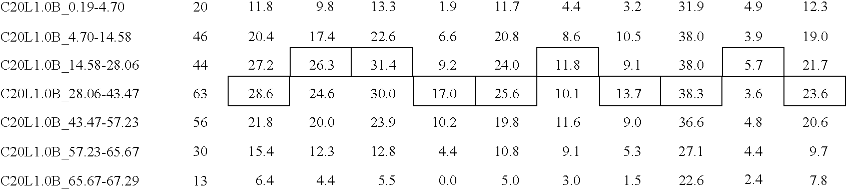

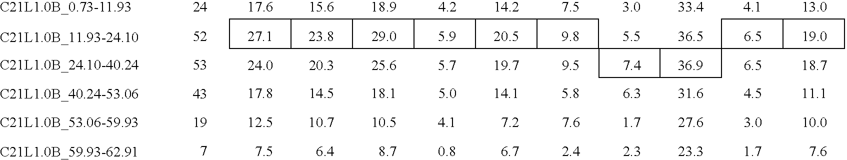

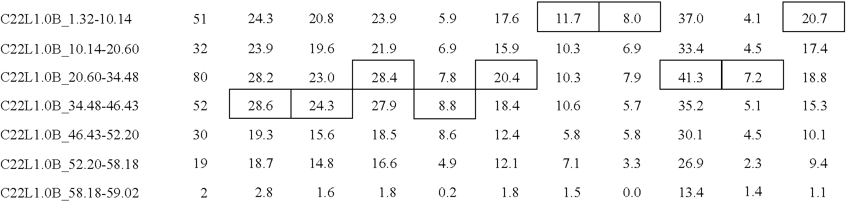

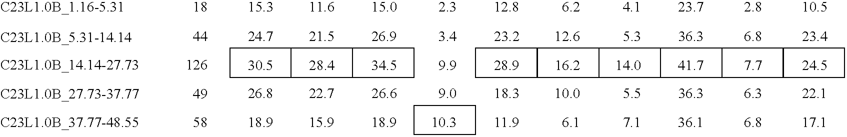

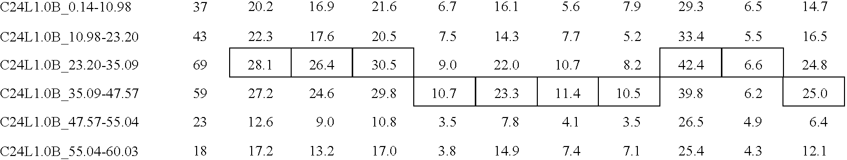

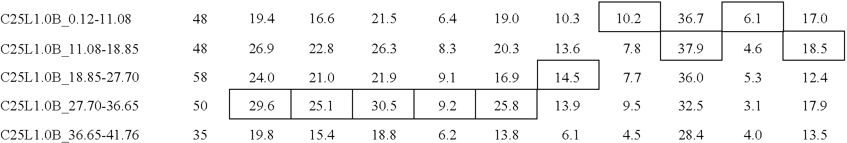

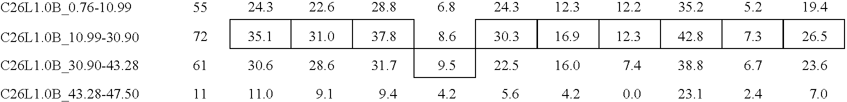

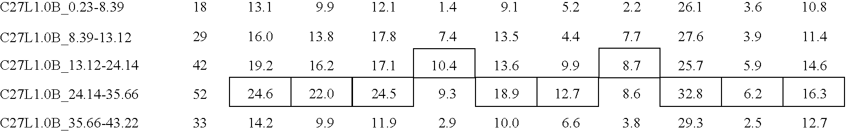

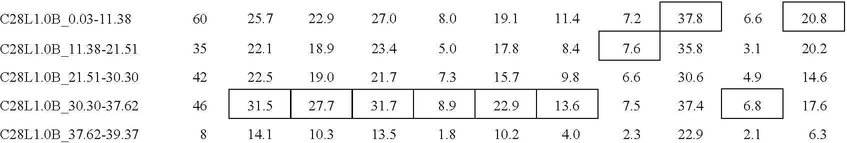

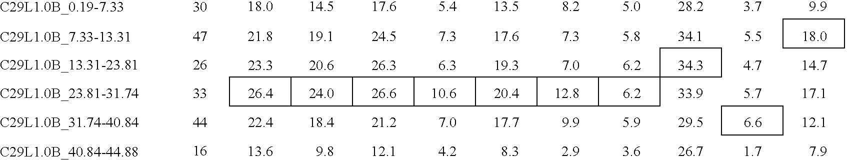

TABLE 3

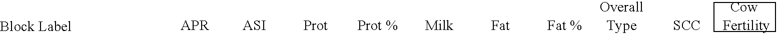

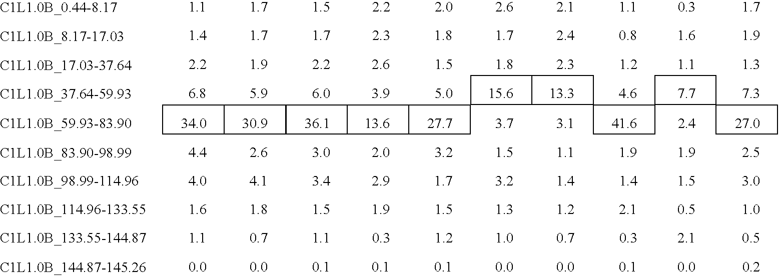

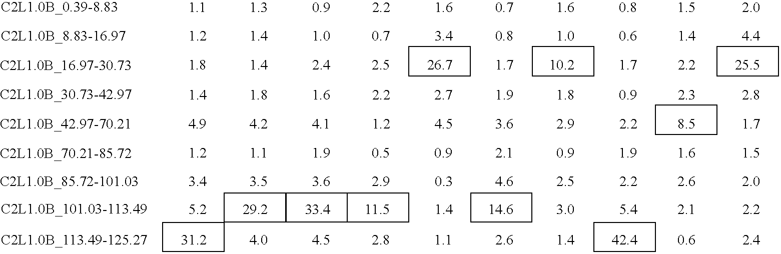

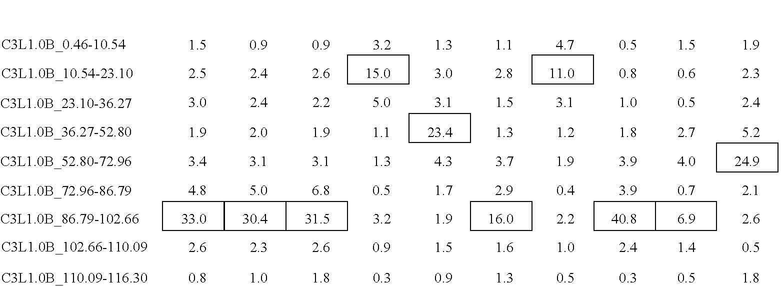

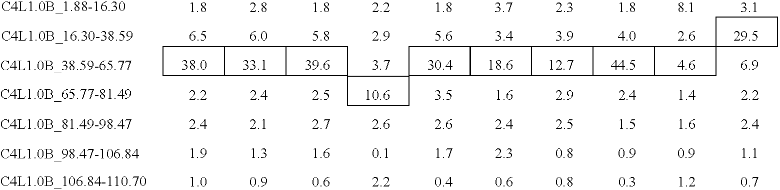

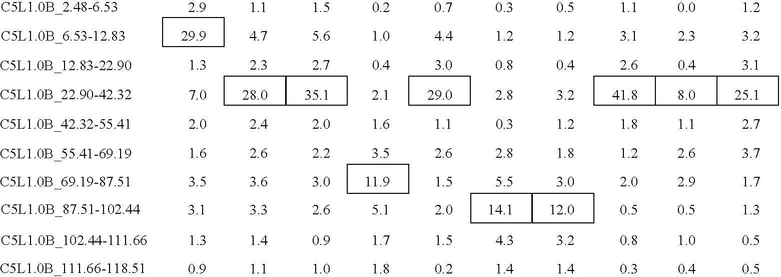

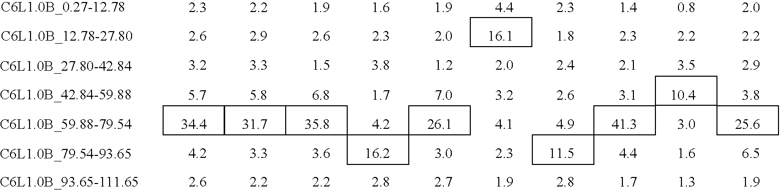

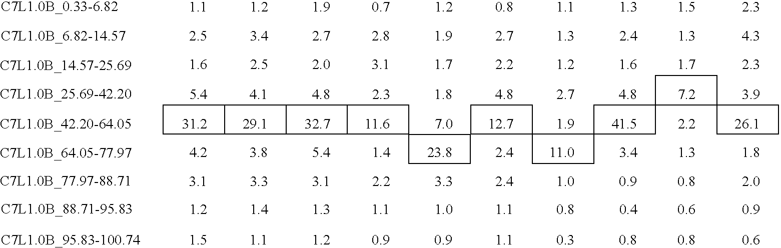

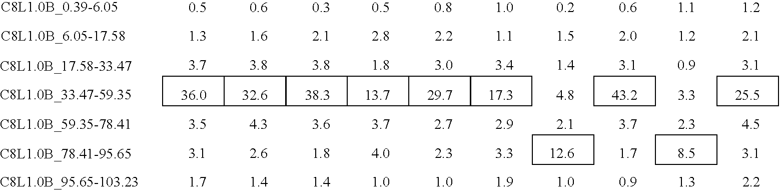

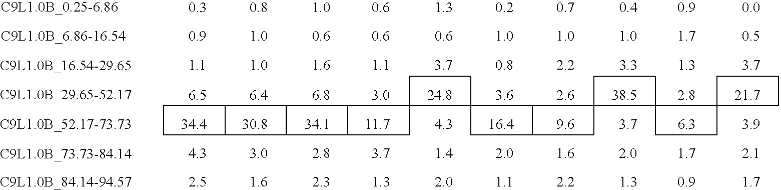

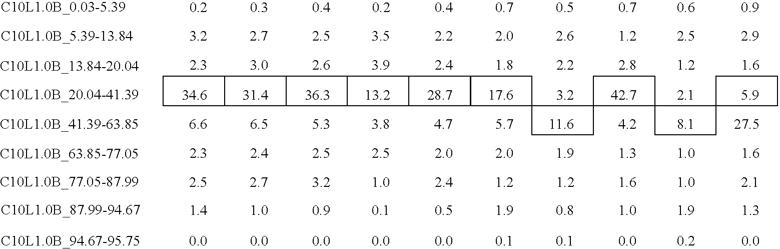

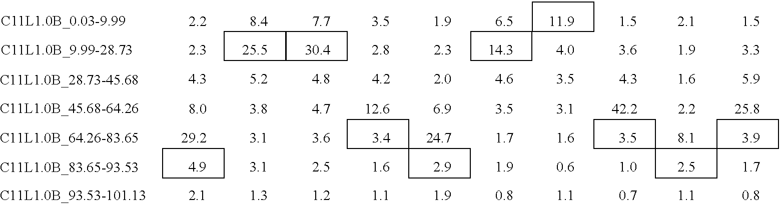

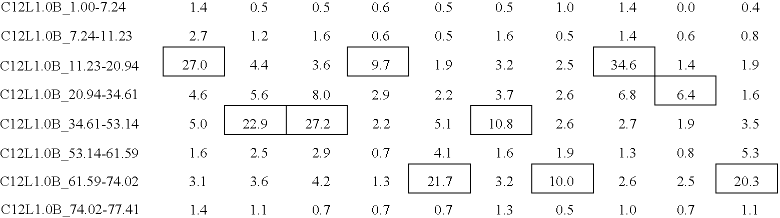

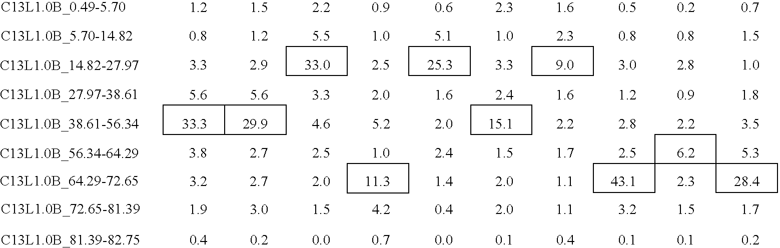

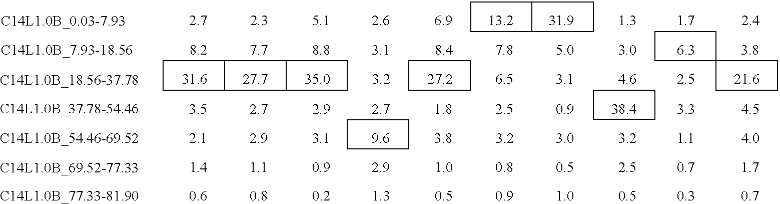

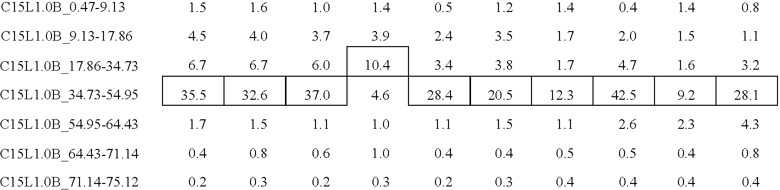

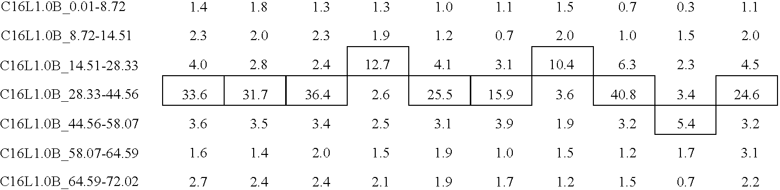

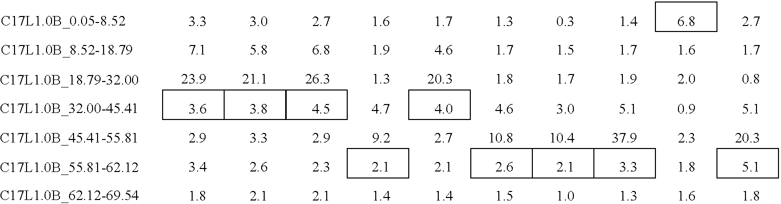

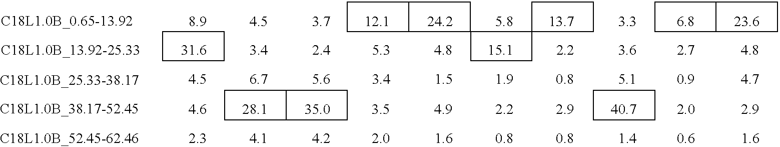

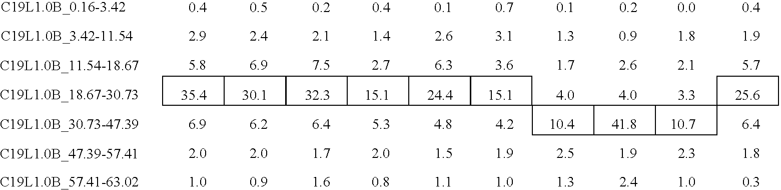

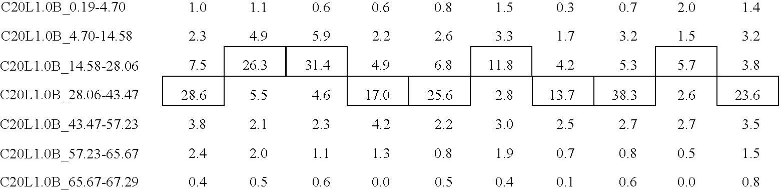

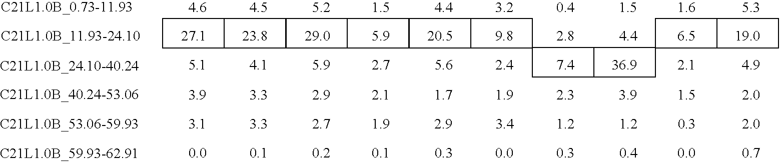

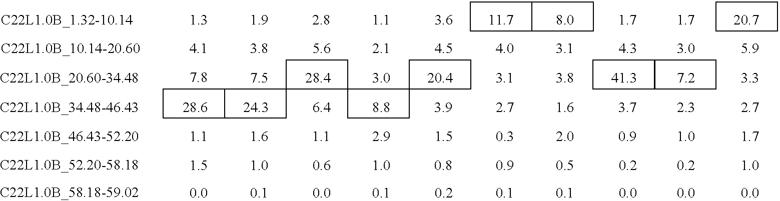

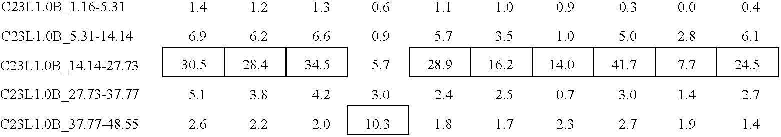

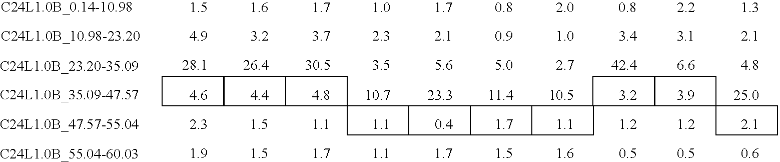

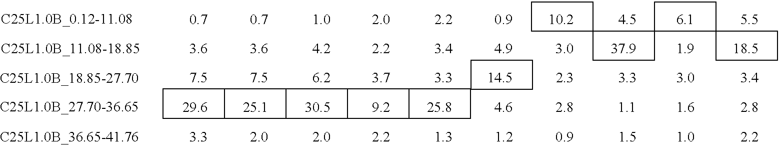

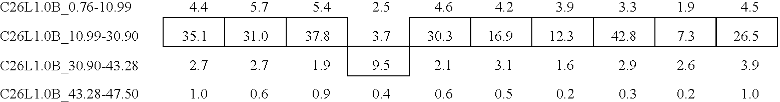

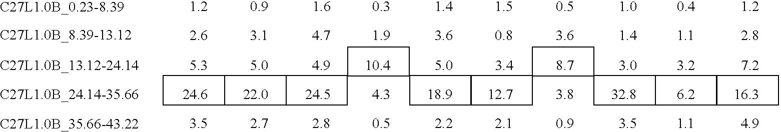

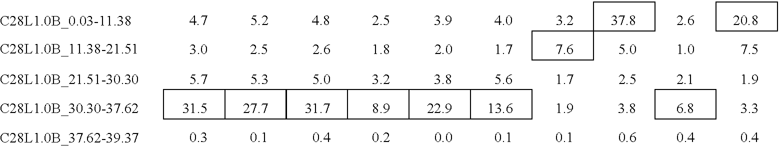

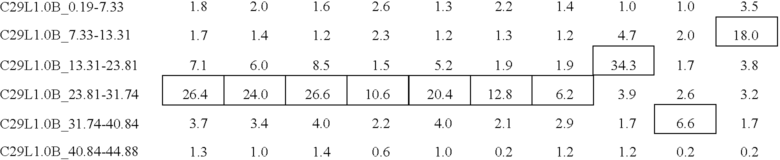

TABLE 4

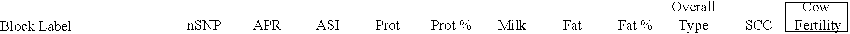

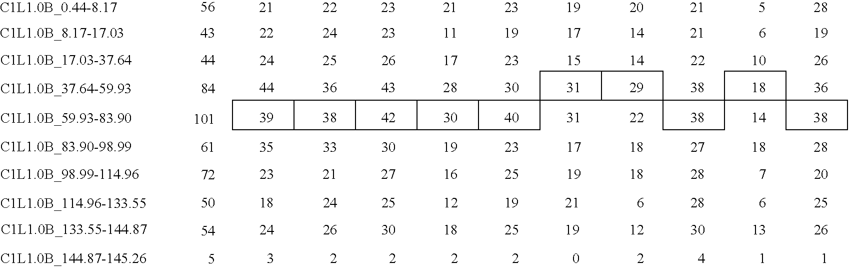

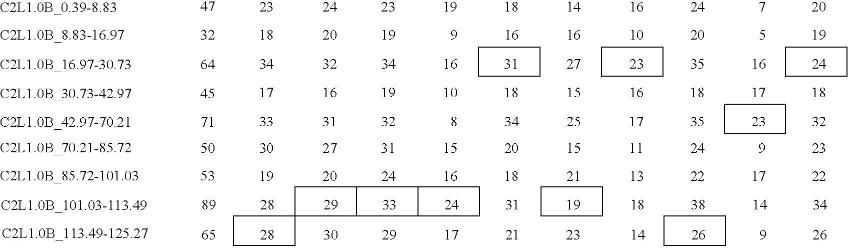

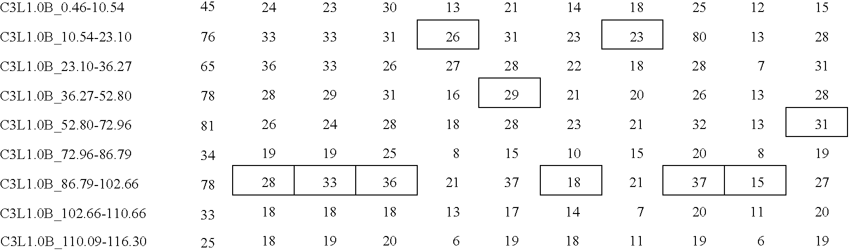

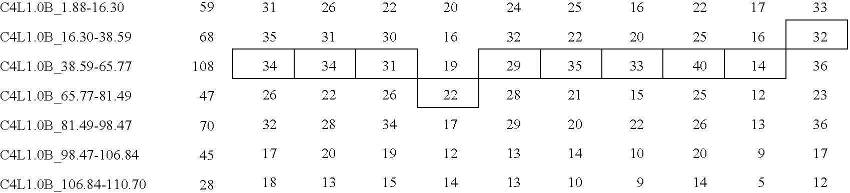

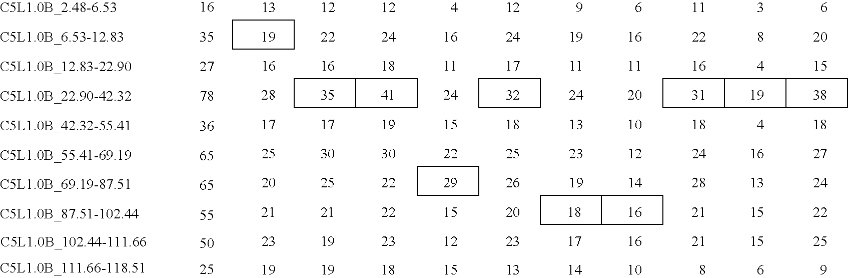

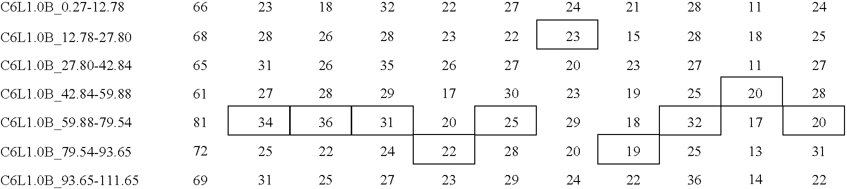

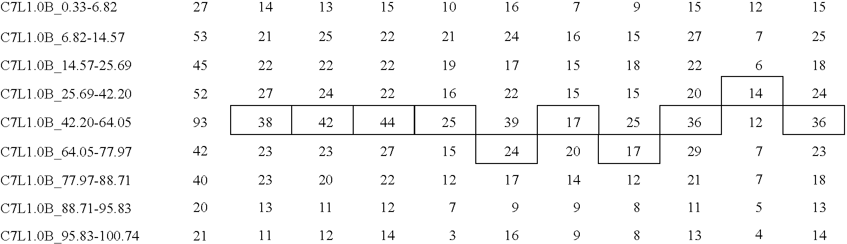

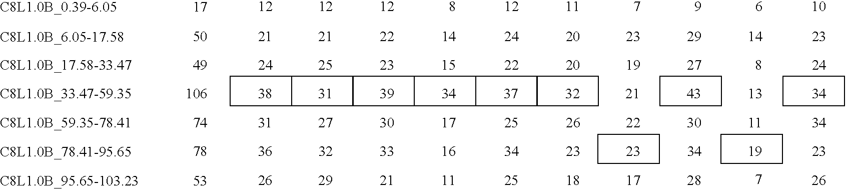

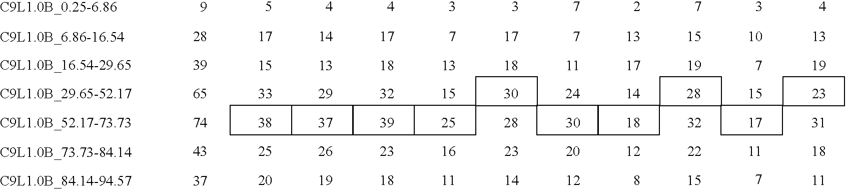

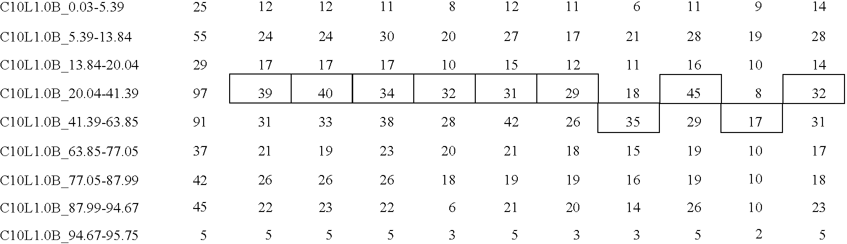

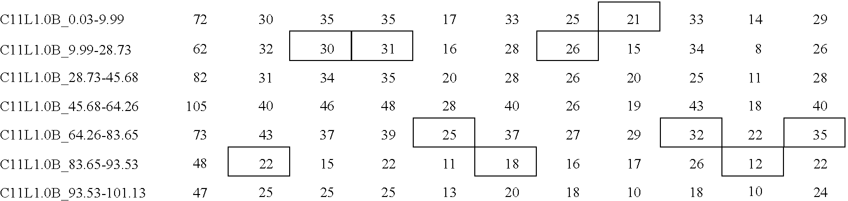

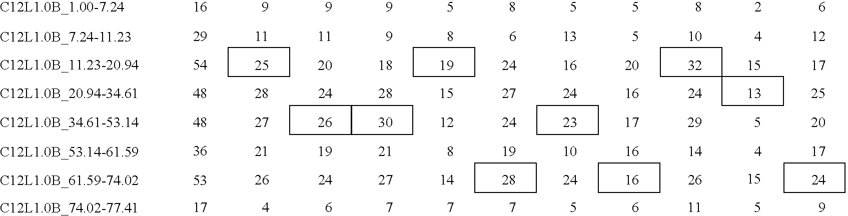

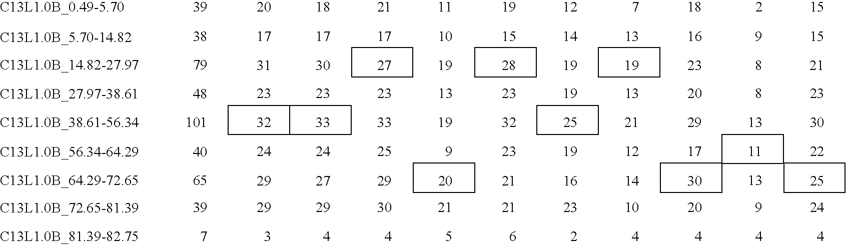

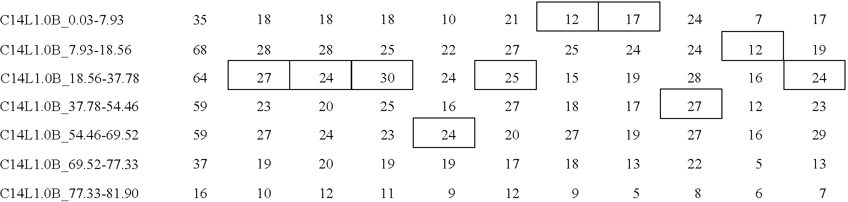

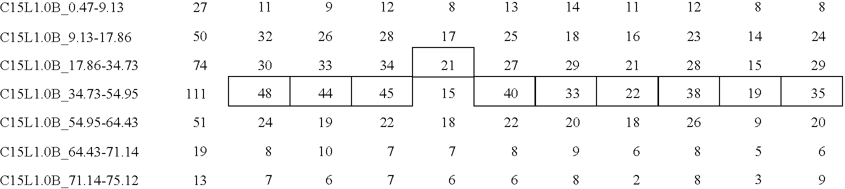

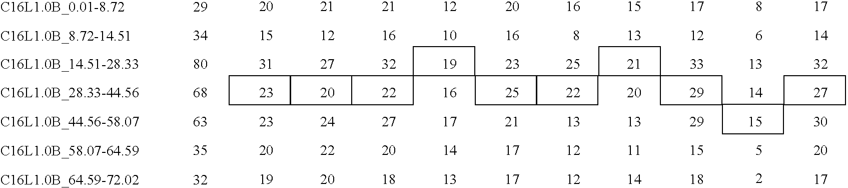

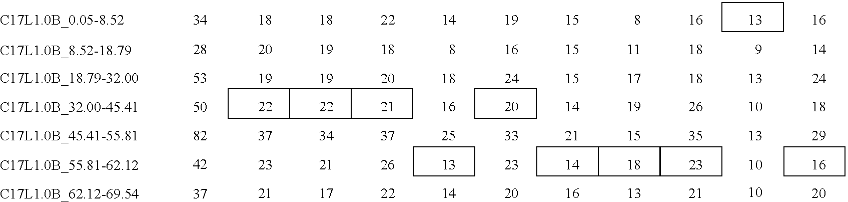

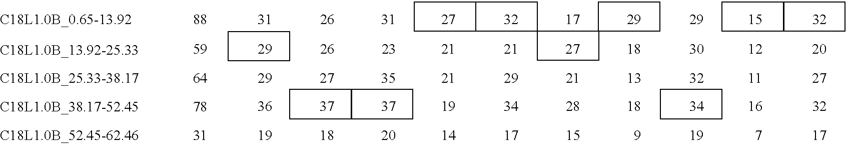

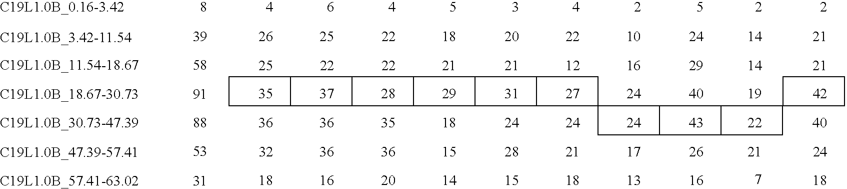

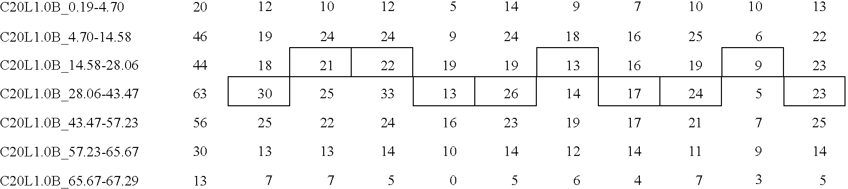

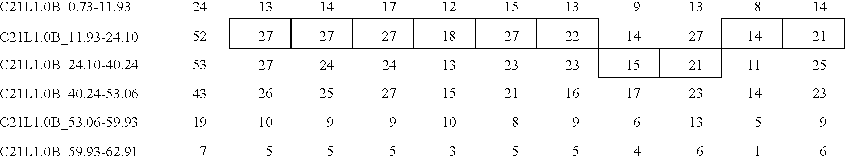

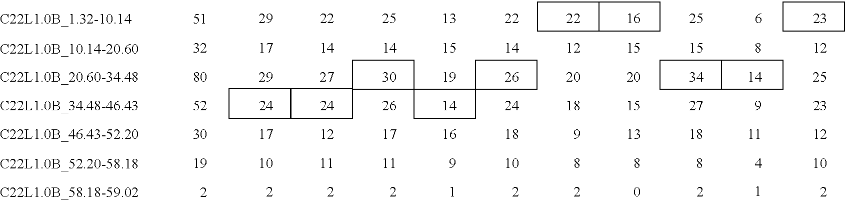

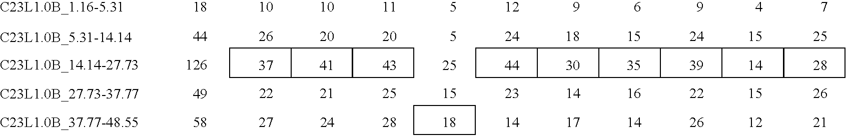

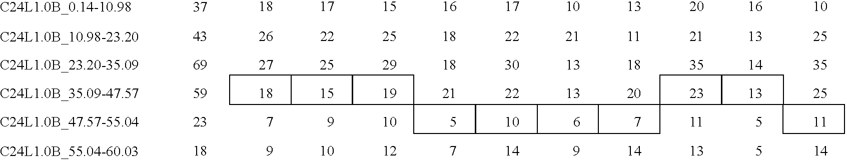

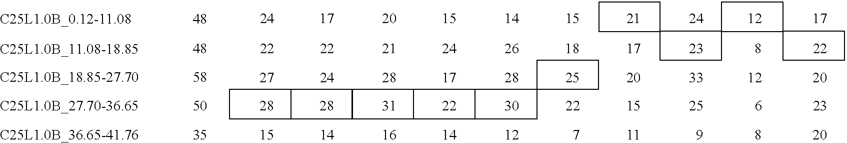

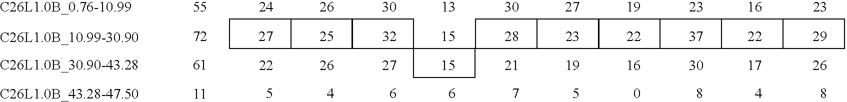

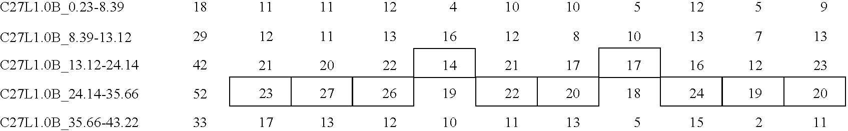

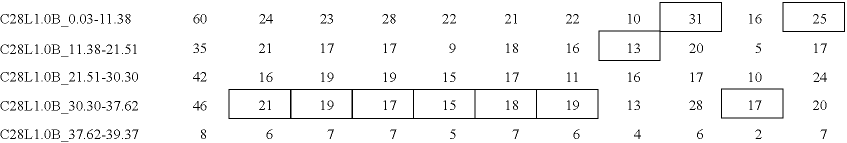

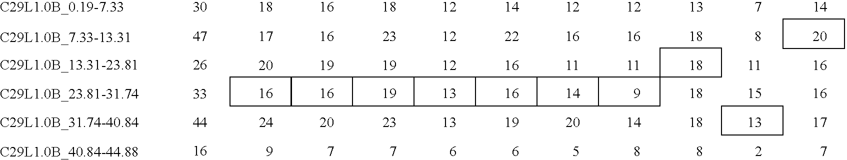

TABLE 5

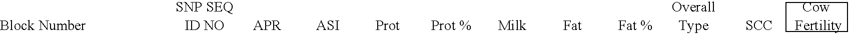

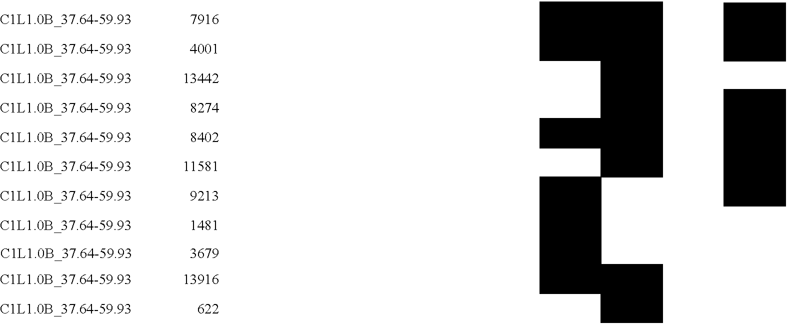

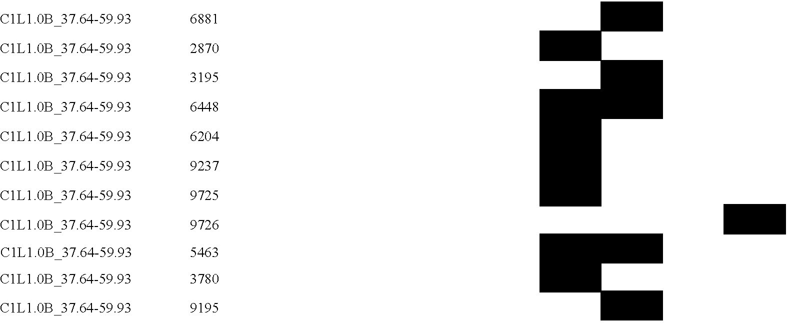

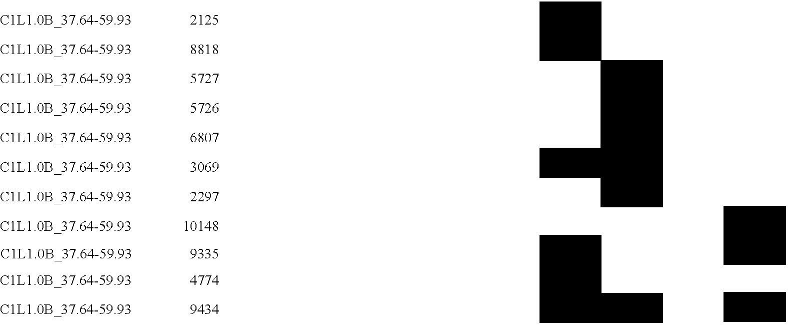

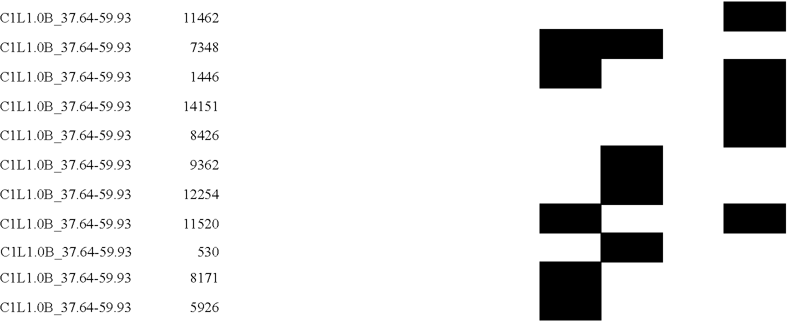

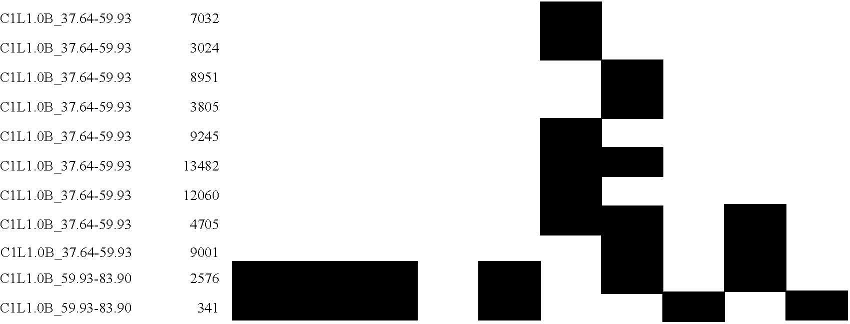

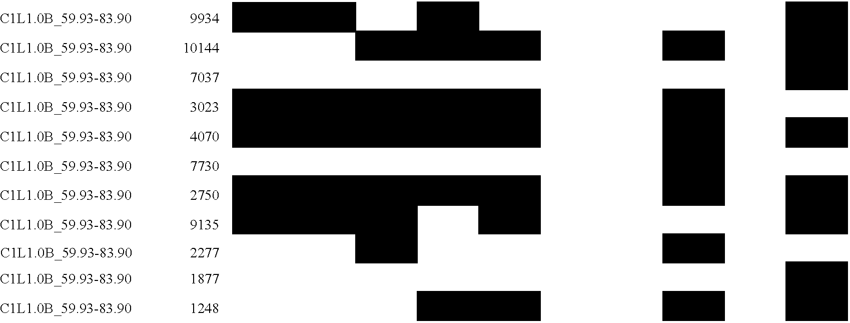

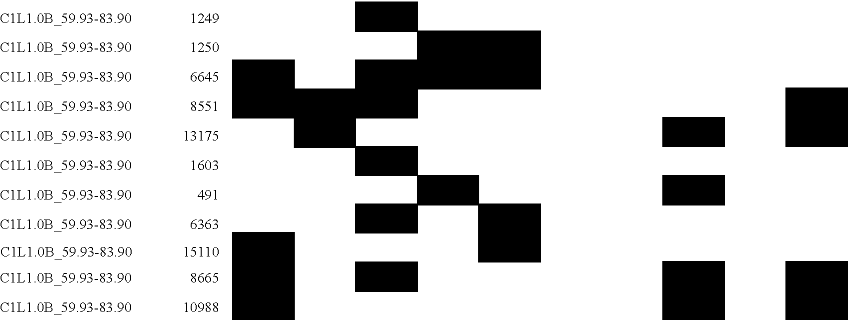

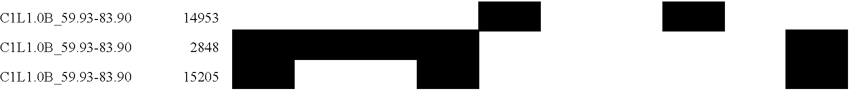

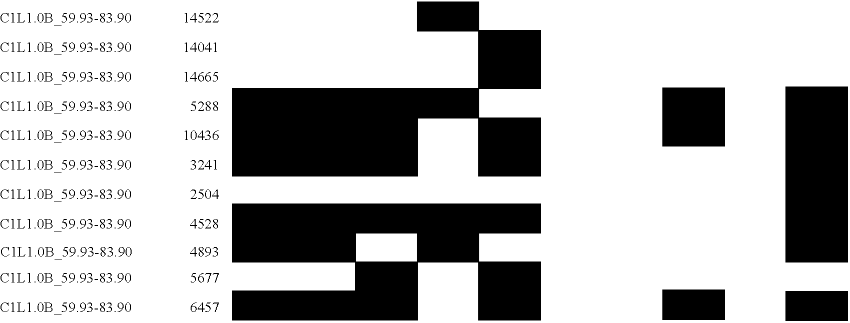

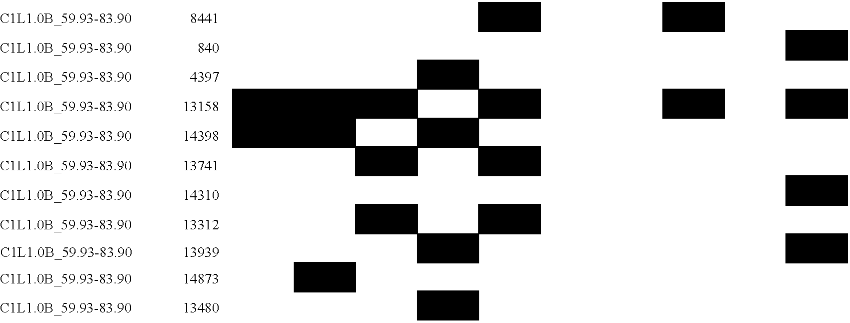

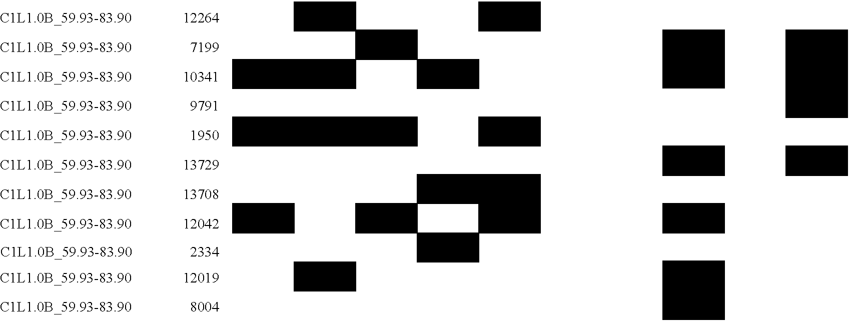

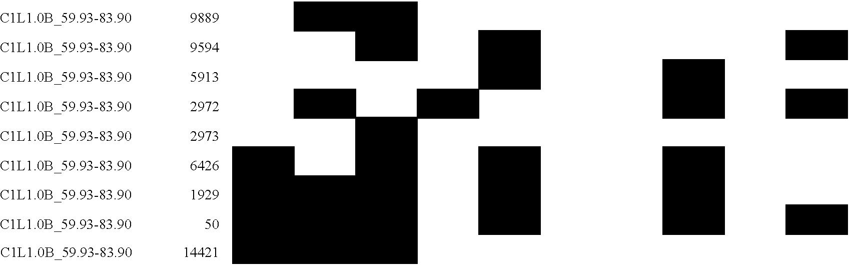

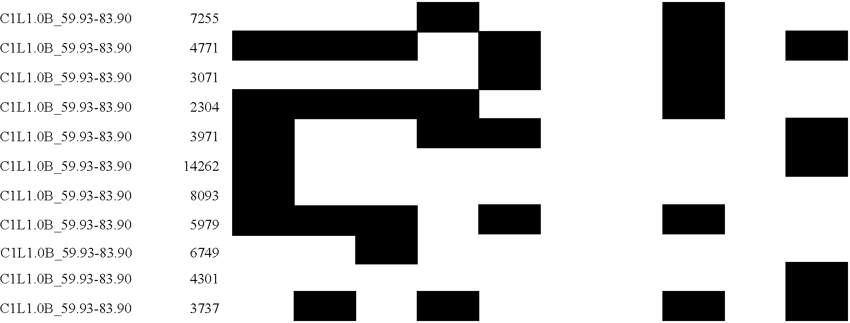

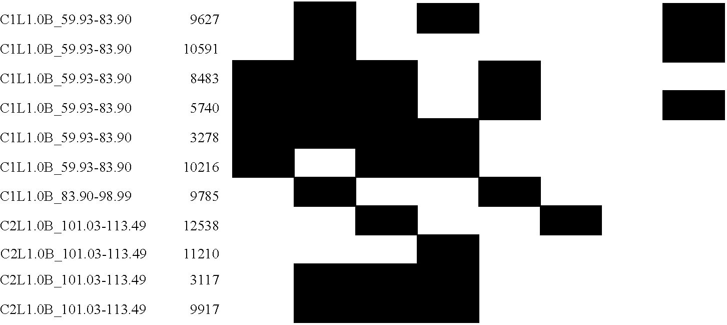

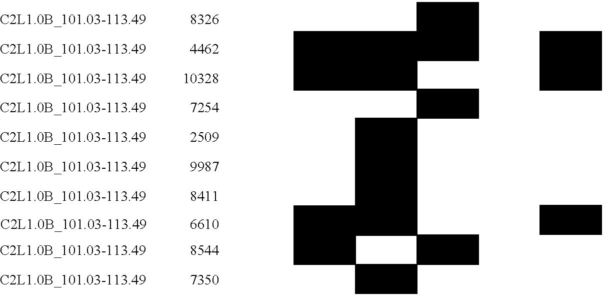

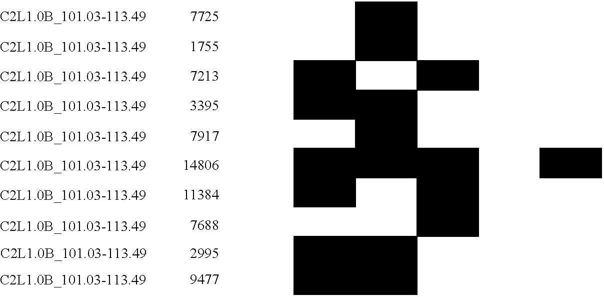

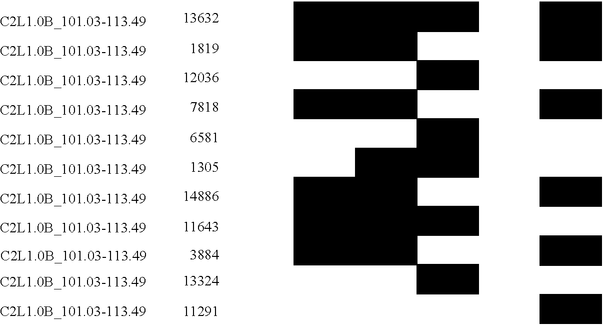

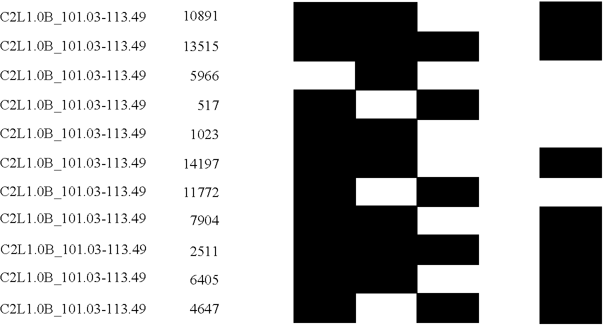

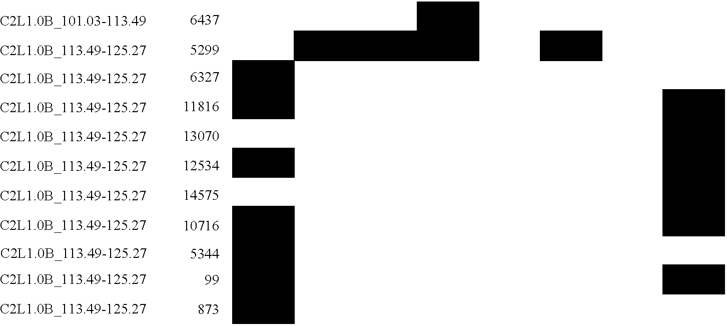

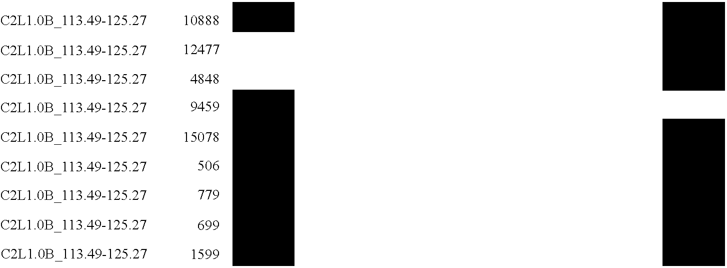

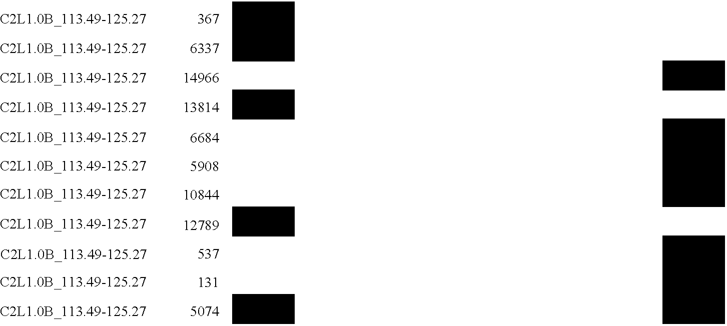

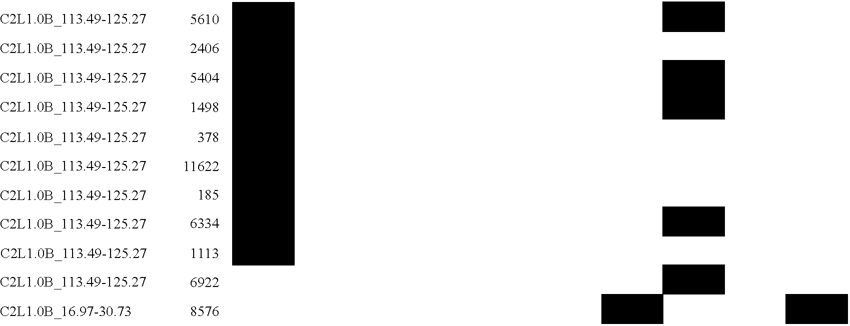

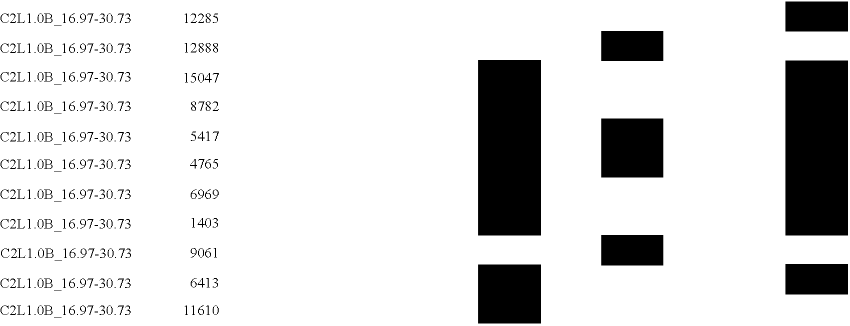

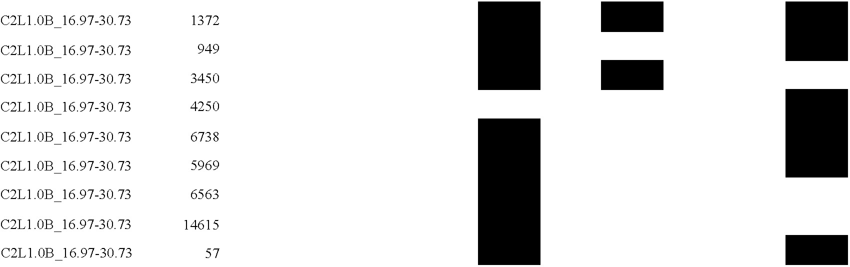

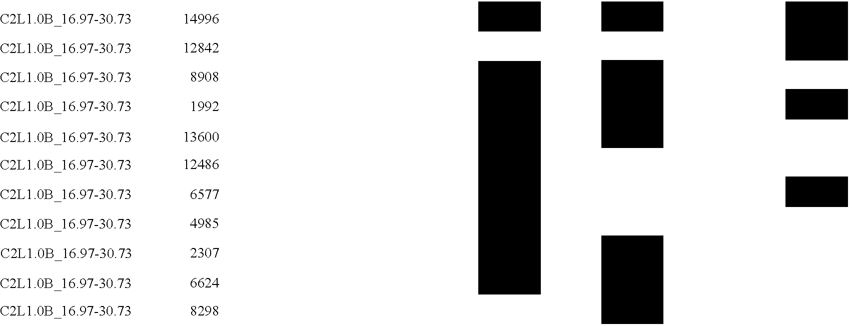

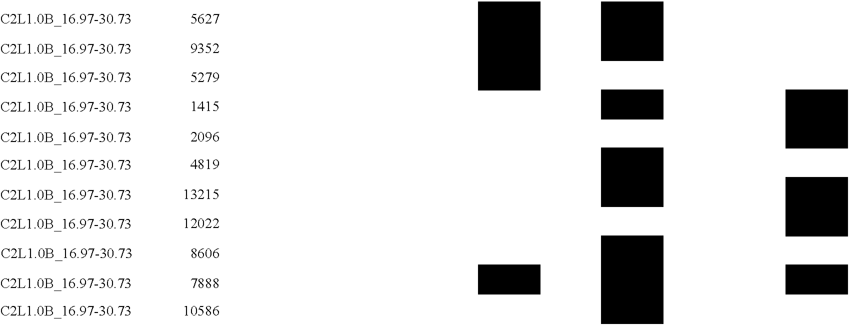

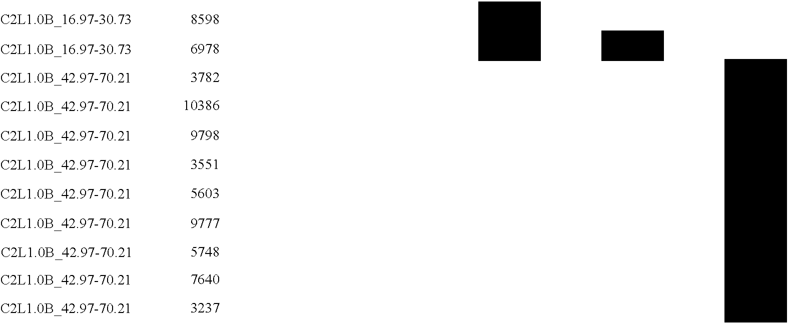

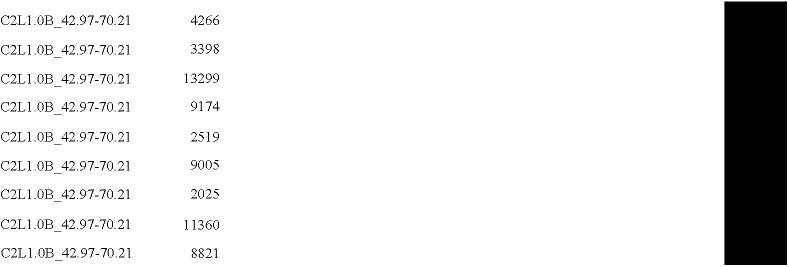

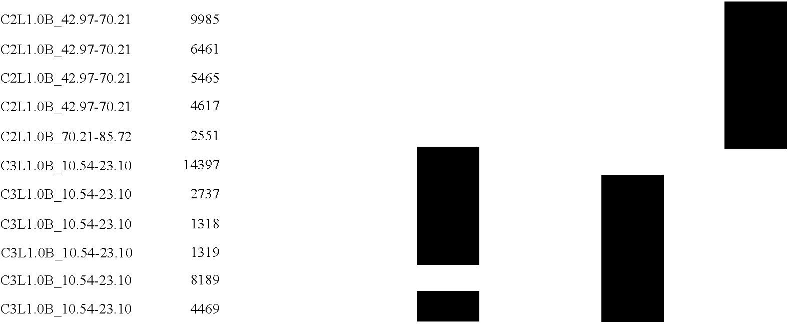

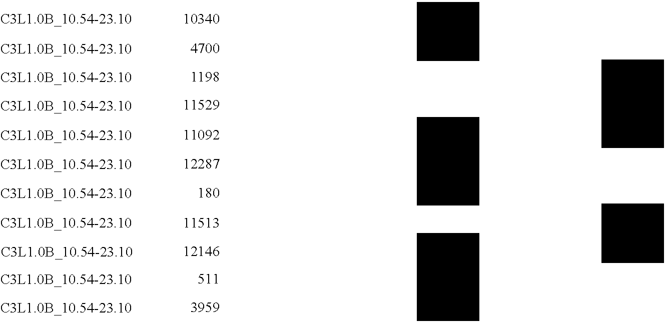

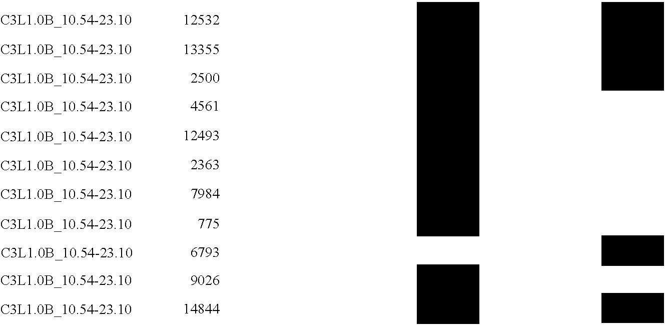

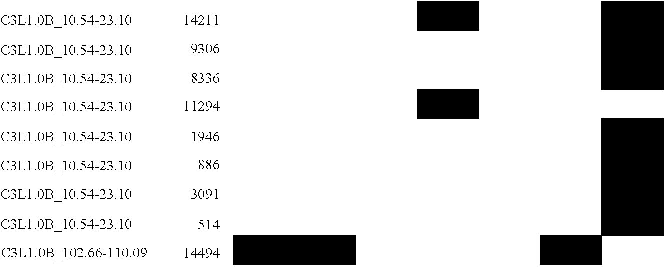

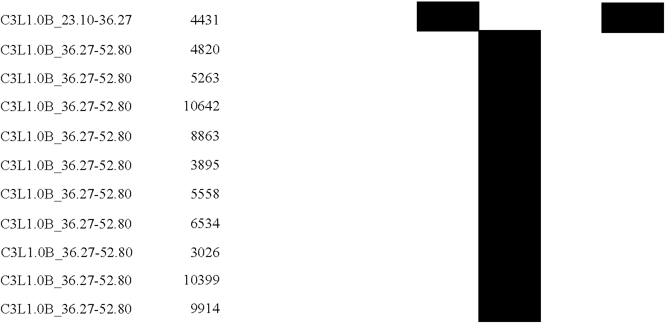

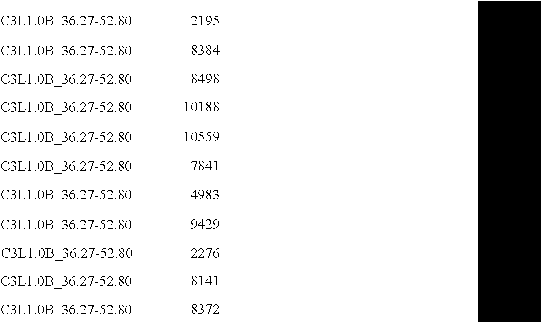

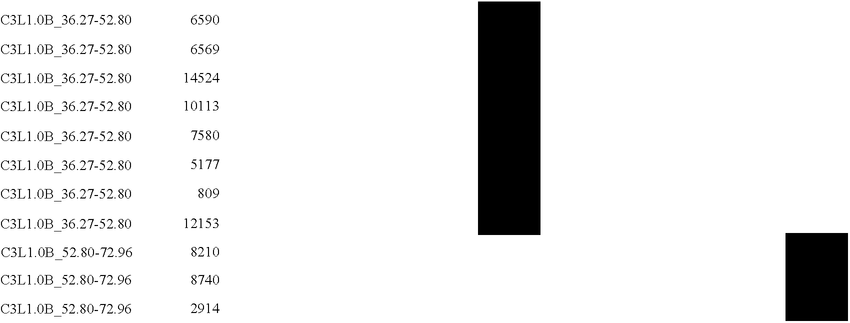

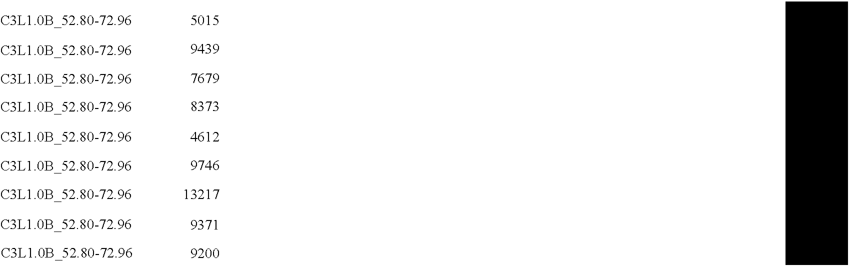

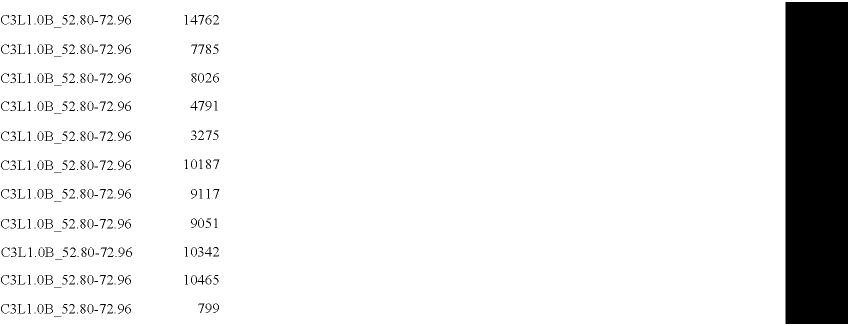

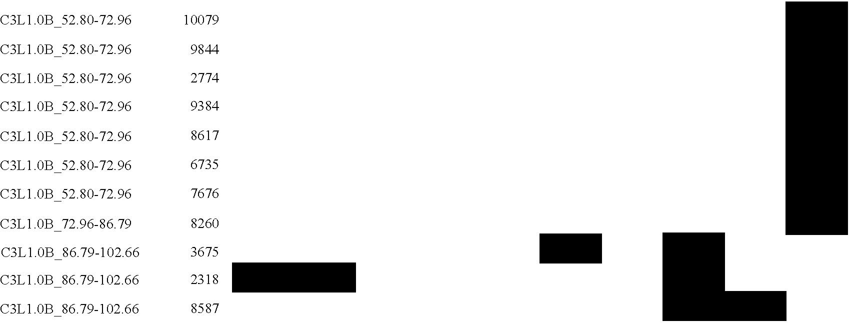

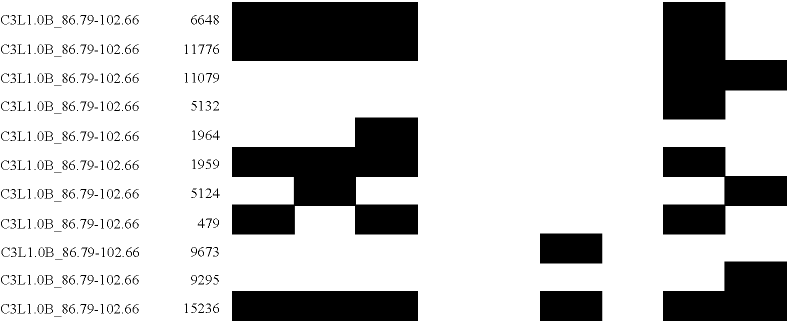

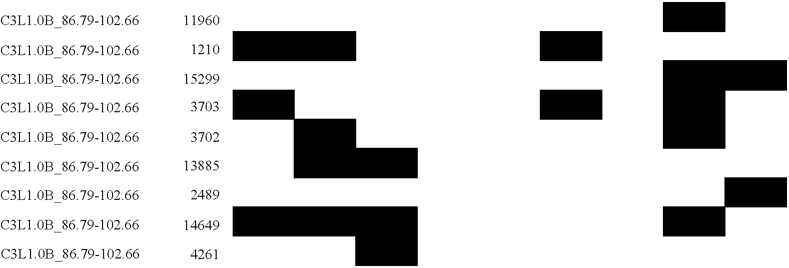

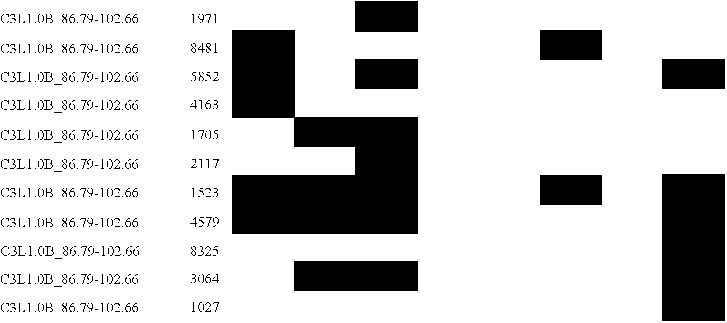

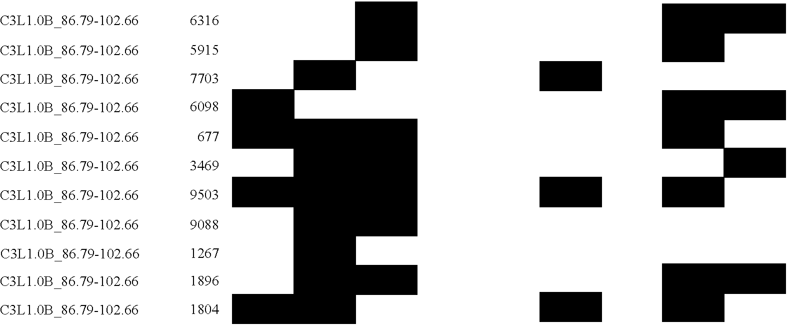

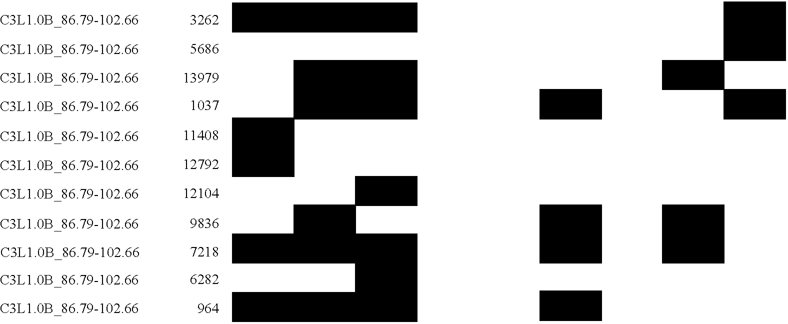

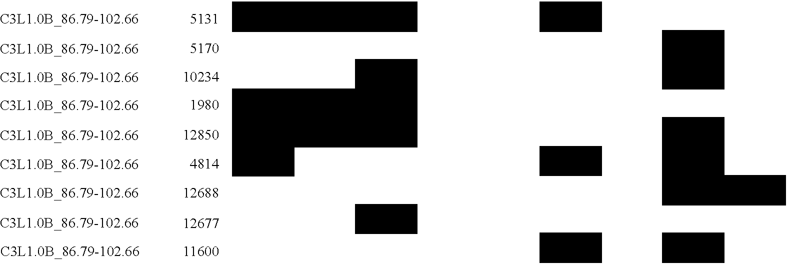

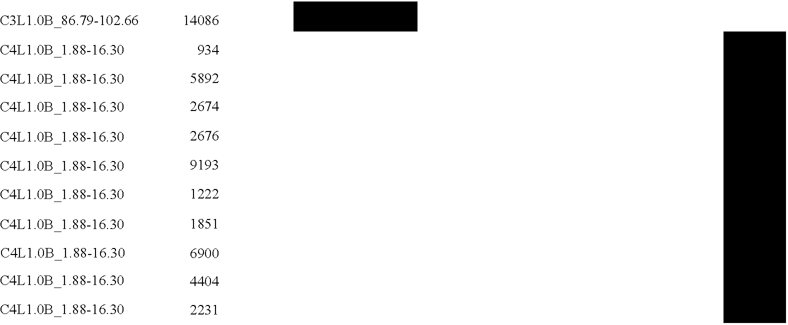

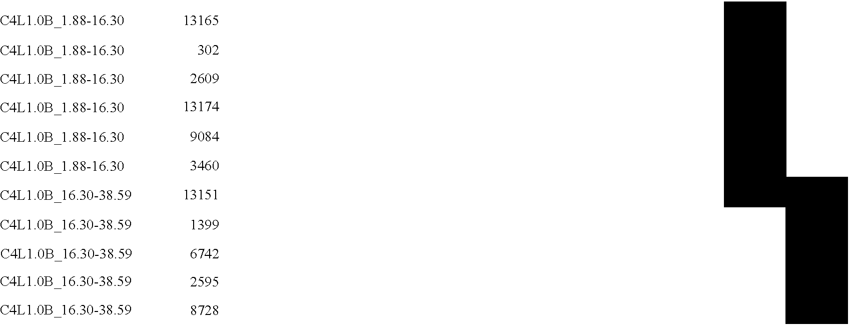

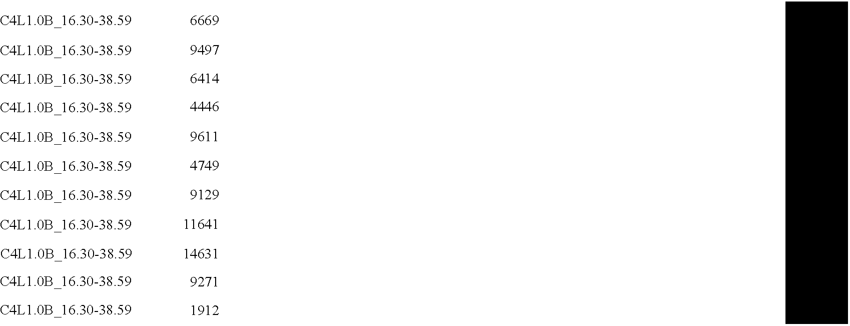

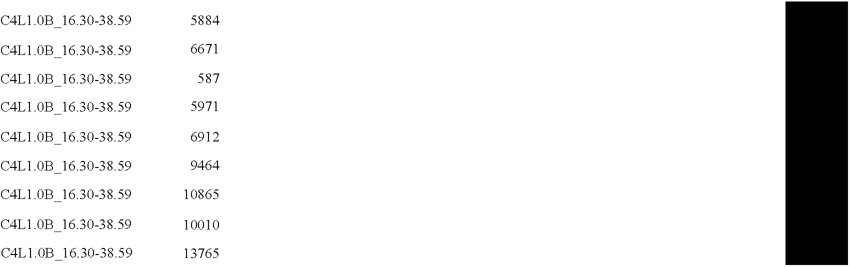

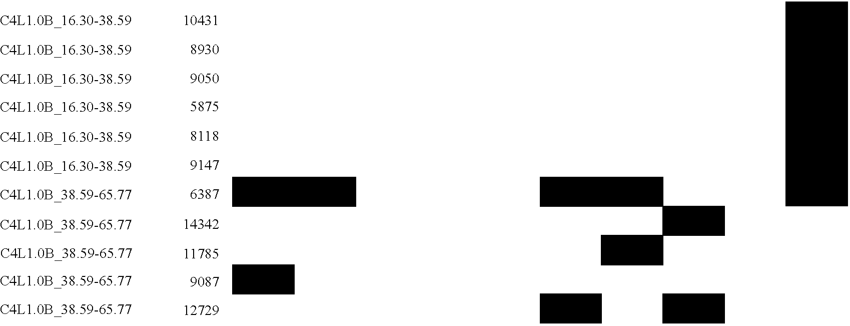

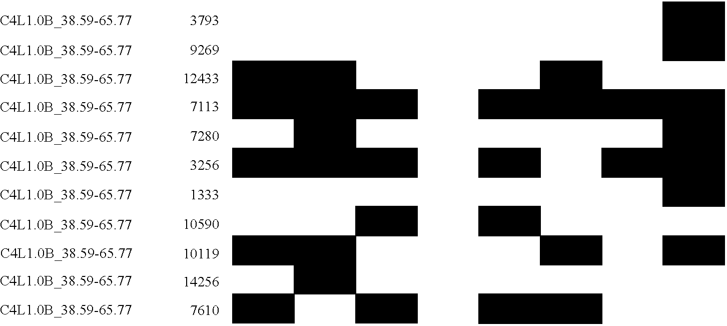

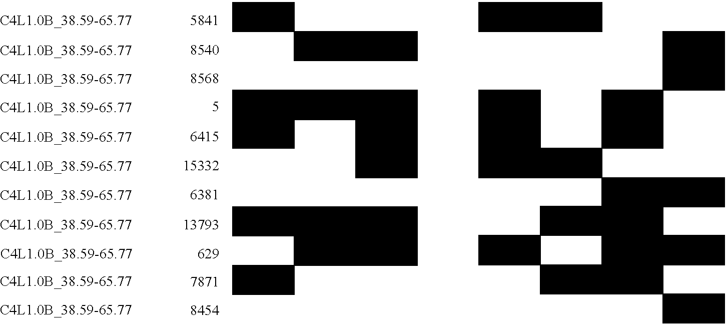

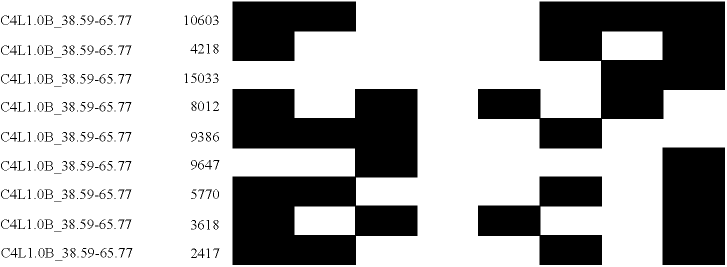

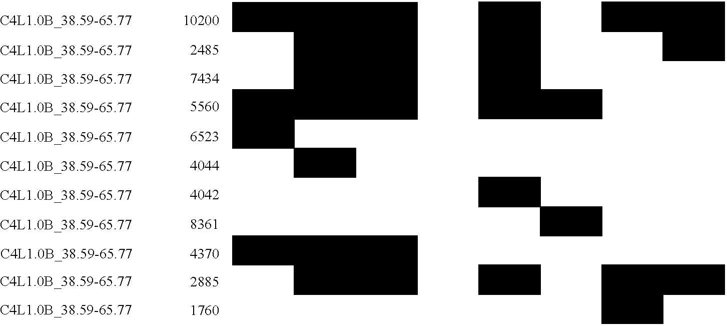

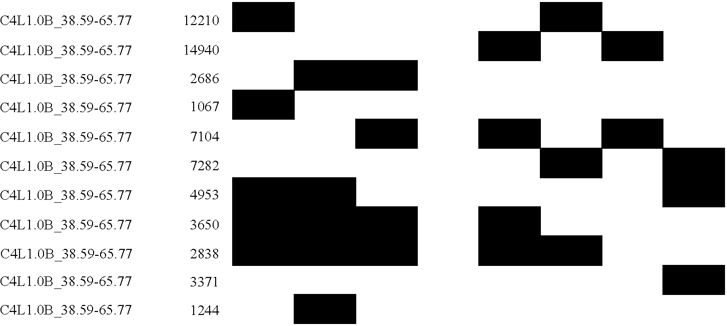

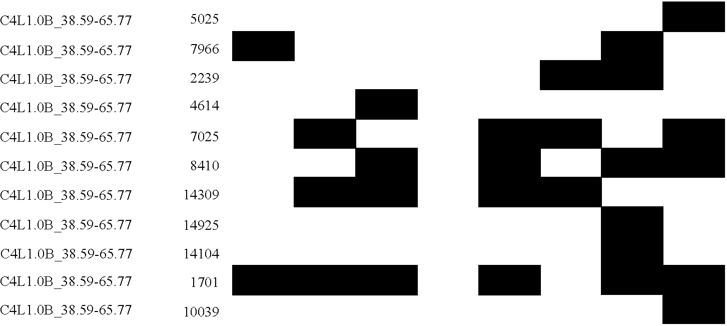

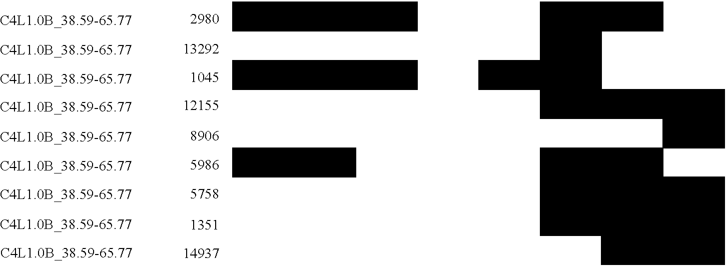

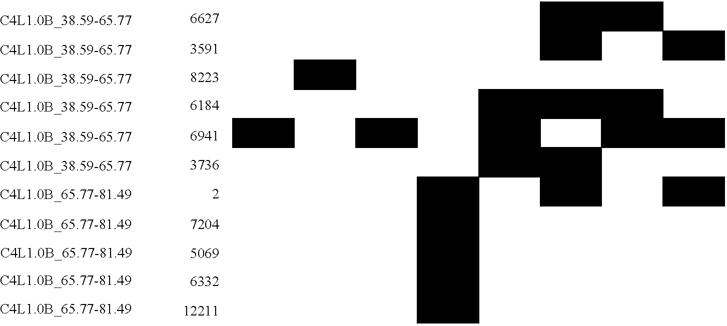

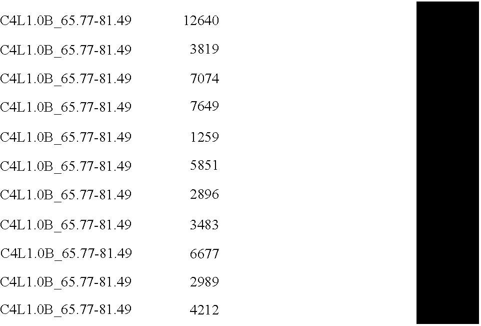

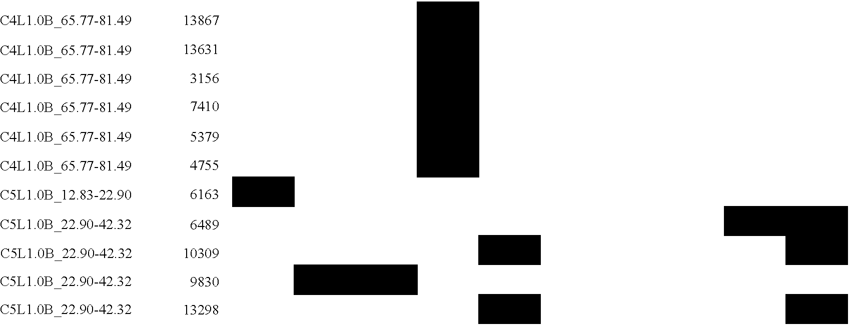

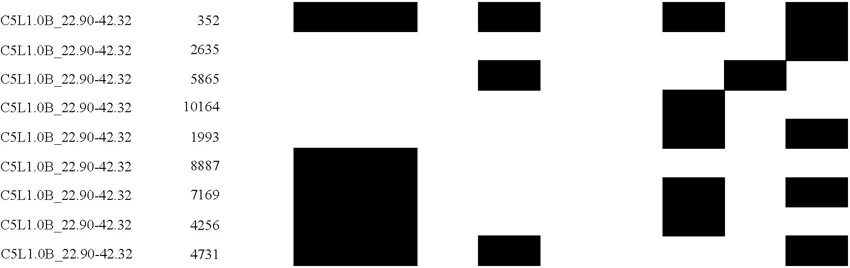

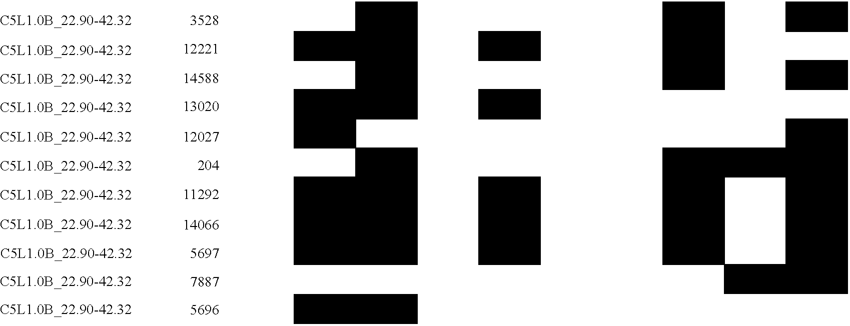

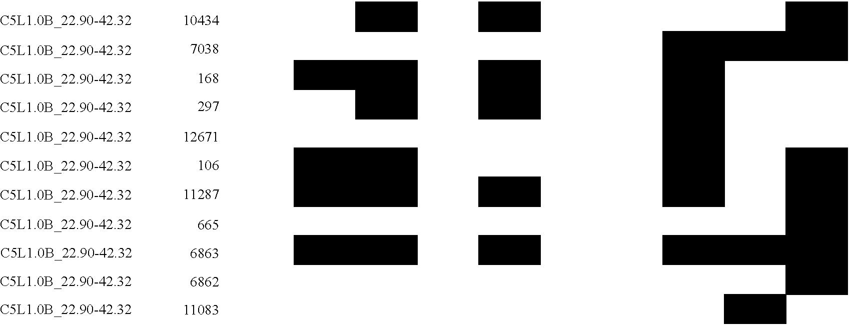

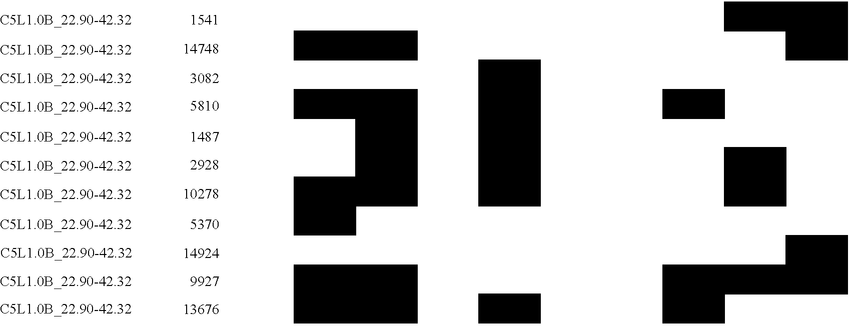

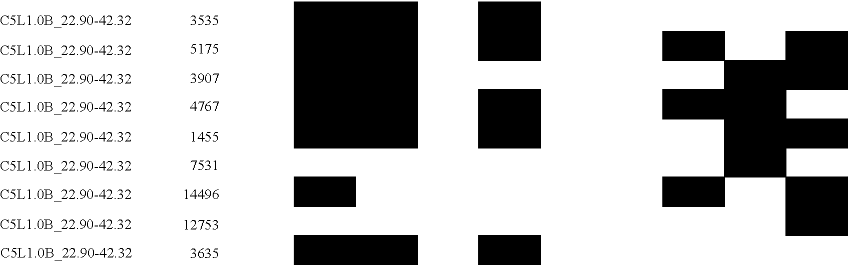

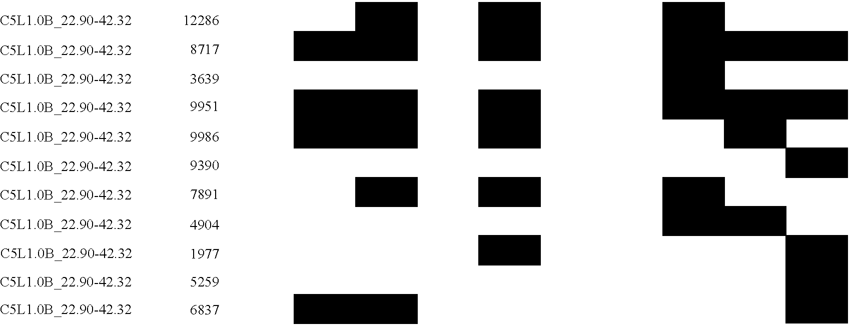

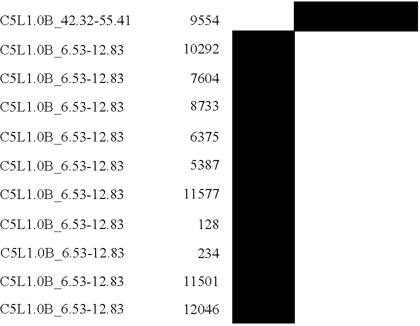

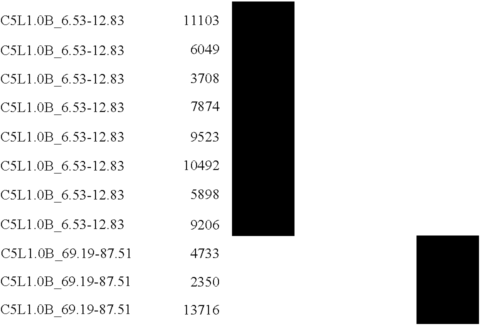

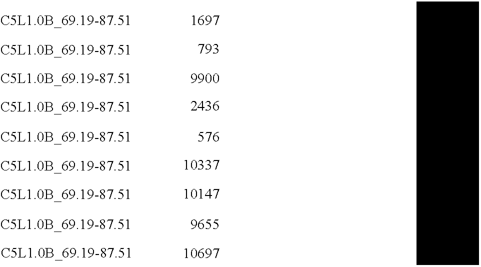

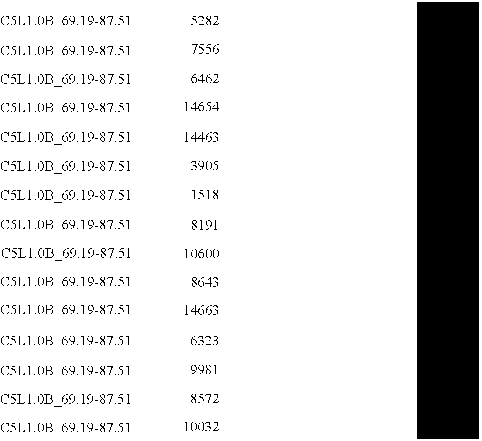

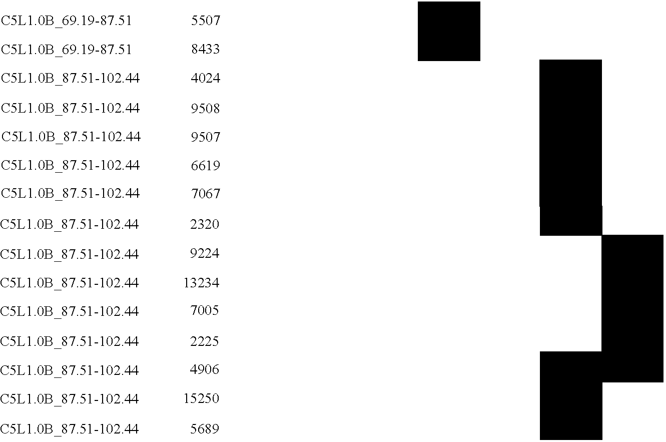

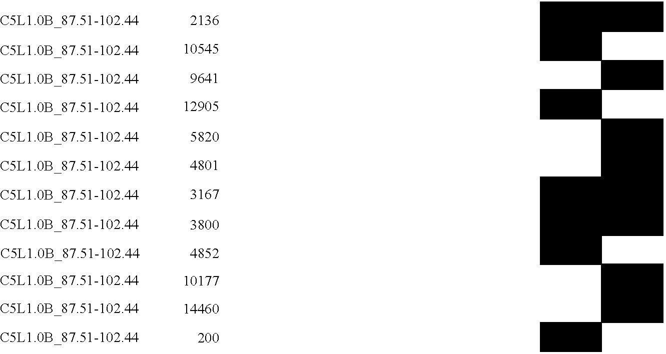

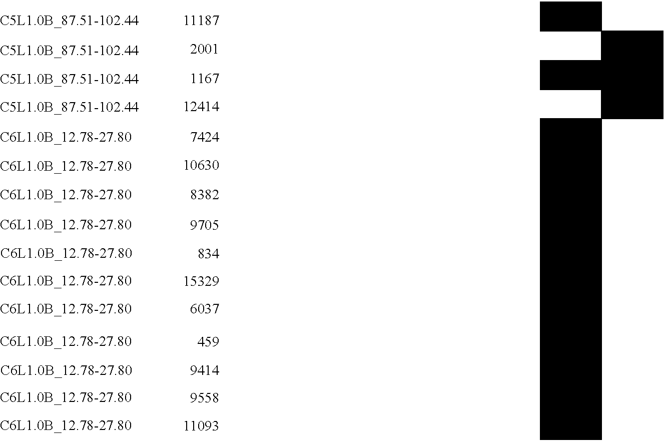

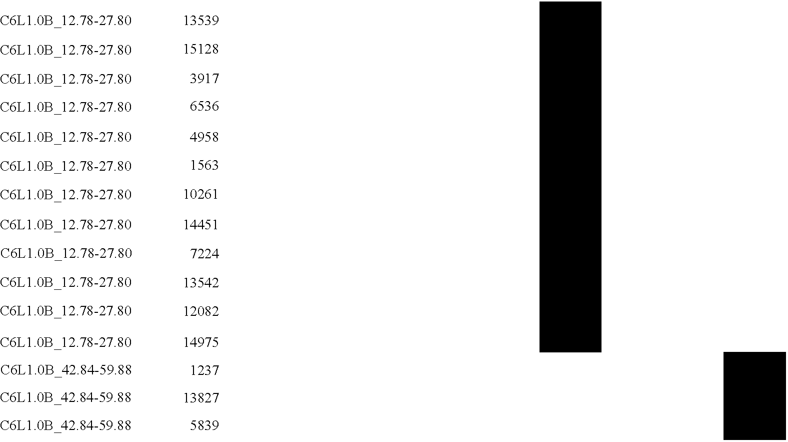

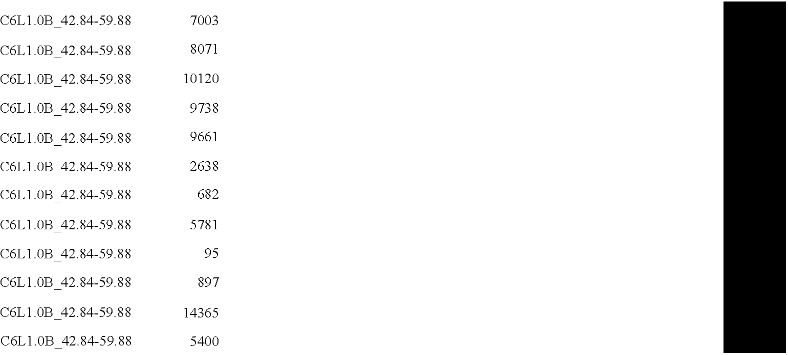

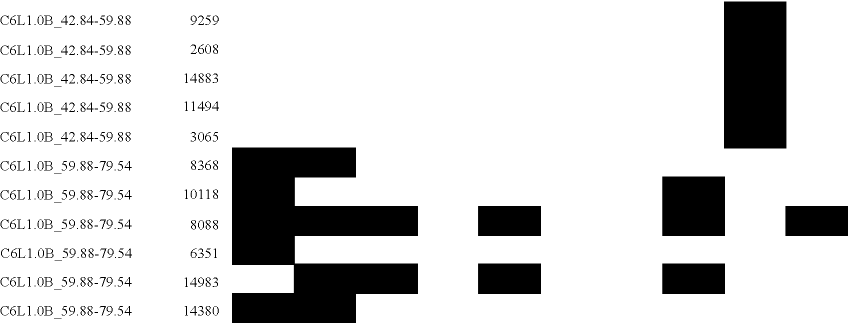

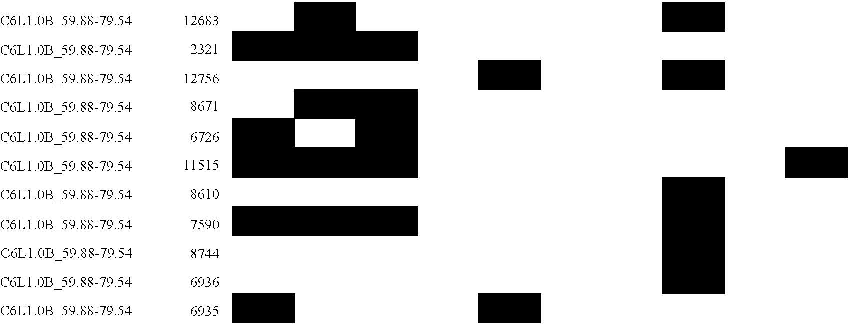

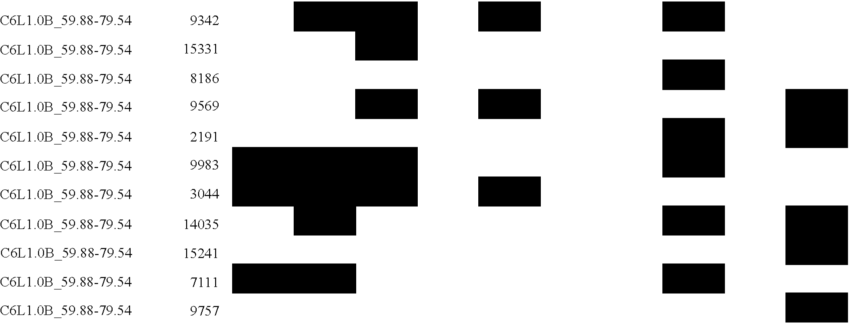

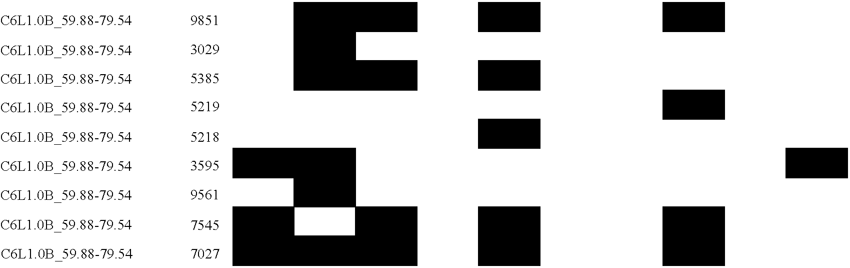

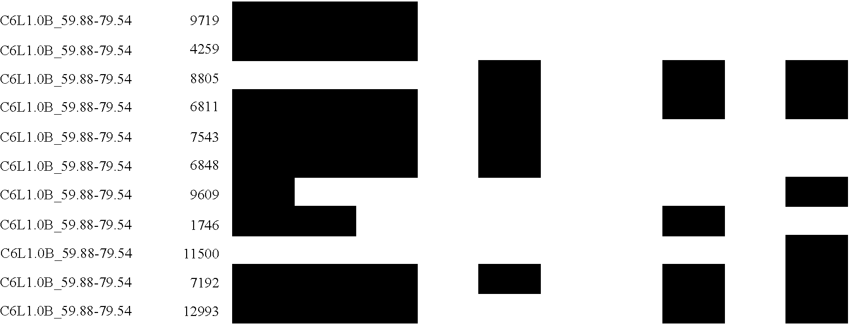

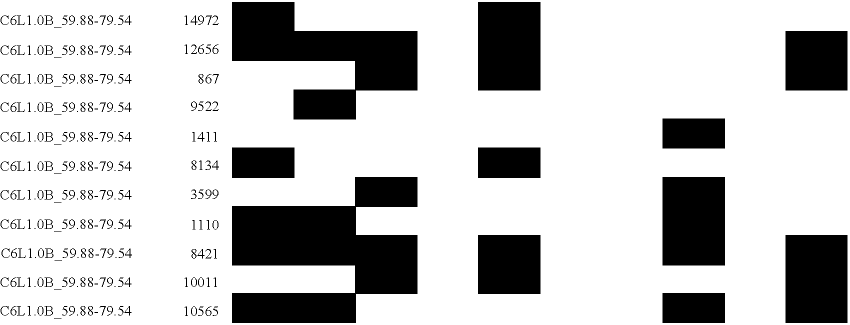

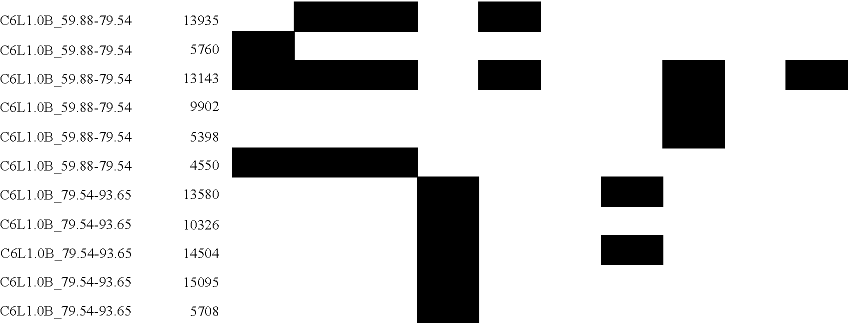

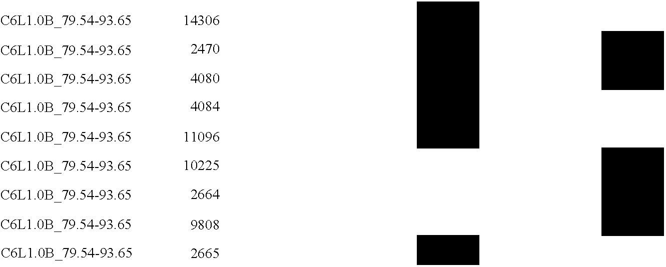

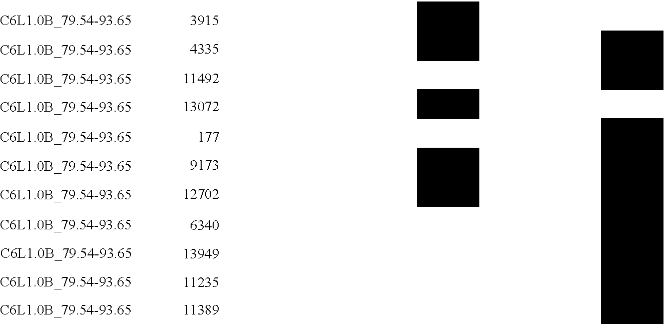

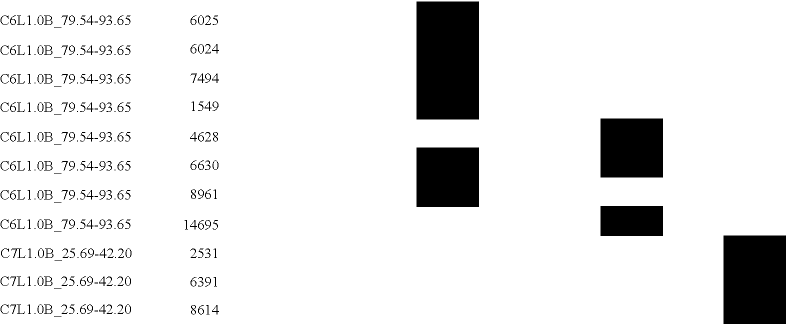

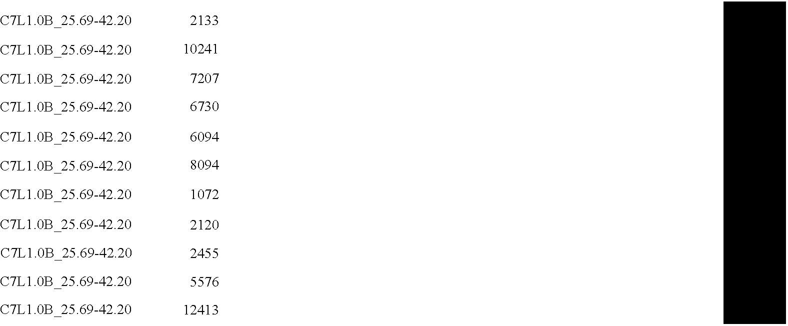

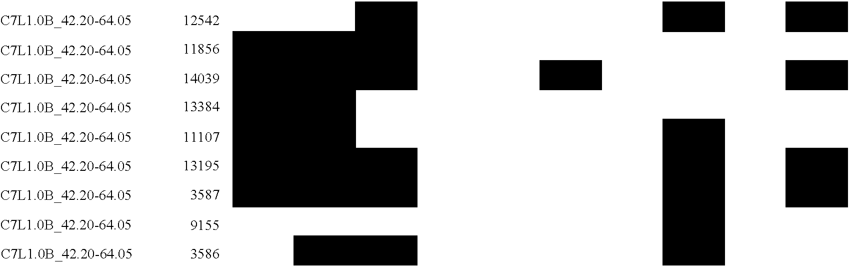

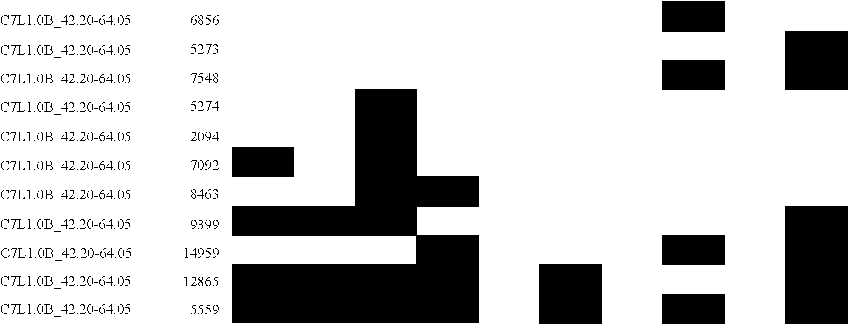

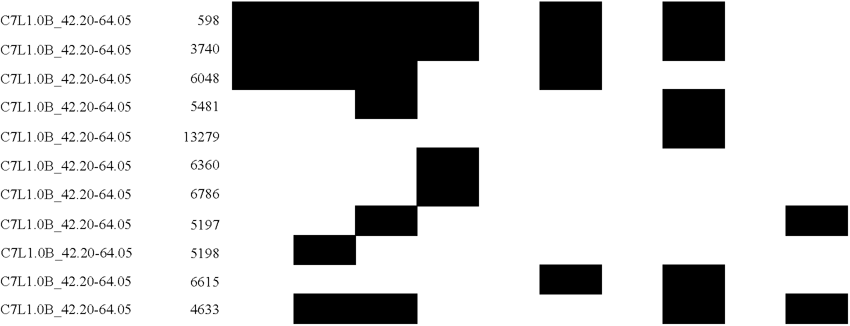

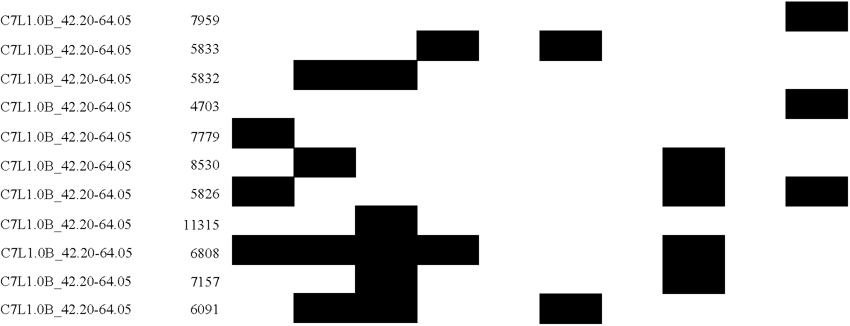

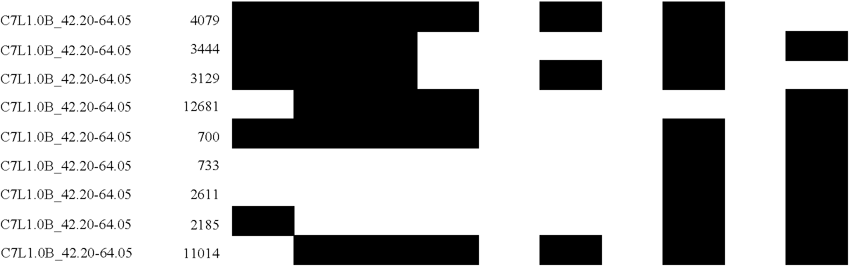

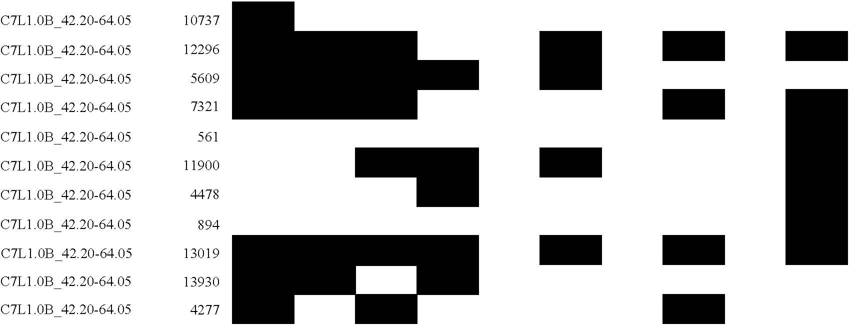

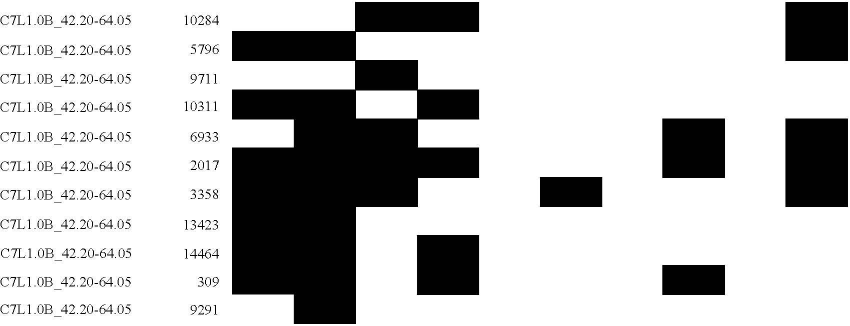

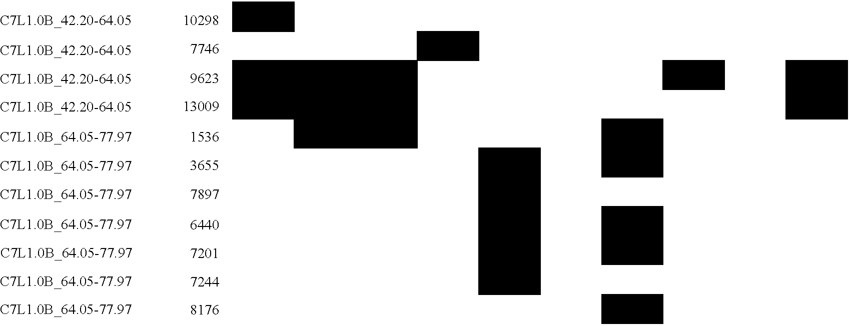

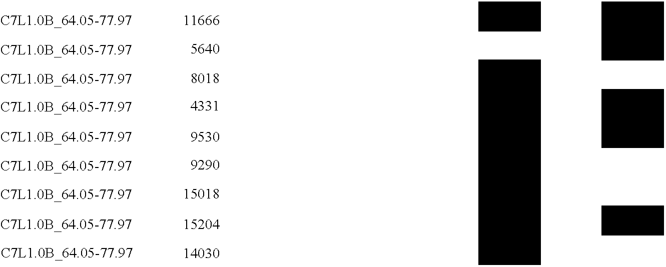

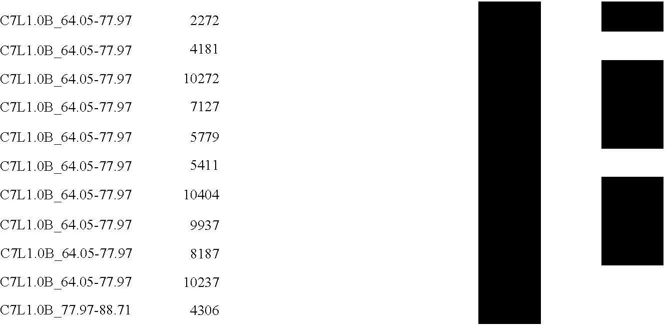

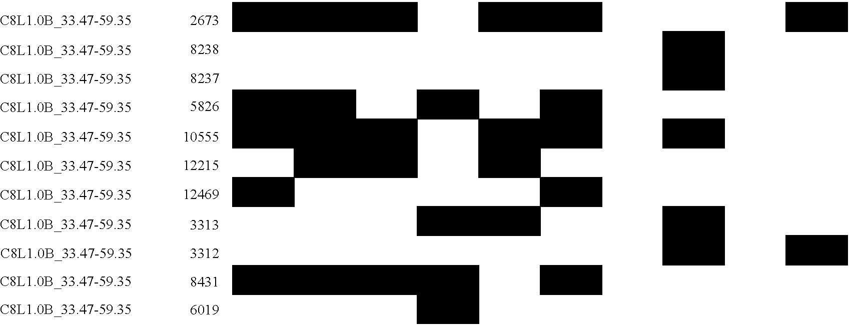

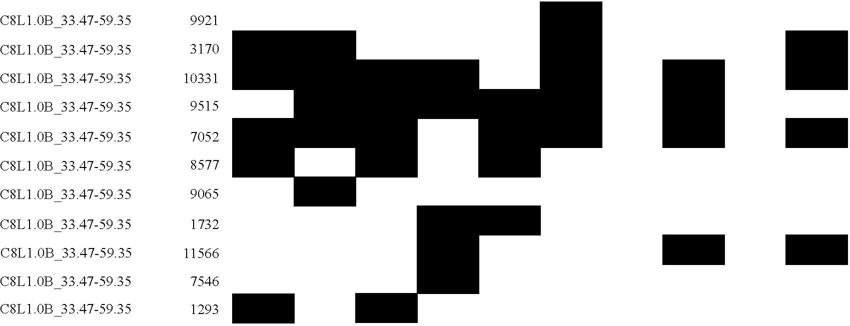

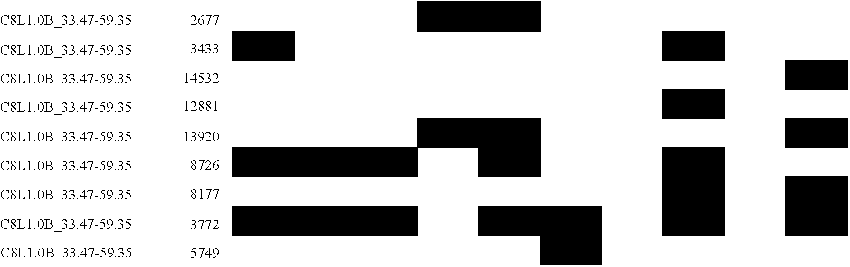

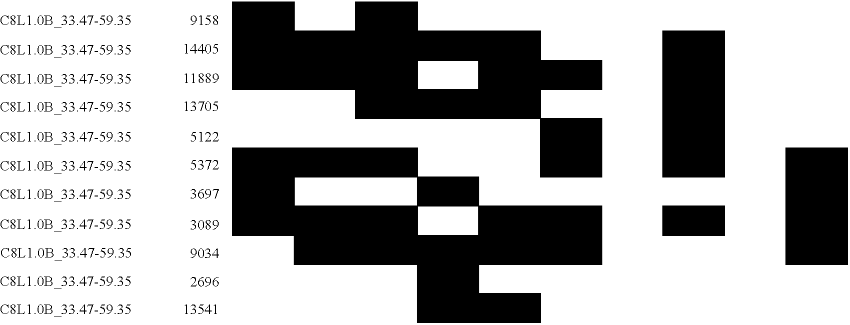

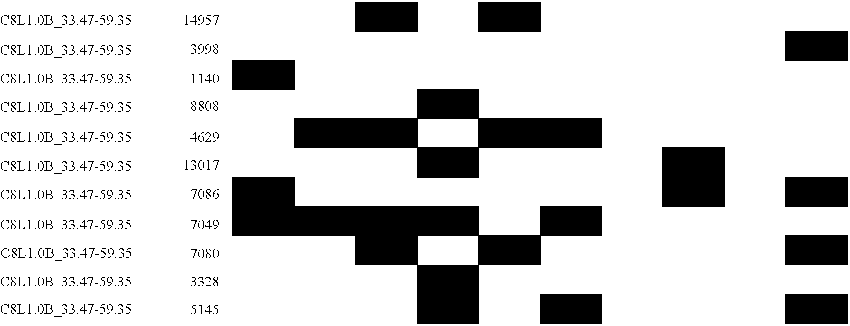

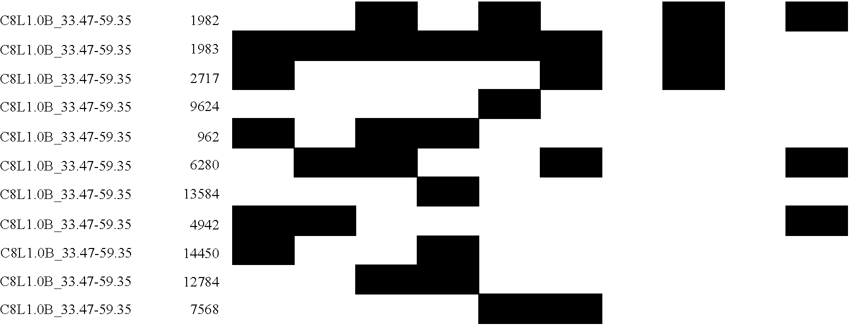

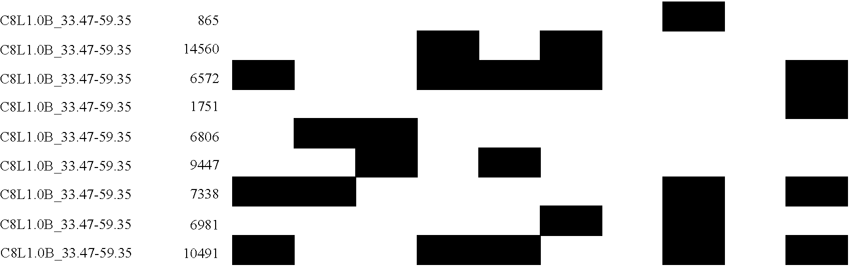

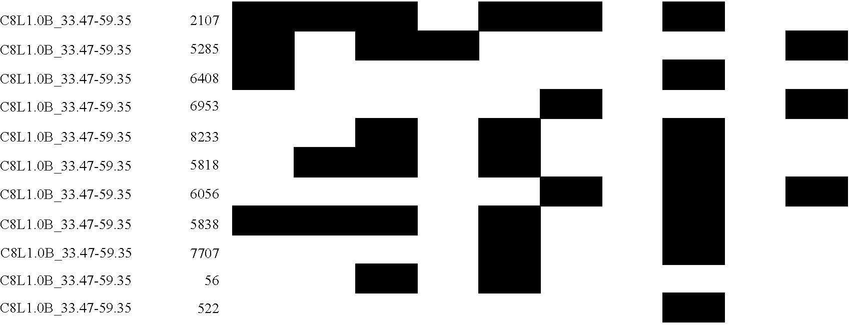

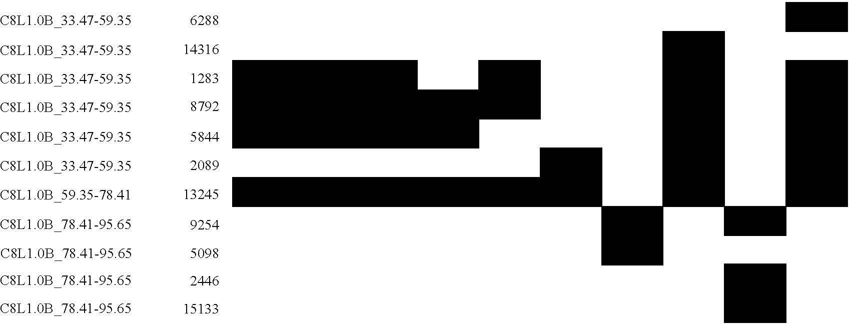

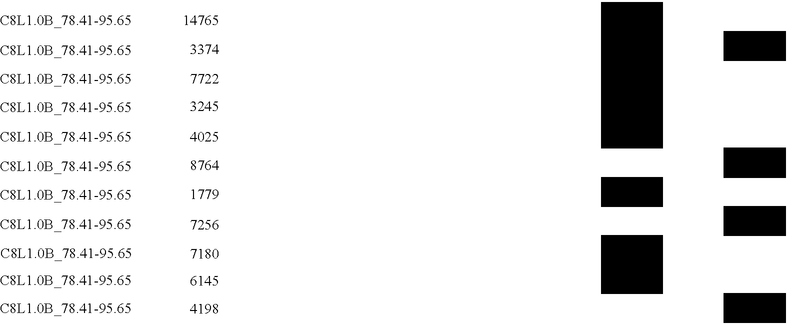

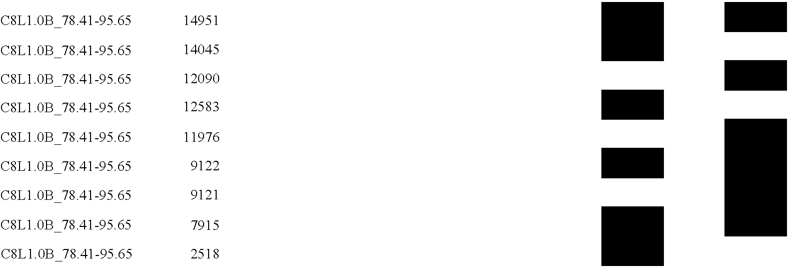

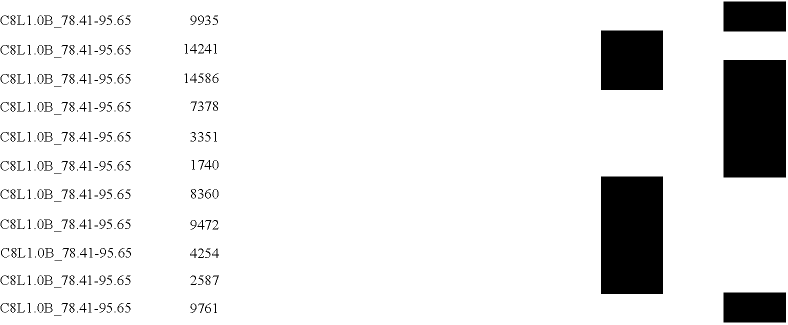

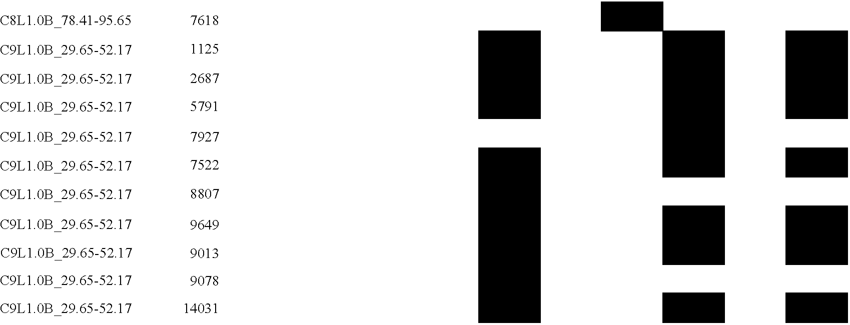

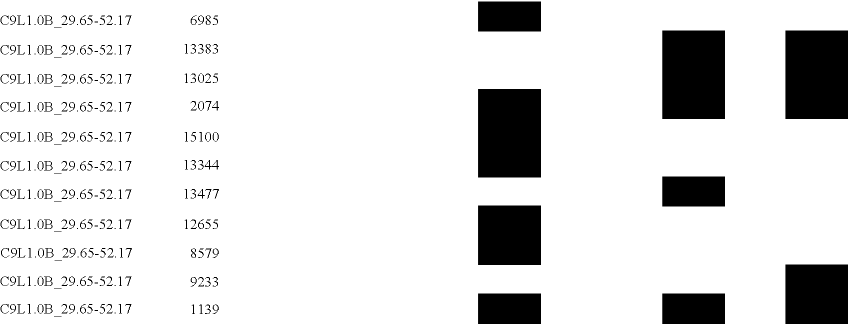

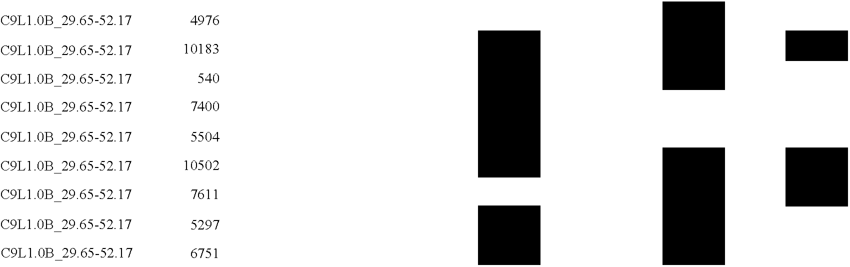

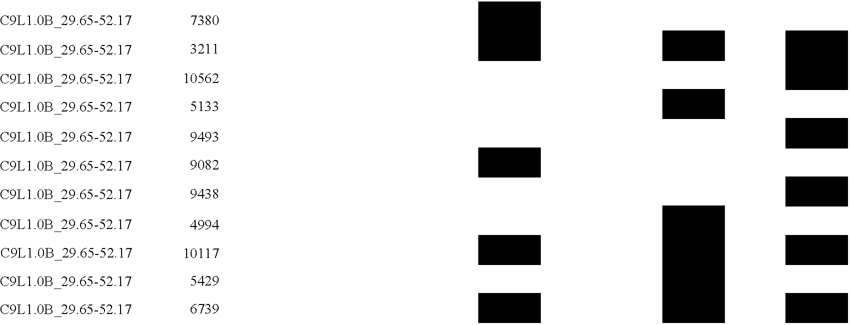

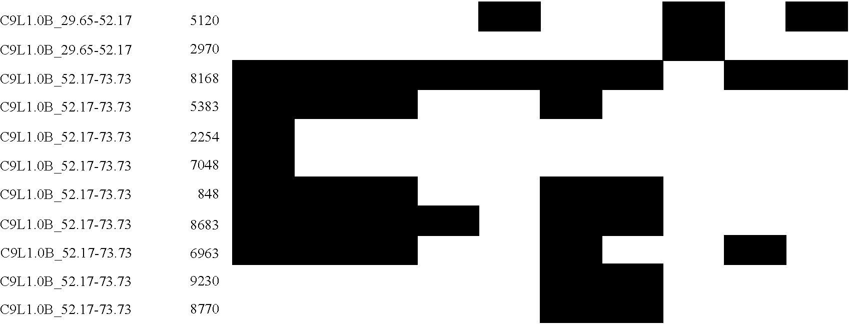

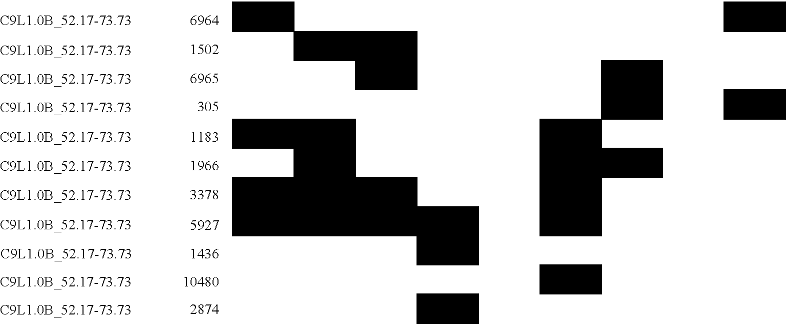

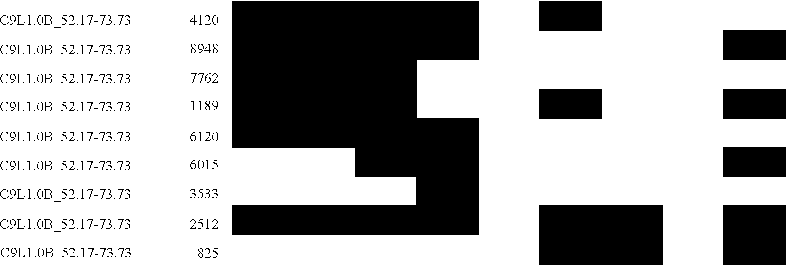

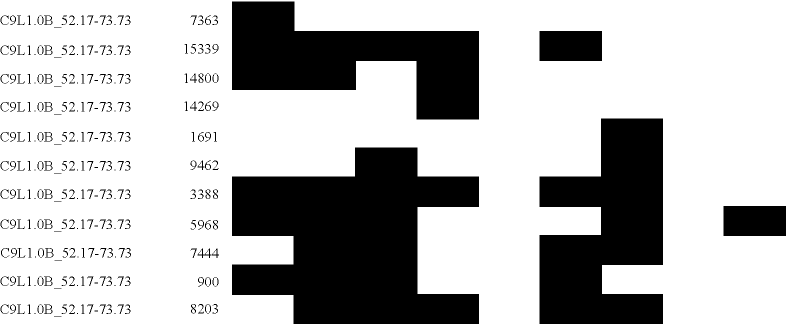

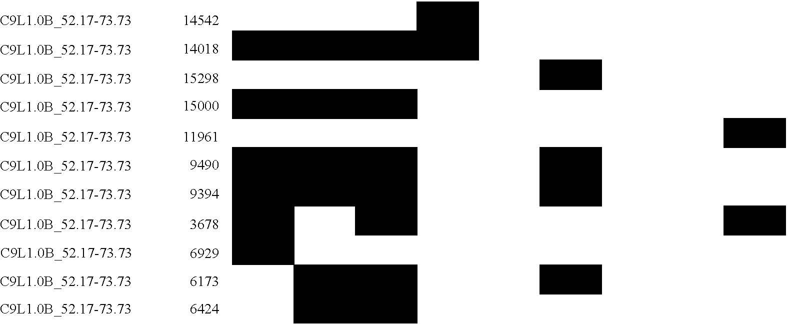

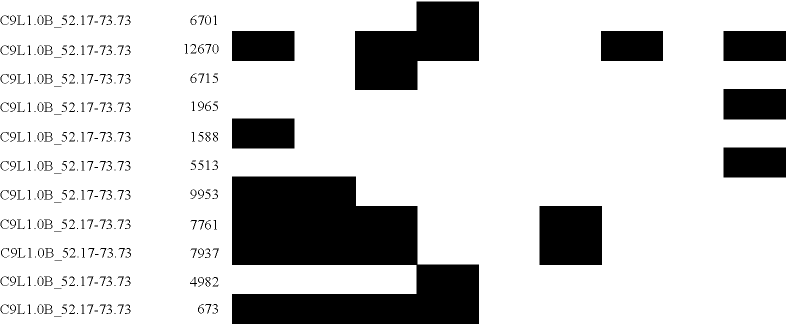

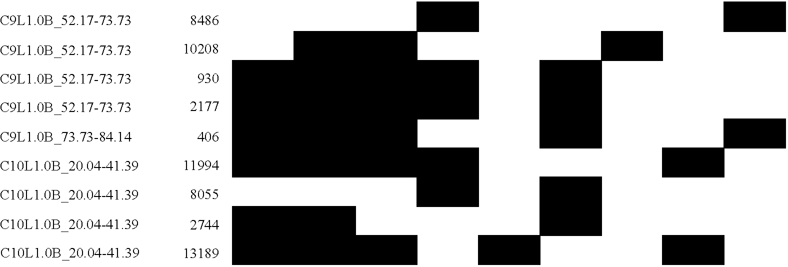

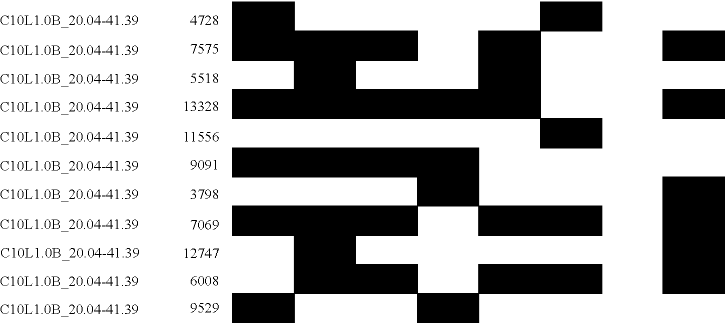

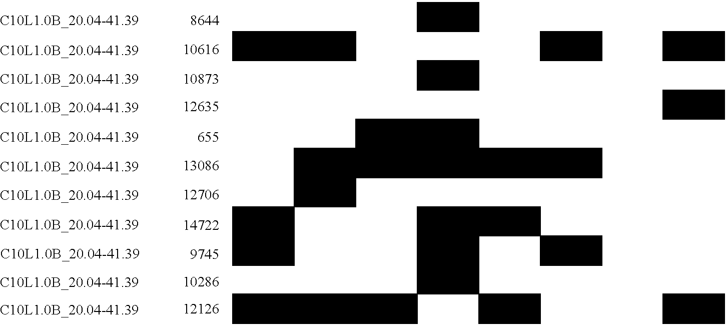

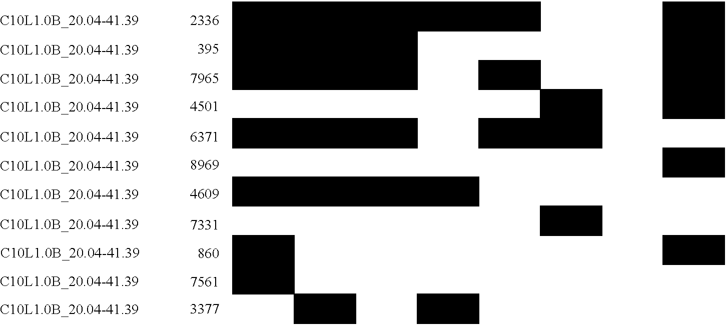

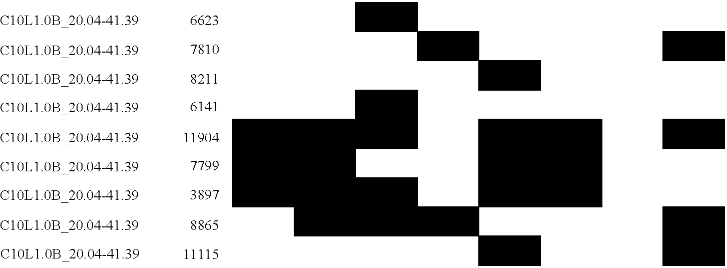

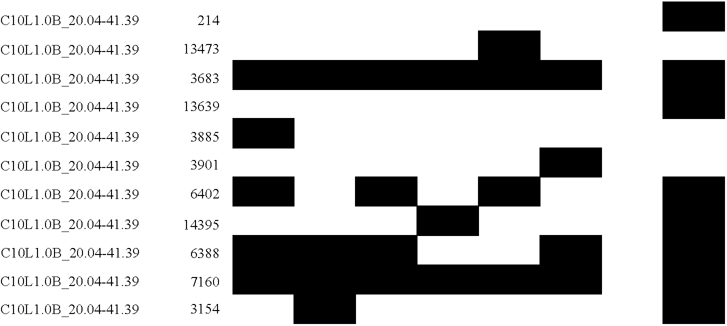

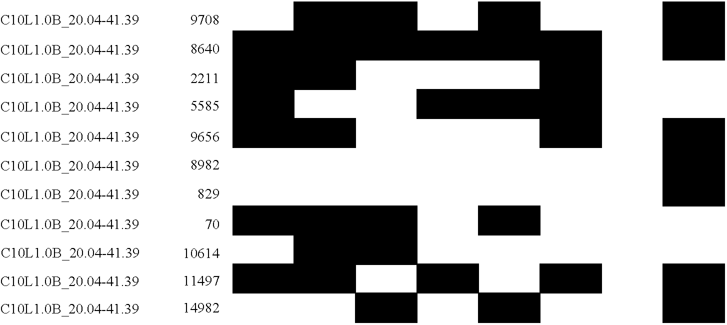

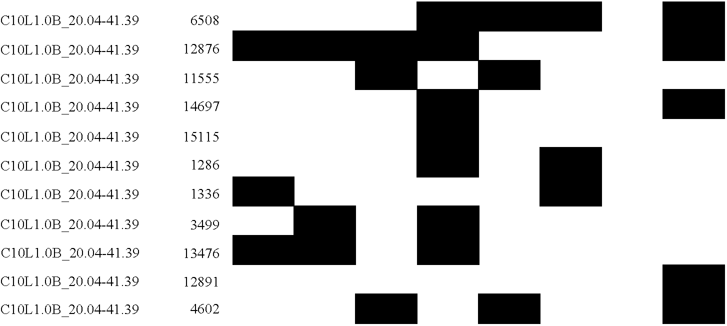

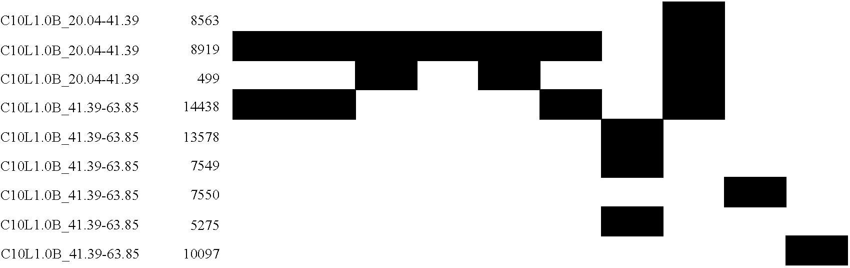

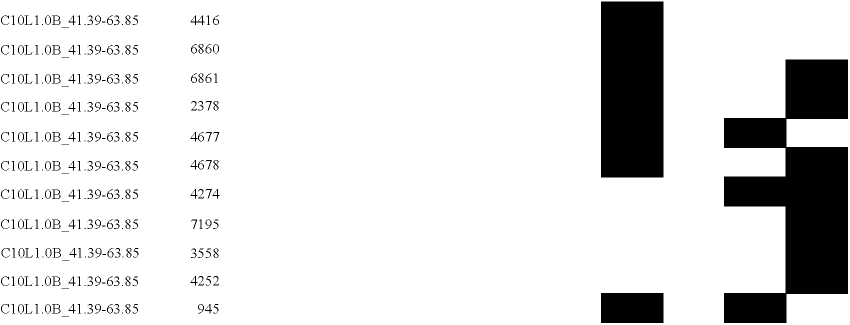

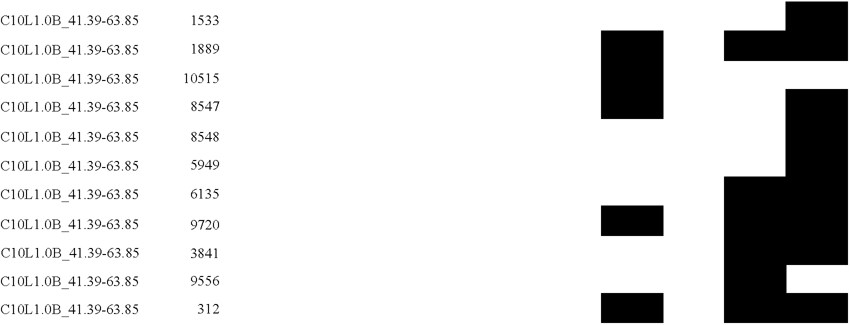

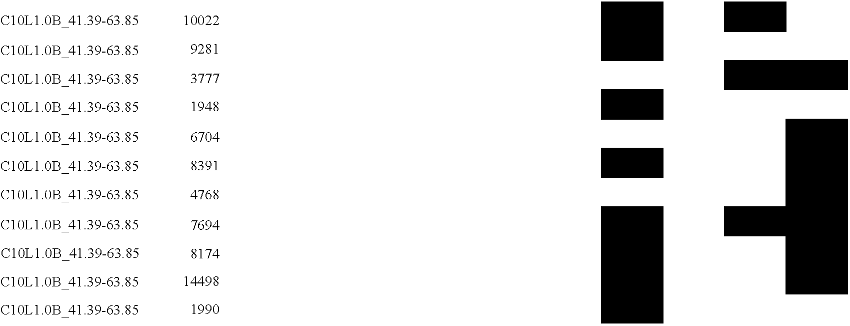

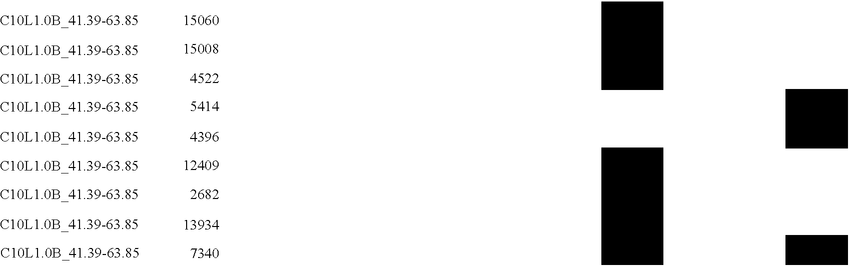

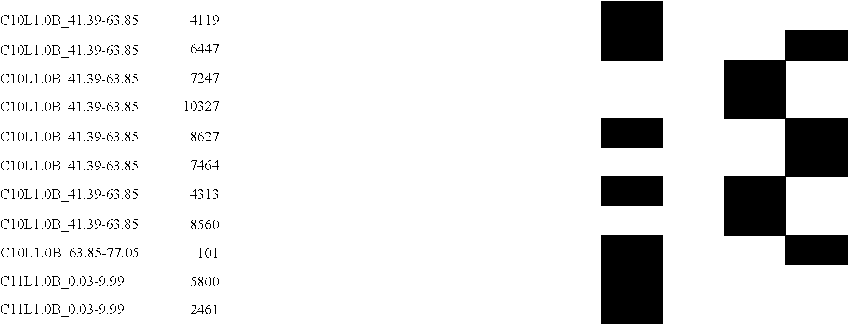

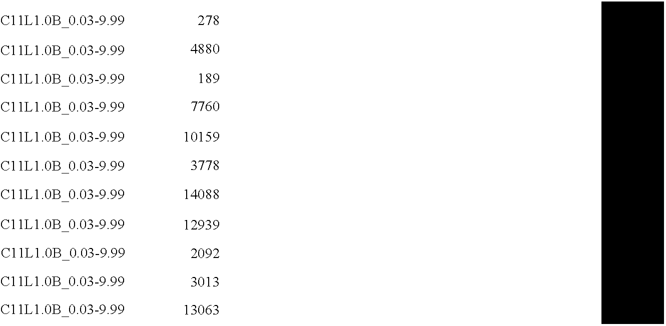

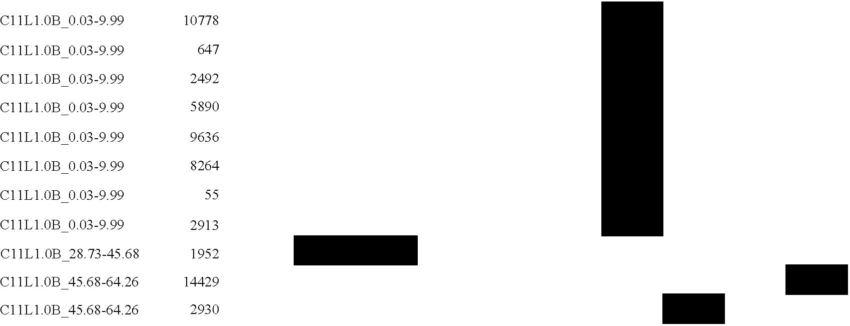

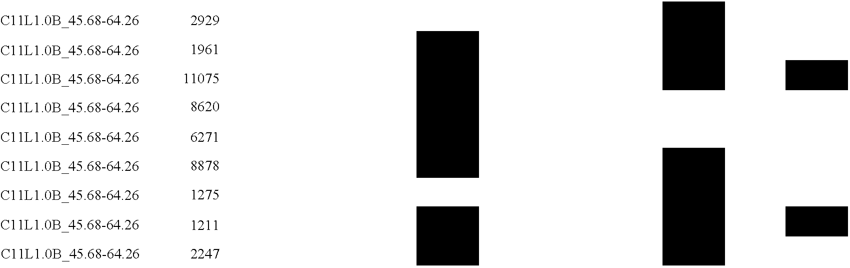

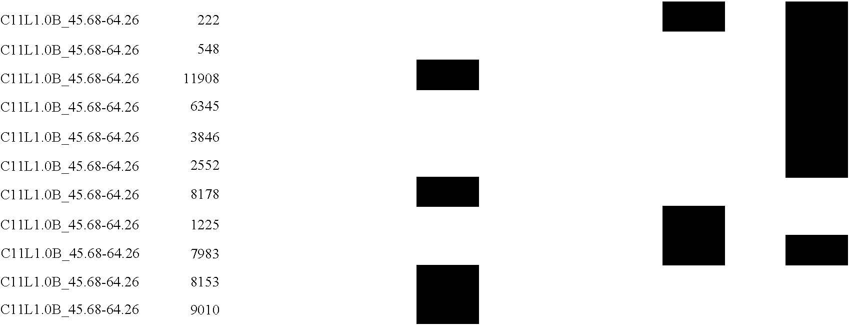

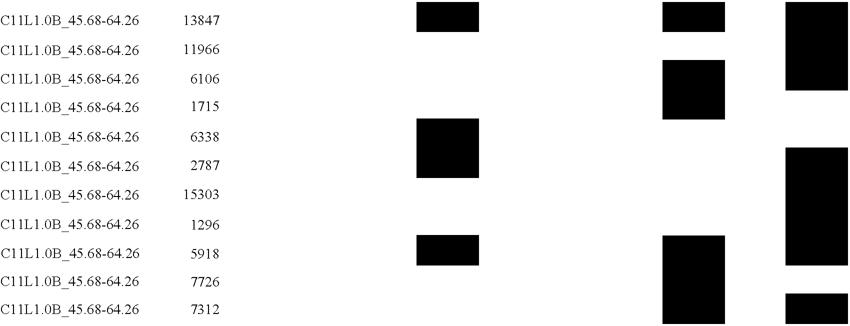

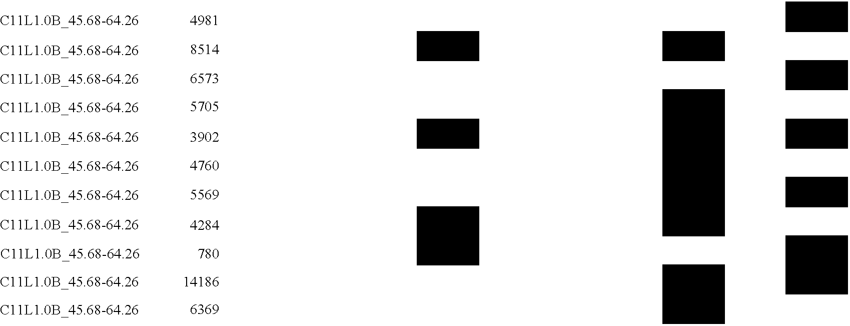

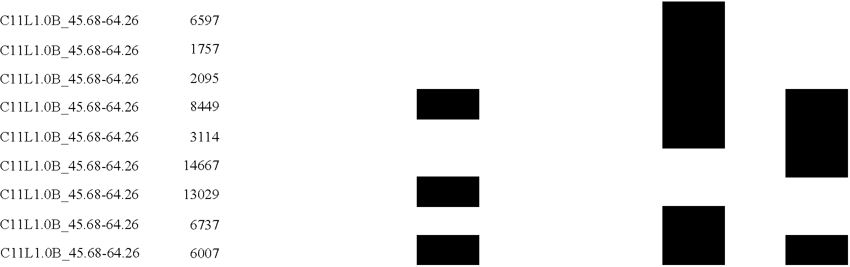

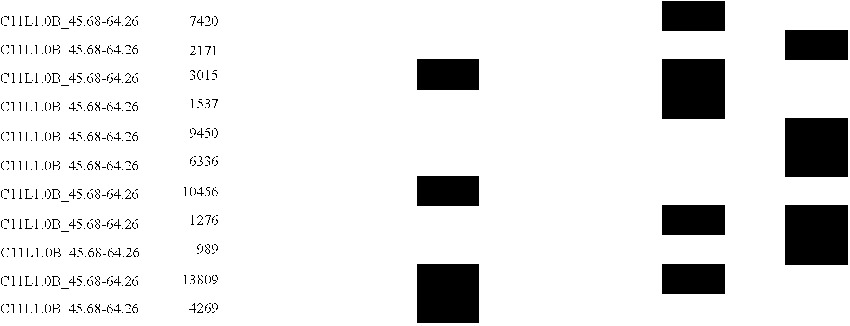

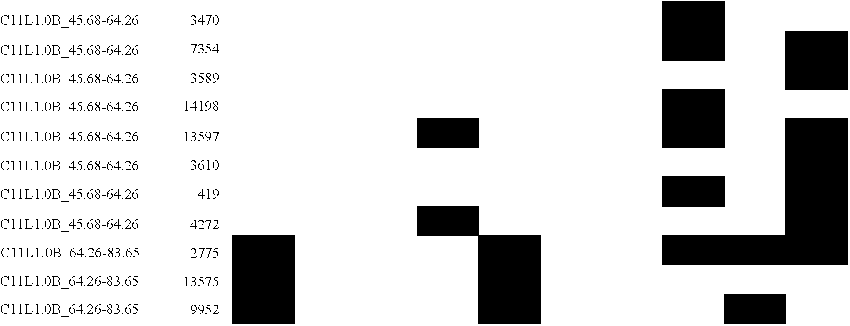

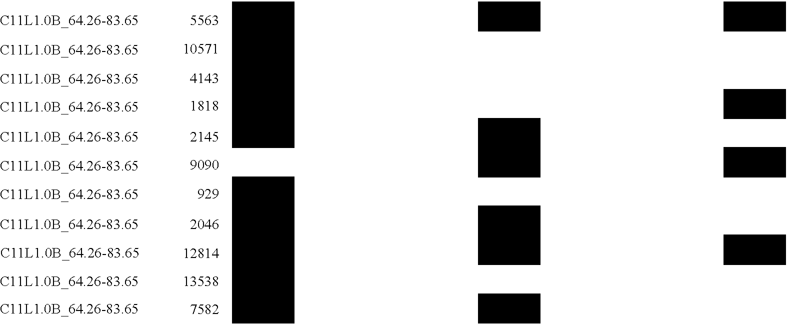

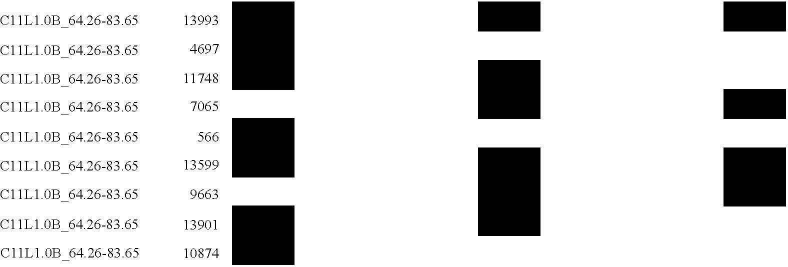

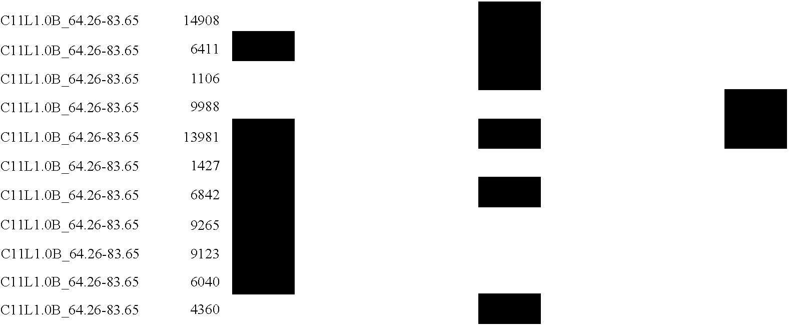

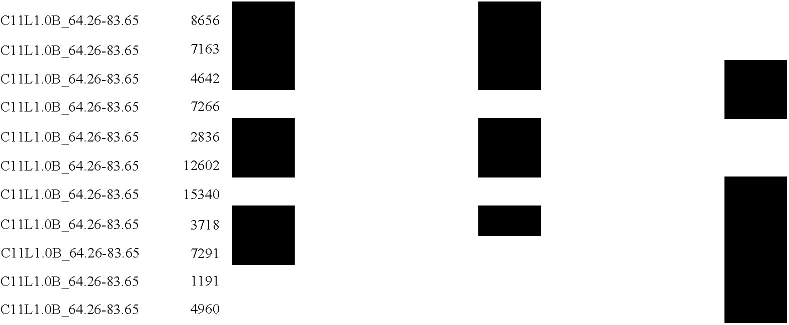

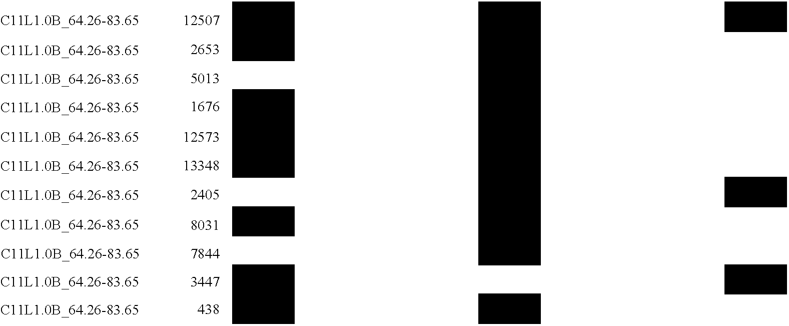

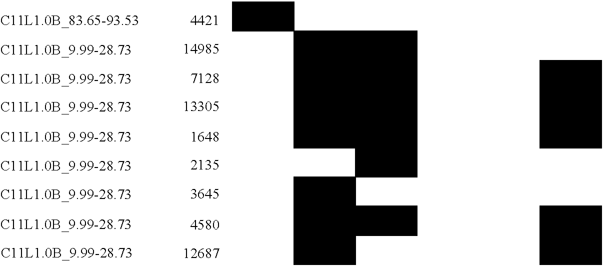

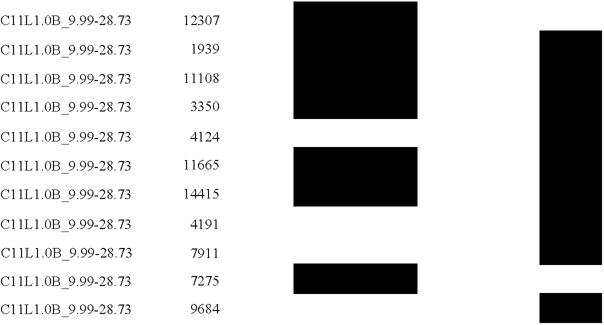

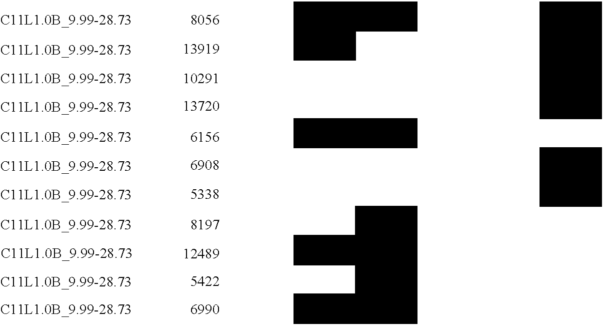

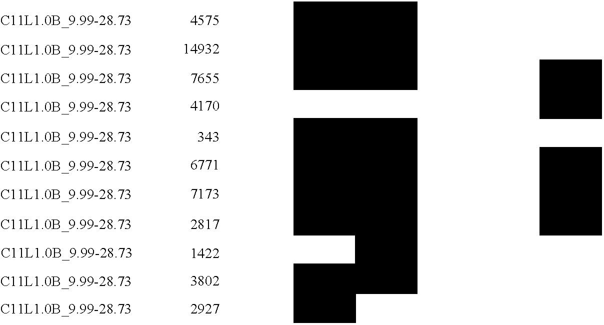

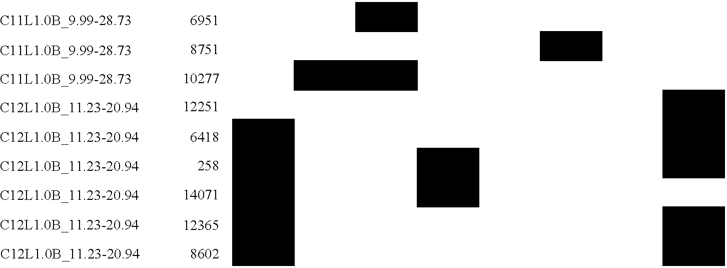

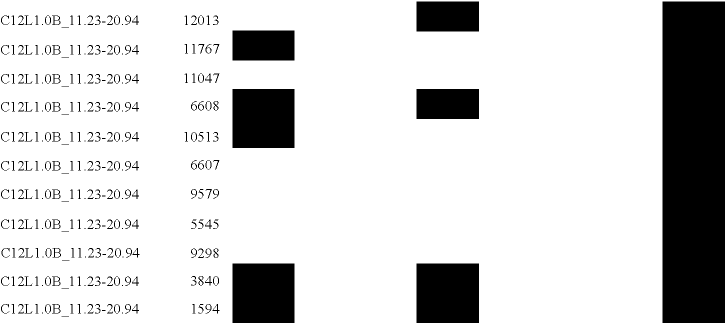

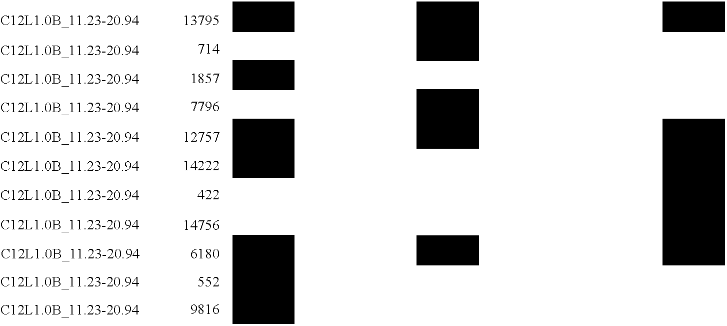

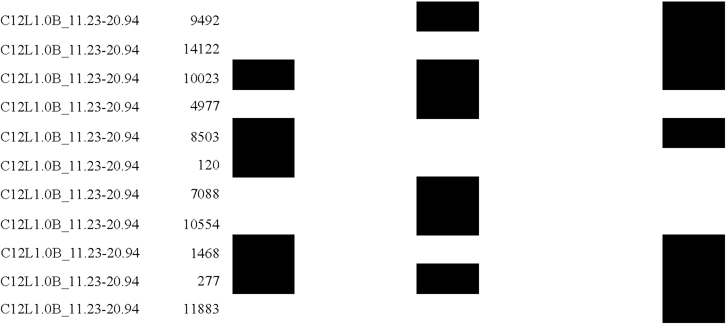

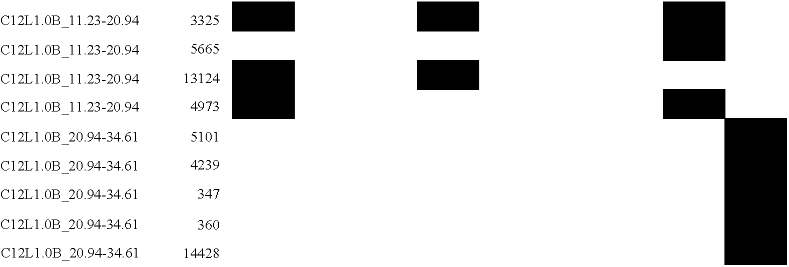

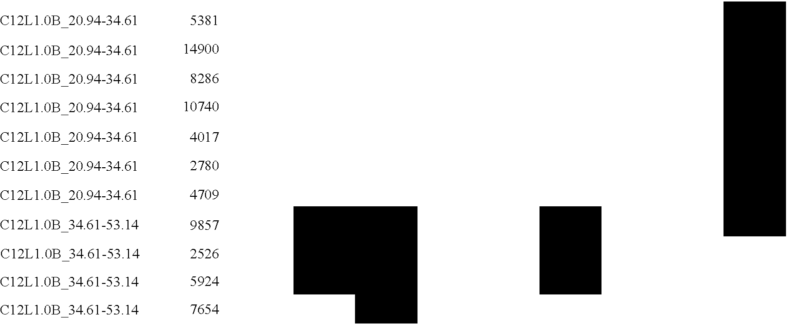

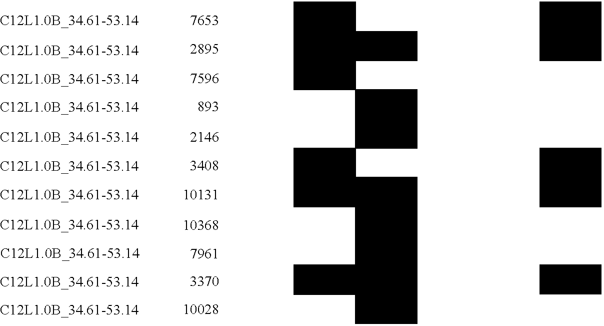

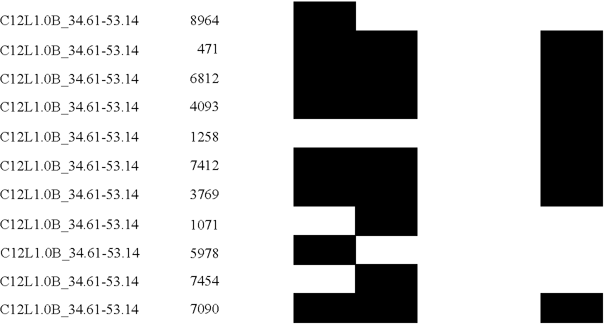

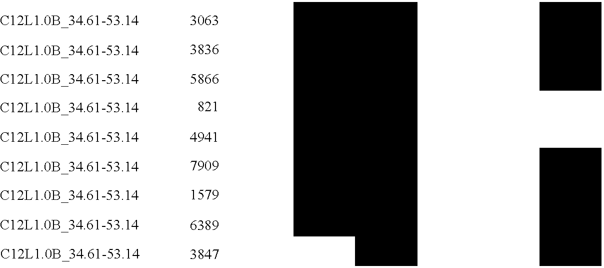

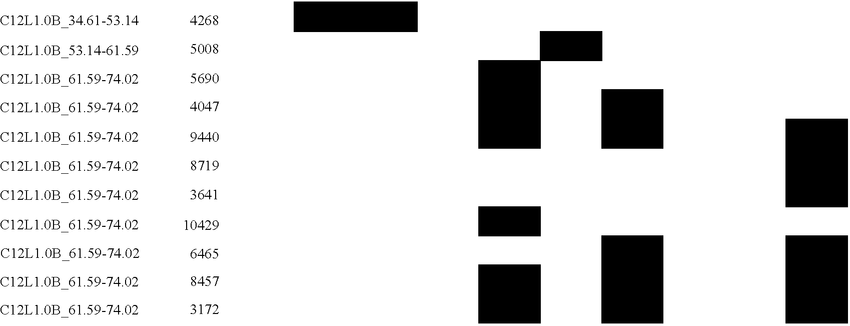

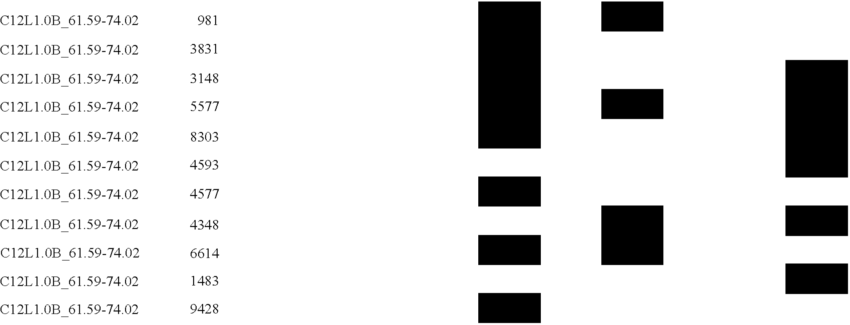

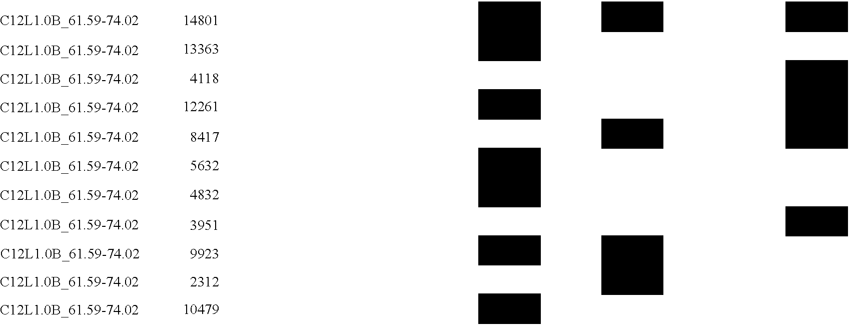

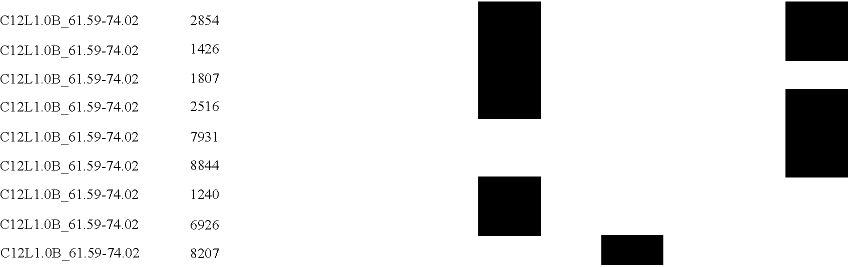

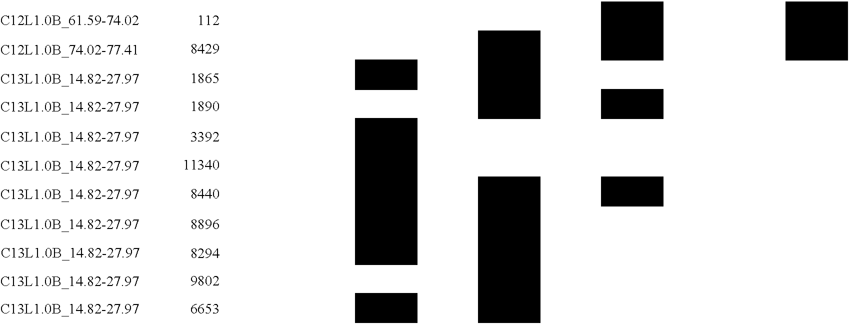

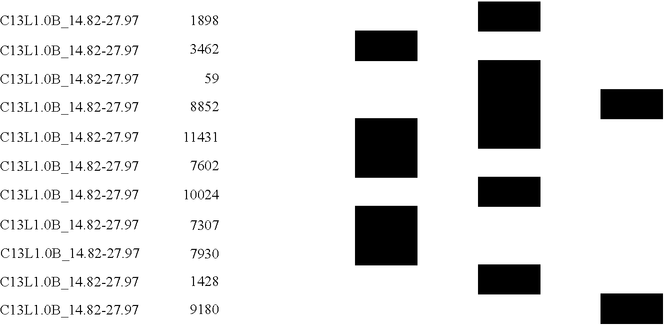

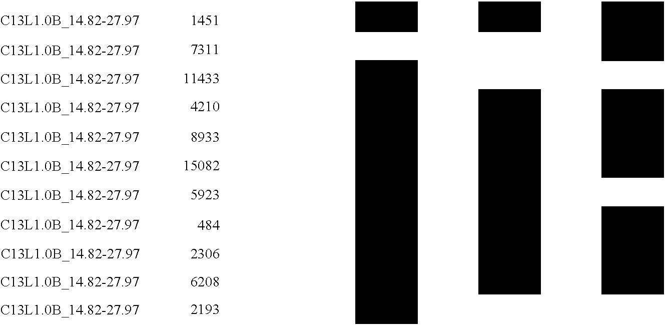

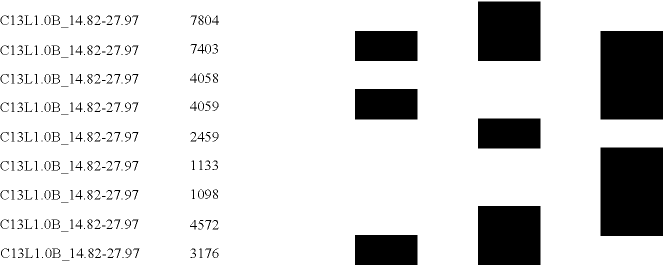

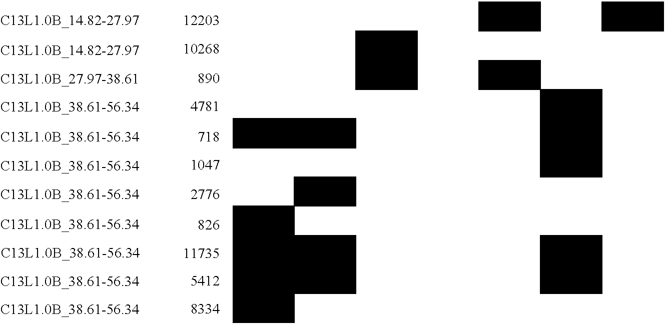

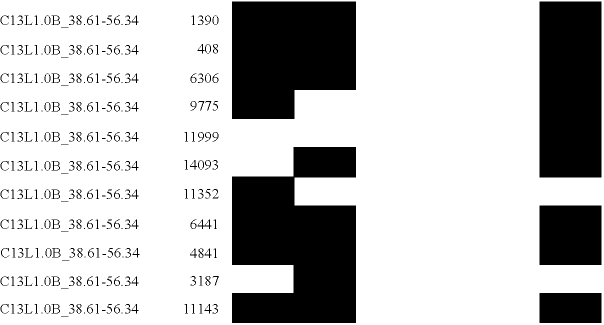

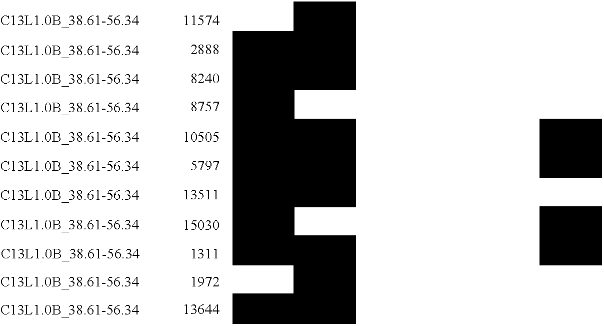

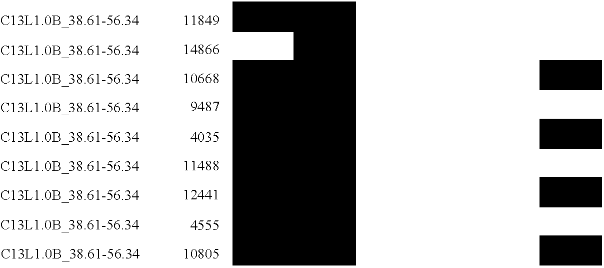

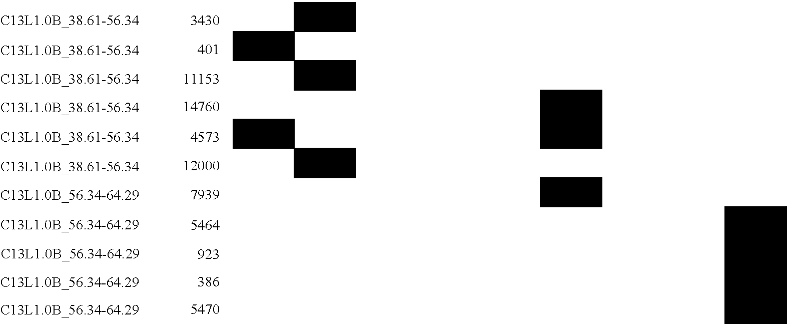

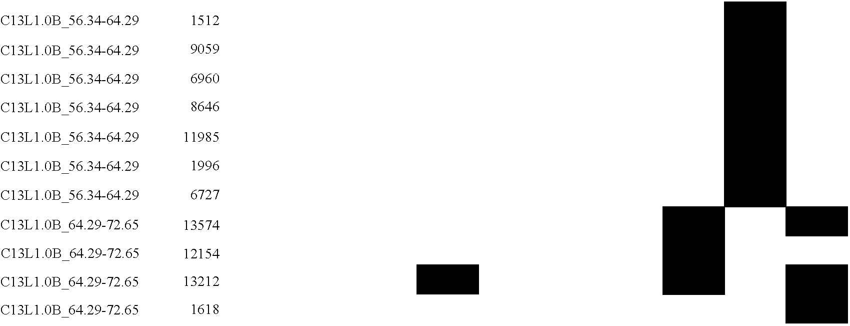

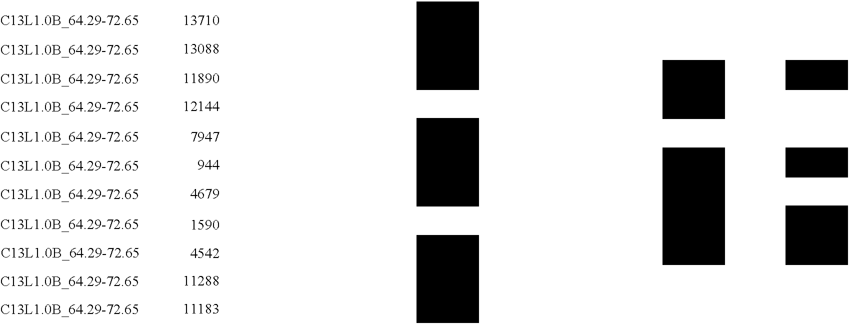

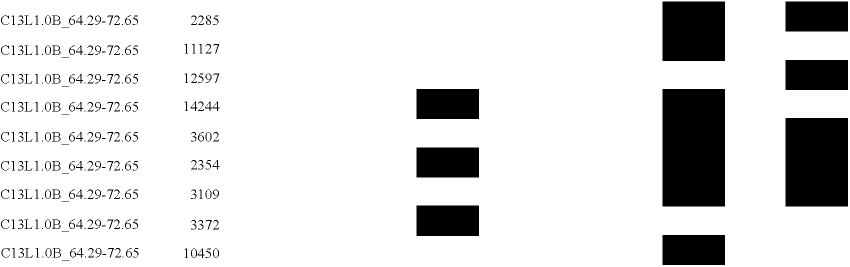

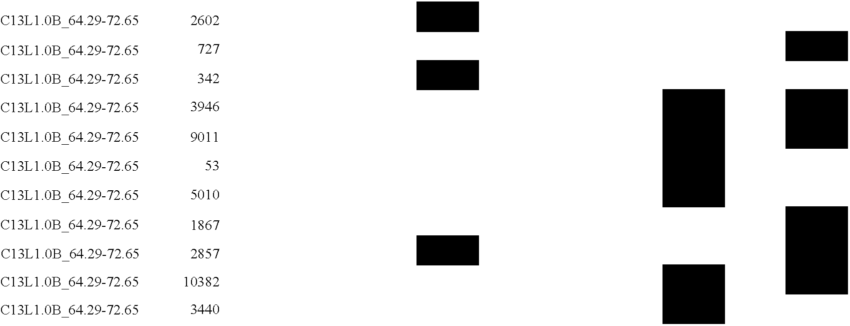

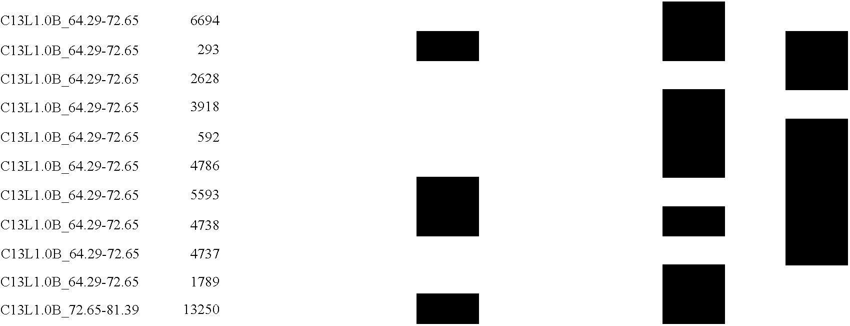

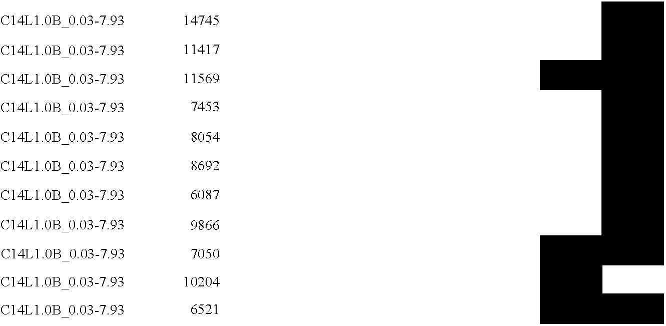

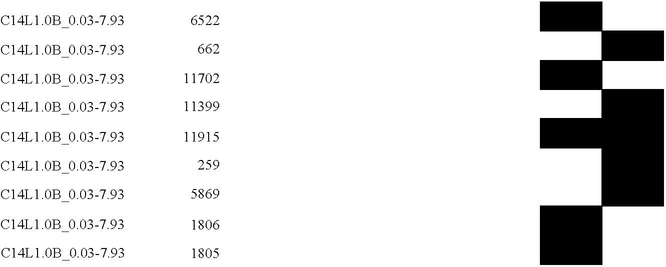

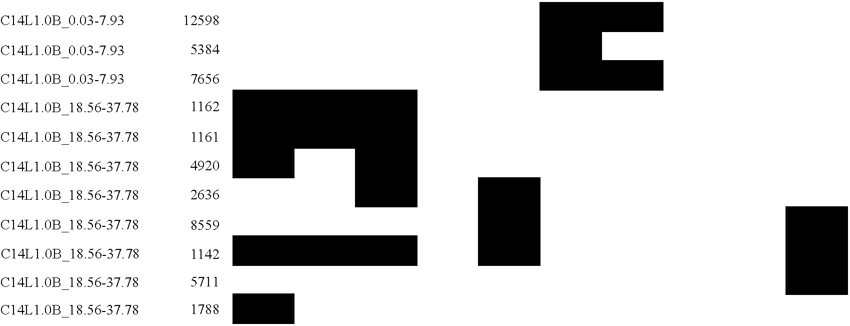

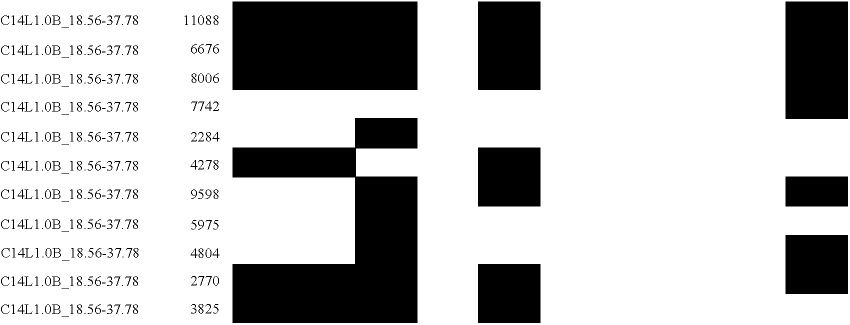

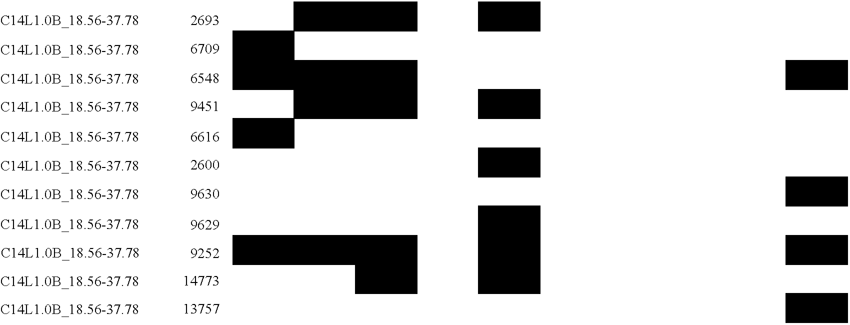

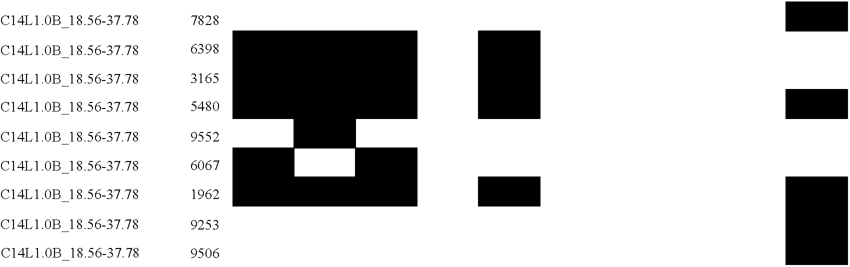

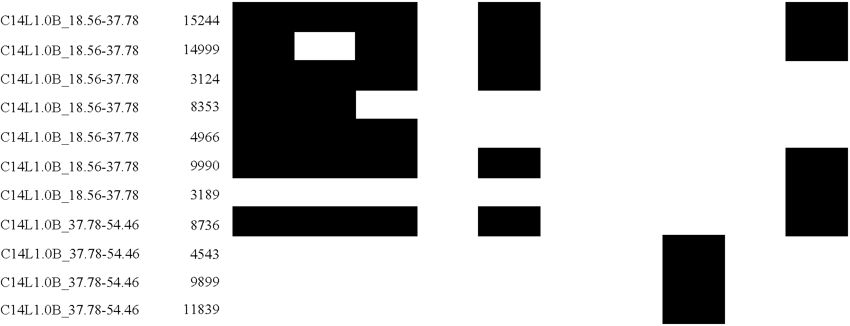

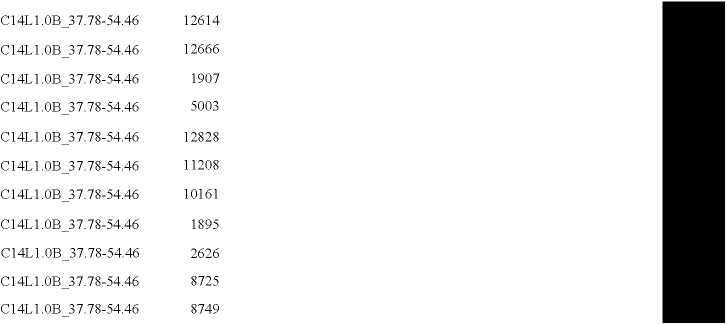

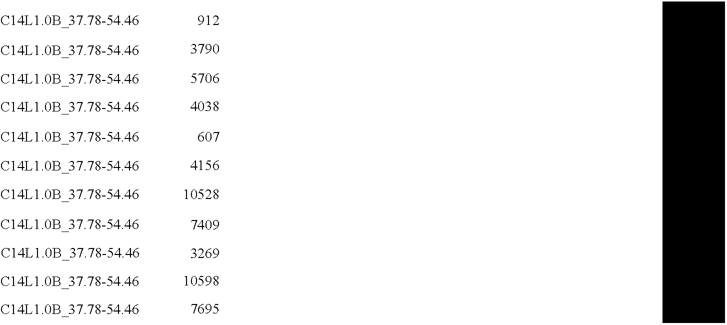

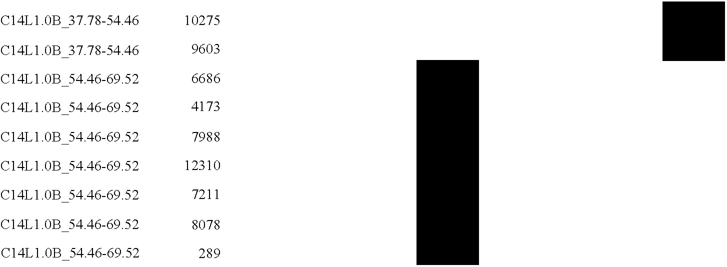

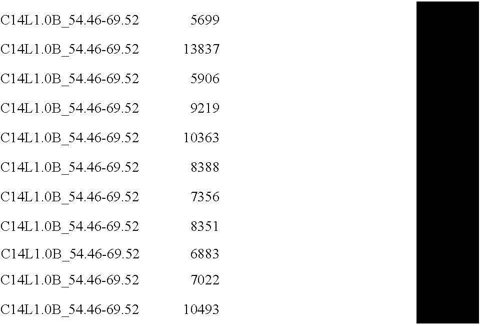

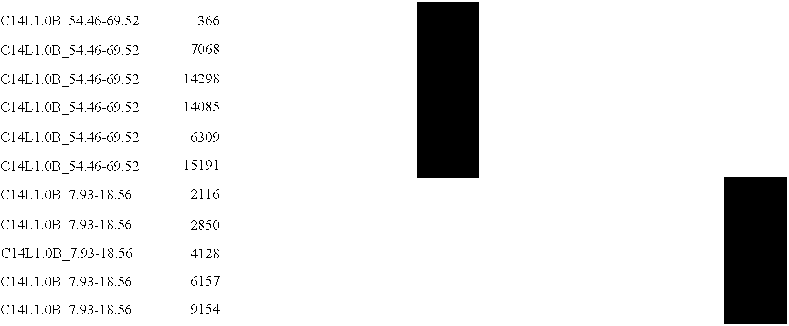

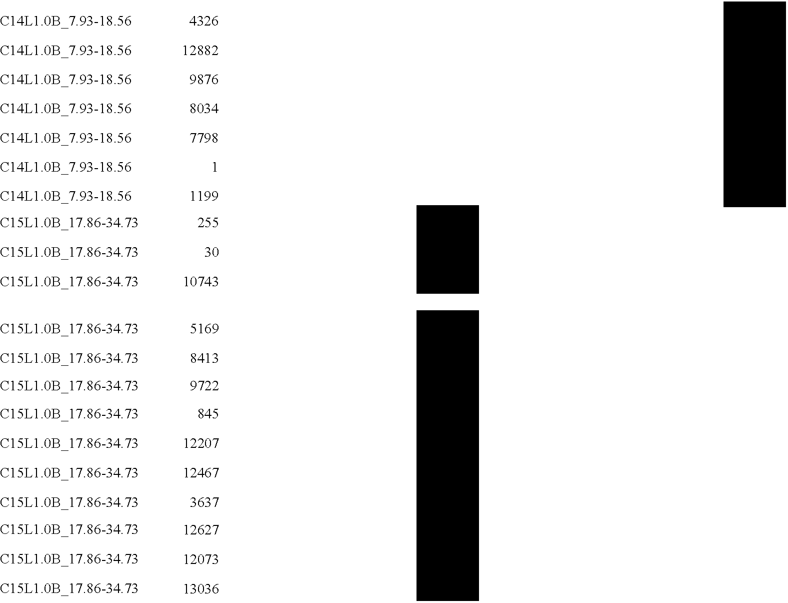

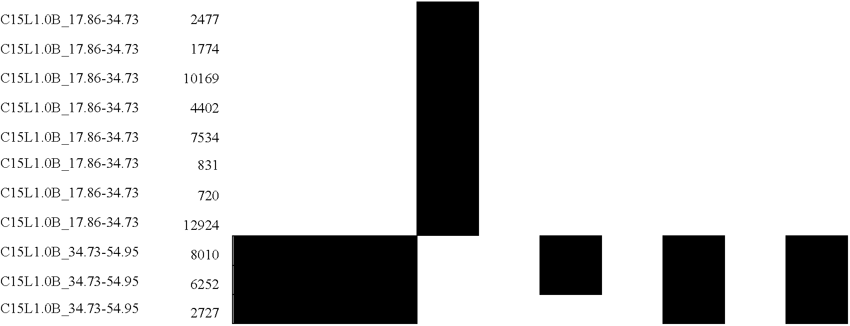

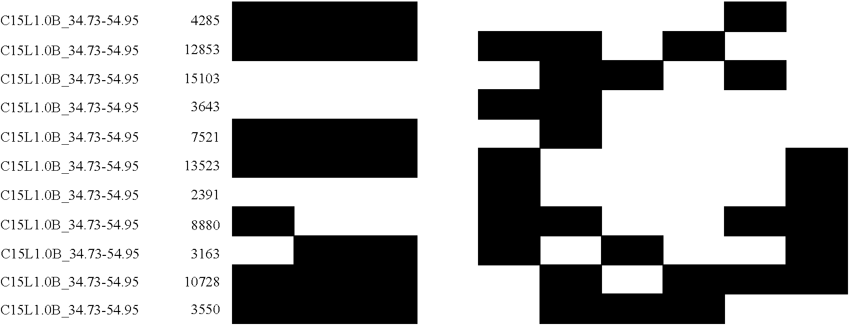

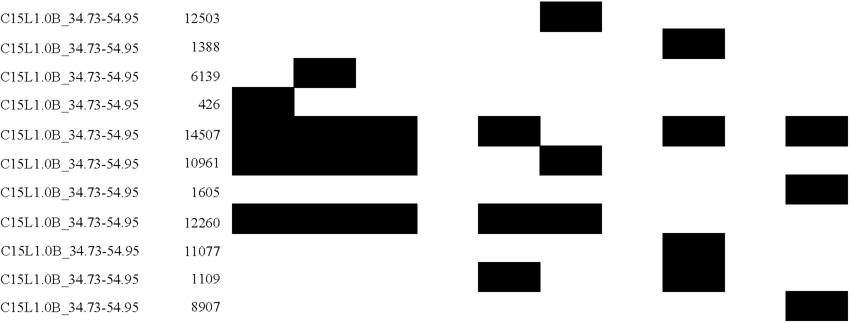

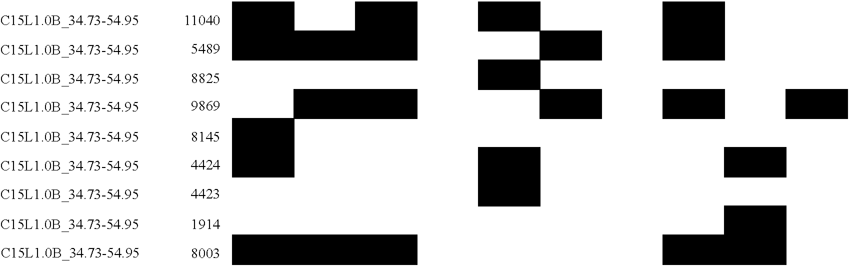

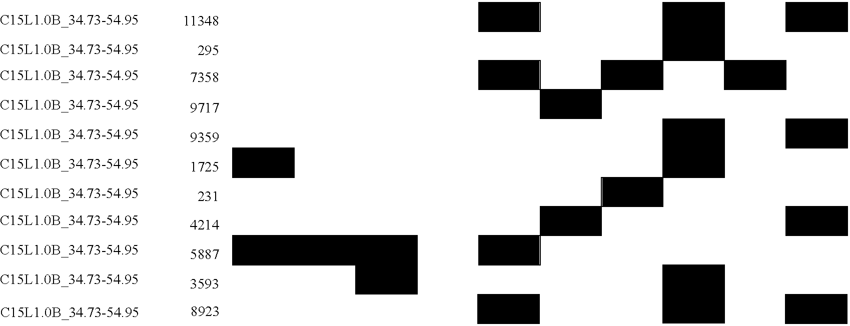

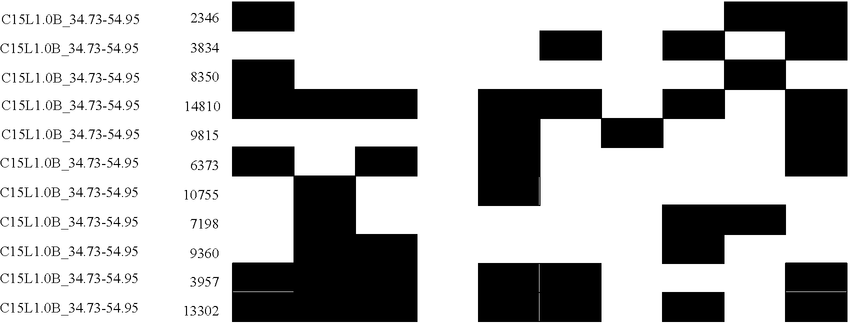

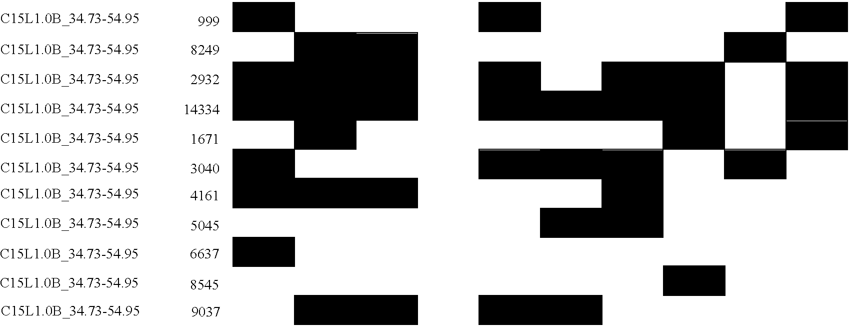

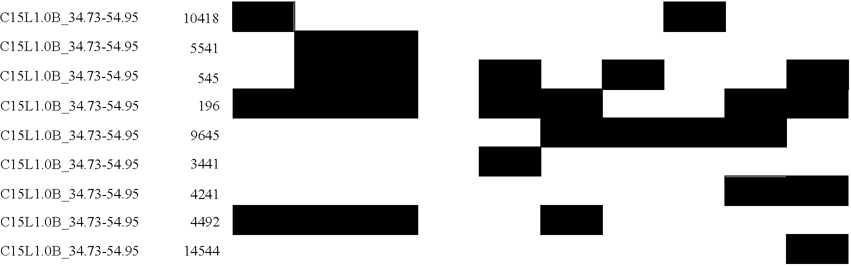

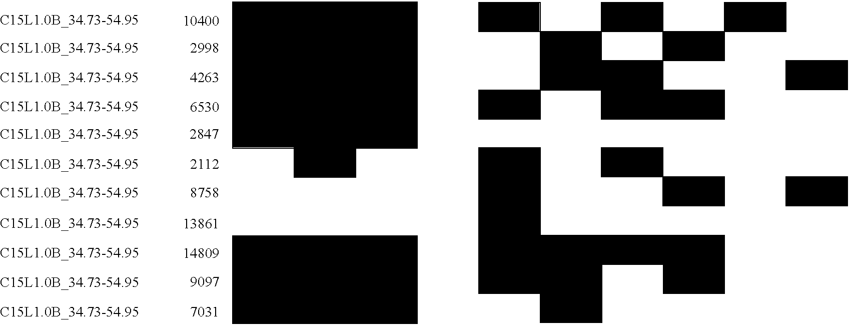

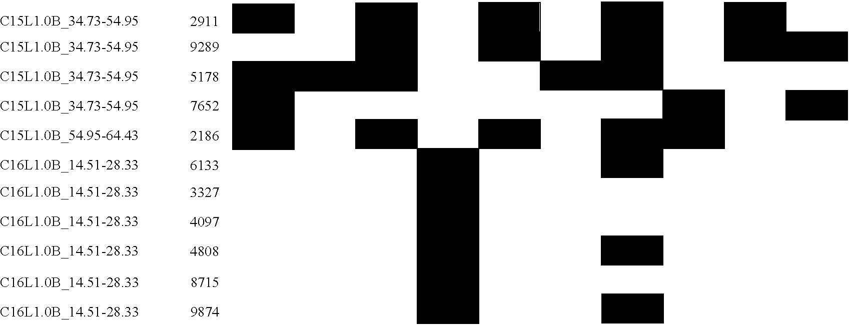

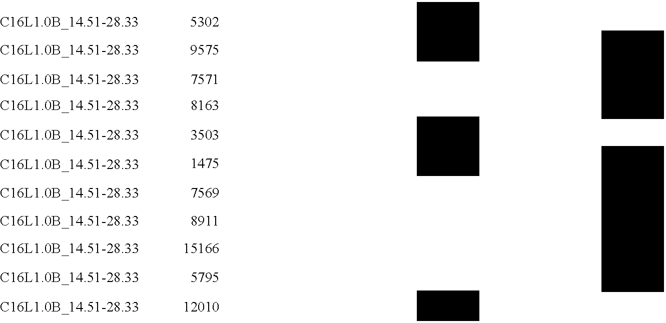

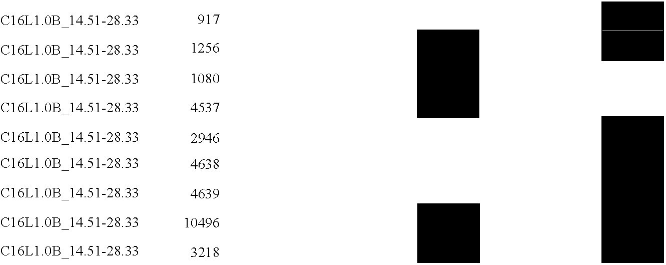

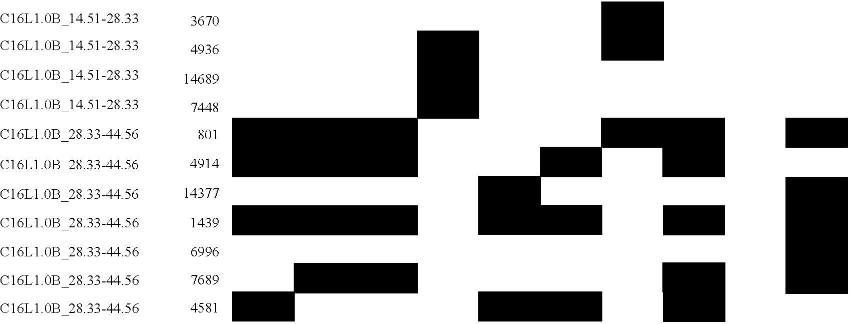

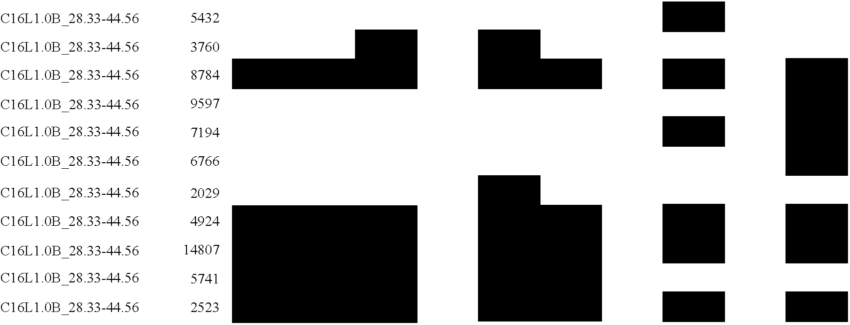

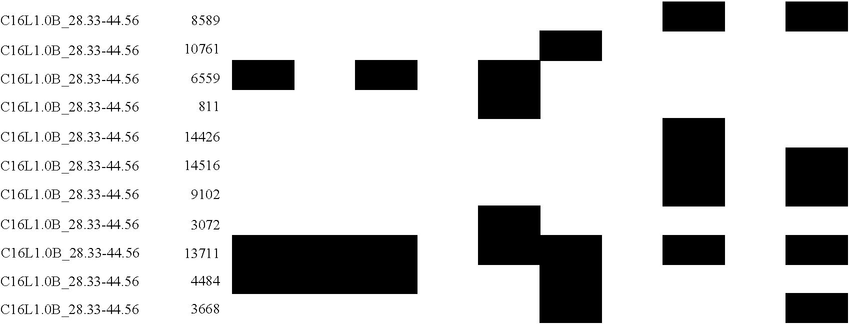

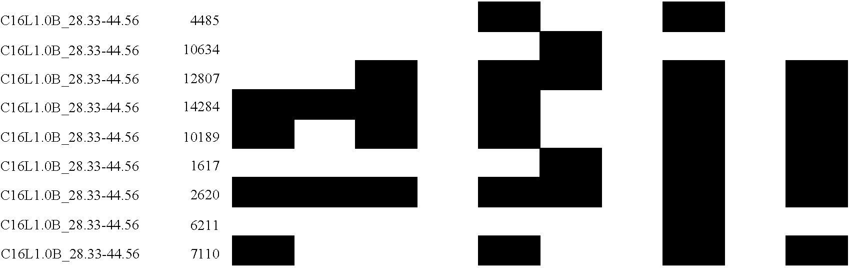

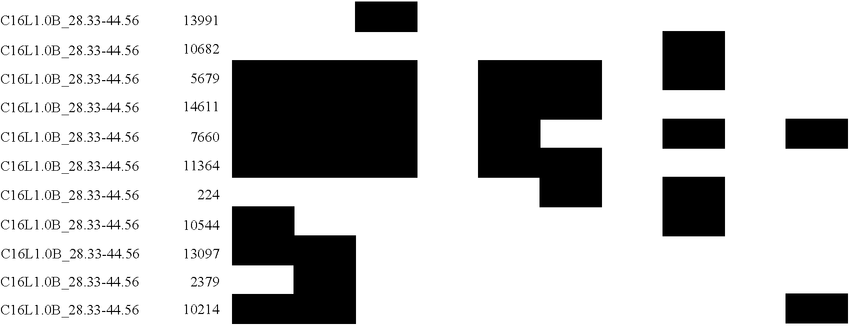

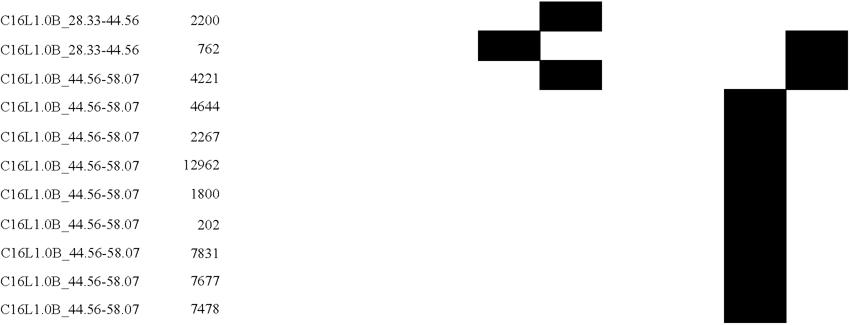

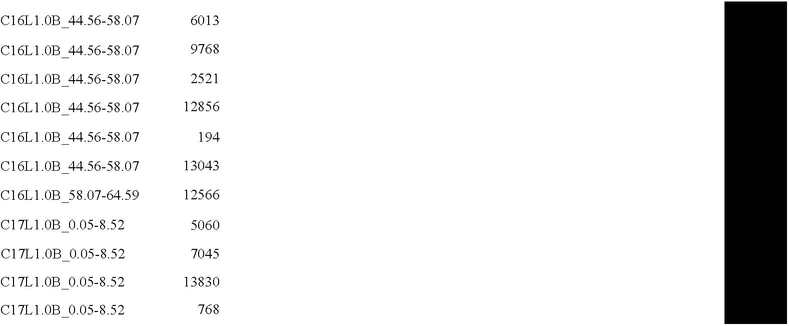

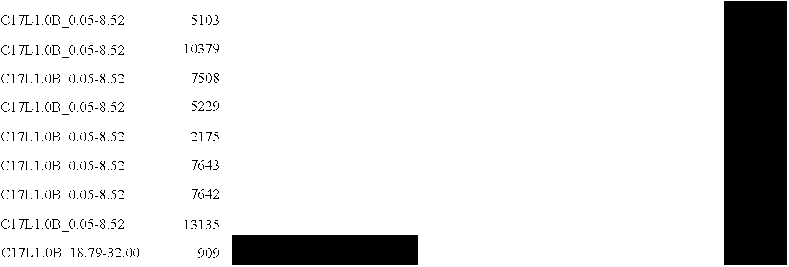

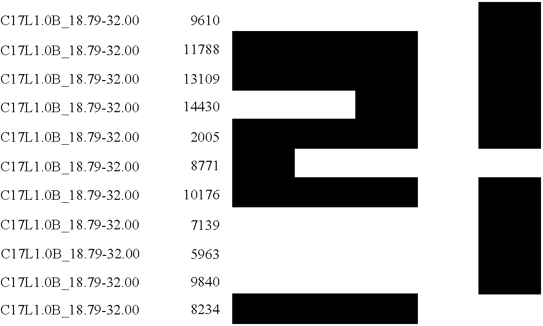

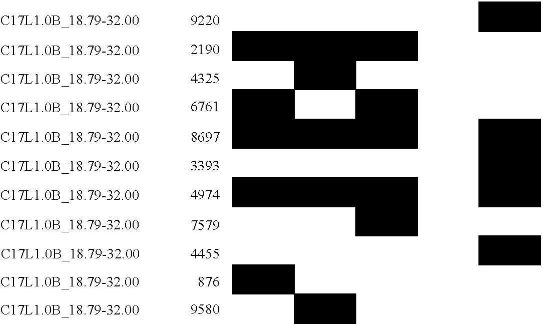

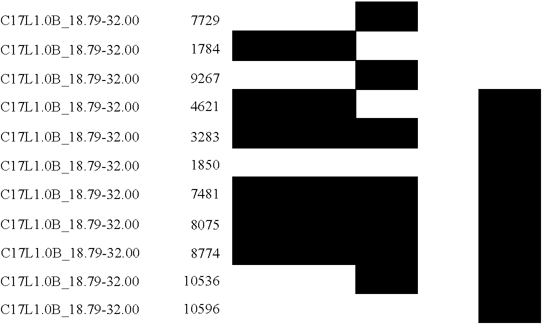

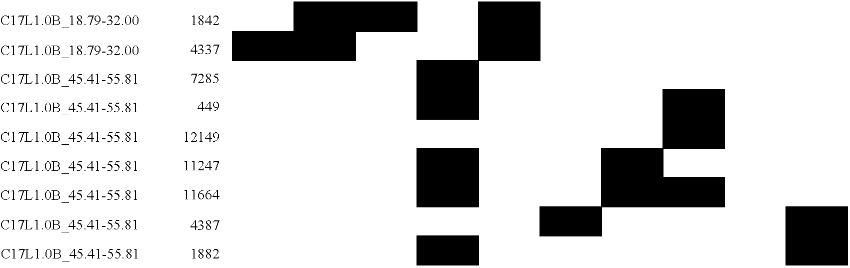

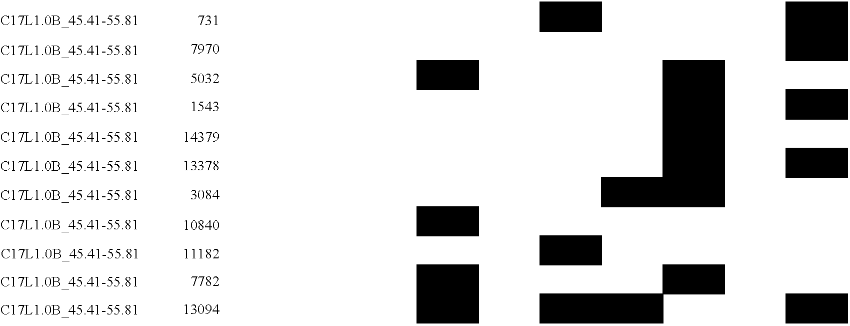

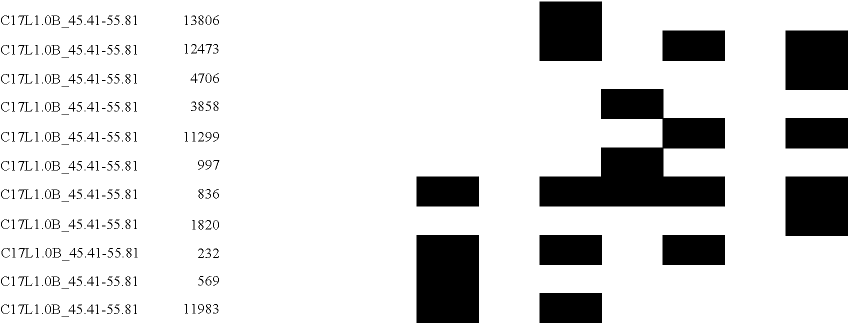

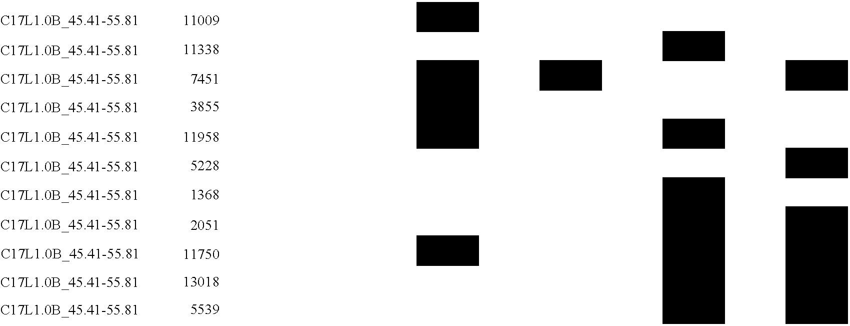

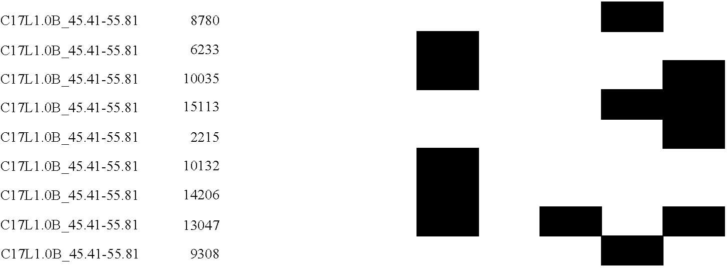

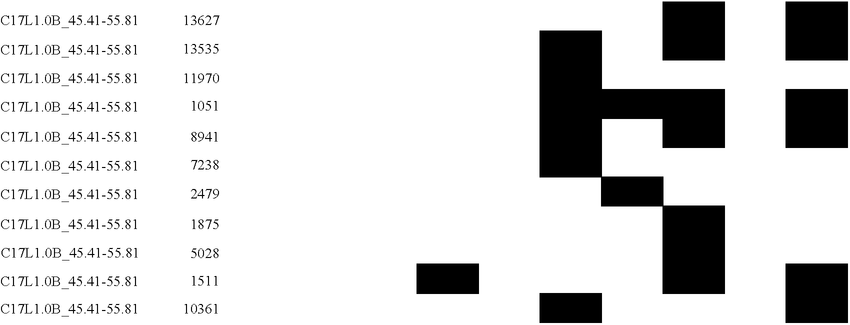

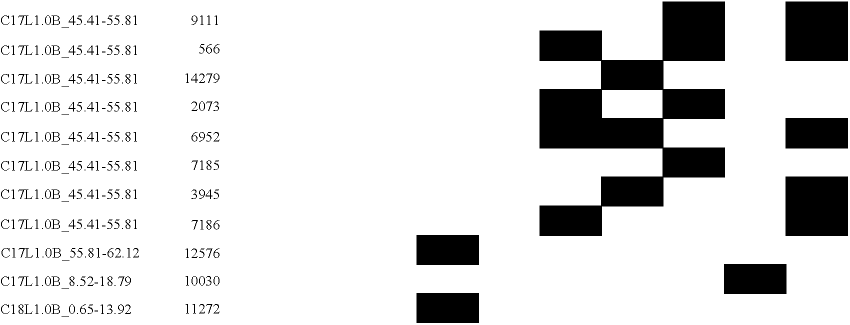

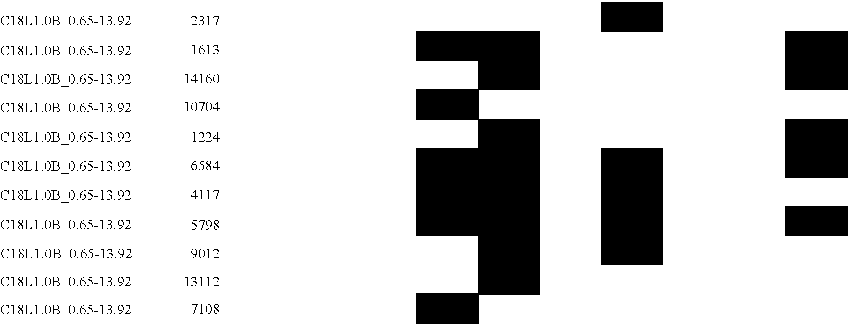

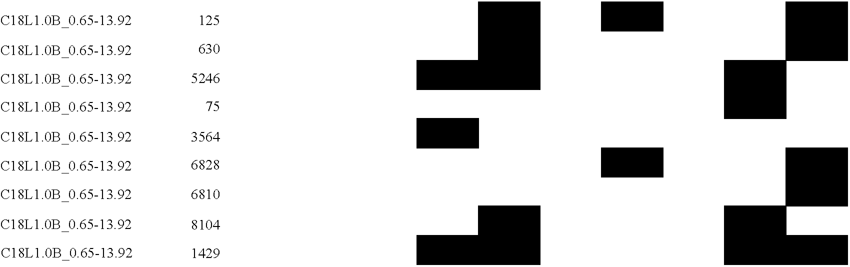

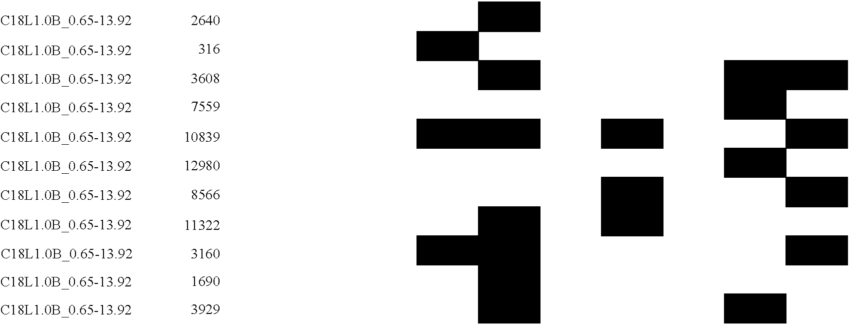

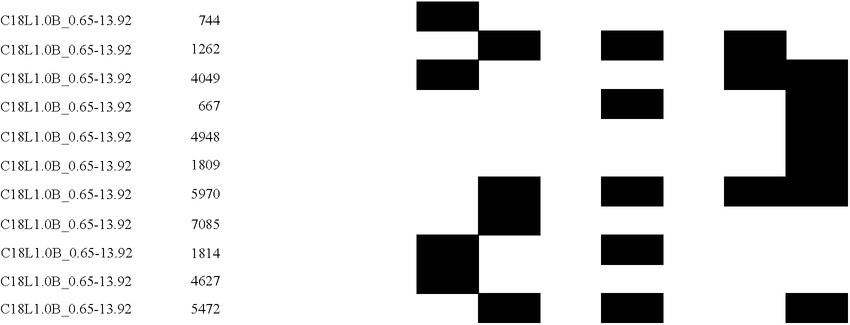

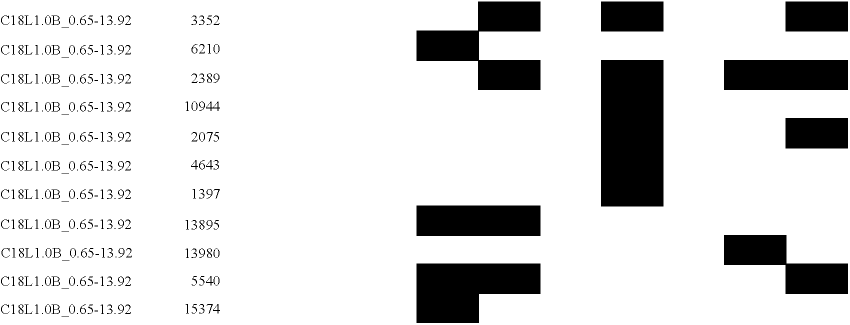

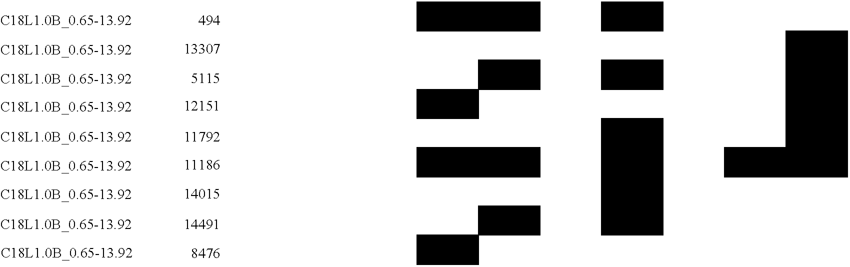

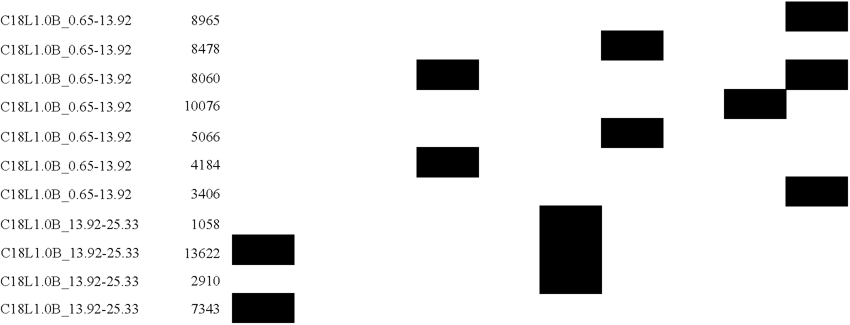

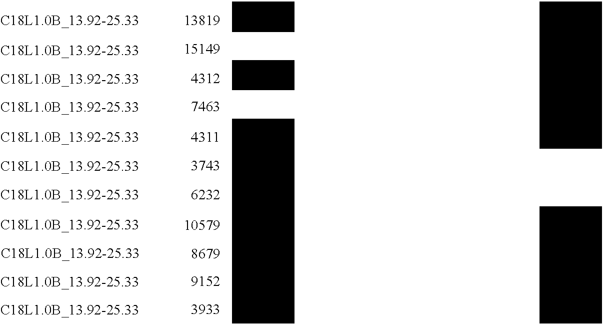

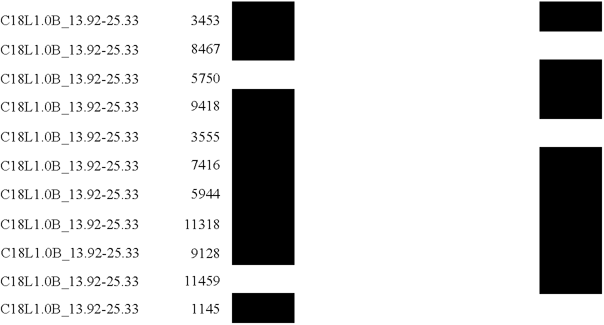

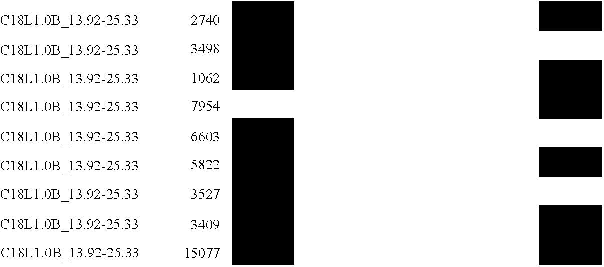

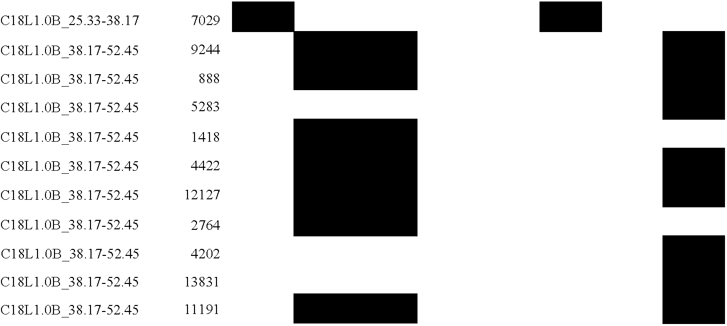

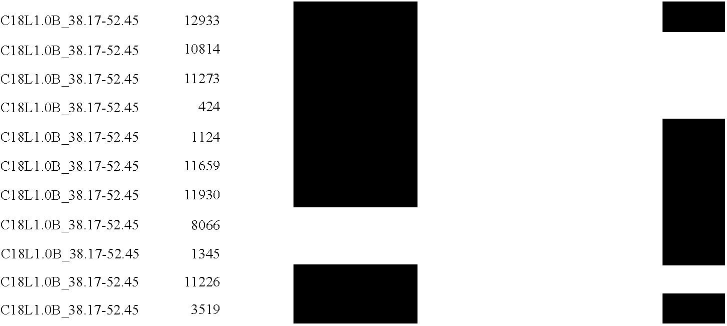

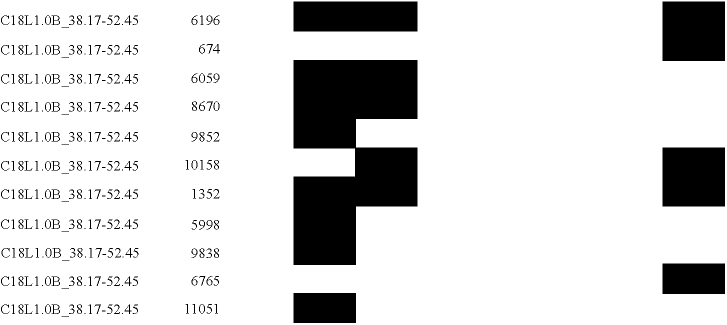

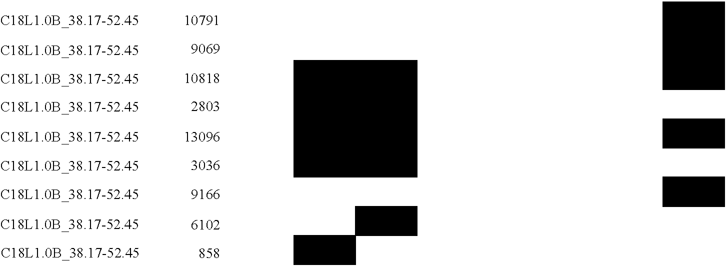

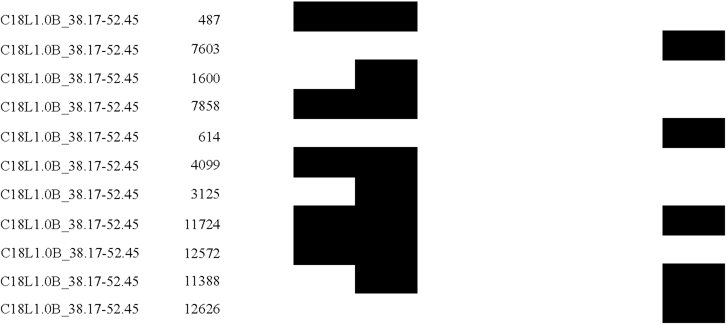

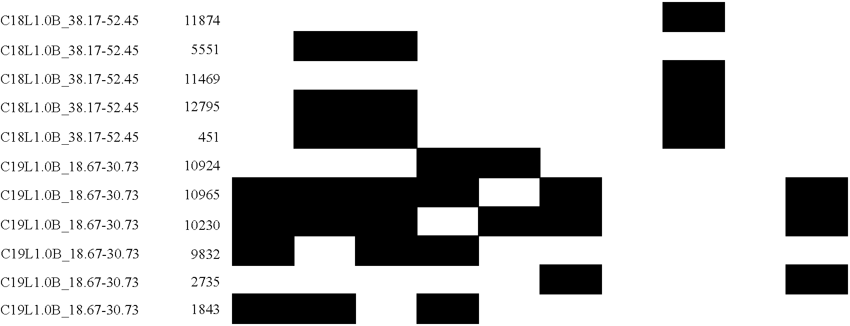

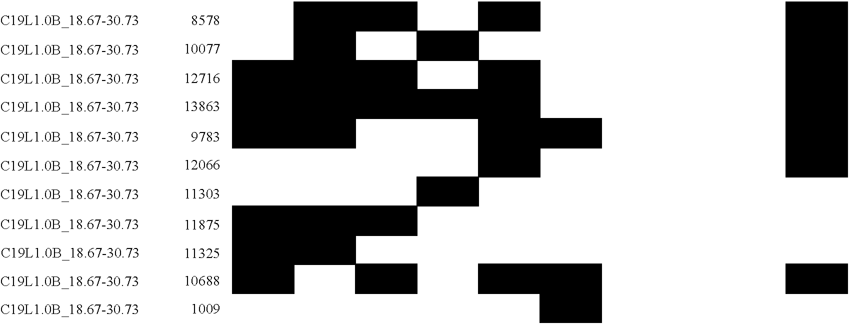

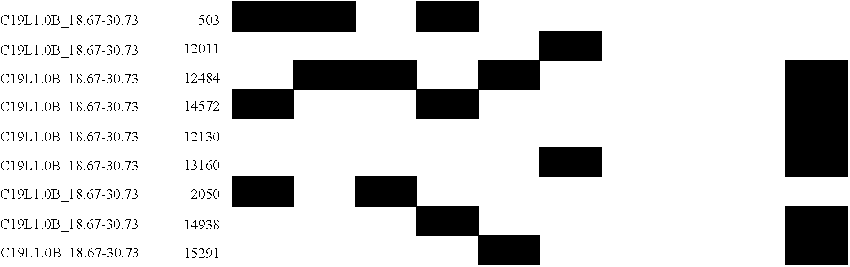

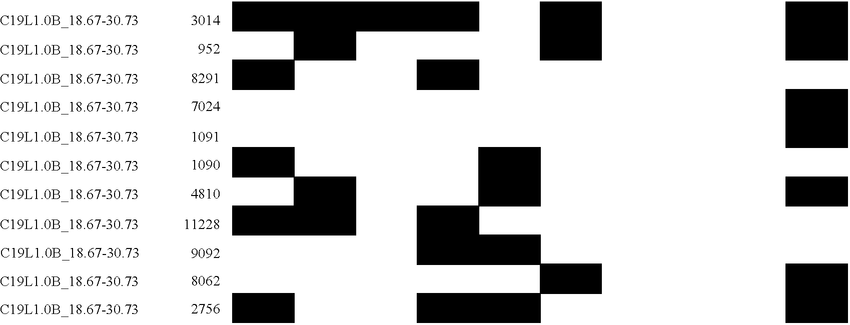

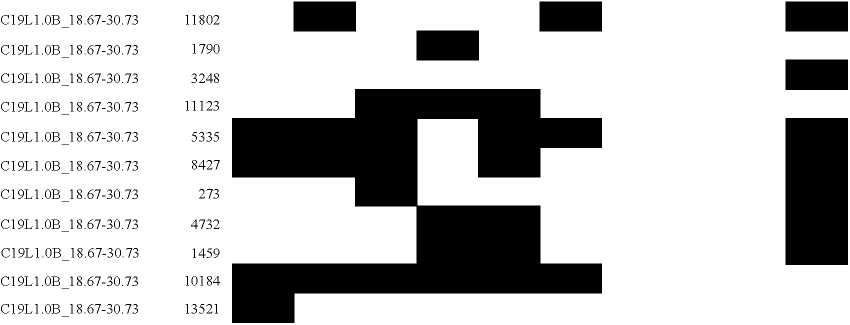

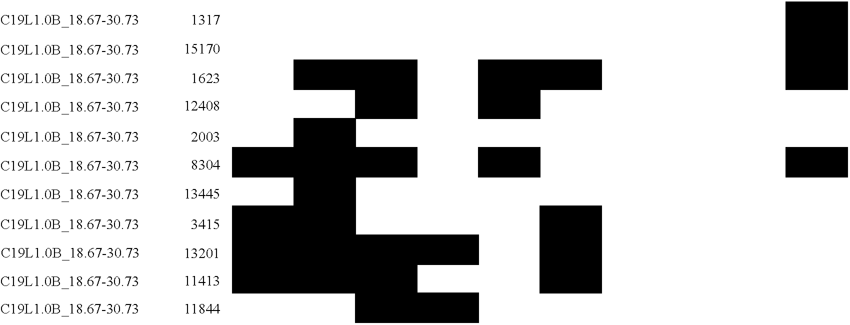

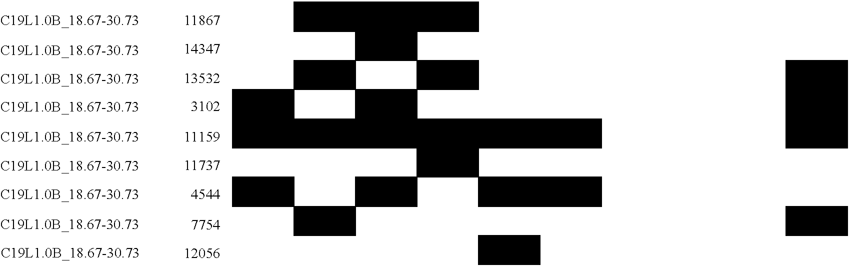

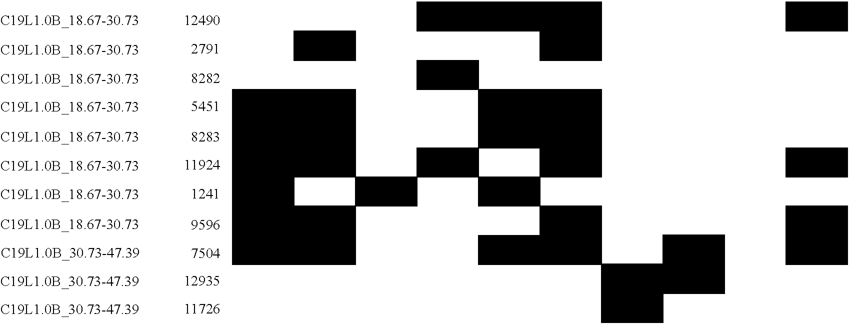

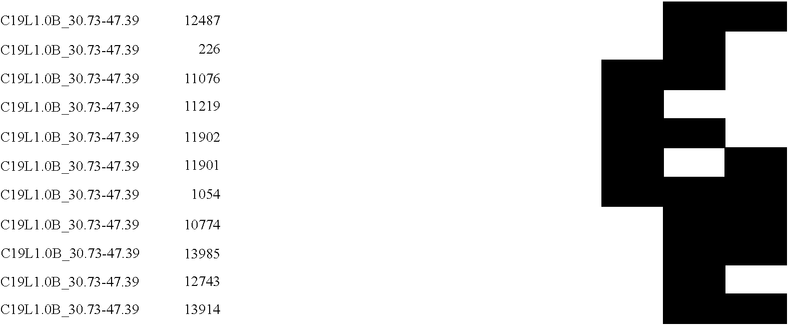

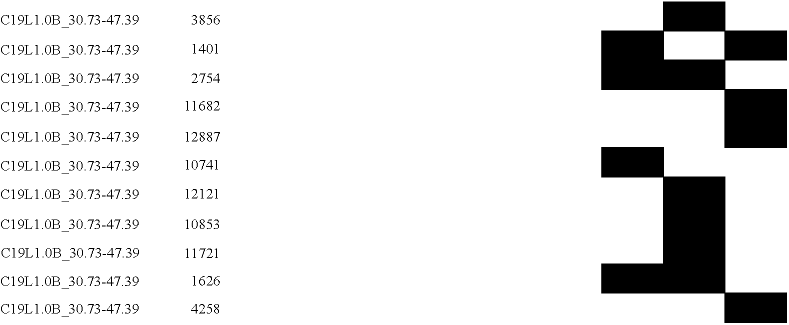

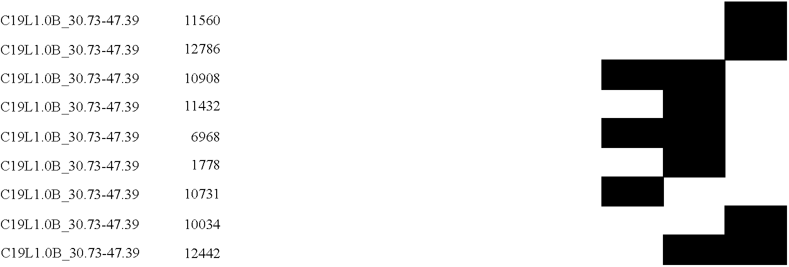

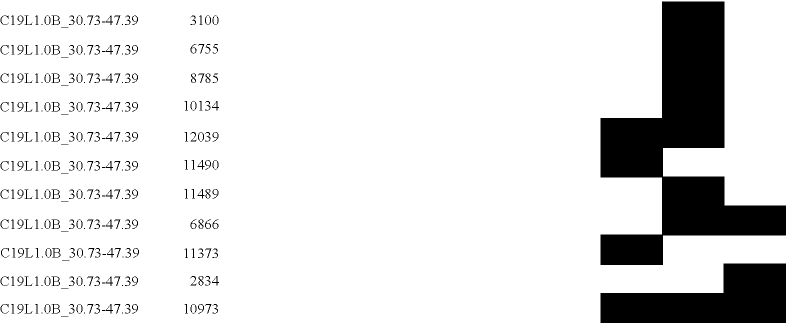

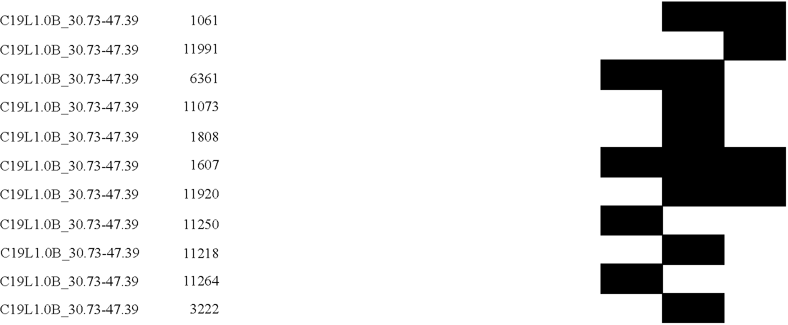

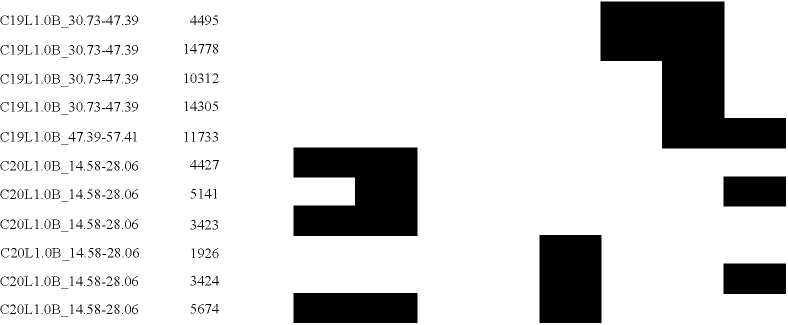

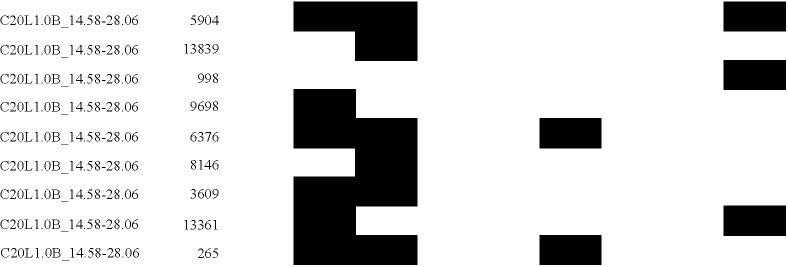

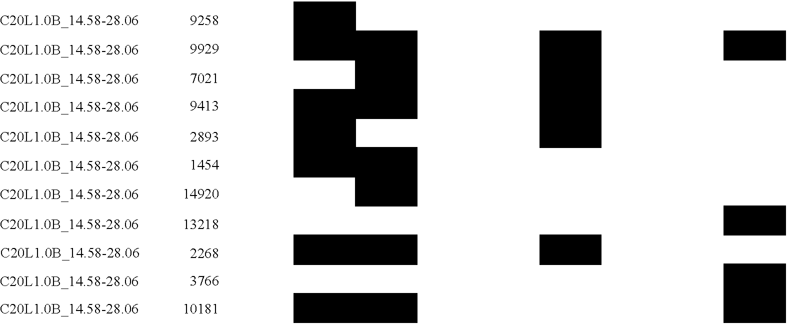

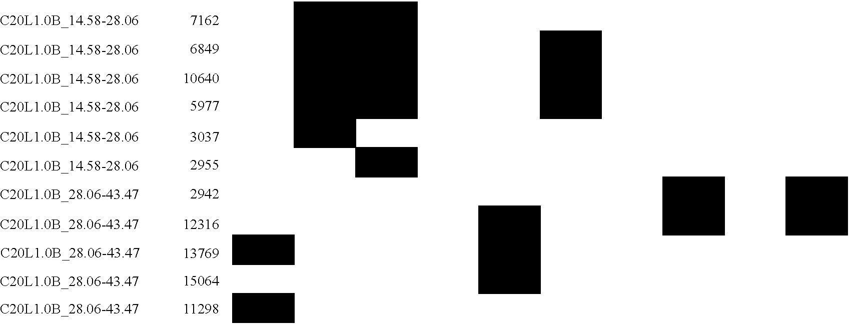

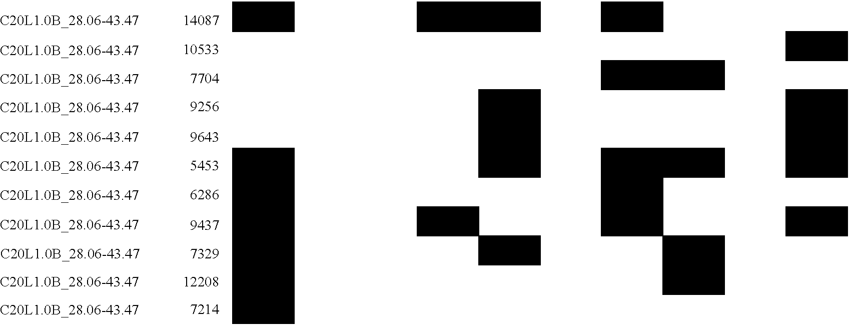

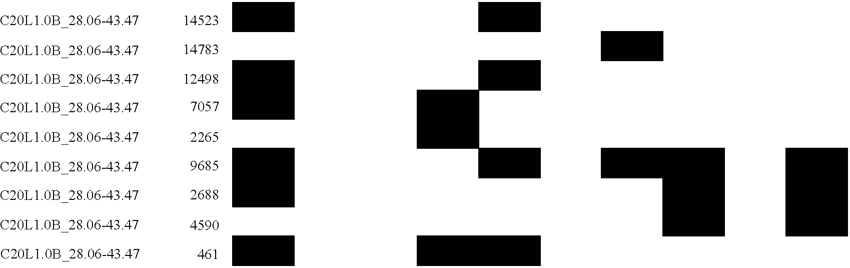

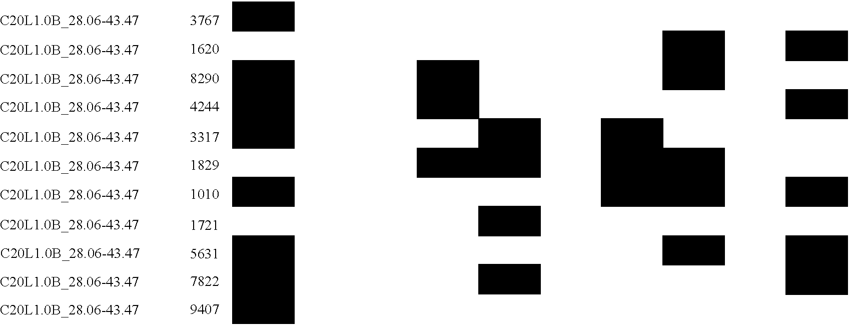

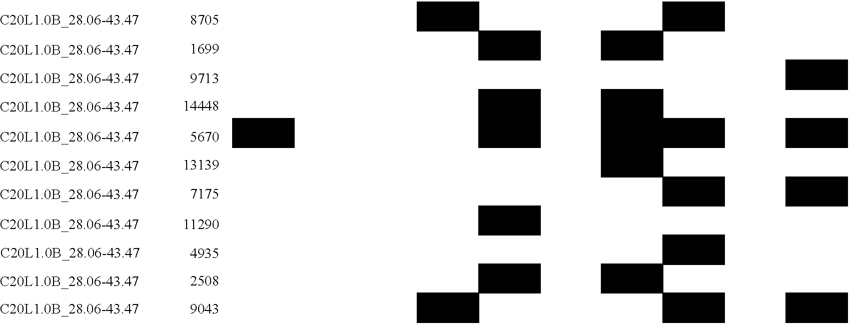

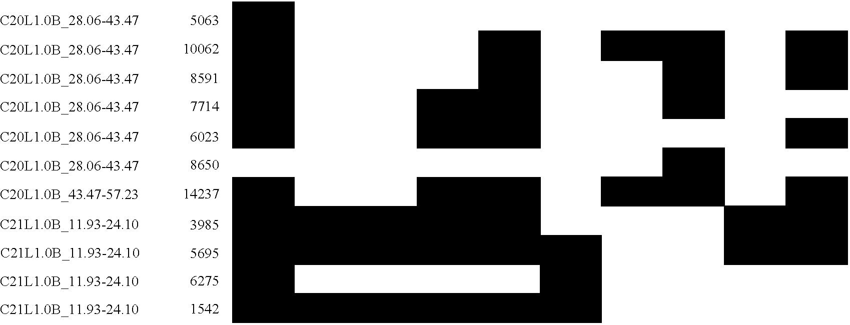

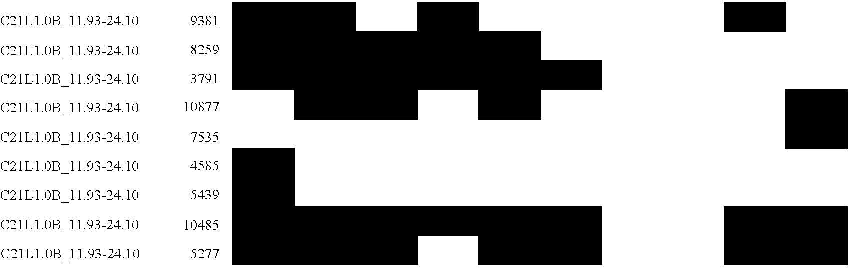

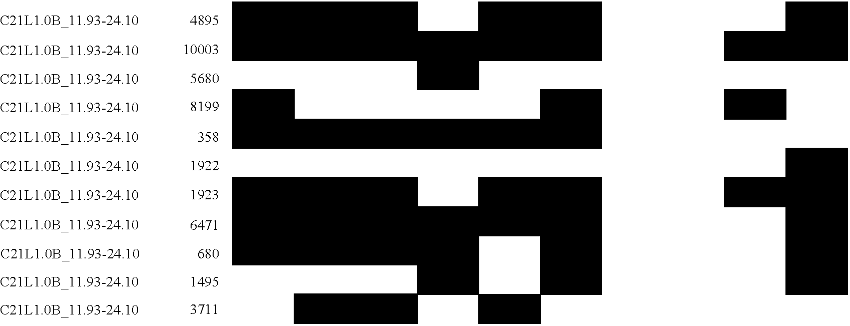

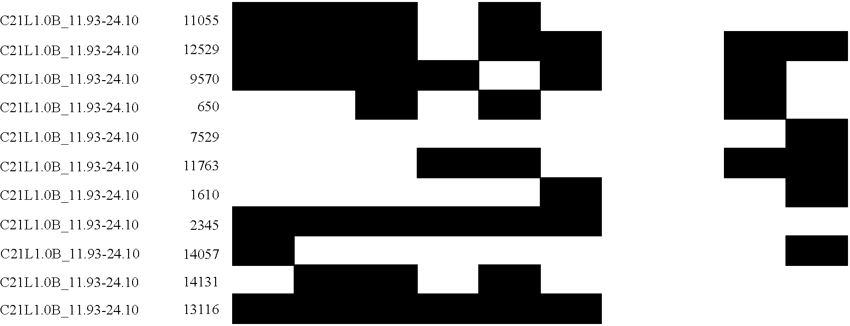

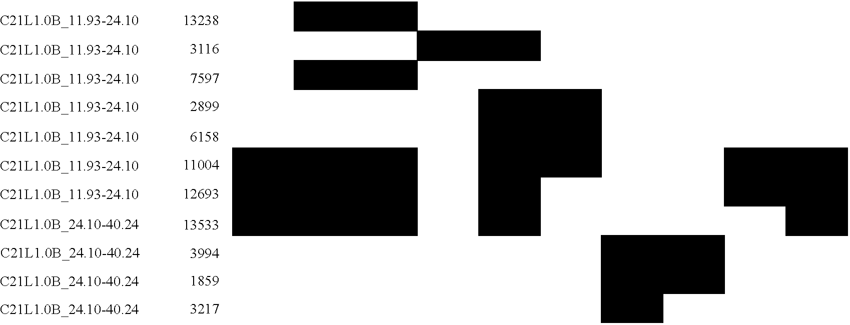

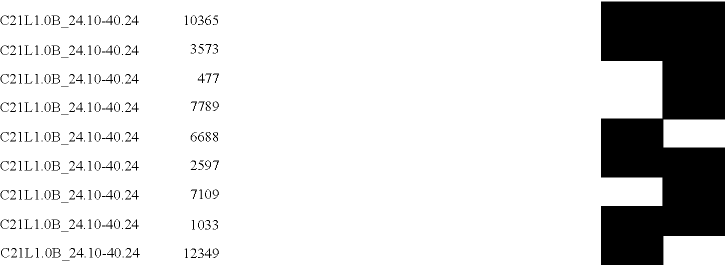

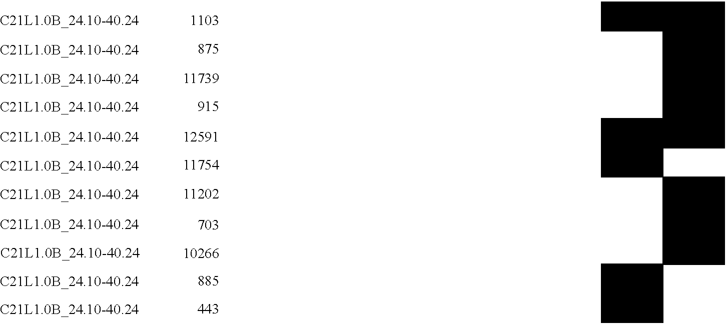

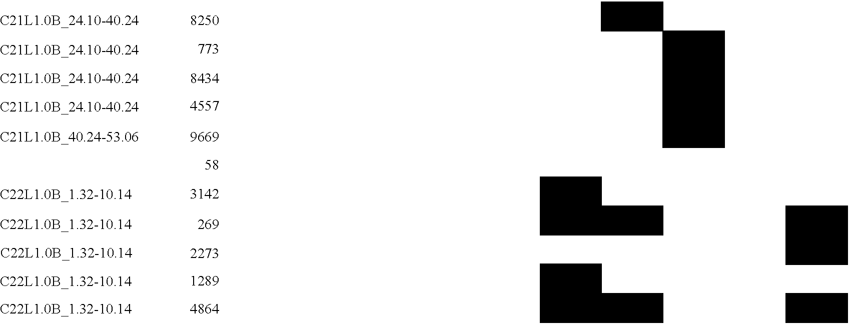

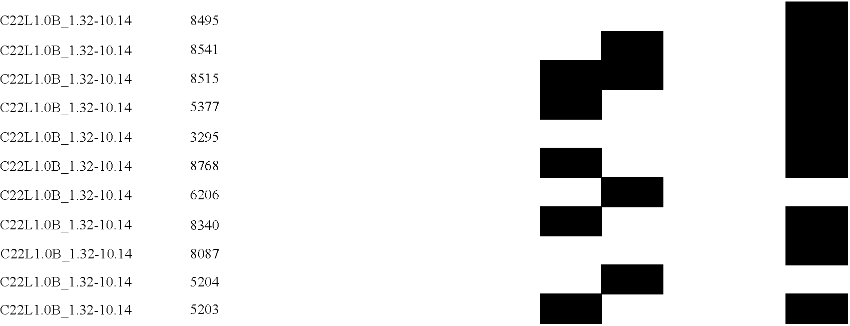

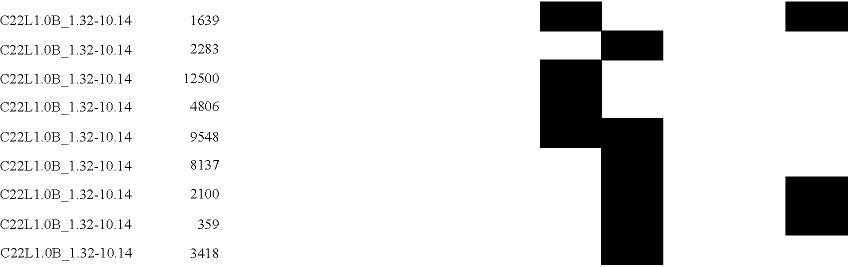

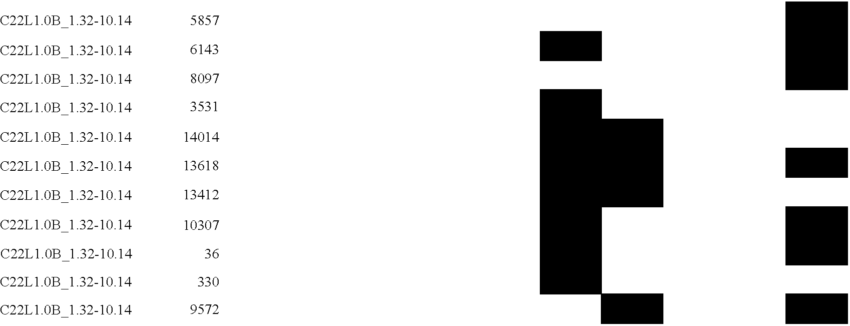

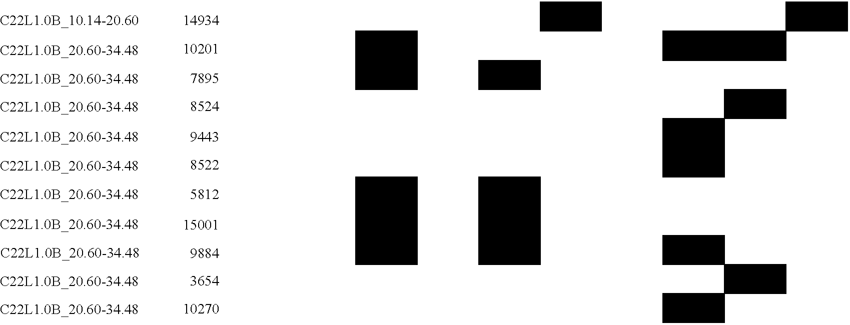

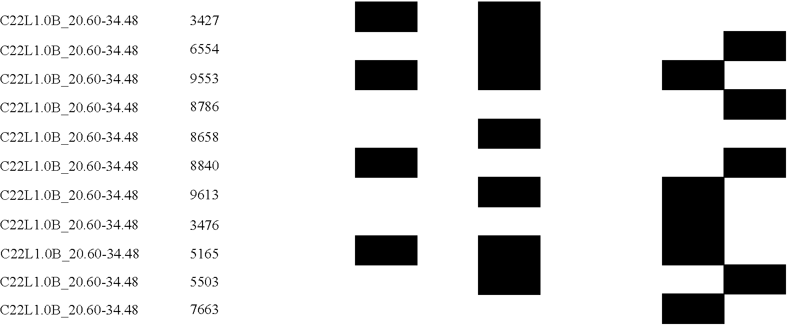

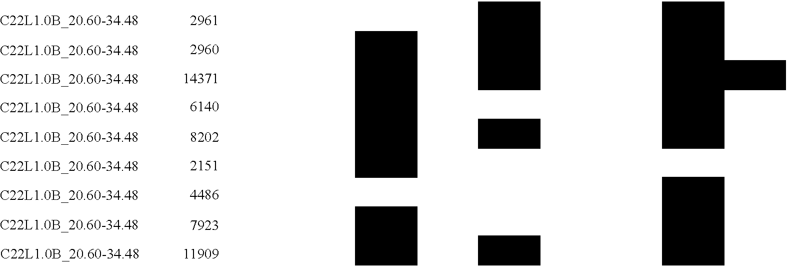

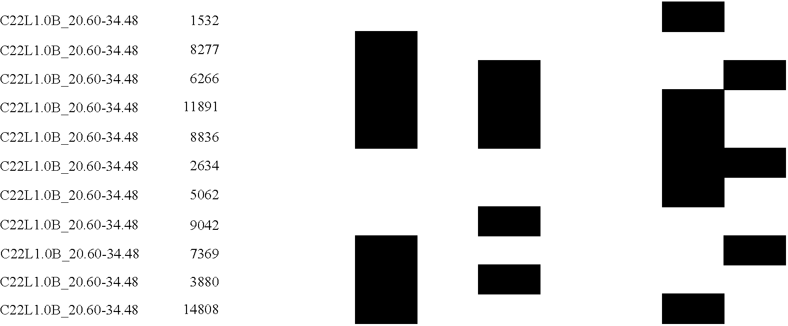

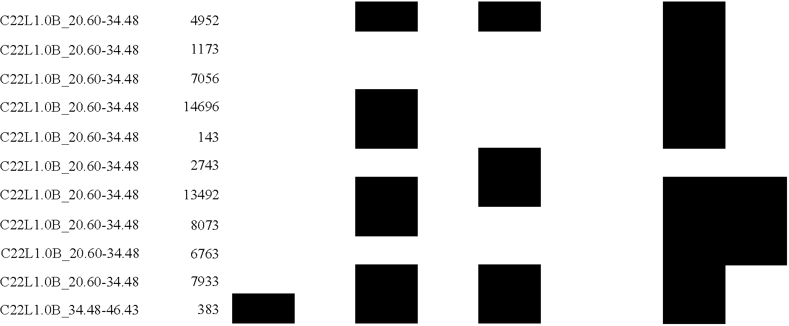

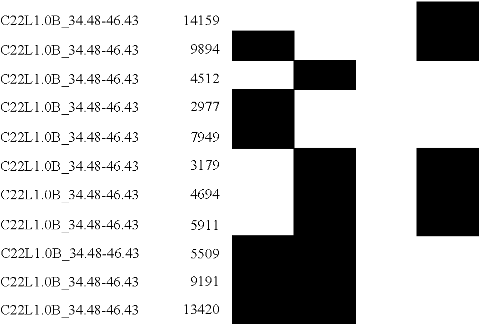

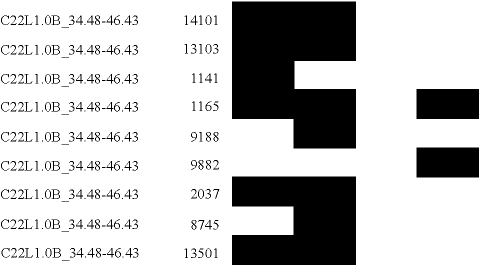

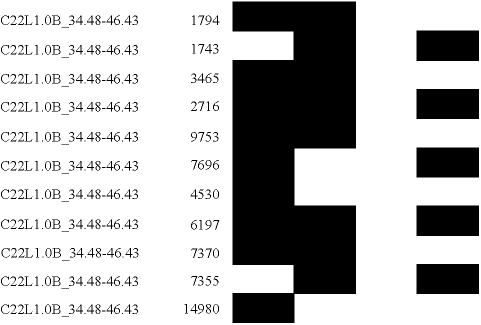

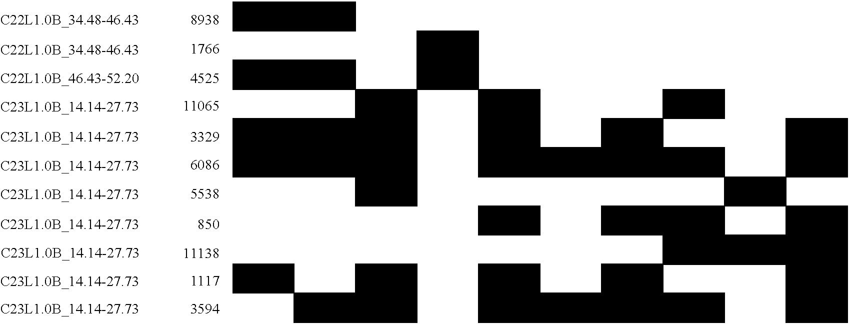

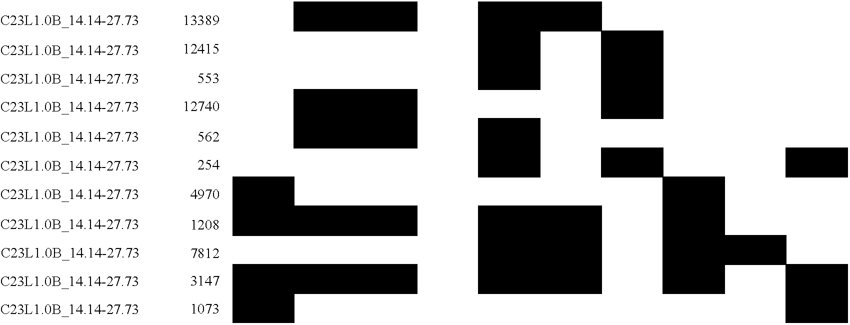

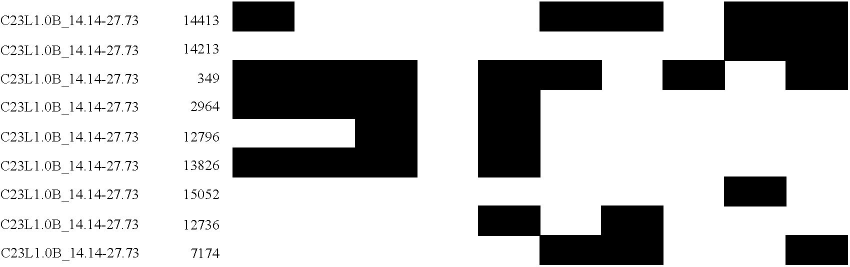

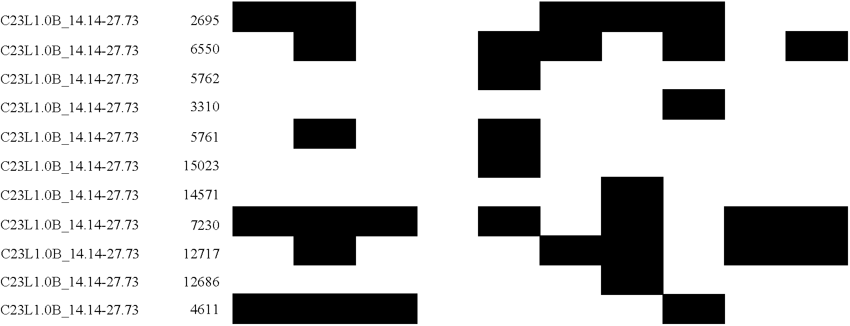

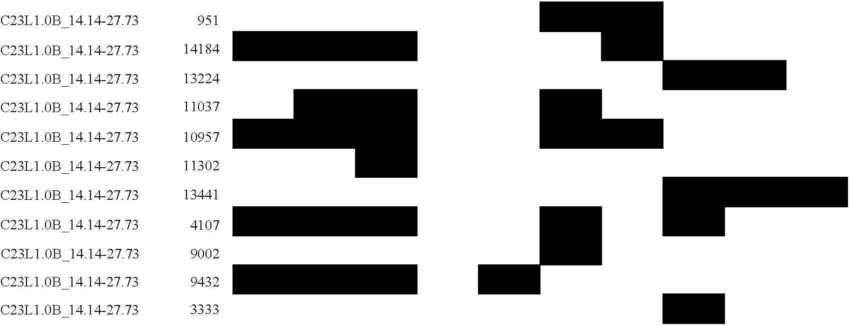

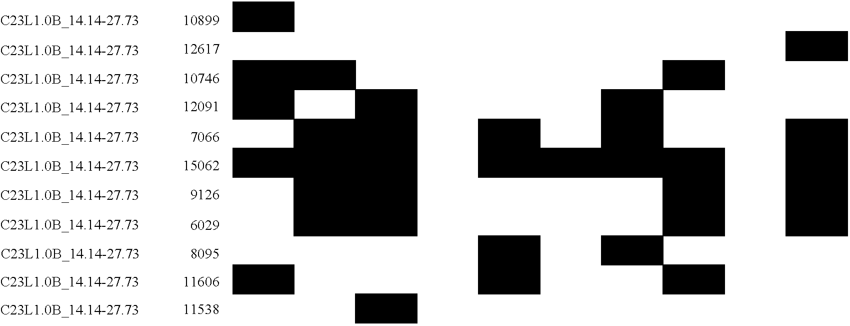

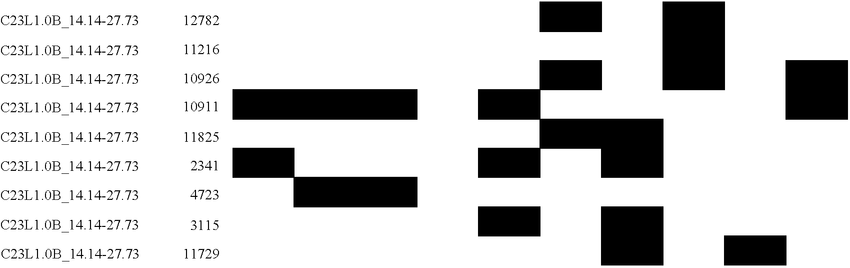

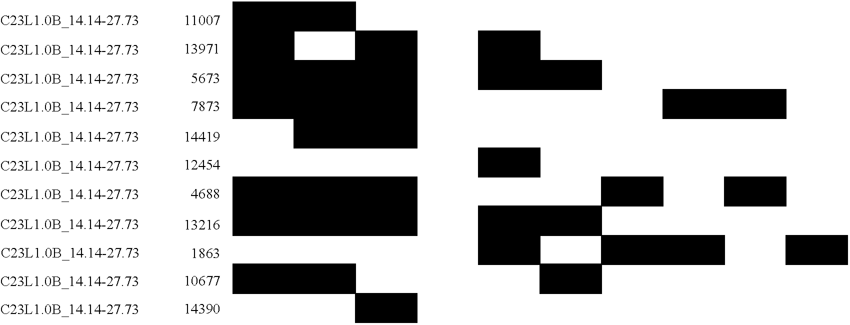

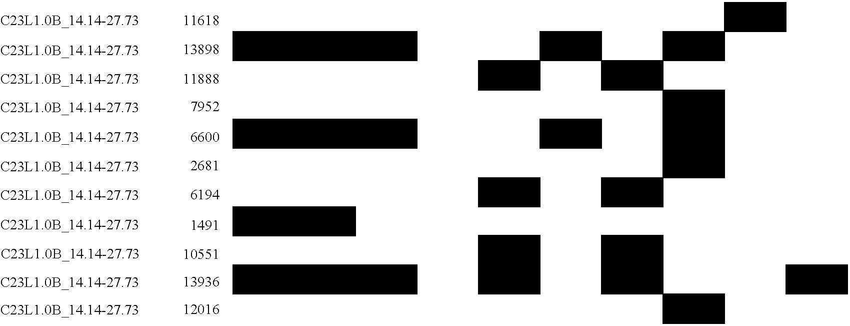

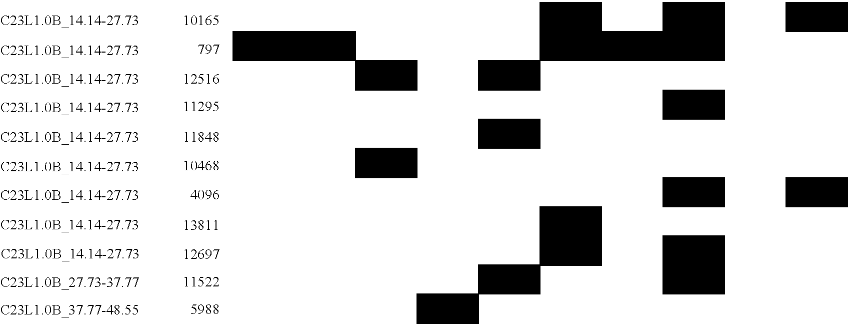

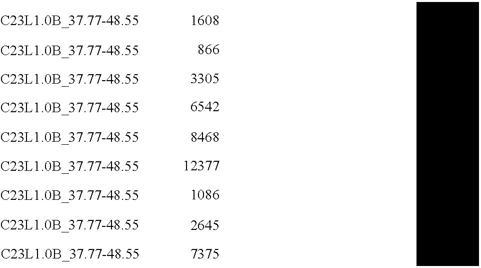

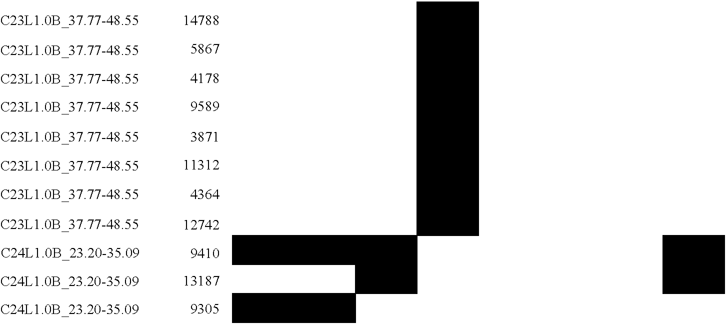

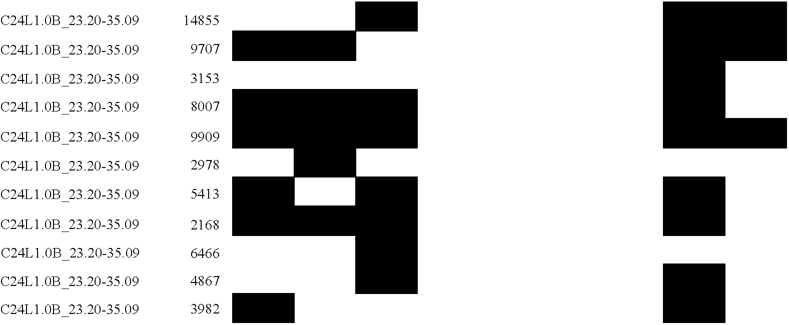

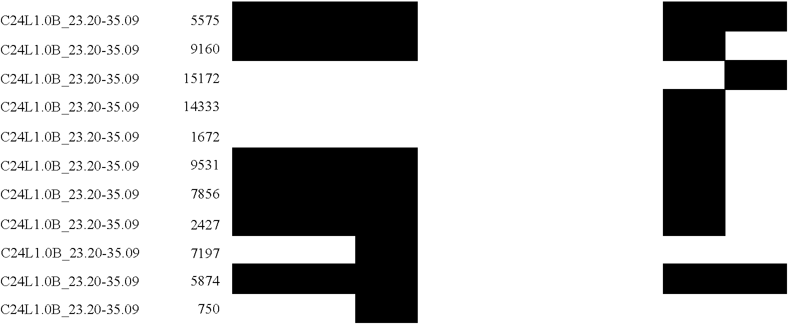

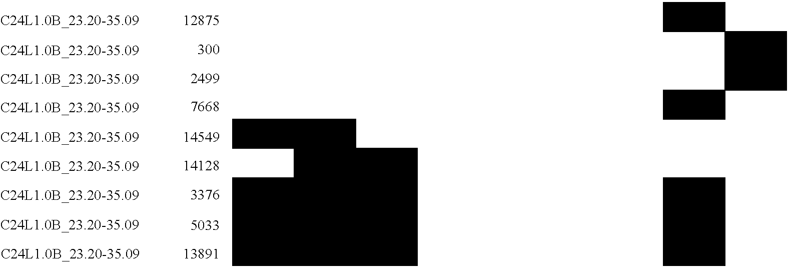

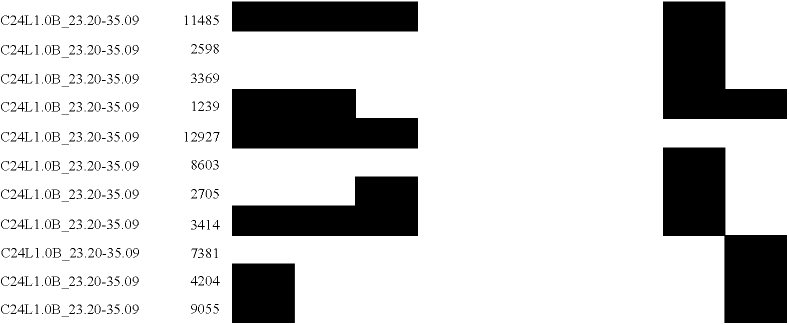

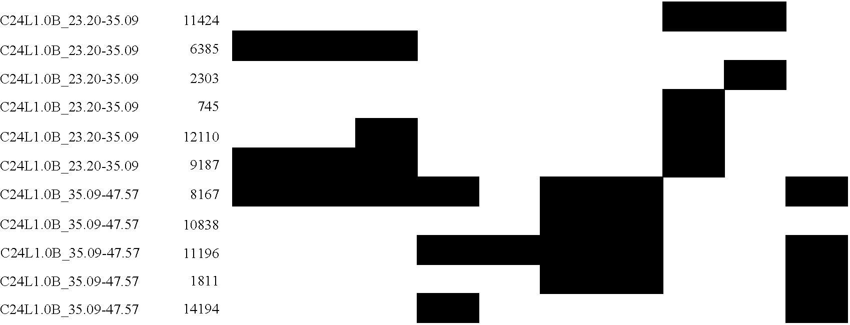

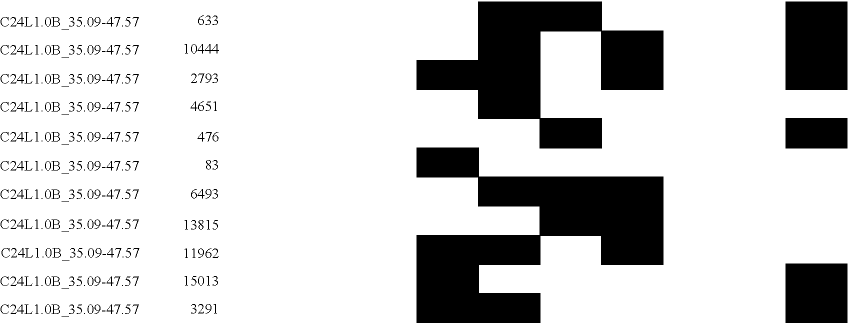

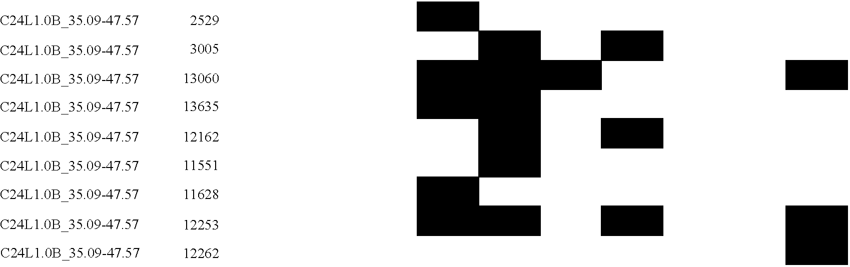

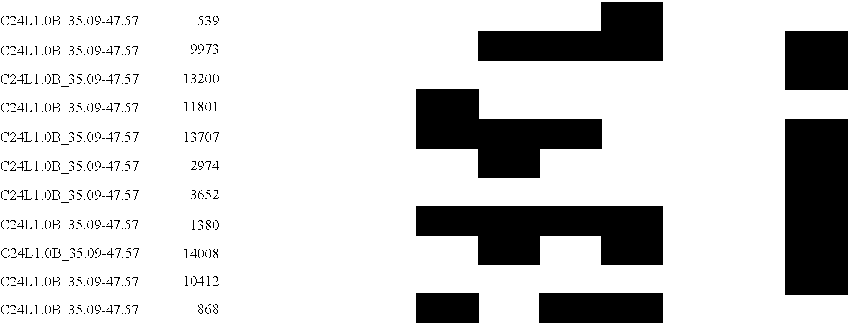

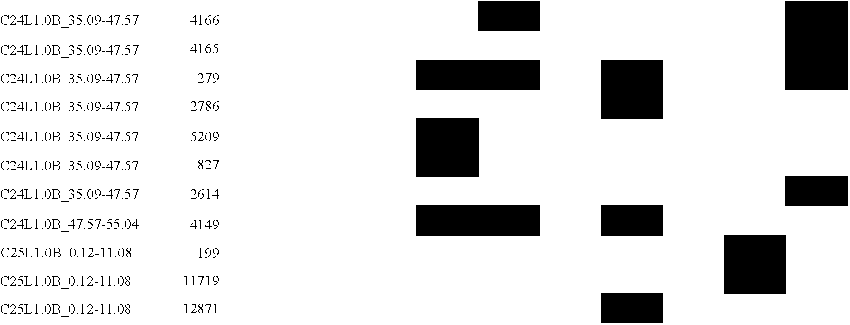

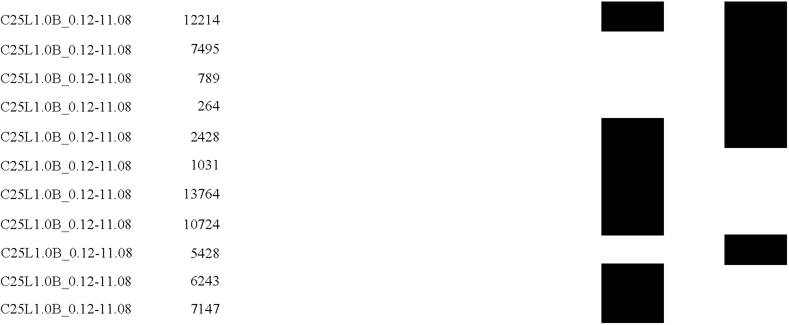

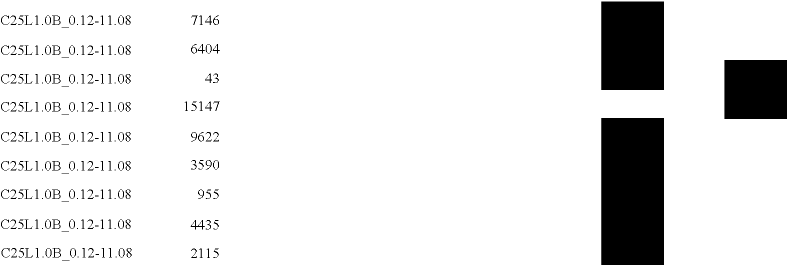

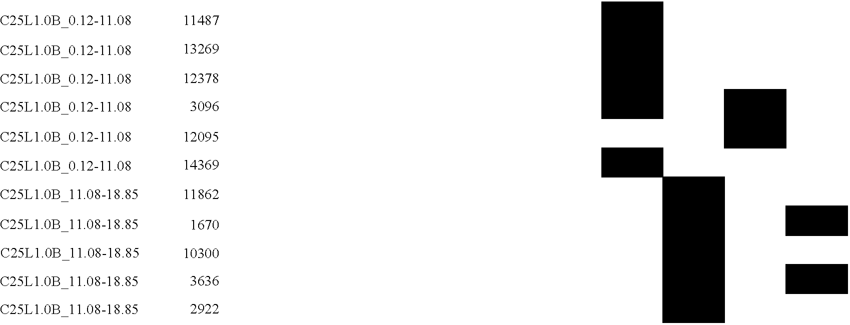

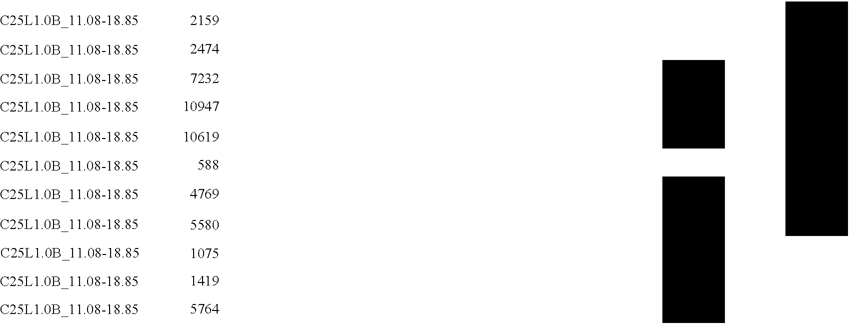

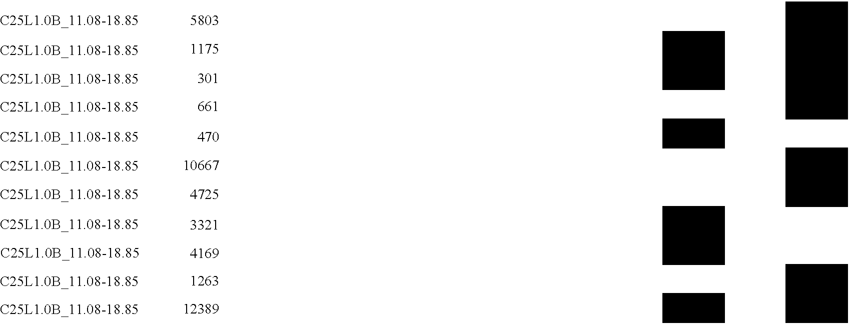

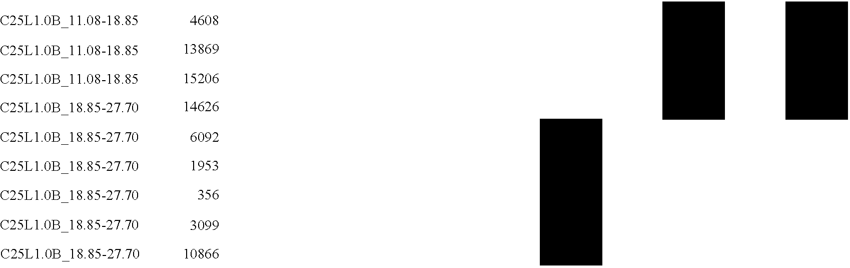

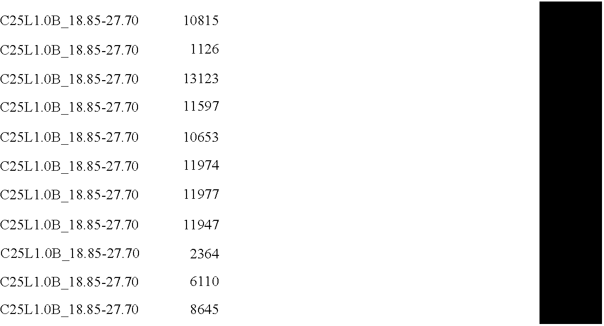

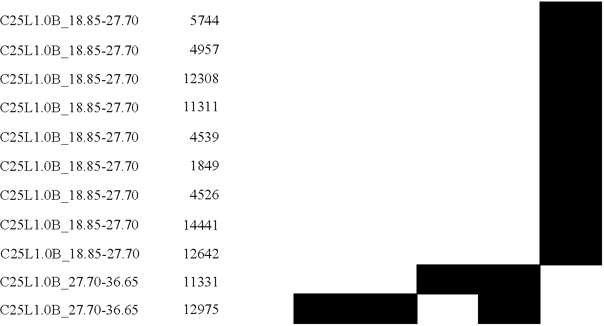

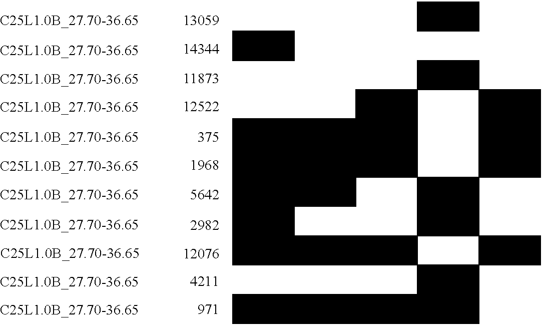

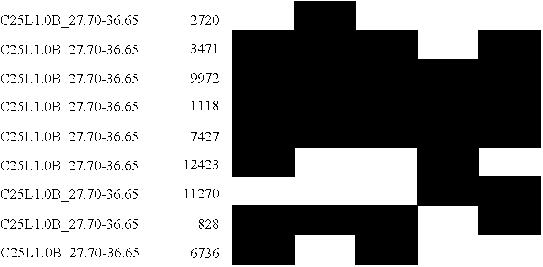

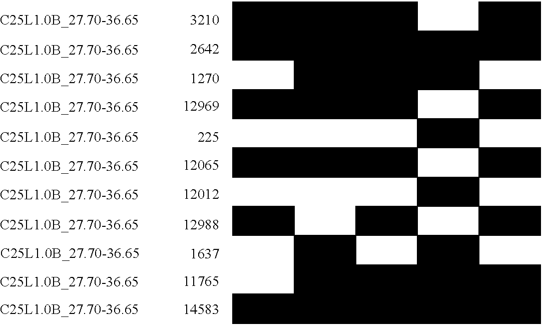

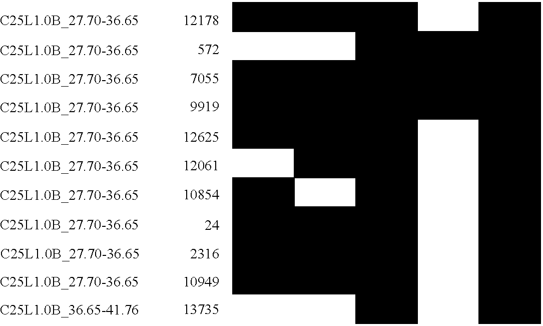

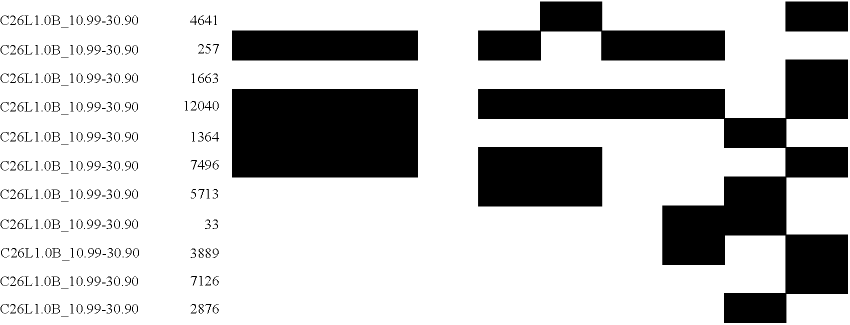

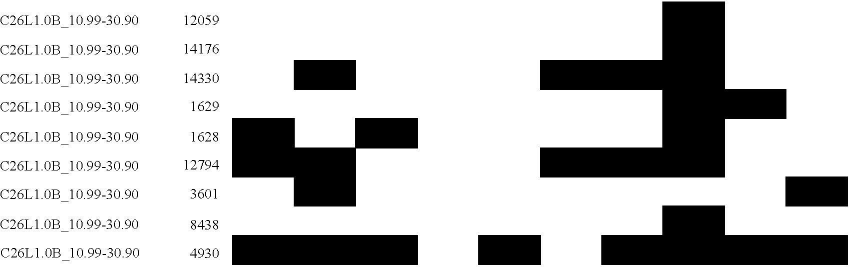

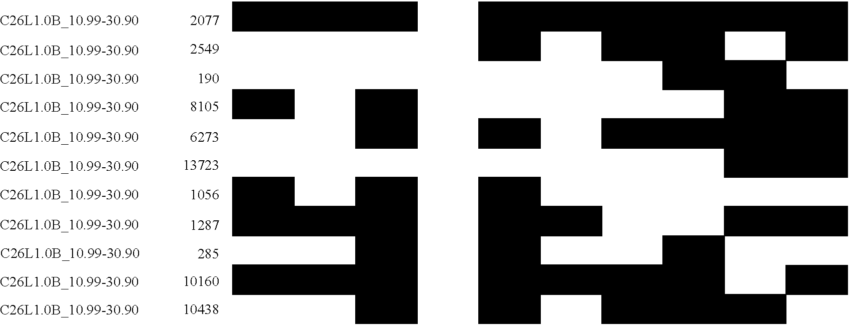

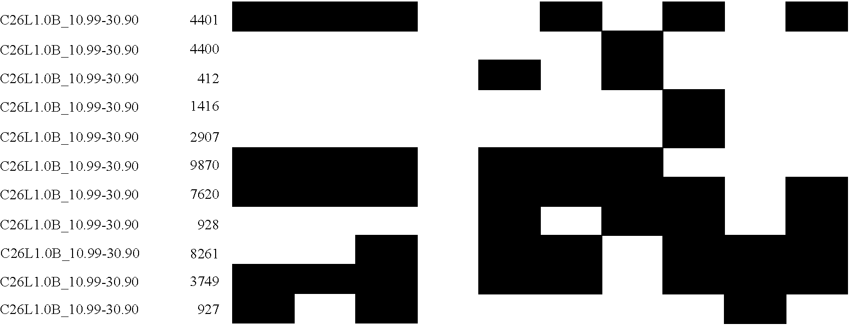

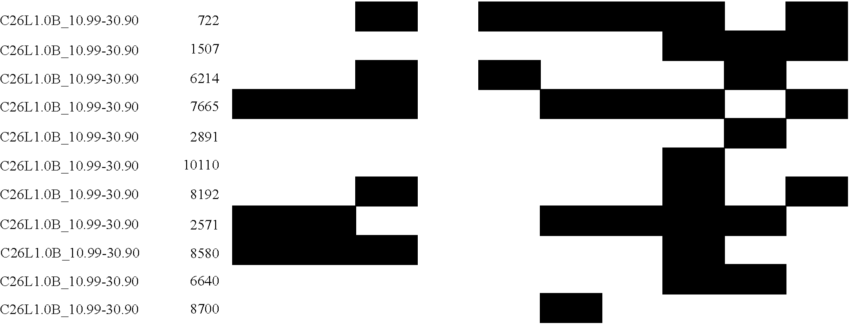

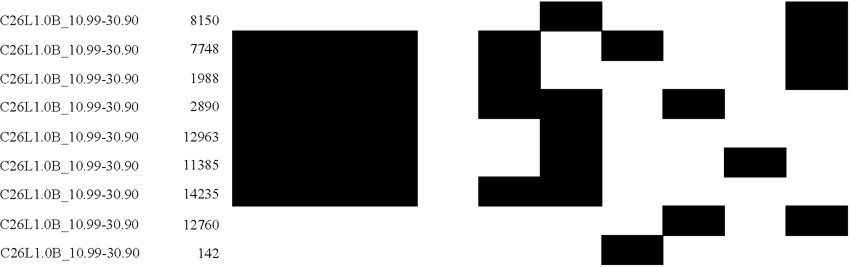

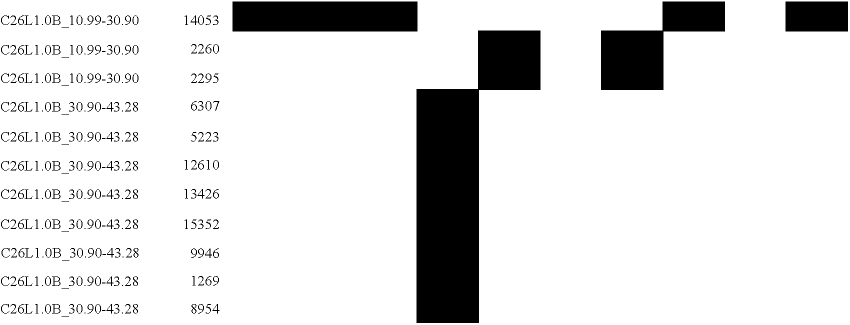

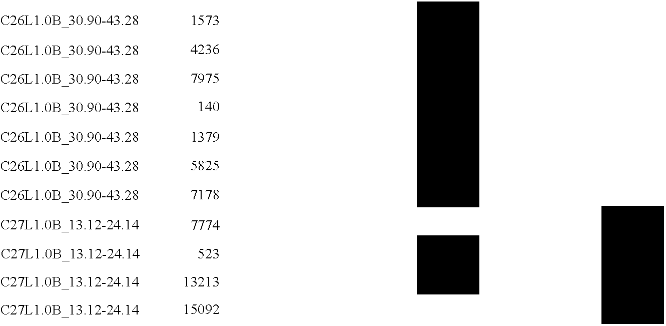

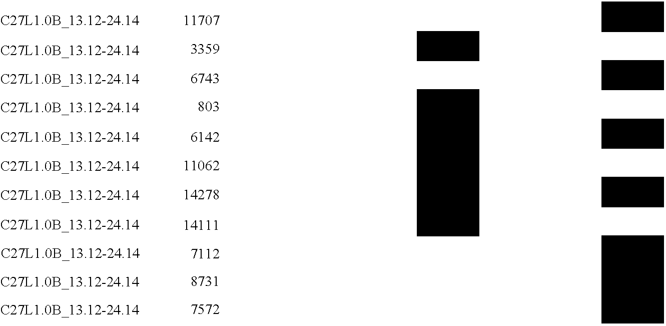

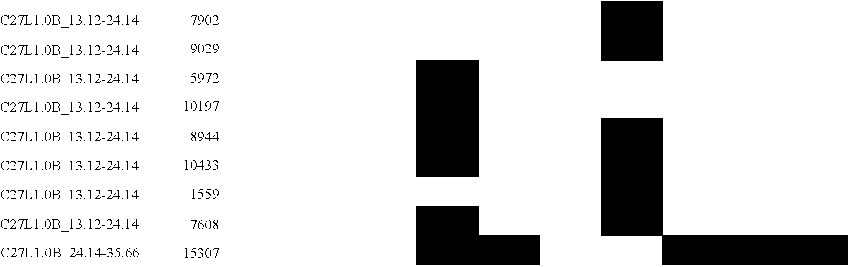

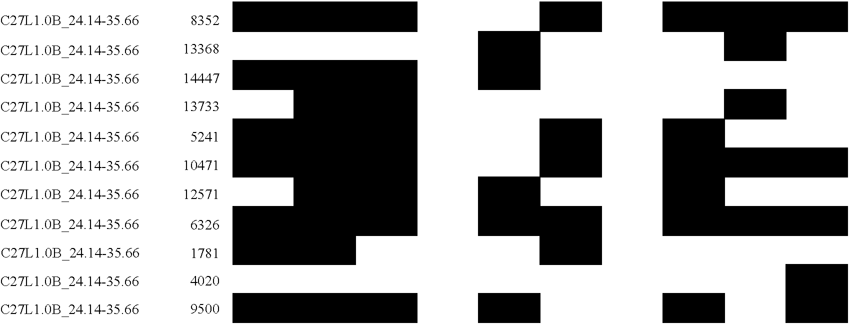

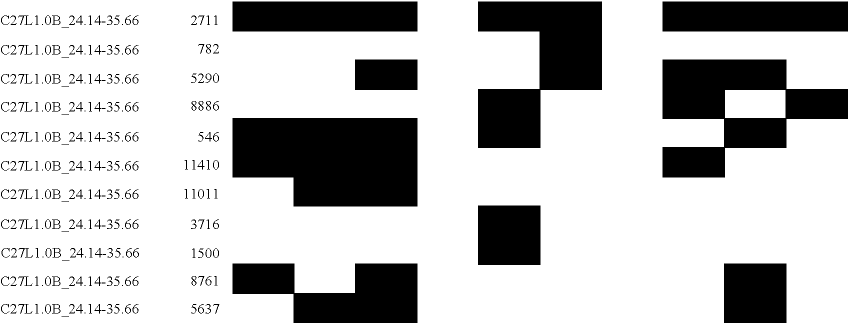

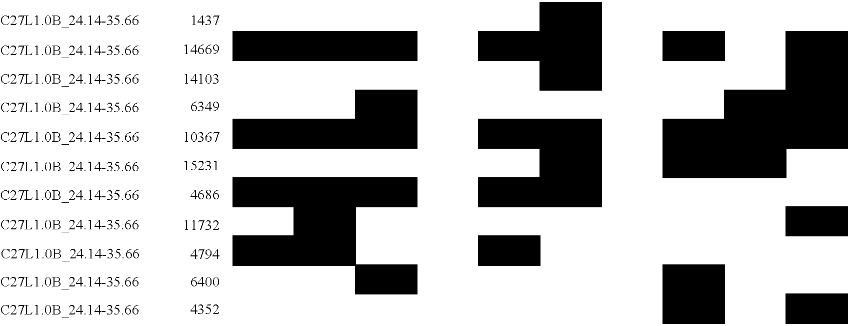

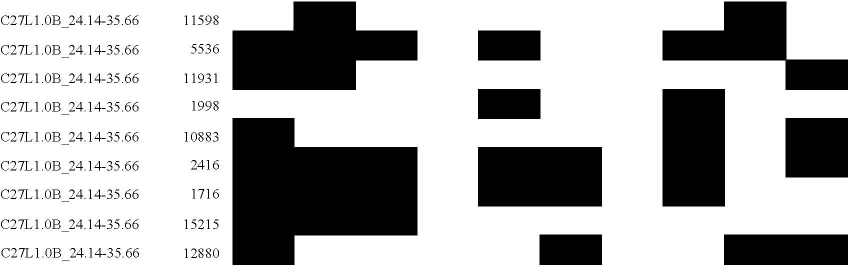

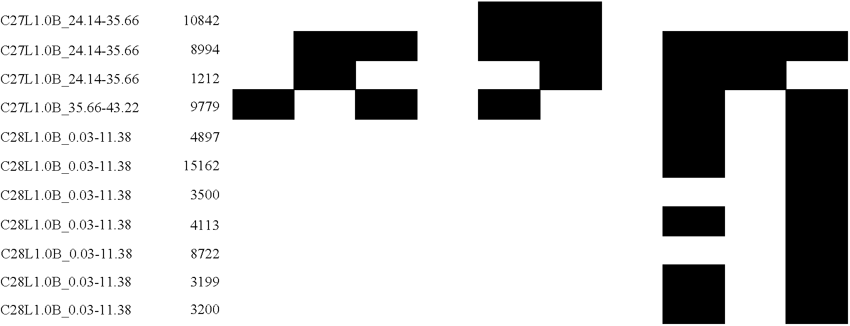

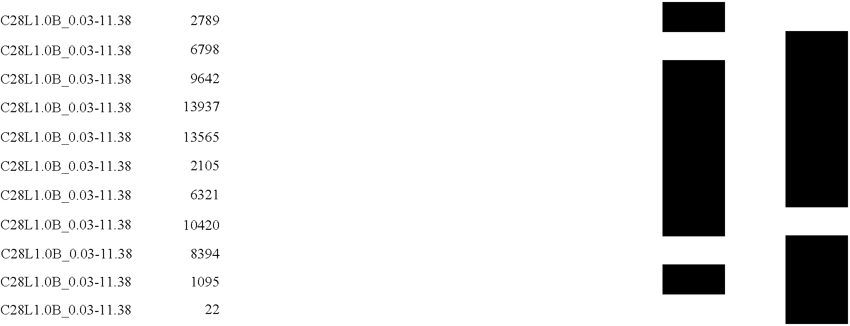

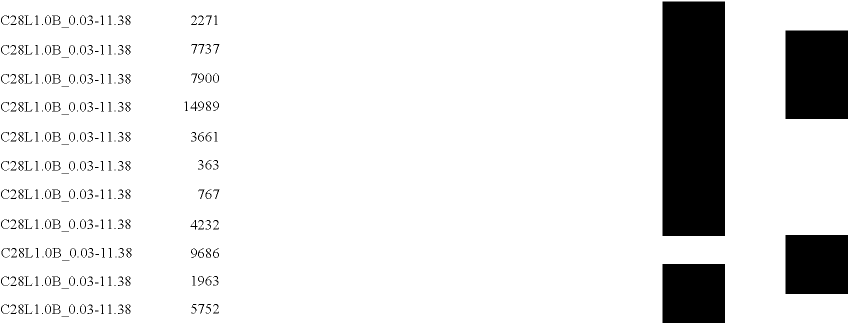

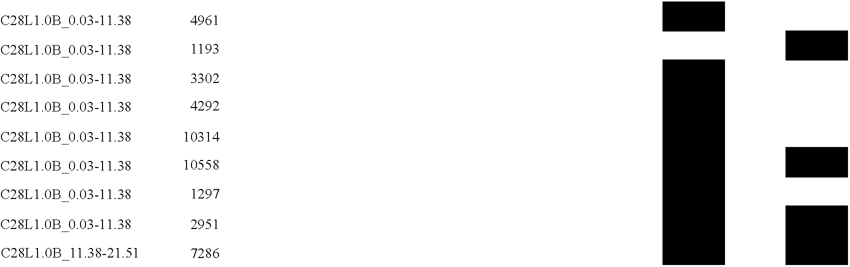

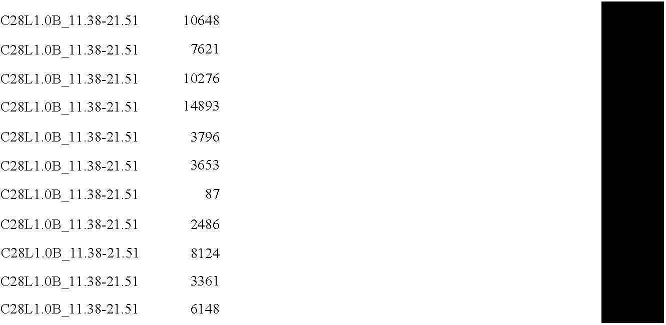

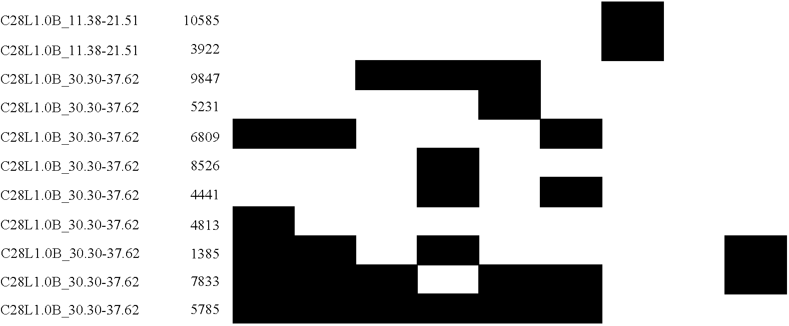

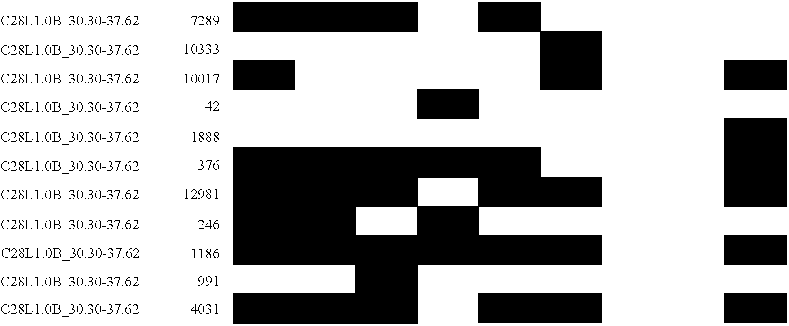

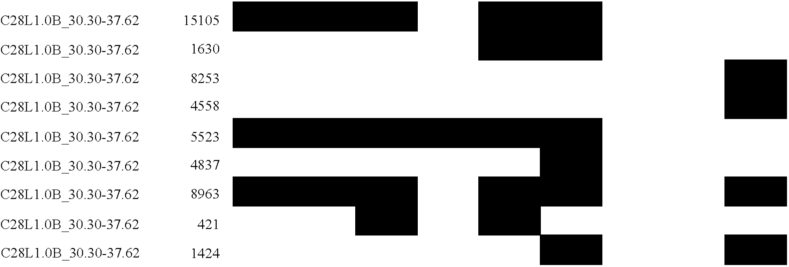

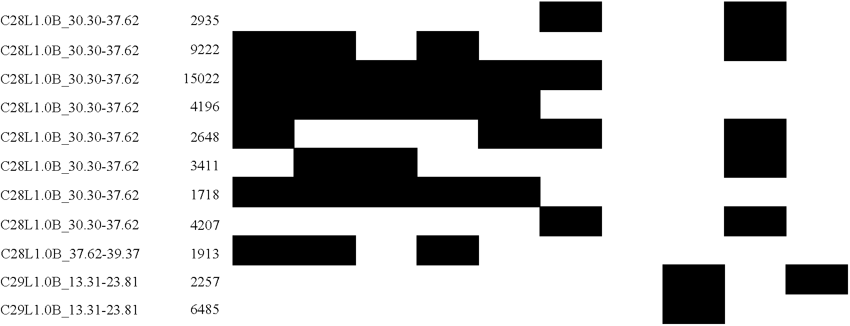

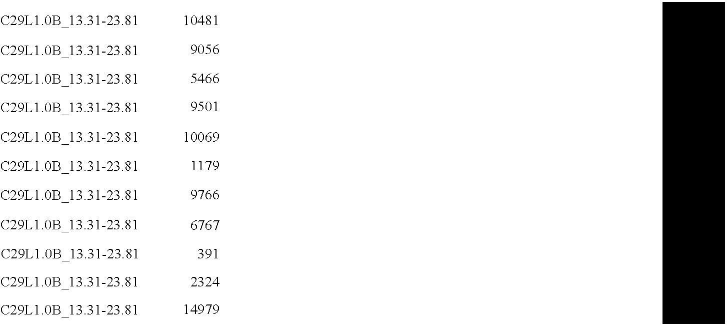

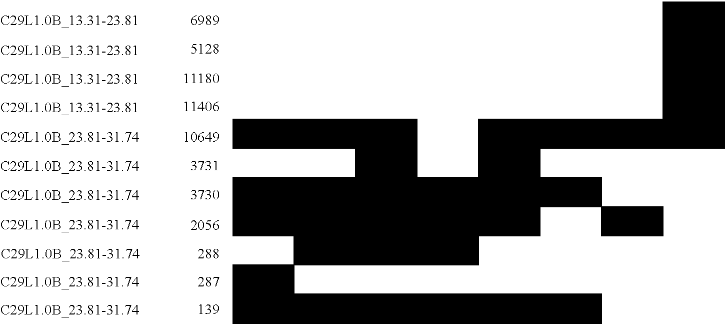

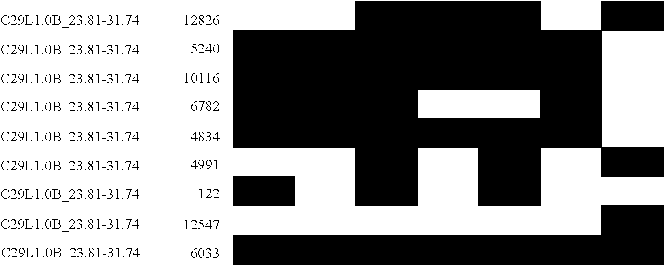

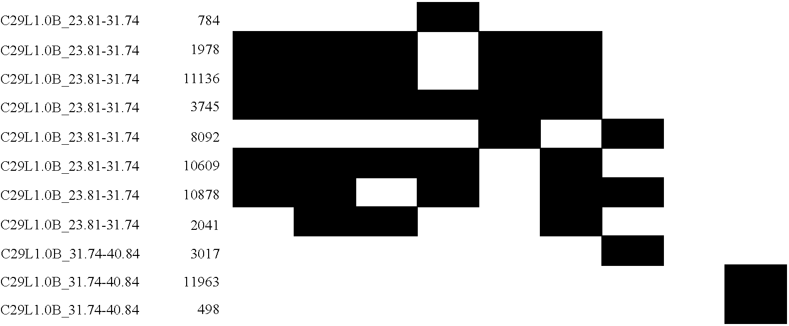

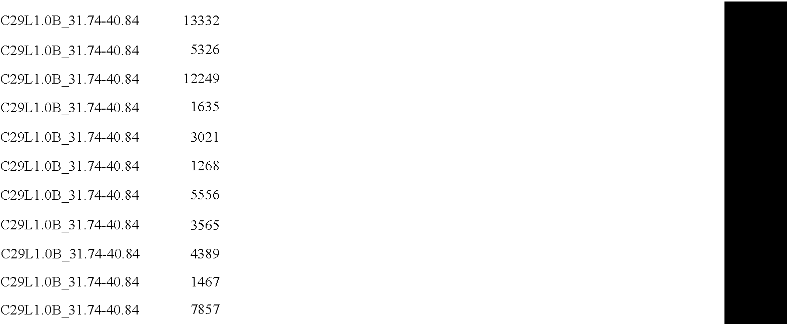

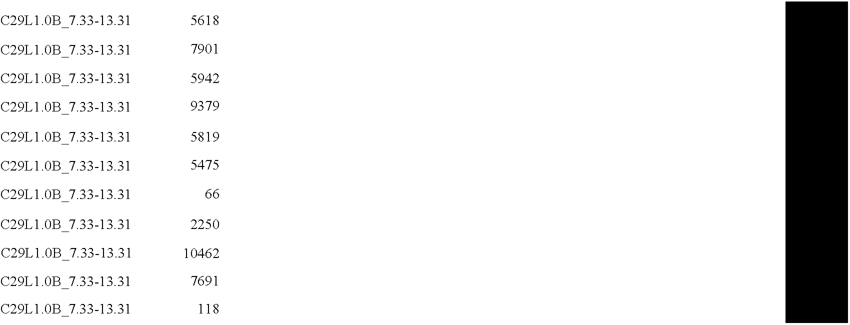

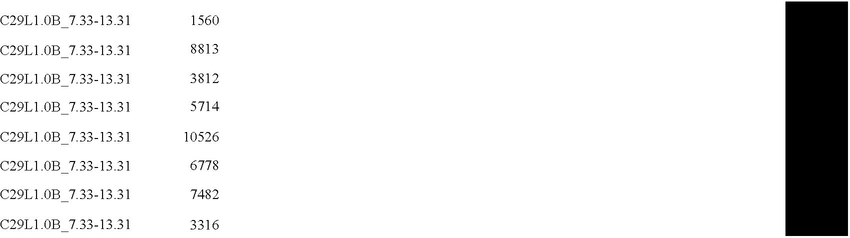

The D′ value was computed between all pairs of 9,195 SNPs, and is shown as a scatter plot of D′ over distance per FIG. 6. In random samples, D′ is equal to p (Morton 2003). FIG. 6 also indicates that there was a decay in LD with increasing distance, but further shows extensive variability in D′ at any one distance. This in part reflects local variations in the extent of LD across the chromosome, which can be represented in LDU maps of individual chromosomes.

The constructed LD map of bovine chromosomes using data for all the 9,195 SNPs genotyped on 1,546 Australian dairy bulls has a total LDU map length of 179 LDUs. Plotting LDU against kilobase locations from bovine assembly 3.1 (Btau3.1) on the X-axis reveals a pattern of steps and plateaus. The larger steps have been shown to correspond to the location of narrow regions of intense recombination (Zhang et al. 2002b), reflecting recombination hot spots. Small steps presumably reflect ancient recombination events. The mean swept radius (1/e) is 13 Mb, which is much larger than the average in humans (Tapper et al. 2005) and indicates much more extensive LD in cattle.

### Example 3

### Structure of LD Along Bovine Chromosomes BTA-1 to BTA-29

A total of 204 LD blocks with tracts of consecutive intervals spanning one LDU were observed along bovine chromosomes. These LD blocks collectively cover all of the chromosomes with a mean LD block size of 13 MB. The location of these blocks is set out in Table 1.

### Example 4

### Traits

Tables 2 to 4 as set out above identify particular LD blocks on each chromosome which are associated with one or more relevant traits. The parameters of the traits are defined as set out below.

For most traits, values against which means and standard deviations are derived have been ascribed within a numerical range for each production trait, with a value of zero being the midpoint of the range, an increasingly negative value being less beneficial and an increasingly positive value being increasingly beneficial. This is shown as follows in the following hypothetical example and applies for a large number of traits.

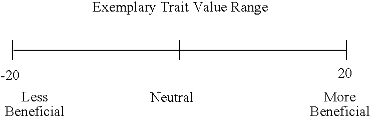

However, for some traits such as Cell Count (indicative of mastitis) and Live Weight (an estimate of feed conversion efficiency), a more negative value is more beneficial on the production trait value range.

In addition, the units attached to the numerical values vary between traits, and include, for example, standard metric units such as weight, length and time, currency units such as dollars, percentages and specifically designed unit ranges, for example, a range of 1 to 9 to describe differences in a particular trait.

Further, some traits are calculated first on a letter value rather than a numerical value, for example, from A to E, with subsequent conversion to a numerical value.

### Example 5

### Australian (Estimated) Breeding Values

ABVs are not an absolute measure of how much an animal will produce. Rather, ABVs are expressed relative to a specific group of animals. The average of this group of animals is referred to as ‘the base’, and is set at zero. This provides a reference point for comparisons between bulls. A separate base is set for each breed and so ABVs are only comparable within breeds.

A new base was generated in February 2005, and is the average ABV of cows born in 2000. For yield traits, cows were included in the new base only if they were born in 2000, were a daughter of an AB bull, are straightbred (e.g., breedcode of JJJJ for Jerseys) and had at least one lactation used in their ABV calculation. For other traits, cow ABVs are not available, so the base was defined as the average of bulls with reliable ABVs born in 1993-1997. This approximates cows born in 2000.

The following sets out the number of cows in the ABV base for yield traits (February 2005).

| Breed | Number of cows in the base |
|---|---|
| Holstein | 124,323 |
| Jersey | 15,388 |
| Guernsey | 417 |

Red Breeds:

| | |
|---|---|
| Illawarra | 1,264 |
| Ayrshire | 590 |
| Aust Red Breed | 1,139 |
| Dairy Shorthorn | 76 |
| Total | 3,069 |
| Brown Swiss | 245 |

### Breed Average ABVs of AI Bulls Born 1985+ as at February 2006

Breed average ABVs of AI bulls born 1985+ as at February 2006

| Trait | Holstein | Jersey | Red Breeds | Guernsey |
|---|---|---|---|---|
| Milking Speed | 90.9 | 90.9 | 89.0 | 89.0 |
| Temperament | 90.8 | 905 | 90.1 | 92.0 |
| Survival | −1.1 | −1.0 | +2.9 | 1.0 |
| Likeability | 91.7 | 91.3 | 88.9 | 89.9 |
| Overall Type | −0.13 | −0.09 | −0.05 | −0.10 |
| Udder depth | 0.01 | 0.02 | −0.01 | −0.05 |
| Pin Set | −0.07 | −0.02 | 0.00 | 0.23 |
| Cell Count | −1.6 | −5.7 | −0.4 | −14.1 |
| Stature | −0.04 | 0.0 | 0.01 | 0.07 |
| Body Depth | 0.03 | 0.01 | −0.01 | 0.08 |
| Chest Width | 0.08 | 0.04 | −0.03 | −0.03 |

### Example 6

### Australian Profit Ranking (APR)

The APR is an index that uses ABVs to estimate a ranking that identifies those bulls that produce the most profitable daughters. The APR is provided in addition to the individual traits so that producers have the option to select on any combination of individual traits.

The Australian Profit Ranking (APR)=Australian Selection Index (ASI)+Milking Speed (MS)+Temperament (TEMP)+Survival (SURV)+Somatic Cell Count (SCC)+Live Weight (LWT)+Fertility (FERT), wherein each component is calculated as per the following:

### Example 7

### Breeding Values for Traits

Breeding values for the following traits were also calculated. These traits include: Protein Yield (kg) (PROT), Protein (w/v) (%)(PROT %), Milk Volume (Litres) (MILK), Fat Yield (kg)(FAT) and Fat % (w/v) (kg) (FAT %).

7.1 Protein % (w/v) and Protein Yield (kg)

Protein content of milk is assessed by automated machines (e.g—Bentley Instruments www. Bentleigh instruments.com; Foss Instruments www.Foss.dk) by infrared scanning of milk specific for N-H amine bond absorption and is reported as percentage (w/v). Protein yield is then calculated accordingly.

### 7.2 Milk Volume (Litres)

A volumetric sample from an on-farm meter is weighed, and milk volume is calculated based on the weight of the volumetric sample and the average density of milk.

7.3 Fat % (w/v) and Fat Yield (kg)

Fat yield is assessed by automated machines (e.g. Bentley Instruments; Foss Instruments; Foss Instruments www.Foss.dk) by infrared scanning of milk specific for C═O and C—H groups and is reported as percentage (w/v). Fat yield is then calculated accordingly.

### Example 8

### Breeding Values for Individual Type Traits

Breeding values for the following type traits were also calculated. These traits include: stature, udder texture, bone quality, angularity, muzzle width, body depth, chest width, pin set, pin width, foot angle, rear leg view, udder depth, fore attachment, rear attachment height, rear attachment width, centre ligament, teat placement, teat length and loin strength

### 8.1 Stature

Stature is measured from the top of the spine in between the hips to the ground. The measurement is precise. The trait is measured on a linear scale of 1-9, each point increase is 3 cm in between the range listed below

| | |
|---|---|
| 1 - Short | 1.30 Metres |
| 5 - Intermediate | 1.42 Metres |
| 9 - Tall | 1.54 Metres |

### 8.2 Udder Texture

Is a measure of the glandular milk making tissue in the udder emphasized by its collapsibility when milked, vein network and softness. Fibrous and fatty tissue in the udder restrict a dairy cow's ability to produce large quantities of milk. A prominent and distinctive vein network on the side of the udder is a reliable indicator of desirable texture. Trait is measured on a linear scale of 1-9, wherein: 1—Fleshy and 9—Soft

### 8.3 Bone Quality

Bone quality is believed to be a reliable indicator of milking ability in a dairy cow. A flat bone is “dense”, and is more desirable in dairy compared with round or coarse bones which are associated with beef rather than dairy production. The trait is measured on a linear scale of 1-9, wherein: 1—Coarse bone and 9—Flat bone

### 8.4 Angularity

Angularity is defined as the angle and openness of the ribs, combined with the flatness of bone in a two year old heifers. Angle and open rib account for 80% of the weighting and bone quality accounts for 20%. The trait is scored on a scale of 1-9, wherein (1-3) Non Angular—Lacks angularity, close ribs, coarse bone, (4-6) Intermediate angle with open rib and (7-9) Very angular open ribbed flat bone.

### 8.5 Muzzle Width

Muzzle width and openness of nostrils as an identified trait is highly important in a country such as Australia where cattle frequently walk vast distances to access feed in extremely warm conditions. The trait is scored on a scale of 1-9, wherein 1—Narrow muzzle and 9—Wide Muzzle

### 8.6 Body Depth

Is the distance between the top of spine and the bottom of the barrel at the last rib—the deepest point. The trait is scored on a scale of 1-9, wherein 1-3 shallow, 4-6 intermediate and 7-9 Deep

### 8.7 Chest Width

Chest width is measured from the inside surface between the front two legs. This trait is measured on a linear scale from 1-9, where each point is equal to 2 cm based on the range listed below as per (1-3) Narrow 13 cm, (4-6) Intermediate and (7-9) Wide 29 cm.

### 8.8 Pin Set

This trait is measured as the angle of the rump structure from hooks (hips) to pins on a linear scale of 1-9

| | | |
|---|---|---|
| 1 High Pins | (4 | cm) |
| 2 | (2 | cm) |
| 3 Level | (0 | cm) |
| 4 Slight slope | (−2 | cm) |
| 5 Intermediate | (−4 | cm) |
| 6 | (−6 | cm) |
| 7 | (−8 | cm) |
| 8 | (−10 | cm) |
| 9 Extreme Slope | (−12 | cm) |

### 8.9 Pin Width

This trait is calculated as the distance between the most posterior point of the pin bones, where 1=10 cm and 9=26 cm and every point between is calculated upon intermediate 2 cm lengths.

### 1-3 Narrow

### 4-6 Intermediate

### 7-9 Wide

### 8.10 Foot Angle

This trait is calculated as the angle at the front of the rear hoof measured from the floor of the hairline at the right hoof. This trait is measured on a linear scale from 1-9, where:

1-3 Very Low angle
4-6 Intermediate angle
7-9 Steep angle
Where 1=15 degrees, 5=45 degrees and 9=65 degrees

### 8.11 Rear Leg Rear View

This trait is the direction of the feet when the viewed from the rear.

### 1—Extreme toe out

### 5—Intermediate toe out

9—Parallel feet

### 8.12 Udder Depth

This trait is calculated as the distance from the lowest part of the udder floor to the hock where:

1 Below hock
2 Level with hock

### 5 Intermediate

### 9 Shallow

### 8.13 Fore Udder Attachment

This trait is calculated as the strength of the attachment of the fore udder to the abdominal wall. This is not a true linear trait.

### 1-3 Weak and Loose

4-6 Intermediate acceptable
7-9 Extremely strong

### 8.14 Rear (Udder) Attachment Height

This trait is calculated as the distance between the bottom of the vulva to the highest point of the milk secreting tissue in relation to the height of the animal. A score of 4 represents the mid point of 29 cm, and each point is worth 2 cm.

| | |
|---|---|
| 1 Very Low | 23 cm |
| 2 | 25 cm |
| 3 | 27 cm |
| 4 Intermediate | 29 cm |
| 5 | 31 cm |
| 6 | 33 cm |
| 7 | 35 cm |
| 8 | 37 cm |
| 9 High | 39 cm |

### 8.15 Rear (Udder) Attachment Width

This trait is calculated wherein the reference point for measurement is at the top of the milk secreting tissue measured on a linear scale of 1 to 9, where 1 is extremely narrow and 9 is extremely wide.

### 8.16 Central Ligament

This trait is calculated as the depth of the cleft measured at the base of the rear udder.

| | | |
|---|---|---|
| 1. Convex to flat floor | (1 | cm) |
| 2. | (0.5 | cm) |
| 3. | (0 | cm) |
| 4. Slight Definition | (−1 | cm) |
| 5. | (−2 | cm) |
| 6. | (−3 | cm) |
| 7. Deep Definition | (−4 | cm) |
| 8. | (−5 | cm) |
| 9. | (−6 | cm) |

### 8.17 Teat Placement

This trait is calculated as the position of the front teat from the centre of the quarter.

1-3 Outside of quarter
4-6 Middle of quarter
7-9 Inside quarter

### 8.18 Teat Length

This trait is calculated as the length of the front teat, where each point is 1 cm and the scale ranges from 1 to 9.

### 1-3 Short

### 4-6 Intermediate

### 7-9 Long

### Example 9

### Breeding Values for Live Weight

Live Weight is reported as a deviation in kilograms of live weight from the base set at zero. Live Weight is based on ABVs measured by breed societies. The predictors and their relative contributions are:

Live Weight=(0.5×stature ABV−mean ABV)+(0.25×Chest Width−mean ABV)+(0.25×Body Depth−mean ABV)

A bull with a lower Live Weight ABV will reduce the live weight of their daughters while a bull with a higher Live Weight ABV will increase the live weight of their daughters compared to the base.

### Example 10

### Breeding Values for Workability

The workability traits are milking speed, temperament and likeability. Each of these traits are scored on a scale from A to E by the dairy farmer, where A is very desirable and E is very undesirable. Satisfactory daughters are those expected to receive scores of C, B or A from the farmer. The metric is expressed as a percentage:

$$\% = \frac{\begin{matrix}number of offspring expected \\ to be satisfactory ( A , B , C )\end{matrix}}{all offspring ranked}$$

### Example 11

### Breeding Values for Somatic Cell Count (SCC)

Somatic Cell Count breeding value is expressed as the % increase or decrease in cell count compared to the average or BASE (set at zero). Thus a bull with lower CC ABV on average has daughters with lower cell count which is an indicator of increased mastitis resistance, and a bull with a higher CC ABV has daughters with higher cell count which is an indicator of mastitis susceptibility.

Cell count can be assessed by laser-based flow cytometry, being a common method for distinguishing between different cell populations and/or counting cell numbers. Briefly, a milk sample is taken and mixed with a fluorescent dye, which disperses the globules and stains DNA in cells. An aliquot of the stained suspension is injected into a laminar stream of carrier fluid. Cells are separated by the stream of carrier fluid and exposed to a laser beam. As the cells pass through the excitation source the stained cell nuclei fluoresce, the signal which is multiplied and cell number calculated. Indicative CC levels are as follows:

| | |
|---|---|
| Over 200,000: | mastitis |
| <200,000: | maximum desired number of cells/ml milk |
| <100,000: | number of cells/ml milk where the cow is considered |
| | to have minimal to no mastitis [ICAR] |

### Example 12

### Breeding Values for Fertility

Daughter fertility ABVs measure the difference between bulls for the percentage of their daughters pregnant by 6 weeks after mating start date. In year-round herds this is equivalent to is the percentage of their daughters pregnant by 100 days after calving. Data is derived from the following records:
Calving dates used to determine calving interval and stage of pregnancy
Mating data is used to determine days to first service

### Example 13

### Breeding Values for Survival

The survival ABV is reported as the percentage of daughters that survive from one year to the next compared to the average/BASE (set at zero). The Survival ABV is based on actual daughter survival and a combination of predictors of survival The predictors and their relative contributions are:

Survival Predictors=(0.5×likeability ABV−mean ABV)+(1.8×Overall Type ABV−mean ABV)+(3.0×Udder Depth ABV−mean ABV)+(2.2×Pin Set ABV−mean ABV)

As the number of daughters scored for survival increases for a bull, the contribution of the predictors is reduced.

### Example 14

### Breeding Values for Calving Ease

The calving ease ABV is expressed as the percentage of ‘normal’ calvings expected when joined to mature cows in the average Australian herd. The calving ease for a bull is based on farmer assessment of the difficulty experienced with the birth of the progeny of the bull, relative to births in the same herd in the same season. In this context, ‘normal’ calving means not subject to a hard pull.

### Example 15

### Mammary System

Mammary System ABV is calculated using the formula below based on linear traits that have been differentially weighted. The differential weighting of each of the linear traits is based on regression analysis and the contribution of these traits to the variance observed in the system overall.

Mammary System ABV=(Udder texture ABV×0.161)+(Fore Attachment ABV×0.4753)+(Rear attachment height ABV×0.454)+(rear attachment width ABV×0.448)+(Centre Ligament ABV×0.355)+(teat placement ABV×0.269)

### Example 16

### Overall Type

The type classification program uses a linear scores system, in which physical characteristics of the cows are actually “measured” (see above). The type scores are then combined into composite traits (score-card traits), which describe more general aspects of the cow conformation. These composite traits tend to receive more attention than individual scores, because they are easier to interpret, and they probably would be the traits considered by dairymen when making culling decisions. (Reference https://upload.mcgill.ca/animal/97r12.pdf)

Overall type is the weighted assessment of eight composite type traits namely: ‘Final Score’, ‘Frame-Capacity’, ‘Rump’, ‘Feet and Legs’, ‘Fore Udder’, ‘Rear Udder’, ‘Mammary System’ and ‘Dairy Character’. The exact means of assessing overall type is confidential amongst those skilled in the art (e.g. Holstein organizations and their trained staff make the assessment). (Reference: https://upload.mcgill.ca/animal/97r12.pdf)

### Example 17

### Australian Selection Index

The Australian Selection Index (ASI) is expressed as the net financial production profit (in $) per cow per year. It includes a consideration of protein, fat and milk volume traits. The formulation is based on the milk payment system whereby farmers are paid by the percentage of protein and fat in milk, with a charge on milk volume.

Australian Selection Index (ASI)=(3.8×Protein ABV)+(0.9×Fat ABV)−(0.048×Milk ABV)

Milking Speed (MS)=1.2×(Milking Speed ABV−mean ABV)

Temperament (TEMP)=2.0×(Temperament ABV−mean ABV)

Survival (SURV)=3.9×(Survival ABV−mean ABV)

SCC=−0.34×(Somatic Cell Count ABV−mean ABV)

LWT=−0.26×(Liveweight ABV−mean ABV)

FERT=3.0×(Daughter Fertility ABV−mean ABV)

### Example 18

### Redundancy of SNPs

As an example of each of the blocks described herein, for Block C1L1.0B_{—}59.93-83.90, 101 SNPs were used to calculate the R² value listed in Table 2.

The SEQ ID NOs of these SNPs were
2576 341 9934 10144 3787 7037 3023 14135 7671 7670 8258 4070 7730 2750 9135 2277 1877 1248 1249 1250 6645 8551 13175 1603 491 6363 15110 4372 8665 10988 14953 2848 15205 14522 14041 14665 5288 10436 3241 2504 4528 4893 5677 8810 6457 8441 840 4397 13158 14398 13741 14310 13406 13312 13939 5546 9315 14873 13480 12264 7199 10341 9791 1950 8926 13729 13708 12042 2334 14234 12019 8004 9889 9594 5913 2972 2973 6426 1929 50 14421 15042 7255 4771 3071 2304 3971 14262 8093 5160 5979 6749 4301 3737 9627 10591 8483 5740 3278 10216 9785

Of these, one SNP (SEQ ID NO:9785) was shared by the next block.

Only 39 of these SNPs for the APR trait, for example, were required to explain all of the R² value. These SNPs were SEQ ID NOS:
2576 341 9934 3023 4070 2750 9135 6645 8551 15110 8665 10988 2848 15205 5288 10436 3241 4528 4893 6457 13158 14398 10341 1950 12042 6426 1929 50 14421 4771 2304 3971 14262 8093 5979 8483 5740 3278 10216

The following residual SNPs (101 minus 39) in the block do not contribute further to the R² value for APR:
10144 3787 7037 7670 8258 4070 7730 2750 2277 1877 1248 1249 1250 13175 1603 491 6363 4372 14953 14522 14041 14665 2504 5677 8810 8441 840 4397 13741 14310 13406 13312 13939 5546 9315 14873 13480 12264 7199 9791 8926 13729 13708 2334 14234 12019 8004 9889 9594 5913 2972 2973 14421 15042 7255 3071 5160 6749 4301 3737 9627 10591

Thus, knowledge of markers within the defined LD block can be used to predict variation in the relevant performance trait by genotyping animals of unknown performance and substituting marker effects to give a cumulative worth of performance index. Animals can be ranked, therefore selected for future performance for a particular purpose.

Any new marker within the defined LD block will considered to be in linkage disequilibrium (LD) and therefore it is redundant in the presence of existing markers or can be used as proxy for the existing markers without loss of utility.

### REFERENCES

Abecasis, G. R., W. O. Cookson and L. R. Cardon, 2001 The power to detect linkage disequilibrium with quantitative traits in selected samples. Am J Hum Genet 68: 1463-1474.
Altshuler, D., L. D. Brooks, A. Chakravarti, F. S. Collins, M. J. Daly et al., 2005 A haplotype map of the human genome. Nature 437: 1299-1320.
Arnheim, N., P. Calabrese, and M. Nordborg. 2003. Hot and cold spots of recombination in the human genome: the reason we should find them and how this can be achieved. Am J Hum Genet 73: 5-16.
Barrett, J. C., B. Fry, J. Maller and M. J. Daly, 2005 Haploview: analysis and visualization of LD and haplotype maps. Bioinformatics 21: 263-265. Epub 2004 August 2005.
Dawson, E., G. R. Abecasis, S. Bumpstead, Y. Chen, S. Hunt, D. M. Beare, J. Pabial, T. Dibling, E. Tinsley, S. Kirby, D. Carter, M. Papaspyridonos, S. Livingstone, R. Ganske, E. Lohmussaar, J. Zernant, N. Tonisson, M. Remm, R. Magi, T. Puurand, J. Vilo, A. Kurg, K. Rice, P. Deloukas, R. Mott, A. Metspalu, D. R. Bentley, L. R. Cardon, and I. Dunham. 2002. A first-generation linkage disequilibrium map of human chromosome 22. Nature 418: 544-548.
De Bakker, P. I., R. Yelensky, I. Pe'er, S. B. Gabriel, M. J. Daly et al., 2005 Efficiency and power in genetic association studies. Nat Genet 37: 1217-1223. Epub 2005 October 1223.
De La Vega, F. M., H. Isaac, A. Collins, C. R. Scafe, B. V. Halldorsson, X. Su, R. A. Lippert, Y. Wang, M. Laig-Webster, R. T. Koehler, J. S. Ziegle, L. T. Wogan, J. F. Stevens, K. M. Leinen, S. J. Olson, K. J. Guegler, X. You, L. H. Xu, H. G. Hemken, F. Kalush, M. Itakura, Y. Zheng, G. de The, S. J. O'Brien, A. G. Clark, S. Istrail, M. W. Hunkapiller, E. G. Spier, and D. A. Gilbert. 2005. The linkage disequilibrium maps of three human chromosomes across four populations reflect their demographic history and a common underlying recombination pattern. Genome Res 15: 454-462.
Fan, J. B., A. Oliphant, R. Shen, B. G. Kermani, F. Garcia, K. L. Gunderson, M. Hansen, F. Steemers, S. L. Butler, P. Deloukas, L. Galver, S. Hunt, C. McBride, M. Bibikova, T. Rubano, J. Chen, E. Wickham, D. Doucet, W. Chang, D. Campbell, B. Zhang, S. Kruglyak, D. Bentley, J. Haas, P. Rigault, L. Zhou, J. Stuelpnagel, and M. S. Chee. 2003. Highly parallel SNP genotyping. Cold Spring Harb Symp Quant Biol 68: 69-78.
Farnir, F., W. Coppieters, J. J. Arranz, P. Berzi, N. Cambisano, B. Grisart, L. Karim, F. Marcq, L. Moreau, M. Mni, C. Nezer, P. Simon, P. Vanmanshoven, D. Wagenaar, and M. Georges. 2000. Extensive genome-wide linkage disequilibrium in cattle. Genome Res 10: 220-227.
Gabriel, S. B., S. F. Schaffner, H. Nguyen, J. M. Moore, J. Roy et al., 2002 The structure of haplotype blocks in the human genome. Science 296: 2225-2229.
Guo, S. W. and E. A. Thompson. 1992. Performing the exact test of Hardy-Weinberg proportion for multiple alleles. Biometrics 48: 361-372.
Hawken, R. J., W. C. Barris, S. M. McWilliam, and B. P. Dalrymple. 2004. An interactive bovine in silico SNP database (IBISS). Mamm Genome 15: 819-827.
Heyen, D. W., J. E. Beever, Y. Da, R. E. Evert, C. Green, S. R. Bates, J. S. Ziegle, and H. A. Lewin. 1997. Exclusion probabilities of 22 bovine microsatellite markers in fluorescent multiplexes for semiautomated parentage testing. Anim Genet 28: 21-27.
Hinds, D. A., L. L. Stuve, G. B. Nilsen, E. Halperin, E. Eskin, D. G. Ballinger, K. A. Frazer, and D. R. Cox. 2005. Whole-genome patterns of common DNA variation in three human populations. Science 307: 1072-1079.
Ke, X., S. Hunt, W. Tapper, R. Lawrence, G. Stavrides, J. Ghori, P. Whittaker, A. Collins, A. P. Morris, D. Bentley, L. R. Cardon, and P. Deloukas. 2004. The impact of SNP density on fine-scale patterns of linkage disequilibrium. Hum Mol Genet 13: 577-588.
Ke, X., W. Tapper, and A. Collins. 2001. LDB2000: sequence-based integrated maps of the human genome. Bioinformatics 17: 581-586.
Kent, W. J. 2002. BLAT—the BLAST-like alignment tool. Genome Res 12: 656-664.
Khatkar, M. S., P. C. Thomson, I. Tammen, J. A. L. Cavanagh, F. W. Nicholas, and H. W. Raadsma. 2005. Extensive linkage disequilibrium in Australian Holstein-Friesian cattle. Genet Sel Evol (Submitted).
Lewontin, R. C. 1964. The interaction of selection and linkage. I. General considerations: heterotic models. Genetics 49: 49-67.
Malecot, G. 1948. Les Mathematiques de l'Hérédité. Maison et Cie, Paris.
Man, W. Y. N. 2004. Pedigree analysis of Holstein Friesians in Australia. PhD thesis University of Sydney.
Maniatis, N., A. Collins, C. F. Xu, L. C. McCarthy, D. R. Hewett, W. Tapper, S. Ennis, X. Ke, and N. E. Morton. 2002. The first linkage disequilibrium (LD) maps: delineation of hot and cold blocks by diplotype analysis. Proc Natl Acad Sci USA 99: 2228-2233.
McRae, A. F., J. C. McEwan, K. G. Dodds, T. Wilson, A. M. Crawford, and J. Slate. 2002. Linkage disequilibrium in domestic sheep. Genetics 160: 1113-1122.
Morton, N. E. 2003. Genetic epidemiology, genetic maps and positional cloning. Philos Trans R Soc Lond B Biol Sci 358: 1701-1708.
Morton, N. E., A. Collins, S. Lawrence, and D. C. Shields. 1992. Algorithms for a location database. Ann Hum Genet 56 (Pt 3): 223-232.
Morton, N. E., W. Zhang, P. Taillon-Miller, S. Ennis, P. Y. Kwok, and A. Collins. 2001. The optimal measure of allelic association. Proc Natl Acad Sci USA 98: 5217-5221.
Nicholas, F. W., W. Barendse, A. Collins, B. P. Dalrymple, J. H. Edwards, S. Gregory, M. Hobbs, M. S. Khatkar, W. Liao, J. F. Maddox, H. W. Raadsma, and K. R. Zenger. 2004. Integrated maps and Oxford grids:maximising the power of comparative mapping. 29 th International Conference on Animal Genetics, Sep. 11-16, 2004, Tokyo, Japan.
Nsengimana, J., P. Baret, C. S. Haley, and P. M. Visscher. 2004. Linkage disequilibrium in the domesticated pig. Genetics 166: 1395-1404.
Pritchard, J. K. and M. Przeworski. 2001. Linkage disequilibrium in humans: models and data. Am J Hum Genet 69: 1-14.
Snelling, W. M., E. Casas, R. T. Stone, J. W. Keele, G. P. Harhay, G. L. Bennett, and T. P. Smith. 2005. Linkage mapping bovine EST-based SNP. BMC Genomics 6: 74.
Stephens, M., and P. Donnelly, 2003 A comparison of bayesian methods for haplotype reconstruction from population genotype data. Am J Hum Genet 73: 1162-1169.
Tapper, W., A. Collins, J. Gibson, N. Maniatis, S. Ennis, and N. E. Morton. 2005. A map of the human genome in linkage disequilibrium units. Proc Natl Acad Sci USA 102: 11835-11839.
Tapper, W. J., N. Maniatis, N. E. Morton, and A. Collins. 2003. A metric linkage disequilibrium map of a human chromosome. Ann Hum Genet 67: 487-494.
Tapper, W. J., N. E. Morton, I. Dunham, X. Ke, and A. Collins. 2001. A sequence-based integrated map of chromosome 22. Genome Res 11: 1290-1295.
Tozaki, T., K. I. Hirota, T. Hasegawa, M. Tomita, and M. Kurosawa. 2005. Prospects for whole genome linkage disequilibrium mapping in thoroughbreds. Gene 346: 127-132.
Wall, J. D., and J. K. Pritchard, 2003 Haplotype blocks and linkage disequilibrium in the human genome. Nat Rev Genet 4: 587-597.
Zhang, K., Z. Qin, T. Chen, J. S. Liu, M. S. Waterman et al., 2005 HapBlock: haplotype block partitioning and tag SNP selection software using a set of dynamic programming algorithms. Bioinformatics 21: 131-134. Epub 2004 August 2027.
Zhang, W., A. Collins, N. Maniatis, W. Tapper, and N. E. Morton. 2002. Properties of linkage disequilibrium (LD) maps. Proc Natl Acad Sci USA 99: 17004-17007.

### WEB REFERENCES

http://www.adhis.com.au/; Australian Dairy Herd Improvement Scheme (ADHIS)
http://www.livestockgenomics.csiro.au/ibiss/; The Interactive Bovine In Silico SNP (IBISS) database
http://www.hgsc.bcm.tmc.edu/projects/bovine/; The International Bovine Genome Sequencing Project
http://www.illumina.com; Illumina Inc., San Diego, Calif.
http://medvet.angis.org.au/ldb/; Bovine Location DataBase (bLDB)
http://www.gramene.org/cmap/; CMap—the Comparative Map Viewer
http://www.marc.usda.gov/; Bovine linkage map database
http://cedar.genetics.soton.ac.uk/pub/PROGRAMS/LDMAP; The LDMAP program
http://wbiomed.curtin.edu.au/genepop; Genepop
http://dga.jouy.inra.fr/sgqa/diffusions/pedig/pedigE.htm; pedig programes for pedigree analysis
https://upload.mcgill.ca/animal/97r12.pdf and https://upload.mcgill.ca/animal/97r12.pdf for overall type assessment.

1. A method for predicting a phenotype in a bovine animal, the method comprising analysing a nucleic acid sample from said animal for the presence of at least one genetic marker known to reside in an LD block in any one of bovine chromosomes BTA-I to BTA-29, wherein said LD block is associated with said phenotype, and wherein the phenotype is selected from the group consisting of Australian profit ranking (APR), Australian selection index (ASR), protein yield (PROT), protein percent (PROT %), milk volume (MILK), fat yield (FAT), fat percent (FAT %), breeding value overall type (Overall Type), somatic cell count (SCC), and breeding value cow fertility (Cow Fertility).

2. A method of selecting a bovine animal for a phenotype comprising analysing a nucleic acid sample from said animal for the presence of at least one genetic marker known to reside in an LD block in any one of bovine chromosomes BTA-I to BTA-29, wherein said LD block is associated with said phenotype, and wherein the phenotype is selected from the group consisting of Australian profit ranking (APR), Australian selection index (ASR), protein yield (PROT), protein percent (PROT %), milk volume (MILK), fat yield (FAT), fat percent (FAT %), breeding value overall type (Overall Type), somatic cell count (SCC), and breeding value cow fertility (Cow Fertility), and selecting the animal based on the presence or absence of the at least one genetic marker.

3. A method according to claim 1, wherein the phenotype is Australian profit ranking (APR) and the LD block is selected from the group consisting of C1L1.0B_{—}59.93-83.90, C2L1.0BJ 13.49-125.27, C3L1.0B_{—}86.79-102.66, C4L1.0B_{—}38.59-65.77, C5L1.0B_{—}6.53-12.83, C6L1.0B_{—}59.88-79.54, C7L1.0B_{—}42.20-64.05, C8L1.0B_{—}33.47-59.35, C9L1.0B_{—}52.17-73.73, C10L1.0B_{—}20.04-41.39, C11L1.0B_{—}83.65-93.53, C12L1.0B_{—}11.23-20.94, C13L1.0B_{—}38.61-56.34, C14L1.0B_{—}18.56-37.78, 25 C15L1.0B_{—}34.73-54.95, C16L1.0B_{—}28.33-44.56, C17L1.0B_{—}32.00-45.41, C18L1.0BJ3.92-25.33, C19L1.0BJ8.67-30.73, C20L1.0B_{—}28.06-43.47, C21L1.0BJ 1.93-24.10, C22L1.0B_{—}34.48-46.43, C23L1.0BJ4.14-27.73, C24L1.0BJ5.09-47.57, C25L1.0B_{—}27.70-36.65, C26L1.0BJ 0.99-30.90, C27L1.0B_{—}24.14-35.66, C28L1.0B_{—}30.30-37.62 and C29L1.0BJ23.81-31.74.

4. A method according to claim 1, wherein the phenotype is Australian Selection Index (ASI) and the LD block is selected from the group consisting of C1L1.0B_{—}59.93-83.90, C2L1.0BJ01.03-113.49, C3L1.0BJ6.79-102.66, C4L1.0B_{—}38.59-65.77, C5L1.0B_{—}22.90-42.32, C6L1.0B_{—}59.88-79.54, C7L1.0B_{—}42.20-64.05, C8L1.0B_{—}33.47-59.35, C9L1.0B_{—}52.17-73.73, C10L1.0B_{—}20.04-41.39, C11L1.0B_{—}9.99-28.73, C12L1.0B_{—}34.61-53.14, C13L1.0BJ8.61-56.34, C14L1.0BJ 8.56-37.78, C15L1.0B_{—}34.73-54.95, C16L1.0B_{—}28.33-44.56, C17L1.0B_{—}32.00-45.41, C18L1.0B_{—}38.17-52.45, C19L1.0B_{—}18.67-30.73, C20L1.0BJ4.58-28.06, C21L1.0B_{—}11.93-24.10, C22L1.0B_{—}34.48-46.43, C23L1.0BJ4.14-27.73, C24L1.0B_{—}35.09-47.57, C25L1.0B_{—}27.70-36.653 C26L1.0B_{—}10.99-30.90, s C27L1.0B_{—}24.14-35.66, C28L1.0B_{—}30.30-37.62 and C29L1.0B_{—}23.81-31.74.

5. A method according to claim 1, wherein the phenotype is protein yield (PROT) and the LD block is selected from the group consisting of C1L1.OB_{—}59.93-83.9O, C2L1.0B_{—}101.03-113.49, C3L1.0B_{—}86.79-102.66, C4L1.0B_{—}38.59-65.77, C5L1.0B_{—}22.90-42.32, C6L1.0B_{—}59.88-79.54, C7L1.0B_{—}42.20-64.05, C8L1.0BJ3.47-59.35, C9L1.0B_{—}52.17-73.73, C1OL1.0B_{—}20.04-41.39, C11L1.0B_{—}9.99-28.73, C12L1.0B_{—}34.61-53.14, C13L1.0B_{—}14.82-27.97, C14L1.0B_{—}18.56-37.78, C15L1.0B_{—}34.73-54.95, C16L1.0B_{—}28.33-44.56, C17L1.0B_{—}32.00-45.41, C18L1.0B_{—}38.17-52.45, C19L1.0B_{—}18.67-30.73, C20L1.0B_{—}14.58-28.06, C21L1.0B_{—}11.93-24.10, C22L1.0B_{—}20.60-34.48, C23L1.0B_{—}14.14-27.73, is C24L1.0B_{—}35.09-47.57, C25L1.0B_{—}27.70-36.65, C26L1.0B_{—}10.99-30.90, C27L1.0B_{—}24.14-35.66, C28L1.0B_{—}30.30-37.62 and C29L1.0B_{—}23.81-31.74.

6. A method according to claim 1, wherein the phenotype is protein percent (PROT %) and the LD block is selected from the group consisting of C1L1.OB_{—}59.93-83.90, C2L1.0B_{—}101.03-113.49, C3L1.0BJ0.54-22.10, C4L1.0B_{—}65.77-81.49, C5L1.0B_{—}69.19-87.51, C6L1.0B_{—}79.54-93.65, C7L1.0B_{—}42.20-64.05, C8L1.0B_{—}33.47-59.35, C9L1.0B_{—}52.17-73.73, C10L1.0B_{—}20.04-41.39, C11L1.0B_{—}64.26-83.65, C12L1.0B_{—}11.23-20.94, C13L1.0B_{—}64.29-72.65, C14L1.0B_{—}54.46-69.52, C15L1.0B_{—}17.86-34.73, C16L1.0B_{—}14.51-28.33, C17L1.0B_{—}55.81-62.12, C18L1.0B_{—}0.65-13.92, C19L1.0B_{—}18.67-30.73, C20L1.0B_{—}28.06-43.47, C21L1.0B_{—}11.93-24.10, C22L1.0B_{—}34.48-46.43, C23L1.0B_{—}37.77-48.55, C24L1.0B_{—}47.57-55.04, C25L1.0B_{—}27.70-36.65, C26L1.0B_{—}30.90-43.28, C27L1.0B_{—}13.12-24.14, C28L1.0B_{—}30.30-37.62 and C29L1.0B_{—}23.81-31.74.

7. A method according to claim 1, wherein the phenotype is milk volume (MILK) and the LD block is selected from the group consisting of C1L1.0B_{—}59.93-83.90, so C2L1.0B_{—}16.97-37.73, C3L1.0B_{—}36.27-52.80, C4L1.0B_{—}38.59-65.77, C5L1.0B_{—}22.90-42.32, C6L1.0B_{—}59.88-79.54, C7L1.0B_{—}64.05-77.97, C8L1.0B_{—}33.47-59.35, C9L1.0B_{—}29.65-52.17, C10L1.0B_{—}20.04-41.39, C11L1.0B_{—}83.65-93.53, C12L1.0B_{—}61.59-74.02, C13L1.0B_{—}14.82-27.97, C14L1.0BJ 8.56-37.78, C15L1.0B_{—}34.73-54.95, C16L1.0B_{—}28.33-44.56, C17L1.0B_{—}32.00-45.41, 35 C18L1.0B_{—}0.65-13.92, C19L1.0BJ8.67-30.73, C20L1.0B_{—}28.06-43.47, C21L1.0B_{—}11.93-24.10, C22L1.0B_{—}20.60-34.48, C23L1.0B_{—}14.14-27.73, C24L1.0B_{—}47.57-55.04, C25L1.0B_{—}27.70-36.65, C26L1.0B_{—}10.99-30.90₃ C27L1.0B_{—}24.14-35.66, C28L1.0B_{—}30.30-37.62 and C29L1.0B_{—}23.81-31.74.

8. A method according to claim 1, wherein the phenotype is fat yield (FAT) and the LD block is selected from the group consisting of C1L1.0B_{—}37.64-59.93, C2L1.0BJ01.03-113.49, C3L1.0B_{—}86.79-102.66, C4L1.0BJ8.59-65.77, C5L1.0B_{—}87.51-102.44, C6L1.0B_{—}12.78-27.80, C7L1.0B_{—}42.20-64.05, C8L1.0B_{—}33.47-59.35, C9L1.0B_{—}52.17-73.73, C10L1.0B_{—}20.04-41.39, C11L1.0B_{—}9.99-28.73, C12L1.0B_{—}34.61-53.14, C13L1.0B_{—}38.61-56.34, C14L1.0B_{—}0.03-7.93, C15L1.0B_{—}34.73-io 54.95, C16L1.0B_{—}28.33-44.56, C17L1.0B_{—}55.81-62.12, C18L1.0B_{—}13.92-25.33, C19L1.0B_{—}18.67-30.73, C20L1.0BJ4.58-8.06, C21L1.0BJ 1.93-24.10, C22L1.0BJ 0.32-10.14, C23L1.0BJ4.14-27.73, C24L1.0B_{—}47.57-55.04, C25L1.0B_{—}18.85-27.70, C26L1.0B_{—}10.99-30.90, C27L1.0B_{—}24.14-35.66, C28L1.0B_{—}30.30-37.62 and C29L1.0B_{—}23.81-31.74.

9. A method according to claim 1, wherein the phenotype is fat percent (FAT %) and the LD block is selected from the group consisting of C1L1.0B_{—}37.64-59.93, C2L1.0B_{—}16.93-30.73, C3L1.0B_{—}10.54-23.10, C4L1.0B_{—}38.59-65.77, C5L1.0B_{—}87.51-102.44, C6L1.0B_{—}79.54-93.65, C7L1.0B_{—}64.05-77.97, C8L1.0B_{—}78.41-95.65, C9L1.0B_{—}52.17-73.73, C10L1.0B_{—}41.39-63.85, C11L1.0BJ).03-9.99, C12L1.0B_{—}61.59-74.02, C13L1.0B_{—}10.82-27.97, C14L1.0B_{—}0.03-7.93, C15L1.0B_{—}34.73-54.95, C16L1.0BJ4.51-28.33, C17L1.0B_{—}55.81-62.12, C18L1.0B_{—}0.65-13.92, C19L1.0B_{—}30.73-47.39, C20L1.0B_{—}28.06-43.47, C21L1.0B_{—}24.10-40.24, C22L1.0BJ.32-10.14, C23L1.0B_{—}14.14-27.73, C24L1.0B_{—}47.57-55.04, C25L1.0BJH2-11.08, C26L1.0BJ 0.99-30.90, C27L1.0BJ3.12-24.14, C28L1.0BJ 1.38-21.51 and C29L1.0B_{—}23.81-31.74.

10. A method according to claim 1, wherein the phenotype is breeding value overall type (OVERALLTYPE) and the LD block is selected from the group consisting of C1L1.0B_{—}59.93-83.90, C2L1.0BJ 13.49-125.27, C3L1.0B_{—}86.79-102.66, C4L1.0B_{—}38.59-65.77, C5L1.0B_{—}22.90-42.32, C6L1.0B_{—}59.88-79.54, C7L1.0B_{—}42.20-64.05, C8L1.0B_{—}33.47-59.35, C9L1.0B_{—}29.65-52.17, C10L1.0B_{—}20.04-41.39, C11L1.0B_{—}64.26-83.65, C12L1.0BJ 1.23-20.94, C13L1.0B_{—}64.29-72.65, C14L1.0B_{—}37.78-54.46, C15L1.0B_{—}34.73-54.95, C16L1.0B_{—}28.33-44.56, C17L1.0B_{—}55.81-62.12, C18L1.0B_{—}38.17-52.45, C19L1.0BJ0.73-47.39, C20L1.0B_{—}28.06-43.47, C21L1.0B_{—}24.10-40.24, C22L1.0B_{—}20.60-34.48, 35 C23L1.0BJ4.14-27.73, C24L1.0B_{—}35.09-47.57, C25L1.0BJ).12-11.08, C26L1.0B_{—}10.99-30.90, C27L1.0B_{—}24.14-35.66, C28L1.0B_{—}0.03-11.38 and C29L1.0B_{—}13.31-23.81.

11. A method according to claim 1, wherein the phenotype is somatic cell count (SCC) and the LD block is selected from the group consisting of C1L1.0B_{—}37.64-59.93, 5 C2L1.0B_{—}42.97-70.21, C3L1.0B_{—}86.79-102.66, C4L1.0B_{—}38.59-65.77, C5L1.0B_{—}22.90-42.32, C6L1.0B_{—}42.84-59.88, C7L1.0B_{—}25.69-42.20, C8L1.0B_{—}78.41-95.65, C9L1.0B_{—}52.17-73.73, C10L1.0B_{—}41.39-63.85, C11L1.0B_{—}83.65-93.53, C12L1.0B_{—}20.94-34.61, C13L1.0B_{—}56.34-64.29, C14L1.0B_{—}7.93-18.56, C15L1.0B_{—}34.73-54.95, C16L1.0B_{—}44.56-58.07, C17L1.0B_{—}0.05-8.52, C18L1.0B_{—}0.65-13.92, C19L1.0B_{—}30.73-47.39, C20L1.0B_{—}14.58-28.06, C21L1.0BJ1.93-24.10, C22L1.0B_{—}20.60-34.48, C23L1.0B_{—}14.14-27.73, C24L1.0B_{—}35.09-47.57, C25L1.0B_{—}0.12-11.08, C26L1.0B_{—}10.99-30.90, C27L1.0B_{—}24.14-35.66, C28L1.0B_{—}30.30-37.62 and C29L1.0BJ1.74-40.84.

12. A method according to claim 1, wherein the phenotype is breeding value cow is fertility (COWFERTILITY) and the LD block is selected from the group consisting of C1L1.OB_{—}59.93-83.9O, C2L1.OBJ 6.97-30.73, C3L1.0B_{—}52.80-72.96, C4L1.0B_{—}16.30-38.59, C5L1.0B_{—}22.90-42.32, C6L1.0B_{—}59.88-79.54, C7L1.0B_{—}42.20-64.05, C8L1.0B_{—}33.47-59.35, C9L1.0B_{—}29.65-52.17, C10L1.0B_{—}20.04-41.39, C11L1.OB_{—}64.26-83.65, C12L1.0B_{—}61.59-74.02, C13L1.0B_{—}64.29-72.65, C14L1.0BJ8.56-37.78, 20 C15L1.0B_{—}34.73-54.95, C16L1.0B_{—}28.33-44.56, C17L1.0B_{—}55.81-62.12, C18L1.0B_{—}0.65-13.92, C19L1.0B_{—}18.67-30.73, C20L1.0B_{—}28.06-43.47, C21L1.0B_{—}11.93-24.10, C22L1.0BJL32-10.14, C23L1.0B_{—}14.14-27.73, C24L1.0B_{—}47.57-55.04, C25L1.0B_{—}11.08-18.85, C26L1.0B_{—}10.99-30.90, C27L1.0B_{—}24.14-35.66, C28L1.0B_{—}0.03-11.38 and C29L1.0B_{—}7.33-13.31.

13. A method according to claim 1, wherein the at least one genetic marker known to reside in an LD block is selected from the group consisting of a single nucleotide polymorphism (SNP), a haplotype, a microsatellite (simple tandem repeat STR, simple sequence repeat SSR), a restriction fragment length polymorphism (RFLP), an amplified fragment length polymorphism (AFLP), and an insertion-deletion polymorphism (INDEL).

14. A method according to claim 1, wherein the step of analysing the nucleic acid sample for the presence of at least one genetic marker known to reside in an LD block comprises random amplified polymorphic DNA (RAPD), ligase chain reaction, insertion/deletion analysis or direct sequencing of the gene.

15. A method according to claim 1, wherein the bovine is selected from the group comprising Angus, Shorthorn, Limosin, Fresian, Wagyu, Jersey and Holstein or a cross of any two or more thereof.

16. A method according to claim 1, wherein the bovine is a Holstein or a 5 Holstein/Fresian.

17. A linkage disequilibrium unit (LDU) map of any one or more of bovine chromosomes BTA-I to BTA-29, wherein said map comprises a plurality of chromosomal regions, and wherein said regions are defined by their co-inheritance across generations substantially as entire linkage disequilibrium (LD) blocks.

18. A linkage disequilibrium unit (LDU) map according to claim 17, wherein the chromosomal regions comprise a plurality of genetic markers.

19. A linkage disequilibrium unit (LDU) map according to claim 18, wherein the plurality of genetic markers is. of high density across the chromosomal regions.

20. A linkage disequilibrium unit (LDU) map according to claim 17, wherein is the relative order and orientation of the genetic markers within each LD block is substantially conserved across generations.

21. A linkage disequilibrium unit (LDU) map according to claim 17, wherein the map has an LDU stringency of 1.0.
